(19) United States
(12) Patent Application Publication
(10) Pub. No.: US 2025/0326777 A1
(43) Pub. Date: Oct. 23, 2025

(54) COMPOUNDS, COMPOSITIONS AND METHODS THEREOF

(71) Applicant: HEPAITECH (BEIJING) BIOPHARMA TECHNOLOGY CO., LTD., Beijing (CN)

(72) Inventors: Ruichao Richard Shen, Belmont, MA (US); Xiaoguang Lei, Beijing (CN); Xiaoming Wang, Beijing (CN); Yulong Li, Beijing (CN); Ruoyu He, Beijing (CN)

(21) Appl. No.: 19/121,125

(22) PCT Filed: Oct. 20, 2023
(86) PCT No.: PCT/CN2023/125554

§ 371 Date: Apr. 14, 2025

(30) Foreign Application Priority Data
Oct. 21, 2022 (WO) PCT/CN2022/126550
Feb. 21, 2023 (WO) PCT/CN2023/077383

(51) Int. Cl. C07F 9/572; A61K 31/343; A61K 31/352; A61K 31/381; A61K 31/404; A61K 31/4155; A61K 31/423; A61K 31/4245; A61K 31/428; A61K 31/575; A61K 31/675; C07D 209/24; C07D 263/57; C07D 277/66; C07D 307/79; C07D 311/04; C07D 333/52; C07D 405/10; C07D 413/10

(52) U.S. Cl. CPC C07F 9/5728; A61K 31/343; A61K 31/352; A61K 31/381; A61K 31/404; A61K 31/4155; A61K 31/423; A61K 31/4245; A61K 31/428; A61K 31/575; A61K 31/675; C07D 209/24; C07D 263/57; C07D 277/66; C07D 307/79; C07D 311/04; C07D 333/52; C07D 405/10; C07D 413/10

(57) ABSTRACT

Among other things, the present disclosure provides compounds, e.g., of formula I or salts thereof. In some embodiments, the present disclosure provides methods for modulating MRGPRX4 activity. In some embodiments, the present disclosure provides methods for preventing or treating conditions, disorders or diseases, e.g., MRGPRX4-associated conditions, disorders or diseases.

### CROSS-REFERENCE TO RELATED APPLICATIONS

This application claims priority to PCT Application Nos. PCT/CN2022/126550, filed on Oct. 21, 2022, and PCT/CN2023/077383, filed on Feb. 21, 2023, the entirety of each of which is incorporated herein by reference.

### TECHNICAL FIELD

In some embodiments, the present disclosure provide compounds and compositions, among other things, useful for modulating MRGPRX4 activities. In some embodiments, provided compounds and compositions are useful for preventing or treating MRGPRX4-associated conditions, disorders or diseases, e.g., pruritus. In some embodiments, the present disclosure provides technologies for preparing provided compounds and compositions.

### BACKGROUND

Compounds are useful for many purposes including modulating biological functions and activities. It has been reported that Mas-related G-protein coupled receptor X4 (MRGPRX4) has a number of biological functions and can be associated with various conditions, disorders or diseases. For example, patients with liver condition, disorder or disease such as primary biliary cholangitis (PBC), primary sclerosing cholangitis (PSC), or progressive familial intrahepatic cholestasis (PFIC), often suffer from refractory pruritus, which severely affects quality of life and potentially leads to lassitude, fatigue, depression, and even suicidal thoughts. Recently MRGPRX4 and its agonism by bile acid and derivatives thereof have been reported to be likely associated with pruritus occurred in these diseases (e.g., Meixiong et al. MRGPRX4 is a G protein-coupled receptor activated by bile acids that may contribute to cholestatic pruritus, PNAS, 2019, 116 (21), 10525-10530; Yu et al. MRGPRX4 is bile acid receptor for human cholestatic itch, eLife, 2019, 8, e48431).

MRGPRX4 has been reported to express in at least human dorsal root ganglion (hDRG) neurons and to co-expresses with itch receptor HRH1. It has been reported that bile acids or MRGPRX4 specific agonists in the skin can activate MRGPRX4 in itch-related primary fibers, elicits Ca²⁺ responses, and induce itch in human subjects.

Small compounds that can allegedly act as MRGPRX4 modulators have been disclosed in, e.g., WO 2020/198537, US 2021/0032213, WO 2021/211839, WO 2022/061008, etc.

### SUMMARY

In some embodiments, the present disclosure provides various compounds, e.g., compounds having the structure of formula I or salts thereof, and compositions and methods thereof. In some embodiments, provided compounds are useful as MRGPRX4 modulators. In some embodiments, provided technologies (e.g., compounds, compositions, methods, etc.) are useful for preventing or treating various conditions, disorders or diseases. In some embodiments, a condition, disorder or disease is a MRGPRX4-associated condition, disorder or disease. In some embodiments, a condition, disorder or disease is or comprises itch. In some embodiments, a condition, disorder or disease is or comprises pruritus. In some embodiments, a condition, disorder or disease is associated with administration of another therapeutic agent, e.g., a FXR agonist, a bile acid or an analog or derivative thereof, etc.

In some embodiments, the present disclosure provides a compound, wherein the compound has the structure of formula I:

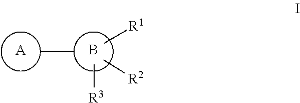

or a pharmaceutically acceptable salt thereof, wherein:
R¹ is —C(O)OH or an isostere thereof, optionally protected —CHO or R^{d6}; or R¹ is —C(O)OR¹¹, —P(O)(OR¹²)(OR¹³), —C(O)N(R¹⁴)SO₂R¹⁵, —C(O)NR¹⁶R¹⁷, —CN,

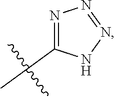 [SMILES: *c1nnn[nH]1]

halogen, or

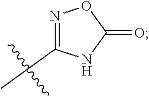 [SMILES: *c1noc(=O)[nH]1]

R^{d6} is —CH(OR)₂;
each of R² and R³ is independently R′, —OR′, halogen, CN, —NO₂, or —N(R′)₂;
Ring A is

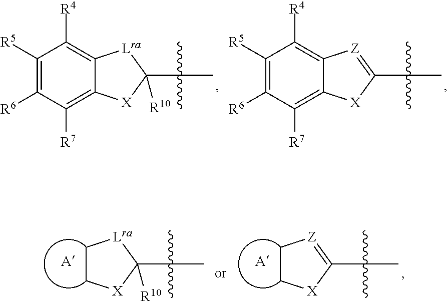

wherein Ring A′ is an optionally substituted 5-10 membered aromatic ring having 0-4 heteroatoms;
L^{ra} is optionally substituted —(CH₂)ₙ—;
n is 1, 2 or 3;
X is —O—, —S—, —N(R⁸)—, or optionally substituted —CH₂—;
Z is —N═ or —C(R⁹)═;
each of R⁴, R⁵, R⁶, R⁷ and R⁹ is independently R′, —OR′, halogen, —CN, —NO₂, or —N(R′)₂;
Ring B is an optionally substituted ring selected from a 6-10 membered aryl ring and a 5-10 membered heteroaryl ring having 1-6 heteroatoms;
each of R⁸, R¹⁰, R¹, R¹², R¹³, R¹⁴, R¹⁵, R¹⁶, and R¹⁷ is independently R′;
each R¹ is independently R, OR, —C(O)R, C(O)OR, or —S(O)₂R;
each R is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

In some embodiments, the present disclosure provides a compound, wherein the compound has the structure of formula I or a salt thereof, wherein R¹ is —C(O)OH or an isostere thereof, optionally protected-CHO or R^{d6}; or R¹ is —C(O)OR¹, —P(O)(OR¹²)(OR¹³), —C(O)N(R¹⁴)SO₂R¹⁵, —C(O)NR¹⁶R¹⁷, —CN,

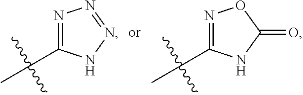 [SMILES: *c1nnn[nH]1 ; *c1noc(=O)[nH]1]

and each other variable is independently as described herein.

In some embodiments, the present disclosure provides a compound, wherein the compound has the structure of formula I or a pharmaceutically acceptable salt thereof, wherein Ring A′ is an optionally substituted 5-6 membered aromatic ring having 0-4 heteroatoms, and each other variable is independently as described herein.

In some embodiments, the present disclosure provides a pharmaceutical composition of a provided compounds. In some embodiments, the present disclosure provides a pharmaceutical composition comprising a provided compound or a pharmaceutically acceptable salt thereof, and a pharmaceutically acceptable carrier. In some embodiments, the present disclosure provides a pharmaceutical composition delivering a provided compound or a pharmaceutically acceptable salt thereof, and a pharmaceutically acceptable carrier.

Provided technologies are useful for many purposes. For example, in some embodiments, the present disclosure provides a method for preventing a condition, disorder or disease, comprising administering or delivering to a subject susceptible thereto an effective amount a provided compound. For example, in some embodiments, the present disclosure provides a method for treating a condition, disorder or disease, comprising administering or delivering to a subject suffering therefrom an effective amount a provided compound. In some embodiments, a condition, disorder or disease is a MRGPRX4-associated condition, disorder or disease. In some embodiments, a condition, disorder or disease is associated with MRGPRX4 activation. In some embodiments, a condition, disorder or disease is associated with MRGPRX4 activation by an agent. In some embodiments, a condition, disorder or disease is associated with MRGPRX4 interaction with an agent. In some embodiments, a condition, disorder or disease is associated with administration of an agent. In some embodiments, a condition, disorder or disease is associated with delivery of an agent. In some embodiments, an agent is a therapeutic agent. In some embodiments, an agent can bind to MRGPRX4. In some embodiments, an agent can activate MRGPRX4. In some embodiments, an agent is a FXR agonist. In some embodiments, an agent is a bile acid or an analog or derivative thereof. In some embodiments, a condition, disorder or disease is pruritus.

In some embodiments, the present disclosure provides a method for preventing a condition, disorder or disease, comprising administering or delivering to a subject susceptible thereto an effective amount a provided compound and another agent. In some embodiments, the present disclosure provides a method for treating a condition, disorder or disease, comprising administering or delivering to a subject suffering therefrom an effective amount a provided compound and another agent. For example, in some embodiments, a condition, disorder or disease is associated with Farnesoid X receptor (FXR), and an another agent is a FXR agonist. In some embodiments, a condition, disorder or disease is associated with TGR5, and an another agent is a TGR5 agonist. In some embodiments, an another agent can activate MRGPRX4. In some embodiments, an another agent is a FXR agonist. In some embodiments, an another agent is a bile acid or an analog or derivative thereof. In some embodiments, a condition, disorder or disease is a liver condition, disorder or disease. For example, in some embodiments, a condition, disorder or disease is nonalcoholic steatohepatitis (NASH).

In some embodiments, the present disclosure provides technologies, e.g., methods, reagents, etc., for preparing a compound of Formula (I) or a pharmaceutical acceptable salt thereof.

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included. Unless otherwise clear from context, isomers of compounds are included. As those skilled in the art, compounds may be provided, administered, or delivered in various forms, e.g., salts (e.g., pharmaceutically acceptable salts), solvates, hydrates, esters, prodrugs, tautomers, etc.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1˜4 carbon atoms (e.g., C₁-C₄ for straight chain lower alkyls).

Alkynyl: As used herein, the term “alkynyl” refers to an aliphatic group, as defined herein, having one or more triple bonds.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, each monocyclic ring unit is aromatic. In some embodiments, an aryl group is a biaryl group. The term “aryl” may be used interchangeably with the term “aryl ring.” In certain embodiments of the present disclosure, “aryl” refers to an aromatic ring system which includes, but in is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Characteristic portion: As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of amino acids, in some embodiments, a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, each monocyclic ring unit is aromatic. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4 oxazin-3 (4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N(as in 3,4 dihydro-2H-pyrrolyl), NH (as in pyrrolidinyl), or +NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Leaving Group/LG: As described herein, a leaving group or a LG is an atom or group of atoms that detaches from the main or residual part of a substrate during a reaction or elementary step of a reaction. In some embodiments, LG is a halogen. In some embodiments, LG is —Cl. In some embodiments, LG is —OH.

Optionally Substituted: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents include halogen; —(CH₂)₀₋₄R^{o}; —(CH₂)₀₋₄OR^{o}; —O(CH₂)₀₋₄R^{o}, O—(CH₂)₀₋₄C(O)OR^{o}; —(CH₂)₀₋₄CH(OR^{o})₂; —(CH₂)₀₋₄Ph, which may be substituted with R^{o}; —(CH₂)₀₋₄ O(CH₂)₀₋₁Ph which may be substituted with R^{o}; —CH═CHPh, which may be substituted with R^{o}; —(CH₂)₀₋₄ O(CH₂)₀-1-pyridyl which may be substituted with R^{o}; —NO₂; —CN; —N₃; —(CH₂)₀₋₄N(R^{o}); —(CH₂)₀₋₄ N(R^{o})C(O)R^{o}; —N(R^{o})C(S) R^{o}; —(CH₂)₀₋₄N(R^{o})C(O)NR₂^{o}; —N(R^{o})C(S)NR₂^{o}; —(CH₂)₀₋₄N(R^{o})C(O)OR^{o}; —N(R^{o})N(R^{o})C(O)R^{o}; —N(R^{o})N(R^{o})C(O)NR₂^{o}; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR₃^{o}; —(CH₂)₀₋₄C(O)R^{o}; —OC(O)(CH₂)₀₋₄SR, —SC(S)SR^{o}; —(CH₂)₀₋₄SC(O) R^{o}; —(CH₂)₀₋₄C(O)NR₂^{o}; —C(S)NR₂^{o}; —C(S) SR^{o}; —SC(S) SR^{o}, —(CH₂)₀₋₄ C(O)NR₂^{o}; —C(O)N(OR^{o}) R^{o}; —C(O)C(O)R^{o}; —C(O)CH₂C(O) R^{o}; —C(NOR°) R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)NR₂^{o}; —(CH₂)₀₋₄S(O) R^{o}; —N(R^{o})S(O)₂NR₂^{o}; —N(R^{o})S(O)₂R^{o}; —N(OR^{o})R^{o}; —C(NH) NR₂^{o}; —P(O)₂R^{o}; —P(O)R₂^{o}; —OP(O)R₂^{o}; —OP(O)(OR₂^{o}; —SiR₃^{o}; —OSiR₃^{o}; —(C₁₋₄ straight or branched)alkylene)O—N(R₂^{o}); or —(C₁₋₄ straight or branched)alkylene)C(O)O—N(R₂^{o}), wherein each R^{o} may be substituted as defined below and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀-1 (C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{o}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O) R^{•}, (CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR₂^{•}, —NO₂, —SiR₃^{•}, —OSiR₃^{•}, —C(O)SR^{•}, —(C₁₋₄ straight or branched alkylene)C(O)OR^{•}, or -SSR^{•}wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{o} include ═O and ═S.

Suitable divalent substituents include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{o}, -(haloR^{•}), —OH, —OR^{•}, O(haloR^{•}), —CN, —C(O)OH, —C(O)OR^{•}, —NH₂, —NHR, —NR₂^{•}, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen include —R^{†}, —NR₂^{†}, —C(O)R^{†}, —C(O)OR^{†}, —C(O)C(O)^{R}†, —C(O)CH₂C(O)R^{†}, —S(O)₂R^{†}, —S(O)₂NR¹², —C(S)NR₂^{†}, —C(NH)NR₂^{†}, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR₂^{•}, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises two or more acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9 (10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4 dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5 dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, 0-nitrophenoxyacetamide, acetoacetamide, (N′-dithiobenzyloxycarbonylamino) acetamide, 3-(p-hydroxyphenyl) propanamide, 3-(o-nitrophenyl) propanamide, 2-methyl-2-(0-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N(1-isopropyl-4-nitro-2-oxo-3-pyroolin-3-yl)amine, quaternary ammonium salts, N-benzylamine, N-di (4 methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fem), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten) carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, 0-nitrobenzenesulfenamide (Nps), 2,4 dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6, -trimethyl-4-methoxybenzenesulfonamide (Mtr), 2,4,6 trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4 dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl(p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4 methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p chlorophenyl, p methoxyphenyl, 2,4 dinitrophenyl, benzyl, p-methoxybenzyl, 3,4 dimethoxybenzyl, o-nitrobenzyl, p-nitrobenzyl, p-halobenzyl, 2,6 dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri (p-methoxyphenyl)methyl, 4 (4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9 phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t butyldimethylsilyl (TBDMS), t-butyldiphenylsilyl (TBDPS), tribenzylsilyl, tri-p-xylylsilyl, triphenylsilyl, diphenylmethylsilyl (DPMS), +butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4 oxopentanoate (levulinate), 4,4 (ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4 dimethoxybenzyl carbonate, alkyl o-nitrobenzyl carbonate, alkyl p-nitrobenzyl carbonate, alkyl S benzyl thiocarbonate, 4-ethoxy-1-napththyl carbonate, methyl dithiocarbonate, 2-iodobenzoate, 4 azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4 bis(1,1-dimethylpropyl) phenoxyacetate, chlorodiphenylacetate, isobutyrate, monosuccinoate, (E)-2-methyl-2-butenoate, 0-(methoxycarbonyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, 1-(N,N-dimethylamino)ethylidene derivative, a-(N,N′-dimethylamino)benzylidene derivative, 2-oxacyclopentylidene ortho ester, di-t-butylsilylene group (DTBS), 1,3-(1,1,3,3 tetraisopropyldisiloxanylidene) derivative (TIPDS), tetra-t-butoxydisiloxane-1,3 diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl(trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl(MMTr), 4,4′-dimethoxytrityl, (DMTr) and 4,4′,4″-trimethoxytrityl(TMTr), 2-cyanoethyl(CE or Cne), 2-(trimethylsilyl)ethyl (TSE), 2-(2-nitrophenyl)ethyl, 2-(4-cyanophenyl)ethyl 2-(4-nitrophenyl)ethyl(NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy) trityl, 1, diphenylcarbamoyl, levulinyl, 2-(dibromomethyl)benzoyl(Dbmb), 2-(isopropylthiomethoxymethyl)benzoyl(Ptmt), 9-phenylxanthen-9-yl(pixyl) or 9-(p-methoxyphenyl) xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl(CE or Cne), 2-trimethylsilylethyl, 2-nitroethyl, 2-sulfonylethyl, methyl, benzyl, o-nitrobenzyl, 2-(p-nitrophenyl)ethyl(NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” or “test subject” refers to any organism to which a compound or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. One of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

As those skilled in the art will appreciate, methods and compositions described herein relating to provided compounds generally also apply to pharmaceutically acceptable salts of such compounds.

### CERTAIN EMBODIMENTS OF COMPOUNDS

Among other things, the present disclosure provides compounds that are useful for various purposes. In some embodiments, a provided compound has the structure of formula I:

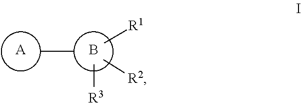

or a pharmaceutically acceptable salt thereof, wherein each variable is independently as described herein.

Certain embodiments for various variables in various formulae (e.g., formula I and formulae in various schemes) are described herein as examples. Those skilled in the art reading the present disclosure will be able to select an embodiment for each variable and combine them; such combinations are within the scope the present disclosure. Those skilled in the art also appreciate that embodiments described for one variable (e.g., R) may be utilized for other variables that can be such variable (e.g., R′, R², R³, R⁴, R⁵, R⁶, R⁷, R⁸, R⁹, R¹⁰, R¹¹, R¹², R¹³, R¹⁴, R¹⁵, R¹⁶, R¹⁷, etc. that can be R).

### Ring A

In some embodiments, Ring A

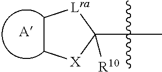

wherein each variable is independently as described herein. In some embodiments, Ring A′ is an optionally substituted phenyl ring. In some embodiments, Ring A is

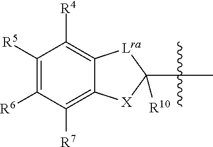

wherein each variable is independently as described herein. In some embodiments, L^{ra} is optionally substituted —(CH₂)ₙ—. In some embodiments, Ring A is

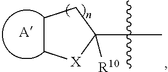

wherein each variable is independently as described herein. In some embodiments, Ring A is

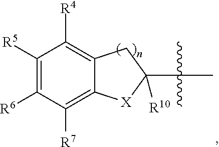

wherein each variable is independently as described herein. In some embodiments, Ring A is optionally substituted

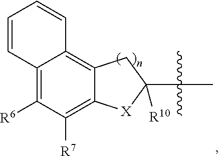

wherein each variable is independently as described herein. In some embodiments, Ring A is optionally substituted

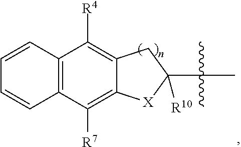

wherein each variable is independently as described herein. In some embodiments, Ring A is optionally substituted

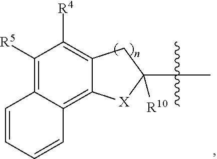

wherein each variable is independently as described herein. In some embodiments, n is 1. In some embodiments, n is 2. In some embodiments, n is 3.

In some embodiments, Ring A is

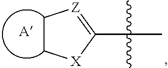

wherein each variable is independently as described herein. In some embodiments, Ring A′ is an optionally substituted phenyl ring. In some embodiments, Ring A is

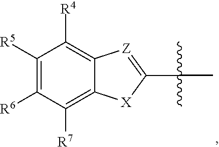

wherein each variable is independently as described herein.

In some embodiments, Ring A is optionally substituted

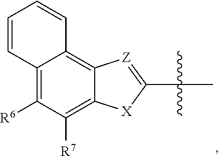

wherein each variable is independently as described herein. In some embodiments, Ring A is optionally substituted

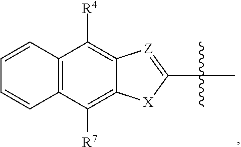

wherein each variable is independently as described herein. In some embodiments, Ring A is optionally substituted

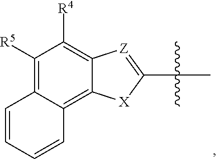

wherein each variable is independently as described herein.

In some embodiments, Ring A is

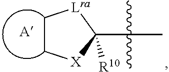

wherein each variable is independently as described herein. In some embodiments, Ring A is

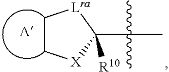

wherein each variable is independently as described herein. In some embodiments, Ring A is

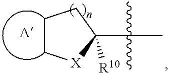

wherein each variable is independently as described herein. In some embodiments, Ring A is

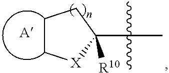

wherein each variable is independently as described herein. In some embodiments, Ring A is

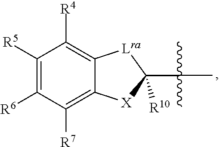

wherein each variable is independently as described herein. In some embodiments, Ring A is

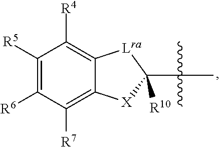

wherein each variable is independently as described herein. In some embodiments, Ring A is

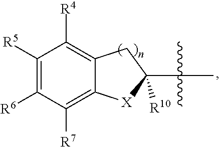

wherein each variable is independently as described herein. In some embodiments, Ring A is

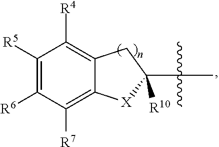

wherein each variable is independently as described herein. In some embodiments, Ring A is optionally substituted

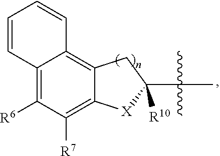

wherein each variable is independently as described herein. In some embodiments, Ring A is optionally substituted

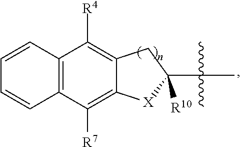

wherein each variable is independently as described herein. In some embodiments, Ring A is optionally substituted

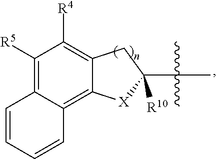

wherein each variable is independently as described herein.

Among other things, the present disclosure provides stereochemically pure, e.g., enantiomerically pure, compounds with purities as described herein. In some embodiments, a compound is stereochemically pure. In some embodiments, a compound is enantiomerically pure. In some embodiments, a provided composition is enriched for one enantiomer over the other, or with respect to a chiral center, for one configuration over the other. For example, in some embodiments, the percentage of an enantiomer, or one configuration with respect to a chiral center, is about or at least about 60%, 65%, 70%, 75%, 80%, 85%, 90%, 95%, 96%, 97%, 98%, 99%, or 99.5%. In some embodiments, it is about or at least about 60%. In some embodiments, it is about or at least about 65%. In some embodiments, it is about or at least about 70%. In some embodiments, it is about or at least about 75%. In some embodiments, it is about or at least about 80%. In some embodiments, it is about or at least about 85%. In some embodiments, it is about or at least about 90%. In some embodiments, it is about or at least about 95%. In some embodiments, it is about or at least about 96%. In some embodiments, it is about or at least about 97%. In some embodiments, it is about or at least about 98%. In some embodiments, it is about or at least about 99%. In some embodiments, it is about or at least about 99.5%. In some embodiments, the(S) enantiomer or configuration is enriched. In some embodiments, the (R) enantiomer or configuration is enriched. In some embodiments, configuration of a stereogenic center is shown in a chemical structure next to such a stereogenic center, e.g., as “R”, “S”, “(R)”, “(S)”, etc., which configuration is typically determined by commercial software like ChemDraw when such software is utilized to prepare such a chemical structure. In some embodiments, configuration of a stereogenic center is not shown. Those skilled in the art can readily determine configurations of stereogenic centers according to common practices in the art.

In some embodiments, it is observed that the S configuration of a chiral carbon to which R¹⁰ is bonded can provide higher desired activities, e.g., MRGPRX4 inhibition, over the R configuration (for R/S configuration for this carbon center, the following order is utilized from priority one to four: X, Ring B, L^{ra} (e.g., —(CH₂)ₙ, and R¹⁰ (e.g., H)). For example, in some embodiments, 52A (the S enantiomer) provided much higher MRGPRX4 inhibition activity than 52B. See, e.g., Table 1.

### Ring A′

In some embodiments, Ring A′ is an optionally substituted phenyl ring. In some embodiments, Ring A′ is an optionally substituted 5-10 membered aromatic ring having 0-4 heteroatoms. In some embodiments, Ring A′ is an optionally substituted 5-6 membered aromatic ring having 1-4 heteroatoms. In some embodiments, Ring A′ is a 6 membered aromatic ring having 1-2 heteroatoms. In some embodiments, Ring A′ is a 6 membered aromatic ring having 1-2 heteroatoms independently selected from O, N, and S. In some embodiments, Ring A′ is a 6 membered aromatic ring having a heteroatom selected from O, N, and S. In some embodiments, Ring A′ is a 6 membered aromatic ring having a nitrogen atom. In some embodiments, Ring A′ is a 6 membered aromatic ring having 2 heteroatoms independently selected from O, N, and S. In some embodiments, Ring A′ is a 6 membered aromatic ring having 2 heteroatoms each of which is N. In some embodiments, Ring A′ is a 6 membered aromatic ring having 3 heteroatoms. In some embodiments, Ring A′ is a 6 membered aromatic ring having 3 heteroatoms independently selected from O, N, and S. In some embodiments, Ring A′ is a 6 membered aromatic ring having 3 heteroatoms wherein one is N. In some embodiments, Ring A′ is a 5 membered aromatic ring having 1-2 heteroatoms. In some embodiments, Ring A′ is a 5 membered aromatic ring having 1-2 heteroatoms independently selected from O, N, and S. In some embodiments, Ring A′ is a 5 membered aromatic ring having an oxygen atom. In some embodiments, Ring A′ is a 5 membered aromatic ring having a nitrogen atom. In some embodiments, Ring A′ is a 5 membered aromatic ring having a sulfur atom. In some embodiments, Ring A′ is a 5 membered aromatic ring having 2 heteroatoms independently selected from O, N, and S. In some embodiments, Ring A′ is a 5 membered aromatic ring having 2 heteroatoms one of which is N. In some embodiments, Ring A′ is a 5 membered aromatic ring having 2 heteroatoms independently selected from N and S. In some embodiments, Ring A′ is a 5 membered aromatic ring having 2 heteroatoms independently selected from N and O. In some embodiments, Ring A′ is a 5 membered aromatic ring having 2 heteroatoms independently selected from O and S. In some embodiments, Ring A′ is a 5 membered aromatic ring having 3 heteroatoms. In some embodiments, Ring A′ is a 5 membered aromatic ring having 3 heteroatoms independently selected from O, N, and S. In some embodiments, a ring has a single heteroatom. In some embodiments, a ring has two or more heteroatoms at least one of which is nitrogen. In some embodiments, each is nitrogen. In some embodiments, all heteroatoms are the same. In some embodiments, at least one heteroatom is different from the other heteroatom(s).

In some embodiments, Ring A′ is an optionally substituted 9 membered aromatic ring having 1-4 heteroatoms. In some embodiments, Ring A′ is an optionally substituted 9-membered aromatic ring having 4 heteroatoms independently selected from O, N, and S. In some embodiments, Ring A′ is an optionally substituted 9-membered aromatic ring having 3 heteroatoms independently selected from O, N, and S. In some embodiments, Ring A′ is an optionally substituted 9-membered aromatic ring having 3 heteroatoms one of which is N. In some embodiments, Ring A′ is an optionally substituted 9-membered aromatic ring having 3 heteroatoms independently selected from O and N. In some embodiments, Ring A′ is an optionally substituted 9-membered aromatic ring having 3 heteroatoms independently selected from O and S. In some embodiments, Ring A′ is an optionally substituted 9-membered aromatic ring having 3 heteroatoms independently selected from N and S. In some embodiments, Ring A′ is an optionally substituted 9-membered aromatic ring having 2 heteroatoms independently selected from O, N, and S. In some embodiments, Ring A′ is an optionally substituted 9-membered aromatic ring having 2 heteroatoms one of which is N. In some embodiments, Ring A′ is an optionally substituted 9-membered aromatic ring having 2 heteroatoms independently selected from O and N. In some embodiments, Ring A′ is an optionally substituted 9-membered aromatic ring having 2 heteroatoms independently selected from O and S. In some embodiments, Ring A′ is an optionally substituted 9-membered aromatic ring having 2 heteroatoms independently selected from N and S. In some embodiments, Ring A′ is an optionally substituted 9-membered aromatic ring having 2 heteroatoms both of which are N. In some embodiments, Ring A′ is an optionally substituted 9-membered aromatic ring having 1 heteroatom selected from O, N. and S. In some embodiments, Ring A′ is an optionally substituted 9-membered aromatic ring having a nitrogen atom. In some embodiments, Ring A′ is an optionally substituted 9-membered aromatic ring having an oxygen atom. In some embodiments, Ring A′ is an optionally substituted 9-membered aromatic ring having an sulfur atom. In some embodiments, a ring has a single heteroatom. In some embodiments, a ring has two or more heteroatoms at least one of which is nitrogen. In some embodiments, each is nitrogen. In some embodiments, all heteroatoms are the same. In some embodiments, at least one heteroatom is different from the other heteroatom(s).

In some embodiments, Ring A′ is an optionally substituted 10 membered aromatic ring having 1-4 heteroatoms. In some embodiments, Ring A′ is an optionally substituted 10-membered aromatic ring having 4 heteroatoms independently selected from O, N, and S. In some embodiments, Ring A′ is an optionally substituted 10-membered aromatic ring having 3 heteroatoms independently selected from O, N, and S. In some embodiments, Ring A′ is an optionally substituted 10-membered aromatic ring having 3 heteroatoms all of which are N. In some embodiments, Ring A′ is an optionally substituted 10-membered aromatic ring having 2 heteroatoms independently selected from O, N, and S. In some embodiments, Ring A′ is an optionally substituted 10-membered aromatic ring having 2 heteroatoms one of which is N. In some embodiments, Ring A′ is an optionally substituted 10-membered aromatic ring having 2 heteroatoms independently selected from O and N. In some embodiments, Ring A′ is an optionally substituted 10-membered aromatic ring having 2 heteroatoms independently selected from O and S. In some embodiments, Ring A′ is an optionally substituted 10-membered aromatic ring having 2 heteroatoms independently selected from N and S. In some embodiments, Ring A′ is an optionally substituted 10-membered aromatic ring having 2 heteroatoms both of which are N. In some embodiments, Ring A′ is an optionally substituted 10-membered aromatic ring having 1 heteroatom selected from O, N, and S. In some embodiments, Ring A′ is an optionally substituted 10-membered aromatic ring having a nitrogen atom. In some embodiments, Ring A′ is an optionally substituted 10-membered aromatic ring having an oxygen atom. In some embodiments, Ring A′ is an optionally substituted 10-membered aromatic ring having a sulfur atom. In some embodiments, a ring has a single heteroatom. In some embodiments, a ring has two or more heteroatoms at least one of which is nitrogen. In some embodiments, each is nitrogen. In some embodiments, all heteroatoms are the same. In some embodiments, at least one heteroatom is different from the other heteroatom(s).

In some embodiments, Ring A′ is an optionally substituted bivalent naphthyl ring. In some embodiments, Ring A′ is optionally substituted

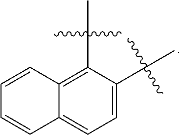 [SMILES: *c1ccc2ccccc2c1*]

In some embodiments, Ring A′ is

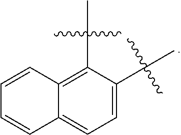 [SMILES: *c1ccc2ccccc2c1*]

### X

In some embodiments, X is —O—, —S—, or —N(R⁸)—, wherein R⁸ is as described herein. In some embodiments, X is —O—. In some embodiments, X is —S—. In some embodiments, X is —N(R⁸)—, wherein R⁸ is as described herein. In some embodiments, X is optionally substituted —CH₂—.

### R⁸

In some embodiments, R⁸ is R′ as described herein. In some embodiments, R⁸ is R as described herein. In some embodiments, R⁸ is H. In some embodiments, R⁸ is not H. In some embodiments, R⁸ is an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R⁸ is optionally substituted C₁-C₆ aliphatic. In some embodiments, R⁸ is optionally substituted C₁-C₆ alkyl. In some embodiments, R⁸ is optionally substituted C₁-C₄ alkyl. In some embodiments, R⁸ is methyl. In some embodiments, R⁸ is ethyl. In some embodiments, R⁸ is propyl. In some embodiments, R⁸ is isopropyl

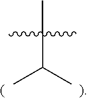 [SMILES: *C(C)C]

In some embodiments, R⁸ is butyl. In some embodiments, R⁸ is isobutyl

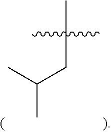 [SMILES: *CC(C)C]

In some embodiments, R⁸ is optionally substituted C₃-C₈ cycloalkyl. In some embodiments, R⁸ is optionally substituted

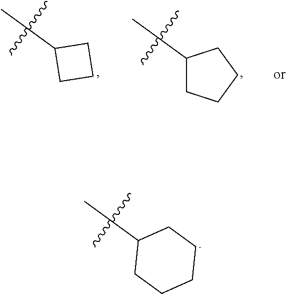 [SMILES: *C1CCC1 ; *C1CCCC1 ; *C1CCCCC1]

In some embodiments, R⁸ is optionally substituted

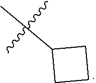 [SMILES: *C1CCC1]

In some embodiments, R⁸ is optionally substituted

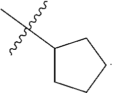 [SMILES: *C1CCCC1]

In some embodiments, R⁸ is optionally substituted

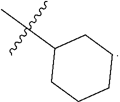 [SMILES: *C1CCCCC1]

In some embodiments, R⁸ is

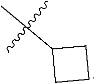 [SMILES: *C1CCC1]

In some embodiments, R⁸ is

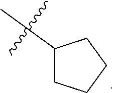 [SMILES: *C1CCCC1]

In some embodiments, R⁸ is

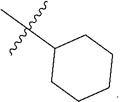 [SMILES: *C1CCCCC1]

In some embodiments, R⁸ is optionally substituted 6-10 membered aryl. In some embodiments, R⁸ is optionally substituted phenyl. In some embodiments, R⁸ is phenyl.

In some embodiments, R⁸ is optionally substituted 6-10 membered aryl-C₁-C₆ aliphatic. In some embodiments, R⁸ is optionally substituted 6-10 membered aryl-C₁-C₆ alkyl. In some embodiments, R⁸ is optionally substituted phenyl-C₁-C₆ alkyl. In some embodiments, R⁸ is optionally substituted

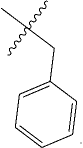 [SMILES: *Cc1ccccc1]

In some embodiments, R⁸ is

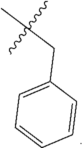 [SMILES: *Cc1ccccc1]

### Z

In some embodiments, Z is —N═ or —C(R⁹)═, wherein R⁹ is as described herein. In some embodiments, Z is —N═. In some embodiments, Z is —C(R⁹)═, wherein R^{o} is as described herein.

### R⁹

In some embodiments, R⁹ is H. In some embodiments, R⁹ is not H. In some embodiments, R⁹ is-R′, —OR′, halogen, —CN, —NO₂, or —N(R′)₂, wherein R¹ is as described herein. In some embodiments, R⁹ is —OR′ wherein R¹ is as described herein.

In some embodiments, R⁹ is halogen. In some embodiments, R⁹ is F. In some embodiments, R⁹ is C₁. In some embodiments, R⁹ is Br. In some embodiments, R⁹ is I.

In some embodiments, R⁹ is —CN.

In some embodiments, R⁹ is —NO₂.

In some embodiments, R⁹ is —N(R′)₂ wherein each R¹ is independently as described herein. In some embodiments, R° is —N(R′)₂ wherein each R¹ is independently R as described herein. In some embodiments, R¹ is —NHR′ wherein R¹ is as described herein.

In some embodiments, R^{o} is R′ as described herein. In some embodiments, R⁹ is R as described herein.

In some embodiments, R⁹ is an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R⁹ is C₁-C₆ aliphatic. In some embodiments, R⁹ is optionally substituted C₁-C₆ alkyl. In some embodiments, R⁹ is optionally substituted C₁-C₄ alkyl. In some embodiments, R⁹ is methyl. In some embodiments, R⁹ is ethyl. In some embodiments, R⁹ is propyl. In some embodiments, R⁹ is isopropyl

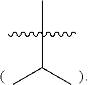

In some embodiments, R⁹ is butyl. In some embodiments, R⁹ is isobutyl

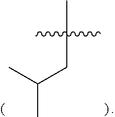

### R¹⁰

In some embodiments, R¹⁰ is R′ as described herein. In some embodiments, R¹⁰ is R as described herein. In some embodiments, R¹⁰ is H.

In some embodiments, R¹⁰ is not H. In some embodiments, R¹⁰ is an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic.

n

In some embodiments, n is 1, 2, or 3. In some embodiments, n is 1. In some embodiments, n is 2. In some embodiments, n is 3.

### R⁴

In some embodiments, R⁴ is H. In some embodiments, R⁴ is not H. In some embodiments, R⁴ is-R′, —OR′, halogen, —CN, —NO₂, or —N(R′)₂, wherein R¹ is as described herein. In some embodiments, R⁴ is —OR′ wherein R¹ is as described herein.

In some embodiments, R⁴ is halogen. In some embodiments, R⁴ is F. In some embodiments, R⁴ is C₁. In some embodiments, R⁴ is Br. In some embodiments, R⁴ is I.

In some embodiments, R⁴ is —CN.

In some embodiments, R⁴ is —NO₂.

In some embodiments, R⁴ is —N(R′)₂ wherein each R¹ is independently as described herein. In some embodiments, R⁴ is —N(R′)₂ wherein each R¹ is independently R as described herein. In some embodiments, R⁴ is —NHR′ wherein R¹ is as described herein.

In some embodiments, R⁴ is R′ as described herein. In some embodiments, R⁴ is R as described herein.

In some embodiments, R⁴ is an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R⁴ is C₁-C₆ aliphatic. In some embodiments, R⁴ is optionally substituted C₁-C₆ alkyl. In some embodiments, R⁴ is optionally substituted C₁-C₄ alkyl. In some embodiments, R⁴ is methyl. In some embodiments, R⁴ is ethyl. In some embodiments, R⁴ is propyl. In some embodiments, R⁴ is isopropyl. In some embodiments, R⁴ is butyl. In some embodiments, R⁴ is isobutyl. In some embodiments, R⁴ is C₁-4 haloalkyl. In some embodiments, R⁴ is —CF₃.

### R⁵

In some embodiments, R⁵ is H. In some embodiments, R⁵ is not H. In some embodiments, R⁵ is-R′, —OR′, halogen, —CN, —NO₂, or —N(R′)₂, wherein R¹ is as described herein. In some embodiments, R⁵ is —OR′ wherein R¹ is as described herein.

In some embodiments, R⁵ is halogen. In some embodiments, R⁵ is F. In some embodiments, R⁵ is C₁. In some embodiments, R⁵ is Br. In some embodiments, R⁵ is I.

In some embodiments, R⁵ is —CN.

In some embodiments, R⁵ is —NO₂.

In some embodiments, R⁵ is —N(R′)₂ wherein each R¹ is independently as described herein. In some embodiments, R⁵ is —N(R′)₂ wherein each R¹ is independently R as described herein. In some embodiments, R⁵ is —NHR′ wherein R¹ is as described herein.

In some embodiments, R⁵ is R′ as described herein. In some embodiments, R⁵ is R as described herein.

In some embodiments, R⁵ is an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R⁵ is C₁-C₆ aliphatic. In some embodiments, R⁵ is optionally substituted C₁-C₆ alkyl. In some embodiments, R⁵ is optionally substituted C₁-C₄ alkyl. In some embodiments, R⁵ is methyl. In some embodiments, R⁵ is ethyl. In some embodiments, R⁵ is propyl. In some embodiments, R⁵ is isopropyl. In some embodiments, R⁵ is butyl. In some embodiments, R⁵ is isobutyl. In some embodiments, R⁵ is C₁₋₄ haloalkyl. In some embodiments, R⁵ is —CF₃.

In some embodiments, R⁴ and R⁵ are taken together with their intervening atoms to form a ring as described herein. In some embodiments, R⁴ and R⁵ are taken together with their intervening atoms to form an optionally substituted phenyl ring. In some embodiments, R⁴ and R⁵ are taken together with their intervening atoms to form an optionally substituted 5- or 6-membered heteroaryl ring having 1-4 (e.g., 1, 2, 3, or 4, etc.) heteroatoms independently selected from nitrogen, oxygen and sulfur.

### R⁶

In some embodiments, R⁶ is H. In some embodiments, R⁶ is not H. In some embodiments, R⁶ is-R′, —OR′, halogen, —CN, —NO₂, or —N(R′)₂, wherein R¹ is as described herein. In some embodiments, R⁶ is —OR′ wherein R¹ is as described herein.

In some embodiments, R⁶ is halogen. In some embodiments, R⁶ is F. In some embodiments. R⁶ is C₁. In some embodiments, R⁶ is Br. In some embodiments, R⁶ is I.

In some embodiments, R⁶ is —CN.

In some embodiments, R⁶ is —NO₂.

In some embodiments, R⁶ is —N(R′)₂ wherein each R¹ is independently as described herein. In some embodiments, R⁶ is —N(R′)₂ wherein each R¹ is independently R as described herein. In some embodiments, R⁶ is —NHR′ wherein R¹ is as described herein.

In some embodiments, R⁶ is R′ as described herein. In some embodiments, R⁶ is R as described herein.

In some embodiments, R⁶ is an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R⁶ is C₁-C₆ aliphatic. In some embodiments, R⁶ is optionally substituted C₁-C₆ alkyl. In some embodiments, R⁶ is optionally substituted C₁-C₄ alkyl. In some embodiments, R⁶ is methyl. In some embodiments, R⁶ is ethyl. In some embodiments, R⁶ is propyl. In some embodiments, R⁶ is isopropyl. In some embodiments, R⁶ is butyl. In some embodiments, R⁶ is isobutyl. In some embodiments, R⁶ is C₁-4 haloalkyl. In some embodiments, R⁶ is —CF₃.

In some embodiments, R⁶ is —OR wherein R is as described herein. In some embodiments, R is H. In some embodiments, R is optionally substituted C₁₋₆ aliphatic. In some embodiments, R is C₁₋₆ alkyl. In some embodiments, R is methyl. In some embodiments, R is —CF₃.

In some embodiments, R⁶ is —C(O)OR wherein R is as described herein. In some embodiments, R⁶ is —C(O)OH.

In some embodiments, R⁶ is —S(O)₂R wherein R is as described herein. In some embodiments, R is C₁₋₆ aliphatic. In some embodiments, R is C₁₋₆ alkyl. In some embodiments, R⁶ is —S(O)₂Me.

### R⁷

In some embodiments, R⁷ is H. In some embodiments, R⁷ is not H. In some embodiments, R⁷ is-R′, —OR′, halogen, —CN, —NO₂, or —N(R′)₂, wherein R¹ is as described herein. In some embodiments, R⁷ is —OR′ wherein R¹ is as described herein.

In some embodiments, R⁷ is halogen. In some embodiments, R⁷ is F. In some embodiments. R⁷ is C₁. In some embodiments, R⁷ is Br. In some embodiments, R⁷ is I.

In some embodiments, R⁷ is —CN.

In some embodiments, R⁷ is —NO₂.

In some embodiments, R⁷ is —N(R′)₂ wherein each R¹ is independently as described herein. In some embodiments, R⁷ is —N(R′)₂ wherein each R¹ is independently R as described herein. In some embodiments, R⁷ is —NHR′ wherein R¹ is as described herein.

In some embodiments, R⁷ is R′ as described herein. In some embodiments, R⁷ is R as described herein.

In some embodiments, R⁷ is an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R⁷ is C₁-C₆ aliphatic. In some embodiments, R⁷ is optionally substituted C₁-C₆ alkyl. In some embodiments, R⁷ is optionally substituted C₁-C₄ alkyl. In some embodiments, R⁷ is methyl. In some embodiments, R⁷ is ethyl. In some embodiments, R⁷ is propyl. In some embodiments, R⁷ is isopropyl. In some embodiments, R⁷ is butyl. In some embodiments, R⁷ is isobutyl. In some embodiments, R⁷ is C₁₋₄ haloalkyl. In some embodiments, R⁷ is —CF₃.

In some embodiments, each of R⁴, R⁵, R⁶, and R⁷ is independently R′, —OR′, halogen, —CN, —NO₂, or —N(R′)₂, wherein R¹ is a described herein.

In some embodiments, each of R⁴, R⁵, R⁶, and R⁷ is H.

In some embodiments, each of R⁴, R⁵, R⁶, and R⁷ is independently not H.

In some embodiments, one of R⁴, R⁵, R⁶, and R⁷ is not H and each of the remaining three of R⁴, R⁵, R⁶, and R⁷ is H. In some embodiments, two of R⁴, R⁵, R⁶, and R⁷ is not H and each of the remaining two of R⁴, R⁵, R⁶, and R⁷ is H.

In some embodiments, each of R⁴, R⁵, and R⁷ is H, and R⁶ is selected from R′, —OR′, halogen, CN, —NO₂, or —N(R′)₂, wherein R¹ is as described herein. In some embodiments, each of R⁴, R⁵, and R⁷ is H, and R⁶ is selected from halogen, —CN, optionally substituted C₁-C₆ alkyl, —OR wherein R is optionally substituted C₁-C₆ alkyl, optionally substituted 6-10 membered aryl, —C(O) OR wherein R is H or optionally substituted C₁-C₆ alkyl, or —S(O)₂R wherein R is H or optionally substituted C₁-C₆ alkyl.

In some embodiments, each of R⁴, R⁵, and R⁷ is H, and R⁶ is halogen. In some embodiments, each of R⁴, R⁵, and R⁷ is H, and R⁶ is F. In some embodiments, each of R⁴, R⁵, and R⁷ is H, and R⁶ is Cl. In some embodiments, each of R⁴, R⁵, and R⁷ is H, and R⁶ is Br. In some embodiments, each of R⁴, R⁵, and R⁷ is H, and R⁶ is I.

In some embodiments, each of R⁴, R⁵, and R⁷ is H, and R⁶ is optionally substituted C₁-C₆ alkyl. In some embodiments, each of R⁴, R⁵, and R⁷ is H, and R⁶ is —CF₃.

In some embodiments, each of R⁴, R⁵, and R⁷ is H, and R⁶ is —OR wherein R is optionally substituted C₁-C₆ alkyl. In some embodiments, each of R⁴, R⁵, and R⁷ is H, and R⁶ is —OMe. In some embodiments, each of R⁴, R⁵, and R⁷ is H, and R⁶ is —OCF₃.

In some embodiments, each of R⁴, R⁵, and R⁷ is H, and R⁶ is optionally substituted 6-10 membered aryl. In some embodiments, each of R⁴, R⁵, and R⁷ is H, and R⁶ is optionally substituted phenyl. In some embodiments, each of R⁴, R⁵, and R⁷ is H, and R⁶ is phenyl.

In some embodiments, each of R⁴, R⁵, and R⁷ is H, and R⁶ is —C(O)OR wherein R is H or optionally substituted C₁-C₆ alkyl. In some embodiments, each of R⁴, R⁵, and R⁷ is H, and R⁶ is —C(O)OH.

In some embodiments, each of R⁴, R⁵, and R⁷ is H, and R⁶ is —S(O)₂R wherein R is H or optionally substituted C₁-C₆ alkyl. In some embodiments, each of R⁴, R⁵, and R⁷ is H, and R⁶ is —SO₂Me.

In some embodiments, Ring A is

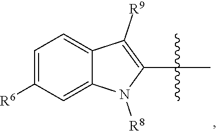

wherein each of R⁶, R⁸, and R⁹ is independently as described herein.

In some embodiments, Ring A is

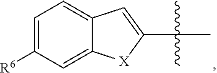

wherein each of X and R⁶ is independently as described herein. In some embodiments, Ring A is

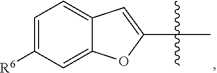

wherein R⁶ is as described herein.

In some embodiments, Ring A is

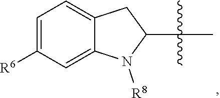

wherein each of R⁶ and R⁸ is independently as described herein. In some embodiments, Ring A is

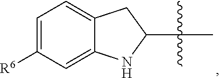

wherein R⁶ is as described herein. In some embodiments, Ring A is

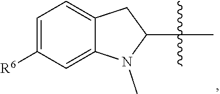

wherein R⁶ is as described herein.

In some embodiments, each of R⁴, R⁶, and R⁷ is H, and R⁵ is selected from R′, —OR′, halogen, —CN, —NO₂, or —N(R′)₂, wherein R¹ is as described herein. In some embodiments, each of R⁴, R⁶, and R⁷ is H, and R⁵ is selected from halogen, —CN, optionally substituted C₁-C₆ alkyl, —OR wherein R is optionally substituted C₁-C₆ alkyl, optionally substituted 6-10 membered aryl, —C(O) OR wherein R is H or optionally substituted C₁-C₆ alkyl, or —S(O)₂R wherein R is H or optionally substituted C₁-C₆ alkyl.

In some embodiments, each of R⁴, R⁶, and R⁷ is H, and R⁵ is optionally substituted C₁-C₆ alkyl. In some embodiments, each of R⁴, R⁶, and R⁷ is H, and R⁵ is —CF₃.

In some embodiments, each of R⁴, R⁶, and R⁷ is H, and R⁵ is halogen. In some embodiments, each of R⁴, R⁶, and R⁷ is H, and R⁵ is F. In some embodiments, each of R⁴, R⁶, and R⁷ is H, and R⁵ is Cl.

In some embodiments, Ring A is

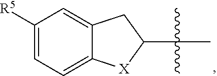

wherein each of X and R⁵ is independently as described herein. In some embodiments, Ring A is

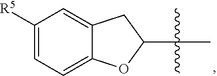

wherein R⁵ is as described herein.

In some embodiments, Ring A is

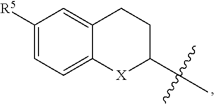

wherein each of X and R⁵ is independently as described herein. In some embodiments, Ring A is

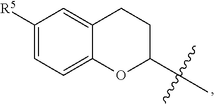

wherein R⁵ is as described herein.

In some embodiments, Ring A is

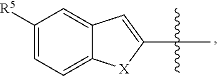

wherein each of X and R⁵ is independently as described herein. In some embodiments, Ring A is

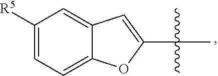

wherein R⁵ is as described herein.

In some embodiments, each of R⁴ and R⁷ is H, and each of R⁵ and R⁶ is independently selected from R′, —OR′, halogen, —CN, —NO₂, and —N(R′)₂, wherein R¹ is as described herein. In some embodiments, each of R⁴ and R⁷ is H, and each of R⁵ and R⁶ is independently selected from halogen, —CN, optionally substituted C₁-C₆ alkyl, —OR wherein R is optionally substituted C₁-C₆ alkyl, optionally substituted 6-10 membered aryl, —C(O) OR wherein R is H or optionally substituted C₁-C₆ alkyl, and —S(O)₂R wherein R is H or optionally substituted C₁-C₆ alkyl.

In some embodiments, each of R⁴ and R⁷ is H, and R⁵ is optionally substituted C₁-C₆ alkyl and R⁶ is halogen. In some embodiments, each of R⁴ and R⁷ is H, and R⁵ is —CF₃ and R⁶ is halogen. In some embodiments, each of R⁴ and R⁷ is H, and R⁵ is —CF₃ and R⁶ is F. In some embodiments, each of R⁴ and R⁷ is H, and R⁵ is —CF₃ and R⁶ is Cl.

In some embodiments, Ring A is

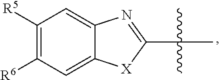

wherein each of X, R⁵, and R⁶ is independently as described herein.

In some embodiments, each of R⁶ and R⁷ is H, and each of R⁴ and R⁵ is independently selected from R′, —OR′, halogen, —CN, —NO₂, and —N(R′)₂, wherein R¹ is as described herein. In some embodiments, each of R⁶ and R⁷ is H, and each of R⁴ and R⁵ is independently selected from halogen, —CN, optionally substituted C₁-C₆ alkyl, —OR wherein R is optionally substituted C₁-C₆ alkyl, optionally substituted 6-10 membered aryl, —C(O) OR wherein R is H or optionally substituted C₁-C₆ alkyl, and —S(O)₂R wherein R is H or optionally substituted C₁-C₆ alkyl.

In some embodiments, each of R⁶ and R⁷ is H, and R⁴ is optionally substituted C₁-C₆ alkyl and R⁵ is halogen. In some embodiments, each of R⁴ and R⁷ is H, and R⁴ is —CF₃ and R⁵ is halogen. In some embodiments, each of R⁶ and R⁷ is H, and R⁴ is —CF₃ and R⁵ is F. In some embodiments, each of R⁶ and R⁷ is H, and R⁴ is —CF₃ and R⁵ is Cl.

In some embodiments, Ring A is

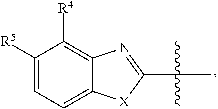

wherein each of X, R⁴, and R⁵ is independently as described herein. In some embodiments, Ring A is

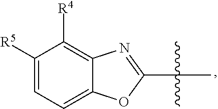

wherein each of R⁴ and R⁵ is independently as described herein.

In some embodiments, each of R⁴ and R⁶ is H, and each of R⁵ and R⁷ is independently selected from R′, —OR′, halogen, —CN, —NO₂, and —N(R′)₂, wherein R¹ is as described herein. In some embodiments, each of R⁴ and R⁶ is H, and each of R⁵ and R⁷ is independently selected from halogen, —CN, optionally substituted C₁-C₆ alkyl, —OR wherein R is optionally substituted C₁-C₆ alkyl, optionally substituted 6-10 membered aryl, —C(O) OR wherein R is H or optionally substituted C₁-C₆ alkyl, and —S(O)₂R wherein R is H or optionally substituted C₁-C₆ alkyl.

In some embodiments, each of R⁴ and R⁶ is H, and R⁵ is optionally substituted C₁-C₆ alkyl and R⁷ is halogen. In some embodiments, each of R⁴ and R⁶ is H, and R⁵ is —CF₃ and R⁷ is halogen. In some embodiments, each of R⁴ and R⁶ is H, and R⁵ is —CF₃ and R⁷ is F. In some embodiments, each of R⁴ and R⁶ is H, and R⁵ is —CF₃ and R⁷ is Cl.

In some embodiments, Ring A is

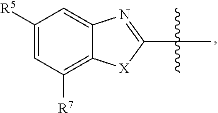

wherein each of X, R⁵, and R⁷ is

independently as described herein. In some embodiments, Ring A is

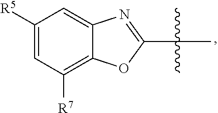

wherein each of R⁵ and R⁷ is independently as described herein.

In some embodiments, Ring A is

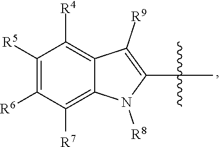

wherein each variable is independently as described herein. In some embodiments, Ring A is

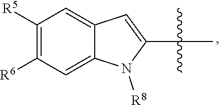

wherein each variable is independently as described herein. In some embodiments, Ring A is

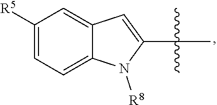

wherein each variable is independently as described herein. In some embodiments, Ring A is

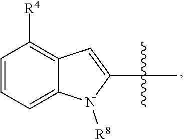

wherein each variable is independently as described herein. In some embodiments, Ring A is

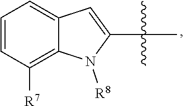

wherein each variable is independently as described herein. In some embodiments, Ring A is

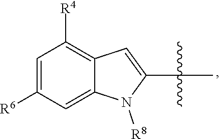

wherein each variable is independently as described herein.

In some embodiments, Ring A is

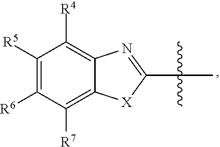

wherein each variable is independently as described herein.

In some embodiments, Ring A is

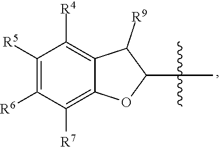

wherein each variable is independently as described herein. In some embodiments, Ring A is

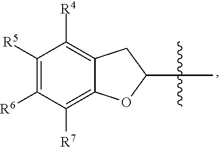

wherein each variable is independently as described herein.

In some embodiments, Ring A

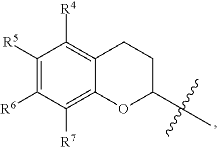

is wherein each variable is independently as described herein.

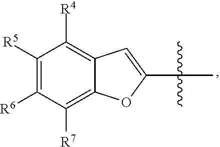

In some embodiments, Ring A is wherein each variable is independently as described herein.

In certain embodiments, the present disclosure provides a compound of Formula (I), or a pharmaceutical acceptable salt thereof, wherein Ring A is selected from:

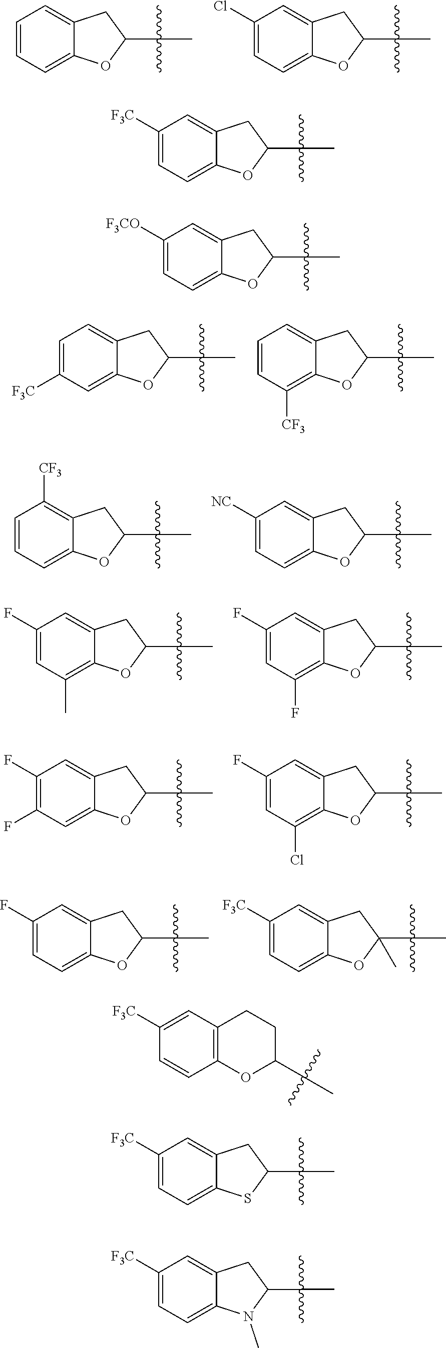

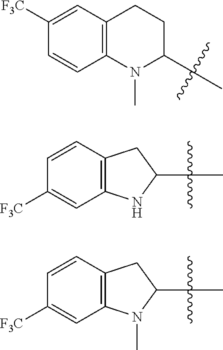

In certain embodiments, the present disclosure provides compounds of Formula (I), or a pharmaceutical acceptable salt thereof, wherein Ring A is selected from:

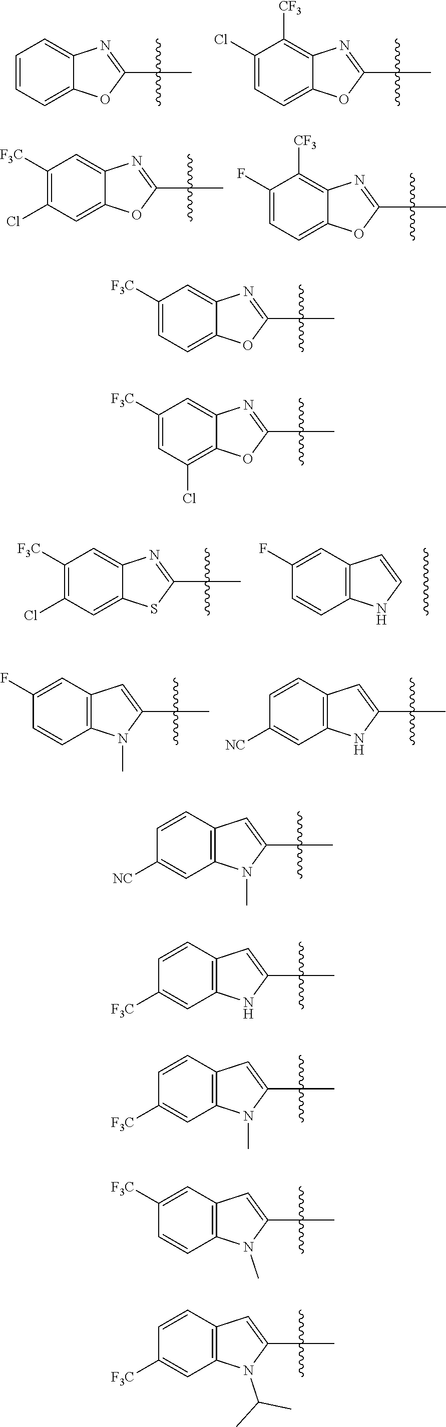

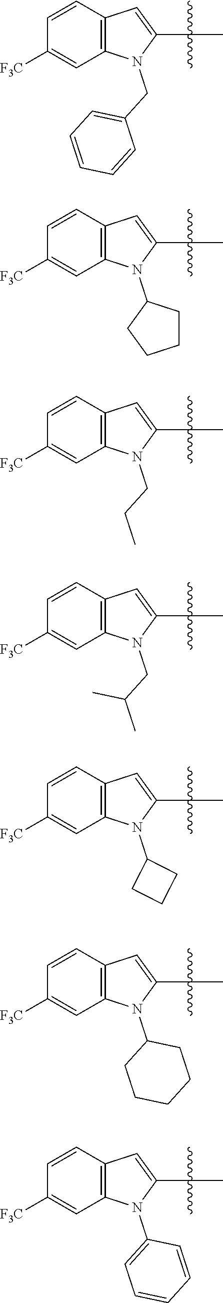

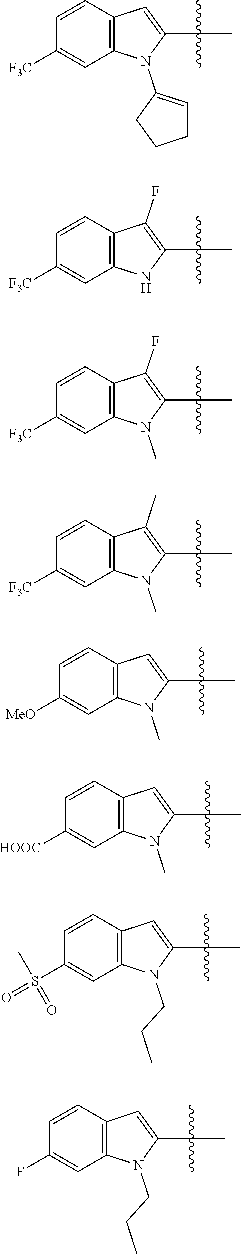

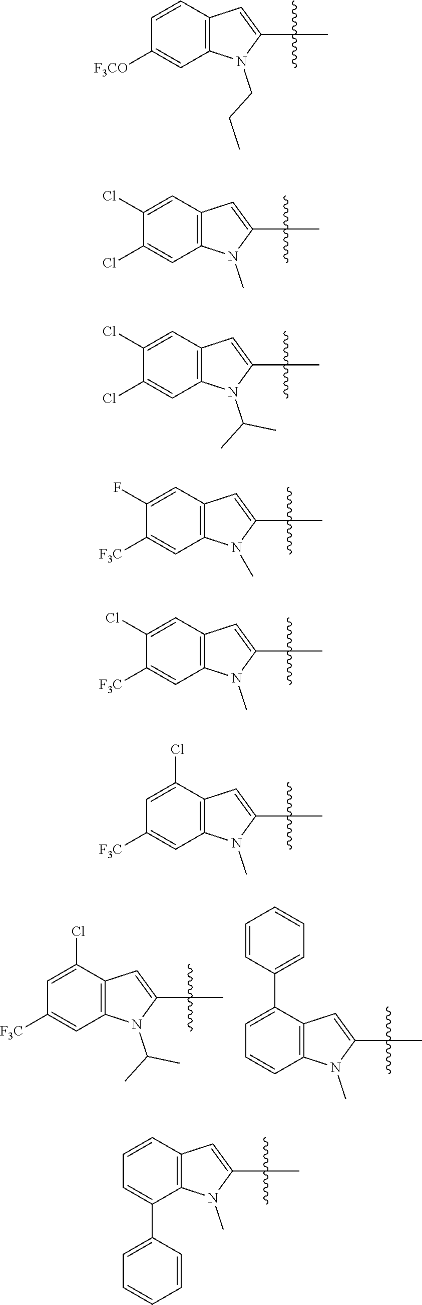

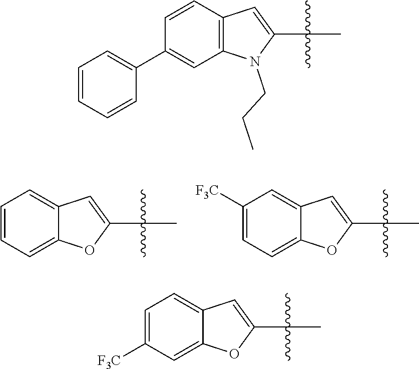

In some embodiments, the present disclosure provides compounds of Formula (I), or a pharmaceutical acceptable salt thereof, wherein Ring A is selected from

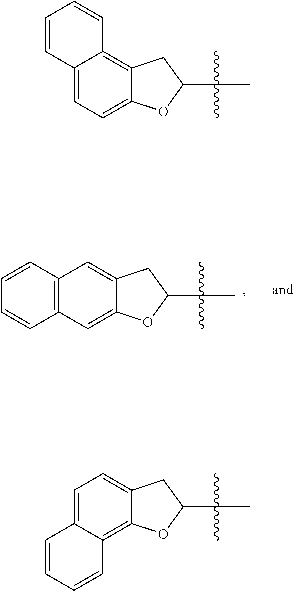 [SMILES: *C1Cc2c(ccc3ccccc23)O1 ; *C1Cc2cc3ccccc3cc2O1 ; *C1Cc2ccc3ccccc3c2O1]

and is optionally substituted.

### Ring B

In some embodiments, Ring B is an optionally substituted (as appreciated by those skilled in the art, in additional to R¹, R² and R³) ring selected from a 6-10 membered aryl ring and 5-10 membered heteroaryl ring having 1-6 heteroatoms.

In some embodiments, Ring B is an optionally substituted 6-10 membered aryl ring. In some embodiments, Ring B is an optionally substituted phenyl ring. In some embodiments, Ring B is a phenyl ring. In some embodiments, R¹ at position o (unless otherwise noted, o, m, and p are independently relative to the carbon bonded to Ring A). In some embodiments, R¹ is at position m. In some embodiments, R¹ is at position p. In some embodiments, R² is at position o. In some embodiments, R² is at position m. In some embodiments, R² is at position p. In some embodiments, R³ is at position o. In some embodiments, R³ is at position m. In some embodiments, R³ is at position p. In some embodiments, R¹ and R² are next to each other. In some embodiments, R¹ and R² are not next to each other. In some embodiments, R² and R³ are next to each other. In some embodiments, R² and R³ are not next to each other. In some embodiments, R¹ and R³ are next to each other. In some embodiments, R¹ and R³ are not next to each other. In some embodiments, Ring B is optionally substituted naphthyl.

In some embodiments, Ring B is optionally substituted 5-10 membered heteroaryl having 1-6 heteroatoms. In some embodiments, Ring B is optionally substituted 5-6 membered heteroaryl having 1-4 heteroatoms. In some embodiments, Ring B is an optionally substituted 9-membered bicyclic heteroaryl ring having 1-6 heteroatoms. In some embodiments, Ring B is an optionally substituted 10-membered bicyclic heteroaryl ring having 1-6 heteroatoms. In some embodiments, there is one heteroatom. In some embodiments, there are two heteroatoms. In some embodiments, there are three heteroatoms. In some embodiments, there are four heteroatoms. In some embodiments, there are five heteroatoms. In some embodiments, there are six heteroatoms. In some embodiments, each heteroatom is independently selected from nitrogen, oxygen and sulfur.

In some embodiments, Ring B is

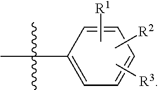

In some embodiments, Ring B is

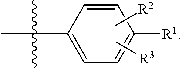

In some embodiments, Ring B is

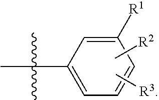

In some embodiments, Ring B is

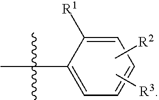

In some embodiments, Ring B is

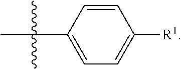

In some embodiments, Ring B is

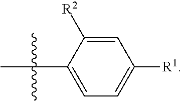

In some embodiments, Ring B is

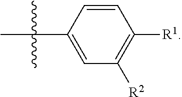

In some embodiments, Ring B is

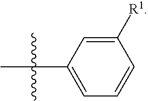

In some embodiments, Ring B is

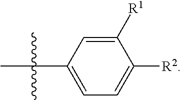

In some embodiments, Ring B is

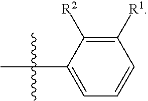

In some embodiments, Ring B is

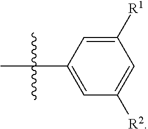

In some embodiments, Ring B is

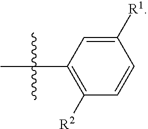

In some embodiments, Ring B is

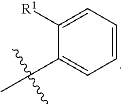

In some embodiments, Ring B is

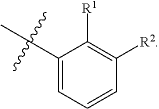

In some embodiments, Ring B is

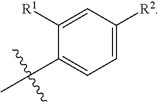

In some embodiments, Ring B is

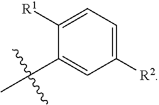

embodiments, Ring B is

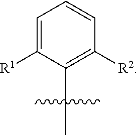

### R¹

In some embodiments, R¹ is —C(O)OR″, —P(O)(OR¹²)(OR¹³), —C(O)N(R¹⁴)SO₂R¹⁵, —C(O)NR¹⁶R¹⁷,

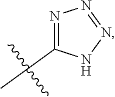 [SMILES: *c1nnn[nH]1]

halogen, or

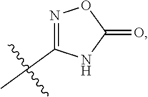 [SMILES: *c1noc(=O)[nH]1]

wherein each variable is independently as described herein.

In some embodiments, R¹ is —C(O)OR¹¹, wherein R¹¹ is as described herein. In some embodiments, R¹ is —P(O)(OR¹²)(OR¹³), wherein each of R¹² and R¹³ is independently as described herein. In some embodiments, R¹ is —C(O)N(R¹⁴)SO₂R¹⁵, wherein each of R′ and R¹⁵ is independently as described herein. In some embodiments, R¹ is —C(O)NR¹⁶R¹⁷, wherein each of R¹⁶ and R¹⁷ is independently as described herein. In some embodiments, R¹ is

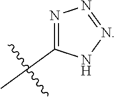 [SMILES: *c1nnn[nH]1]

In some embodiments, R¹ is —C(O)OR¹¹. In some embodiments, R¹¹ is R as described herein. In some embodiments, R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹¹ is C₁₋₆ aliphatic. In some embodiments, R¹¹ is C₁₋₆ alkyl. In some embodiments, R¹¹ is methyl. In some embodiments, R¹¹ is ethyl.

In some embodiments, R¹ is —C(O)OH. In some embodiments, R¹ is —C(O)OR¹¹, wherein R¹¹ is optionally substituted C₁-C₆ alkyl. In some embodiments, R¹ is —C(O)OR¹¹, wherein R¹¹ is optionally substituted 3-10 membered cycloalkyl.

In some embodiments, R is-P(O)(OR¹²)(OR¹³), wherein each of R¹² and R¹³ is independently as described herein.

In some embodiments, R¹² is R′ described herein. In some embodiments, R¹² is R as described herein. In some embodiments, R¹² is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹² is H. In some embodiments, R¹² is optionally substituted C₁₋₆ aliphatic. In some embodiments, R¹² is C₁₋₆ alkyl. In some embodiments, R¹² is methyl. In some embodiments, R¹² is ethyl.

In some embodiments, R¹³ is R′ described herein. In some embodiments, R¹³ is R as described herein. In some embodiments, R¹³ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹³ is H. In some embodiments, R¹³ is optionally substituted C₁₋₆ aliphatic. In some embodiments, R¹³ is C₁₋₆ alkyl. In some embodiments, R¹³ is methyl. In some embodiments, R¹³ is ethyl. In some embodiments, each of R¹² and R¹³ is independently R′ as described herein. In some embodiments, each of R¹² and R¹³ is independently R as described herein.

In some embodiments, R¹ is —P(O)(OR¹²)(OR¹³), wherein each of R¹² and R¹³ is independently H or optionally substituted C₁-C₆ alkyl. In some embodiments, R¹ is —P(O)(OR¹²)(OR¹³), wherein each of R¹² and R¹³ is independently H. In some embodiments, R¹ is —P(O)(OR¹²)(OR¹³), wherein R¹² is H and R¹³ is optionally substituted C₁-C₆ alkyl. In some embodiments, R¹ is —P(O)(OR¹²)(OR¹³), wherein R¹² is H and R¹³ is ethyl. In some embodiments, R¹ is —P(O)(OR¹²)(OR¹³), wherein each of R¹² and R¹³ is independently C₁-C₆ alkyl. In some embodiments, R¹ is —P(O)(OR¹²)(OR¹³), wherein each of R¹² and R¹³ is ethyl.

In some embodiments, R¹ is —C(O)N(R¹⁴)SO₂R¹⁵ wherein each of R¹⁴ and R¹⁵ is independently as described herein. In some embodiments, R¹ is —C(O)NHSO₂R¹⁵ wherein R¹⁵ is as described herein. In some embodiments, R¹ is —CN.

In some embodiments, R¹⁴ is R′ described herein. In some embodiments, R¹⁴ is R as described herein. In some embodiments, R¹⁴ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹⁴ is H. In some embodiments, R¹⁴ is optionally substituted C₁₋₆ aliphatic. In some embodiments, R¹⁴ is C₁₋₆ alkyl. In some embodiments, R¹⁴ is methyl. In some embodiments, R¹⁴ is ethyl.

In some embodiments, R¹⁵ is R′ described herein. In some embodiments, R¹⁵ is R as described herein. In some embodiments, R¹⁵ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹⁵ is H. In some embodiments, R¹⁵ is optionally substituted C₁₋₆ aliphatic. In some embodiments, R¹⁵ is C₁₋₆ alkyl. In some embodiments, R¹⁵ is methyl. In some embodiments, R¹⁵ is ethyl.

In some embodiments, each of R¹⁴ and R¹⁵ is independently R′ as described herein. In some embodiments, each of R¹⁴ and R¹⁵ is independently R as described herein.

In some embodiments, R¹ is —C(O)NR¹⁶R¹⁷ wherein each of R¹⁶ and R¹⁷ is independently as described herein.

In some embodiments, R¹⁶ is R′ described herein. In some embodiments, R¹⁶ is R as described herein. In some embodiments, R¹⁶ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹⁶ is H. In some embodiments, R¹⁶ is optionally substituted C₁₋₆ aliphatic. In some embodiments, R¹⁶ is C₁₋₆ alkyl. In some embodiments, R¹⁶ is methyl. In some embodiments, R¹⁶ is ethyl.

In some embodiments, R¹⁷ is R′ described herein. In some embodiments, R¹⁷ is R as described herein. In some embodiments, R¹⁷ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹⁷ is H. In some embodiments, R¹⁷ is optionally substituted C₁₋₆ aliphatic. In some embodiments, R¹⁷ is C₁₋₆ alkyl. In some embodiments, R¹⁷ is methyl. In some embodiments, R¹⁷ is ethyl.

In some embodiments, each of R¹⁶ and R¹⁷ is independently R′ as described herein. In some embodiments, each of R¹⁶ and R¹⁷ is independently R as described herein.

In some embodiments, R¹ is —C(O)NR¹⁶R¹⁷, wherein each of R¹⁶ and R¹⁷ is independently H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic.

In some embodiments, R¹ is —C(O)NR¹⁶R¹⁷, wherein each of R¹⁶ and R¹⁷ is independently H or optionally substituted C₁-C₆ alkyl. In some embodiments, R¹ is —C(O)NR¹⁶R¹⁷, wherein each of R¹⁶ and R¹⁷ is independently H. In some embodiments, R¹ is —C(O)NR¹⁶R¹⁷, wherein R¹⁶ is H and R¹⁷ is optionally substituted C₁-C₆ alkyl. In some embodiments, R¹ is —C(O)NR¹⁶R¹⁷, wherein R¹⁶ is H and R¹⁷ is methyl. In some embodiments, R¹ is —C(O)NR¹⁶R¹⁷, wherein each of R¹⁶ and R¹⁷ is independently C₁-C₆ alkyl. In some embodiments, R¹ is —C(O)NR¹⁶R¹⁷, wherein each of R¹⁶ and R¹⁷ is methyl.

In some embodiments, R¹ is an isostere of —C(O)OH. In some embodiments, a compound is a carboxylic acid isostere of a carboxylic acid compound, e.g., where R¹ is —C(O)OH. In some embodiments, a carboxylic acid isostere is one described in WO 2020/198537, US 2021/0032213, WO 2021/211839, or WO 2022/061008, the carboxylic acid isosteres of each of which are independently incorporated herein by reference.

In some embodiments, a carboxylic acid isostere is described in Ballatore et al., ChemMedChem. 2013 March; 8 (3): 385-395, the carboxylic acid isosteres of which are incorporated herein by reference.

In some embodiments, a carboxylic acid bioisostere is a hydroxamic acid. In some embodiments, R¹ is —C(O)N(R¹⁴) OH wherein R¹⁴ is as described herein. In some embodiments, R¹ is —C(O)NHOH. In some embodiments, R¹ is —N(OH)C(O)R¹¹ wherein R¹¹ is as described herein. In some embodiments, a carboxylic acid bioisostere is a phosphinic acid. In some embodiments, R¹ is —P(O)H(OR¹²) wherein R¹² is as described herein. In some embodiments, a carboxylic acid bioisostere is a phosphonic acid. In some embodiments, a carboxylic acid is a N-cyanoacetamide. In some embodiments, R¹ is —C(O)NHCN. In some embodiments, a carboxylic acid bioisostere is a sulphonic acid. In some embodiments, R¹ is —S(O)₂OR¹¹ wherein R¹¹ is as described herein. In some embodiments, a carboxylic acid bioisostere is a sulfonamide. In some embodiments, R¹ is —S(O)₂NR¹⁶R¹⁷ wherein each of R¹⁶ and R¹⁷ is independently as described herein. In some embodiments, a carboxylic acid bioisostere is an acylsulfonamide. In some embodiments, R¹ is —S(O)₂N(R¹⁴)C(O)R¹⁵ wherein each of R¹⁴ and R¹⁵ is independently as described herein. In some embodiments, a carboxylic acid bioisostere is a sulfonylurea.

In some embodiments, R¹ is —S(O)₂N(R¹⁴)C(O)NR¹⁶R¹⁷ wherein each of R¹⁴, R¹⁶ and R¹⁷ is independently as described herein. In some embodiments, R¹ is —N(R¹⁶)C(O)(R¹⁴)S(O)₂R¹⁵ wherein each of R¹⁴, R¹⁵ and R¹⁶ is independently as described herein. In some embodiments, R¹ is —S(O)₂NR¹⁶R¹⁷ wherein each of R¹⁶ and R¹⁷ is independently as described herein. In some embodiments, R¹ is —N(R¹⁴)S(O)₂R¹⁵ wherein each of R¹⁴ and R¹⁵ is independently as described herein. In some embodiments, as described herein, R¹⁴ is —H. In some embodiments, R¹⁶ is —H. In some embodiments, a carboxylic acid bioisostere is a tetrazole. In some embodiments, R¹ is

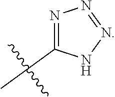 [SMILES: *c1nnn[nH]1]

In some embodiments, a carboxylic acid bioisostere is a thiazolidinedione. In some embodiments, R¹ is optionally substituted

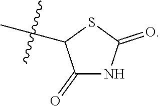 [SMILES: *C1SC(=O)NC1=O]

In some embodiments, a carboxylic acid bioisostere is an oxazolidinedione. In some embodiments, R¹ is optionally substituted

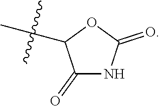 [SMILES: *C1OC(=O)NC1=O]

In some embodiments, a carboxylic acid bioisostere is a 5′-oxo-1,2,4-oxadiazole. In some embodiments, R¹ is optionally substituted

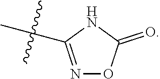 [SMILES: *c1noc(=O)[nH]1]

In some embodiments, a carboxylic acid bioisostere is a 5′-oxo-1,2,4-thiadiazole. In some embodiments, R¹ is optionally substituted

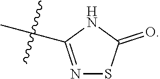 [SMILES: *c1nsc(=O)[nH]1]

In some embodiments, a carboxylic acid bioisostere is a 5′-thioxo-1,2,4-oxadiazole. In some embodiments, R¹ is optionally substituted

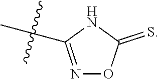 [SMILES: *c1noc(=S)[nH]1]

In some embodiments, a carboxylic acid bioisostere is isothiazole. In some embodiments, R¹ is optionally substituted

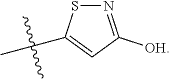 [SMILES: *c1cc(O)ns1]

In some embodiments, a carboxylic acid bioisostere is isoxazole. In some embodiments, R¹ is optionally substituted

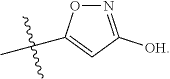 [SMILES: *c1cc(O)no1]

In some embodiments, a carboxylic acid bioisostere is a phenol, wherein the phenyl ring is optionally substituted. In some embodiments, a carboxylic acid bioisostere is phenol. In some embodiments, R¹ is substituted phenyl wherein a substituent is —OH. In some embodiments, R¹ is 4-hydroxylphenyl. In some embodiments, R¹ is 3-methyl-4-hydroxylphenyl. In some embodiments, a carboxylic acid bioisostere is a polyfluorophenol, e.g., difluorophenol. In some embodiments, R¹ is phenyl substituted with one or more fluoro and —OH. In some embodiments, R¹ is phenyl substituted with two or more fluoro and —OH. In some embodiments, R¹ is 3,5-difluoro-4-hydroxylphenyl. In some embodiments, a carboxylic acid isostere is a teramic acid. In some embodiments, R¹ is optionally substituted

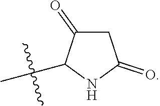 [SMILES: *C1NC(=O)CC1=O]

In some embodiments, a carboxylic acid isostere is a tetronic acid. In some embodiments, R¹ is optionally substituted

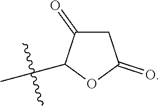 [SMILES: *C1OC(=O)CC1=O]

In some embodiments, a carboxylic acid isostere is a cyclopentane-1,3-dione. In some embodiments, R¹ is optionally substituted

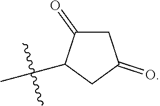 [SMILES: *C1CC(=O)CC1=O]

In some embodiments, a carboxylic acid isostere is a squaric acid. In some embodiments, R¹ is

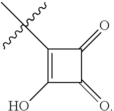 [SMILES: *c1c(O)c(=O)c1=O]

In some embodiments, R¹ is

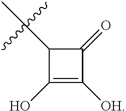 [SMILES: *C1C(=O)C(O)=C1O]

In some embodiments, a carboxylic acid bioisostere is 3-hydroxypyridin-4 (1H)-one. In some embodiments, R¹ is optionally substituted

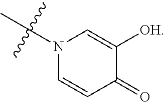 [SMILES: *n1ccc(=O)c(O)c1]

In some embodiments, R¹ is

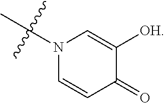 [SMILES: *n1ccc(=O)c(O)c1]

In some embodiments, a carboxylic acid bioisostere is 6-hydroxy-1,3-dioxin-4-one. In some embodiments, R¹ is optionally substituted

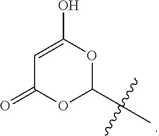 [SMILES: *C1OC(=O)C=C(O)O1]

In some embodiments, R¹ is optionally substituted

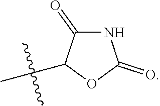 [SMILES: *C1OC(=O)NC1=O]

In some embodiments, R¹ is optionally substituted

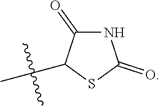 [SMILES: *C1SC(=O)NC1=O]

In some embodiments, R¹ is optionally substituted

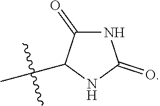 [SMILES: *C1NC(=O)NC1=O]

In some embodiments, R¹ is optionally substituted

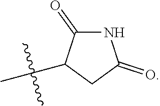 [SMILES: *C1CC(=O)NC1=O]

In some embodiments, R¹ is optionally substituted

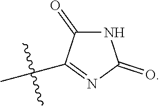 [SMILES: *C1=NC(=O)NC1=O]

In some embodiments, R¹ is optionally substituted

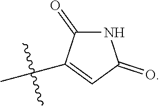 [SMILES: *C1=CC(=O)NC1=O]

In some embodiments, R¹ is

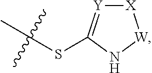

wherein each of X and W is independently-O—, —S—, —N(R⁸), or optionally substituted —CH₂—, and Y is —N═ or —C(R⁹)═, wherein each of R⁸ and R⁹ is independently as described herein, and the —NH— is optionally substituted. In some embodiments, R¹ is

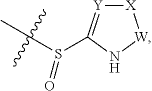

wherein each of X and W is independently-O—, —S—, —N(R⁸), or optionally substituted —CH₂—, and Y is —N═ or —C(R⁹)═, wherein each of R⁸ and R⁹ is independently as described herein, and the —NH—is optionally substituted. In some embodiments, R¹ is

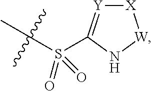

wherein each of X and W is independently —O—, —S—, —N(R⁸), or optionally substituted —CH₂—, and Y is —N═ or —C(R⁹)═, wherein each of R⁸ and R° is independently as described herein, and the —NH— is optionally substituted. In some embodiments, R¹ is

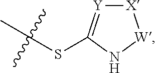

wherein each of X′, Y and W′ is independently-N═ or —C(R⁹)═, wherein R⁹ is as described herein, and the —NH— is optionally substituted. In some embodiments, R¹ is

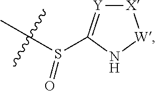

wherein each of X′, Y and W′ is independently-N═ or —C(R⁹)═, wherein R⁹ is as described herein, and the —NH— is optionally substituted. In some embodiments, R¹ is

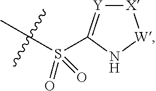

wherein each of X′, Y and W′ is independently-N═ or —C(R⁹)═, wherein R⁹ is as described herein, and the —NH— is optionally substituted. In some embodiments, the —NH— is not substituted. In some embodiments, the —NH— is substituted. In some embodiments, X is —O—. In some embodiments, X is —N(R⁸)— wherein R⁸ is as described herein. In some embodiments, X is optionally substituted —CH₂—. In some embodiments, Y is Z as described herein. In some embodiments, Y is —N═. In some embodiments, Y is —C(R⁹)═. In some embodiments, Y is optionally substituted —CH═. In some embodiments, Y is —CH═. In some embodiments, X′ is Z as described herein. In some embodiments, X′ is —N═. In some embodiments, X′ is-C(R⁹)═. In some embodiments, X′ is optionally substituted —CH═. In some embodiments, X′ is —CH═. In some embodiments, W′ is Z as described herein. In some embodiments, W′ is —N═. In some embodiments, W′ is —C(R⁹)═. In some embodiments, W′ is optionally substituted —CH═. In some embodiments, W′ is —CH═. In some embodiments, a carboxylic acid isostere is a hydroxyquinolinone. In some embodiments, a carboxylic acid isostere is a 3-hydroxyquinolin-2-one. In some embodiments, a carboxylic acid isostere is a 4-hydroxyquinolin-2-one. In some embodiments, a R¹ group described herein is substituted. In some embodiments, it is unsubstituted.

In some embodiments, R¹ is —CHO. In some embodiments, R¹ is protected-CHO.

In some embodiments, R¹ is R^{d6}. In some embodiments, R^{d6} is —CH(OR)₂. In some embodiments, each R is independently not —H. In some embodiments, each R is independently optionally substituted C₁₋₆ aliphatic. In some embodiments, the two R are taken together with their intervening atoms to form an optionally substituted 4-10, e.g., 5-10, 5-6, 4, 5, 6, 7, 8, 9, or 10 membered ring having 0-3 heteroatoms in addition to the intervening atoms. In some embodiments, there are no heteroatoms in addition to the intervening atoms. In some embodiments, a ring is 4-membered. In some embodiments, a ring is 5-membered. In some embodiments, a ring is 6-membered. In some embodiments, a ring is substituted. In some embodiments, a ring is unsubstituted. In some embodiments, a ring is saturated. In some embodiments, a ring is monocyclic. In some embodiments, R^{d6} is optionally substituted

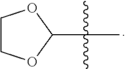 [SMILES: *C1OCCO1]

In some embodiments, R^{d6} is

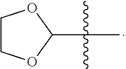 [SMILES: *C1OCCO1]

In some embodiments, R¹ is halogen. In some embodiments, R¹ is F. In some embodiments, R¹ is Cl. In some embodiments, R¹ is Br. In some embodiments, R¹ is I.

In some embodiments, R¹ is

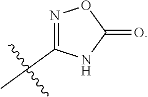 [SMILES: *c1noc(=O)[nH]1]

### R²

In some embodiments, R² is R¹ is as described herein. In some embodiments, R² is R as described herein. In some embodiments, R² is H. In some embodiments, R² is not H. In some embodiments, R² is optionally substituted C₁₋₆ aliphatic. In some embodiments, R² is C₁₋₆ aliphatic. In some embodiments, R² is optionally substituted C₁₋₆ alkyl. In some embodiments, R² is C₁₋₆ alkyl. In some embodiments, R² is methyl. In some embodiments, R² is optionally substituted C₃₋₆ cycloaliphatic. In some embodiments, R² is optionally substituted C₃₋₆ cycloalkyl. In some embodiments, R² is optionally substituted cyclopropyl. In some embodiments, R² is cyclopropyl. In some embodiments, R² is optionally substituted cyclobutyl. In some embodiments, R² is cyclobutyl. In some embodiments, R² is optionally substituted cyclopentyl. In some embodiments, R² is cyclopentyl. In some embodiments, R² is optionally substituted cyclohexyl. In some embodiments, R² is cyclohexyl. In some embodiments, R² is optionally substituted phenyl. In some embodiments, R² is phenyl. In some embodiments, R² is optionally substituted 5-6 membered heteroaryl having 1-4, e.g., 1, 2, 3 or 4 heteroatoms, e.g., independently selected from nitrogen, oxygen and sulfur. In some embodiments, R² is

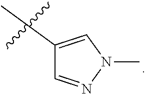 [SMILES: *c1cnn(C)c1]

In some embodiments, R² is R′ wherein R¹ is —C(O)OR. In some embodiments, R² is —C(O)OH. In some embodiments, R² is —C(O)OR wherein R is optionally substituted C₁₋₆ aliphatic.

In some embodiments, R² is —OR′ wherein R¹ is as described herein. In some embodiments, R¹ is R as described herein. In some embodiments, R¹ is H. In some embodiments, R¹ is optionally substituted C₁₋₆ aliphatic. In some embodiments, R¹ is optionally substituted C₁₋₆ aliphatic. In some embodiments, R¹ is methyl.

In some embodiments, R² is halogen. In some embodiments, R² is F. In some embodiments, R² is C₁. In some embodiments, R² is Br.

In some embodiments, R² is —CN. In some embodiments, R² is —NO₂. In some embodiments, R² is —N(R′)₂ wherein each R¹ is independently as described herein. In some embodiments, R² is —NHR′ wherein R¹ is as described herein. In some embodiments, each R¹ is independently R as described herein. In some embodiments, R² is —NH₂.

### R³

In some embodiments, R³ is R¹ is as described herein. In some embodiments, R³ is R as described herein. In some embodiments, R³ is H. In some embodiments, R³ is not H. In some embodiments, R³ is optionally substituted C₁₋₆ aliphatic. In some embodiments, R³ is C₁₋₆ aliphatic. In some embodiments, R³ is optionally substituted C₁₋₆ alkyl. In some embodiments, R³ is C₁₋₆ alkyl. In some embodiments, R³ is methyl. In some embodiments, R³ is optionally substituted C₃₋₆ cycloaliphatic. In some embodiments, R³ is optionally substituted C36 cycloalkyl. In some embodiments, R³ is optionally substituted cyclopropyl. In some embodiments, R³ is cyclopropyl. In some embodiments, R³ is optionally substituted cyclobutyl. In some embodiments, R³ is cyclobutyl. In some embodiments, R³ is optionally substituted cyclopentyl. In some embodiments, R³ is cyclopentyl. In some embodiments, R³ is optionally substituted cyclohexyl. In some embodiments, R³ is cyclohexyl. In some embodiments, R³ is optionally substituted phenyl. In some embodiments, R³ is phenyl. In some embodiments, R³ is optionally substituted 5-6 membered heteroaryl having 1-4, e.g., 1, 2, 3 or 4 heteroatoms, e.g., independently selected from nitrogen, oxygen and sulfur. In some embodiments, R³ is

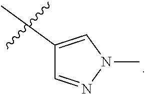 [SMILES: *c1cnn(C)c1]

In some embodiments, R³ is R′ wherein R¹ is —C(O)OR. In some embodiments, R³ is —C(O)OH. In some embodiments, R³ is —C(O)OR wherein R is optionally substituted C₁₋₆ aliphatic.

In some embodiments, R³ is —OR′ wherein R¹ is as described herein. In some embodiments, R¹ is R as described herein. In some embodiments, R¹ is H. In some embodiments, R¹ is optionally substituted C₁₋₆ aliphatic. In some embodiments, R¹ is optionally substituted C₁₋₆ aliphatic. In some embodiments, R¹ is methyl.

In some embodiments, R³ is halogen. In some embodiments, R³ is F. In some embodiments, R³ is C₁. In some embodiments, R³ is Br.

In some embodiments, R³ is —CN. In some embodiments, R³ is —NO₂. In some embodiments, R³ is —N(R′)₂ wherein each R¹ is independently as described herein. In some embodiments, R³ is —NHR′ wherein R¹ is as described herein. In some embodiments, each R¹ is independently R as described herein. In some embodiments, R³ is —NH₂.

In some embodiments, wherein each of R³ and R³ is independently R′, —OR′, halogen, —CN, —NO₂, or —N(R′)₂, wherein each variable is as described herein. In some embodiments, each of R³ and R³ is H. In some embodiments, one of R³ and R³ is H and the other of R³ and R³ is not H. In some embodiments, one of R³ and R³ is H and the other of R³ and R³ is R, —OR′, halogen, —CN, —NO₂, or —N(R′)₂, wherein each variable is as described herein. In some embodiments, each of R³ and R³ is not H. In some embodiments, each of R³ and R³ is R, —OR′, halogen, —CN, —NO₂, or —N(R′)₂, wherein each variable is as described herein.

In some embodiments, Ring B is

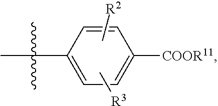

wherein each variable is as described herein.

In some embodiments, Ring B is

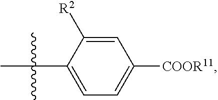

wherein each of R² and R¹¹ is independently R′, —OR′, halogen, —CN, —NO₂, or —N(R′)₂, wherein each variable is as described herein.

In some embodiments, Ring B is

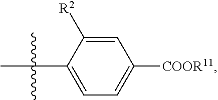

wherein R² is halogen and R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

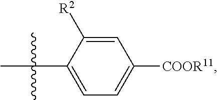

wherein R² is halogen and R¹¹ is H. In some embodiments, Ring B is

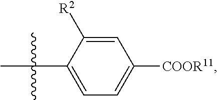

wherein R² is F and R¹¹ is H.

In some embodiments, Ring B is

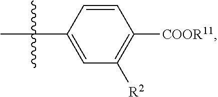

wherein each of R² and R¹¹ is independently R′, —OR′, halogen, —CN, —NO₂, or —N(R′)₂, wherein each variable is as described herein. In some embodiments, Ring B is

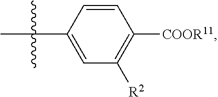

wherein R² is halogen and R¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

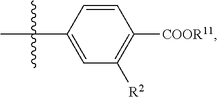

wherein R² is halogen and R¹¹ is H. In some embodiments, Ring B is

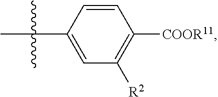

wherein R² is F and R¹¹ is H.

In some embodiments, Ring B is

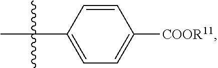

wherein R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered

heteroaryl having 1-6 heteroatoms-C₁-C₆ aliphatic. In some embodiments, Ring B is

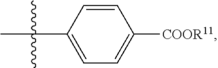

wherein R¹¹ is H.

In some embodiments, Ring B

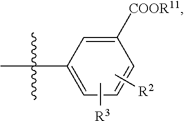

wherein each variable is independently as described herein.

In some embodiments, Ring B is

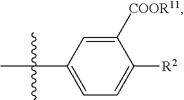

wherein each of R² and R¹¹ is independently R′, OR′, halogen, —CN, —NO₂, or —N(R′)₂, wherein each variable is as described herein. In some embodiments, Ring B is

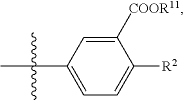

wherein R² is halogen and R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

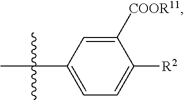

wherein R² is halogen and R¹¹ is H. In some embodiments, Ring B is

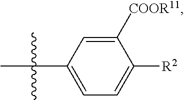

wherein R² is F and R¹¹ is H.

In some embodiments, Ring B is

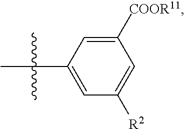

wherein each of R² and R¹¹ is independently R′, —OR′, halogen, —CN, —NO₂, or —N(R′)₂, wherein each variable is independently as described herein. In some embodiments, Ring B is

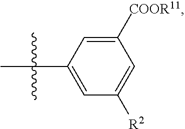

wherein R² is halogen, optionally substituted C₁-C₆ alkyl, —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms and R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

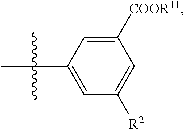

wherein R² is halogen and R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

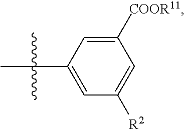

wherein R² is halogen and R¹¹ is H. In some embodiments, Ring B is

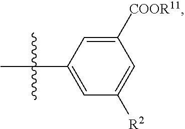

wherein R² is F and R¹¹ is H. In some embodiments, Ring B is

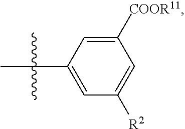

wherein R² is Cl and R¹¹ is H. In some embodiments, Ring B is

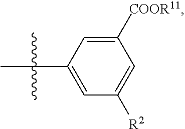

wherein R² is Br and R¹¹ is H.

In some embodiments, Ring B is

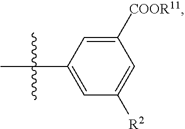

wherein R² is —OR wherein R is optionally substituted C₁-C₆ alkyl and R¹¹ is H. In some embodiments, Ring B is

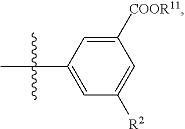

wherein R² is —OMe and R¹¹ is H. In some embodiments, Ring B is

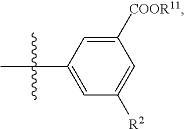

wherein R² is —OMe and R¹¹ is H.

In some embodiments, Ring B is

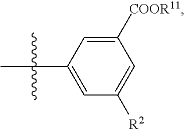

wherein R² is optionally substituted 6-10 membered aryl and R¹¹ is H. In some embodiments, Ring B is

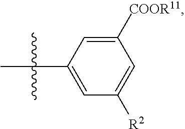

wherein R² is optionally phenyl and R¹¹ is H. In some embodiments, Ring B is

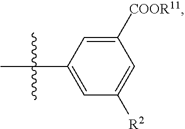

wherein R² is phenyl and R¹¹ is H.

In some embodiments, Ring B is

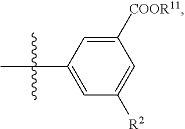

wherein R² is optionally substituted C₁-C₆ alkyl and R¹¹ is H. In some embodiments, Ring B is

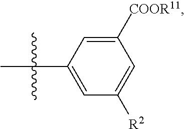

wherein R² is C₁-C₆ alkyl and R¹¹ is H. In some embodiments, Ring B is

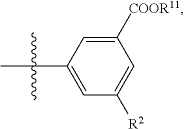

wherein R² is methyl and R¹¹ is H. In some embodiments, Ring B is

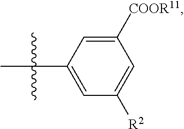

wherein R² is methyl and R¹¹ is H.

In some embodiments, Ring B is

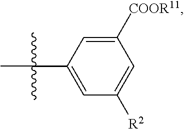

wherein R² is optionally substituted C₃-C₈ cycloalkyl and R¹¹ is H. In some embodiments, Ring B is

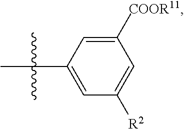

wherein R² is C₃-C₈ cycloalkyl and R¹¹ is H. In some embodiments, Ring B is

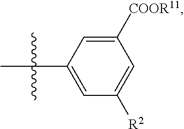

wherein R² is

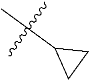 [SMILES: *C1CC1]

and R¹¹ is H. In some embodiments, Ring B

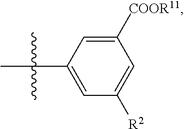

wherein R² is

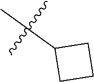 [SMILES: *C1CCC1]

and R¹¹ is H. In some embodiments, Ring B is

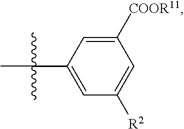

wherein R² is

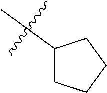 [SMILES: *C1CCCC1]

and R¹¹ is H. In some embodiments, Ring B is

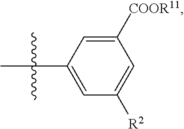

wherein R² is

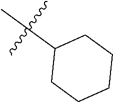 [SMILES: *C1CCCCC1]

### and R¹¹ is H.

In some embodiments, Ring B is

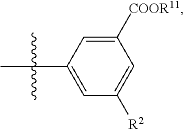

wherein R² is optionally substituted 5-10 membered heteroaryl having 1-6 heteroatoms and R¹′ is H. In some embodiments, Ring B is

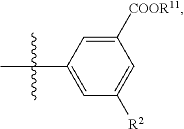

wherein R² is optionally substituted 5 membered heteroaryl having 1-3 heteroatoms independently selected from N, O, and S and R¹¹ is H. In some embodiments, Ring B is

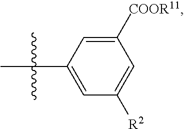

wherein R² is optionally substituted 6 membered heteroaryl having 1-3 heteroatoms independently selected from N, O, and S and R¹ is H. In some embodiments, Ring B is

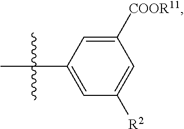

wherein R² is

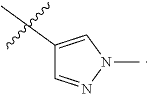 [SMILES: *c1cnn(C)c1]

### and R¹ is H.

In some embodiments, Ring B is

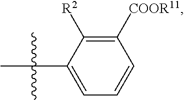

wherein each of R² and R¹¹ is independently R′, OR′, halogen, —CN, —NO₂, or —N(R′)₂, wherein each variable is independently as described herein. In some embodiments, Ring B is

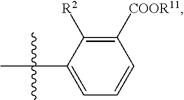

wherein R² is halogen, optionally substituted C₁-C₆ alkyl, —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms and R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

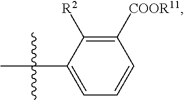

wherein R² is halogen and R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

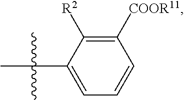

wherein R² is halogen and R¹¹ is H. In some embodiments, Ring B is

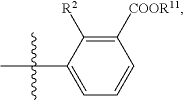

wherein R² is F and R¹ is H. In some embodiments, Ring B is

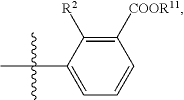

wherein R² is Cl and R¹¹ is H. In some embodiments, Ring B is

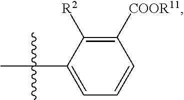

wherein R² is Br and R¹ is H.

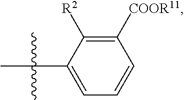

In some embodiments, Ring B is

wherein R² is —OR wherein R is optionally substituted C₁-C₆ alkyl and R¹¹ is H. In some embodiments, Ring B is

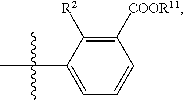

wherein R² is —OMe and R¹¹ is H. In some embodiments, Ring B is

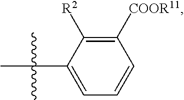

wherein R² is OMe and R¹¹ is H.

In some embodiments, Ring B is

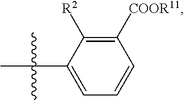

wherein R² is optionally substituted C₁-C₆ alkyl and R¹¹ is H. In some embodiments, Ring B is

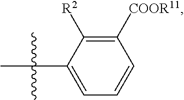

wherein R² is C₁-C₆ alkyl and R¹¹ is H. In some embodiments, Ring B is

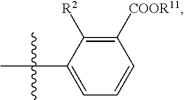

wherein R² is methyl and R¹¹ is H. In some embodiments, Ring B is

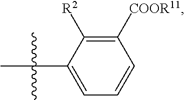

wherein R² is methyl and R¹ is H.

In some embodiments, Ring B is

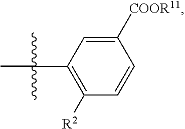

wherein each of R² and R¹¹ is independently R′, —OR′, halogen, —CN, —NO₂, or —N(R′)₂, wherein each variable is independently as described herein. In some embodiments, Ring B is

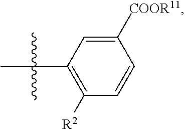

wherein R² is halogen, optionally substituted C₁-C₆ alkyl, OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms and R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

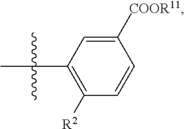

wherein R² is halogen and R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

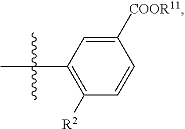

wherein R² is halogen and R¹¹ is H. In some embodiments, Ring B is

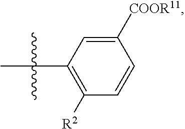

wherein R² is F and R¹¹ is H. In some embodiments, Ring B is

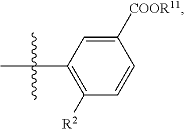

wherein R² is Cl and R¹¹ is H. In some embodiments, Ring B is

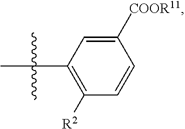

wherein R² is Br and R¹¹ is H.

In some embodiments, Ring B is

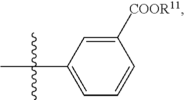

wherein R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

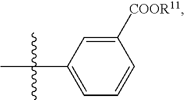

wherein R¹¹ is H.

In some embodiments, Ring B is

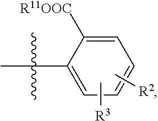

wherein each variable is independently as described herein.

In some embodiments, Ring B is

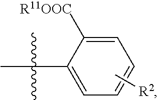

wherein each of R² and R″ is independently R′, —OR′, halogen, —CN, —NO₂, or —N(R′)₂, wherein each variable is as described herein.

In some embodiments, Ring B is

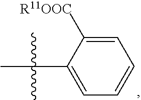

wherein R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

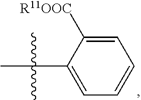

wherein R¹¹ is H.

In some embodiments, Ring B is

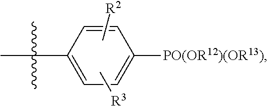

wherein each variable is independently as described herein. In some embodiments, Ring B is

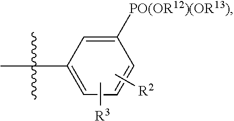

wherein each variable is independently as described herein. In some embodiments, Ring B is

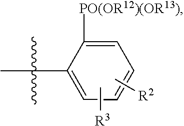

wherein each variable is independently as described herein.

In some embodiments, Ring B is

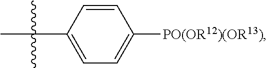

wherein each of R¹² and R¹³ is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

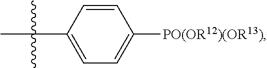

wherein each of R¹² and R¹³ is independently H or optionally substituted C₁-C₆ alkyl. In some embodiments, Ring B is

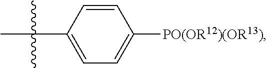

wherein each of R¹² and R¹³ is independently H. In some embodiments, Ring B is

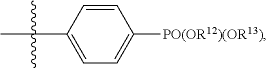

wherein each of R¹² and R¹³ is independently optionally substituted C₁-C₆ alkyl. In some embodiments, Ring B is

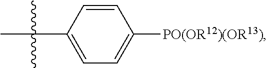

wherein each of R¹² is H and R¹³ is optionally substituted C₁-C₆ alkyl. In some embodiments, Ring B is

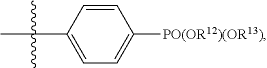

wherein each of R¹² is H and R¹³ is ethyl. In some embodiments, Ring B is

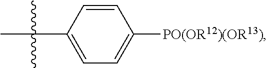

wherein each of R¹² is ethyl and R¹³ is ethyl.

In some embodiments, Ring B is

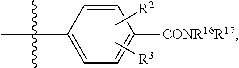

wherein each variable is independently as described herein. In some embodiments, Ring B is

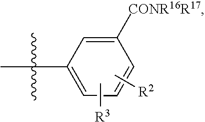

wherein each variable is independently as described herein. In some embodiments, Ring B is

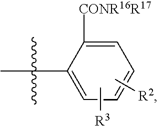

wherein each variable is independently as described herein.

In some embodiments, Ring B is

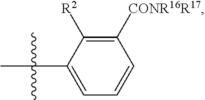

wherein R² is R′, —OR′, halogen, —CN, —NO₂, or —N(R′)₂ wherein each variable is independently as described herein and each of R¹⁶ and R¹⁷ is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic, or R¹⁶ and R¹⁷ are taken together with the nitrogen to form an optionally substituted 3-10 membered ring having, in addition to nitrogen, 0-4 heteroatoms. In some embodiments, Ring B is

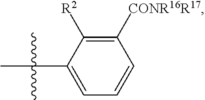

wherein R² is H or halogen and each of R¹⁶ and R¹⁷ is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

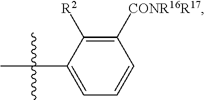

wherein R² is halogen and each of R¹⁶ and R¹⁷ is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

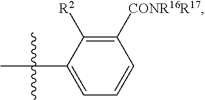

wherein R² is halogen and each of R¹⁶ and R¹⁷ is independently hydrogen or optionally C₁-C₆ alkyl. In some embodiments, Ring B is

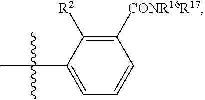

wherein R² is halogen and each of R¹⁶ and R¹⁷ is H. In some embodiments, Ring B is

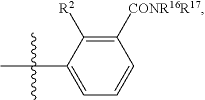

wherein R² is halogen, R¹⁶ is H, and R¹⁷ is optionally C₁-C₆ alkyl. In some embodiments, Ring B is

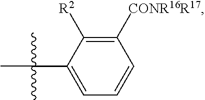

wherein R² is halogen, R¹⁶ is H, and R¹⁷ is methyl. In some embodiments, Ring B is

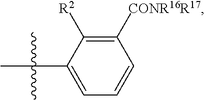

wherein R² is halogen and each of R¹⁶ and R¹⁷ is optionally C₁-C₆ alkyl. In some embodiments, Ring B is

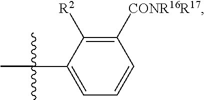

wherein R² is halogen and each of R¹⁶ and R¹⁷ is methyl.

In some embodiments, Ring B is

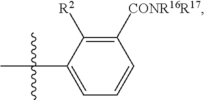

wherein R² is F and each of R¹⁶ and R¹⁷ is independently hydrogen or optionally C₁-C₆ alkyl. In some embodiments, Ring B is

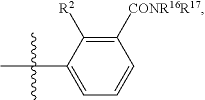

wherein R² is Cl and each of R¹⁶ and R¹⁷ is independently hydrogen or optionally C₁-C₆ alkyl. In some embodiments, Ring B is

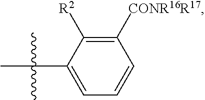

wherein R² is Br and each of R¹⁶ and R¹⁷ is independently hydrogen or optionally C₁-C₆ alkyl.

In some embodiments, Ring B is

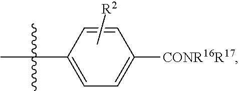

wherein each variable is independently as described herein. In some embodiments, Ring B is

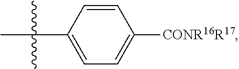

wherein each variable is independently as described herein.

In some embodiments, Ring B is

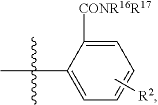

wherein each variable is independently as described herein. In some embodiments, Ring B is

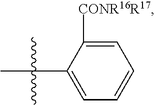

wherein each variable is independently as described herein.

In some embodiments, Ring B

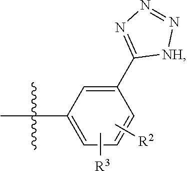

is wherein each variable is independently as described herein. In some embodiments, Ring B is

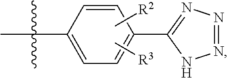

wherein each variable is independently as described herein. In some embodiments, Ring B is

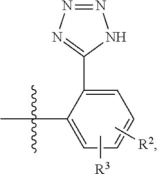

wherein each variable is independently as described herein.

In some embodiments, Ring B is

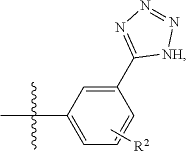

wherein R² is R′, —OR′, halogen, —CN, —NO₂, or —N(R′)₂ wherein each variable is independently as described herein.

In some embodiments, Ring B is

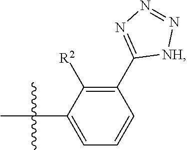

wherein R² is R′, —OR′, halogen, —CN, —NO₂, or —N(R′)₂ wherein each variable is independently as described herein. In some embodiments, Ring B is

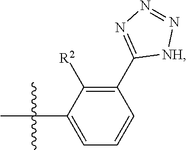

wherein R² is H. In some embodiments, Ring B is

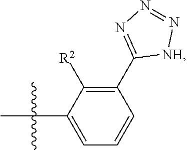

wherein R² is halogen, optionally substituted C₁-C₆ alkyl, —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms and R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

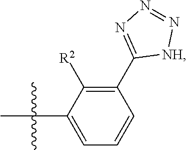

wherein R² is halogen. In some embodiments, Ring B is

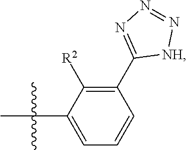

wherein R² is F. In some embodiments, Ring B is

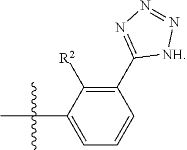

wherein R² is C₁. In some embodiments, Ring B is

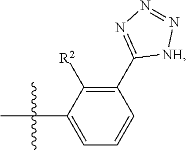

wherein R² is Br. In some embodiments, Ring B is

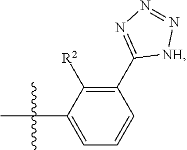

wherein R² is I.

In some embodiments, Ring B is

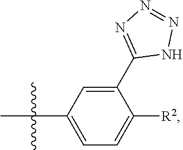

wherein R² is R′, —OR′, halogen, —CN, —NO₂, or —N(R′)₂ wherein each variable is independently as described herein. In some embodiments, Ring B is

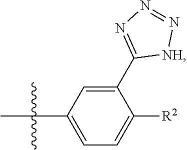

wherein R² is H. In some embodiments, Ring B is

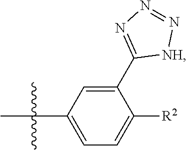

wherein R² is halogen, optionally substituted C₁-C₆ alkyl, —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms and R¹′ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

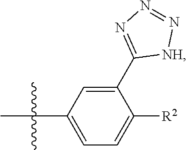

wherein R² is halogen. In some embodiments, Ring B is

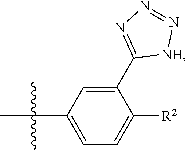

wherein R² is F. In some embodiments, Ring B is

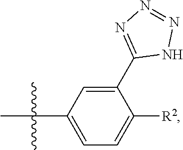

wherein R² is C₁. In some embodiments, Ring B is

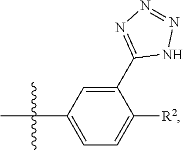

wherein R² is Br. In some embodiments, Ring B is

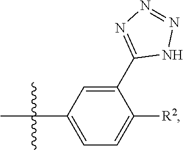

wherein R² is I.

In some embodiments, Ring B is

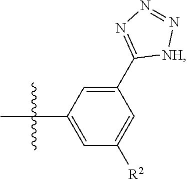

wherein R² is R′, —OR′, halogen, —CN, —NO₂, or —N(R′)₂ wherein each variable is independently as described herein. In some embodiments, Ring B is

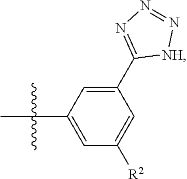

wherein R² is H. In some embodiments, Ring B is

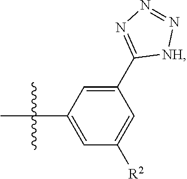

wherein R² is halogen, optionally substituted C₁-C₆ alkyl, —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms and R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, Ring B is

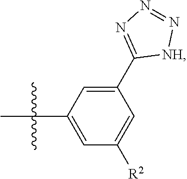

wherein R² is halogen. In some embodiments, Ring B is

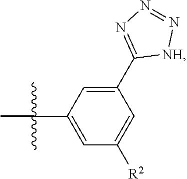

wherein R² is F. In some embodiments, Ring B is

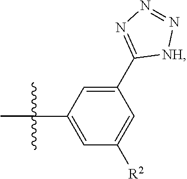

wherein R² is C₁. In some embodiments, Ring B is

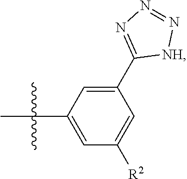

wherein R² is Br. In some embodiments, Ring B is

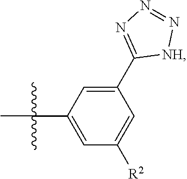

wherein R² is I.

In some embodiments, Ring B is

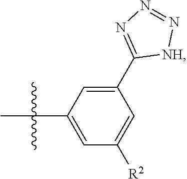

wherein R² is optionally substituted C₁-C₆ aliphatic. In some embodiments, Ring B is

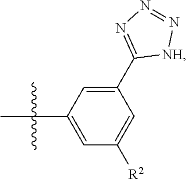

wherein R² is optionally substituted C₁-C₆ alkyl. In some embodiments, Ring B is

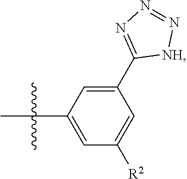

wherein R² is methyl. In some embodiments, Ring B is

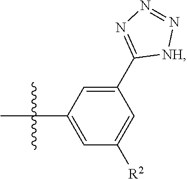

wherein R² is optionally substituted C₃-C₈ cycloalkyl. In some embodiments, Ring B is

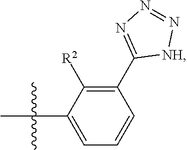

wherein R² is

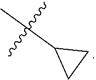 [SMILES: *C1CC1]

In some embodiments, Ring B is

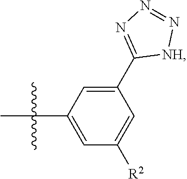

wherein R² is

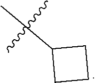 [SMILES: *C1CCC1]

In some embodiments, Ring B is

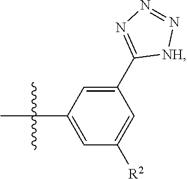

wherein R² is

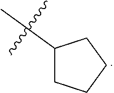 [SMILES: *C1CCCC1]

In some embodiments. In some embodiments, Ring B is

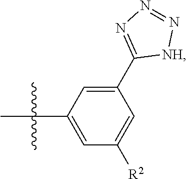

wherein R² is

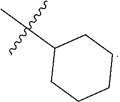 [SMILES: *C1CCCCC1]

In some embodiments, Ring B is

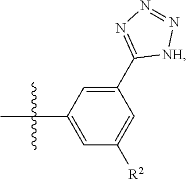

wherein R² is —C(O)OR and each variable is independently as described herein. In some embodiments, Ring B is

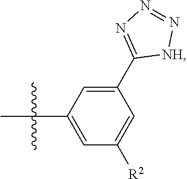

wherein R² is —C(O) OR and R is hydrogen or an optionally substituted C₁-C₆ aliphatic. In some embodiments, Ring B is

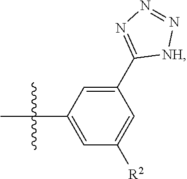

wherein R² is —C(O)OR and R is hydrogen. In some embodiments, Ring B is

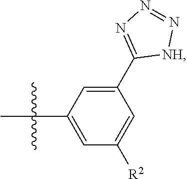

wherein R² is —C(O)OR and R is an optionally substituted group selected from C₁-C₆ aliphatic. In some embodiments, Ring B is

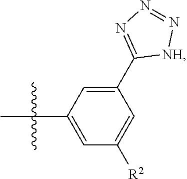

wherein R² is —C(O)OR and R is an optionally substituted group selected from C₁-C₆ alkyl. In some embodiments, Ring B is

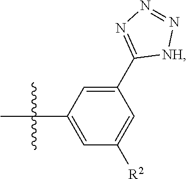

wherein R² is —C(O)OR and R is methyl. In some embodiments, Ring B is

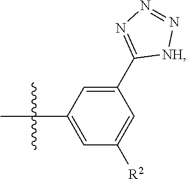

wherein R² is —C(O)OR and R is ethyl.

In some embodiments, Ring B is

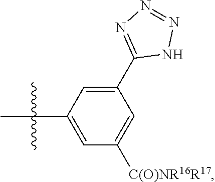

wherein each variable is independently as described herein. In some embodiments, Ring B is

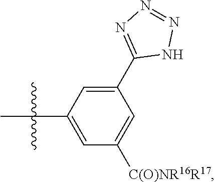

wherein R¹⁶ and R¹⁷ each are independently H or optionally substituted C₁-C₆ aliphatic. In some embodiments, Ring B is

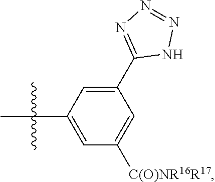

wherein R¹⁶ and R¹⁷ each are H.

In some embodiments, Ring B is

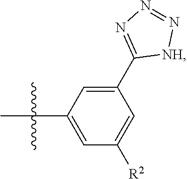

wherein R² is —CN.

In some embodiments, Ring B is

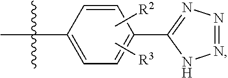

wherein each of R² and R³ is independently halogen, —CN, optionally substituted C₁-C₆ alkyl, —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms.

In some embodiments, Ring B is

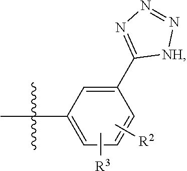

wherein each of R² and R³ is independently halogen, —CN, optionally substituted C₁-C₆ alkyl, —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms. In some embodiments, Ring B is

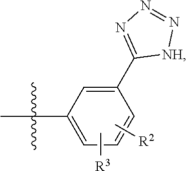

wherein each of R² and R³ is independently halogen and optionally substituted C₁-C₆ alkyl,

In some embodiments, In some embodiments, Ring B is

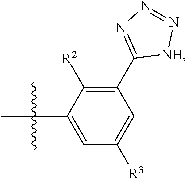

wherein each of R² and R³ is independently halogen and optionally substituted C₁-C₆ alkyl, In some embodiments, Ring B is

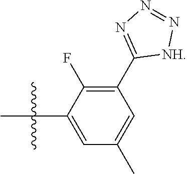 [SMILES: *c1cc(C)cc(-c2nnn[nH]2)c1F]

In some embodiments, Ring B is

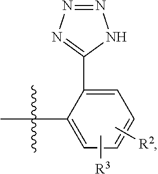

wherein each of R² and R³ is independently halogen, —CN, optionally substituted C₁-C₆ alkyl, —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms.

In certain embodiments, the present disclosure provides a compound of Formula (I), or a pharmaceutical acceptable salt thereof, wherein Ring B is selected from:

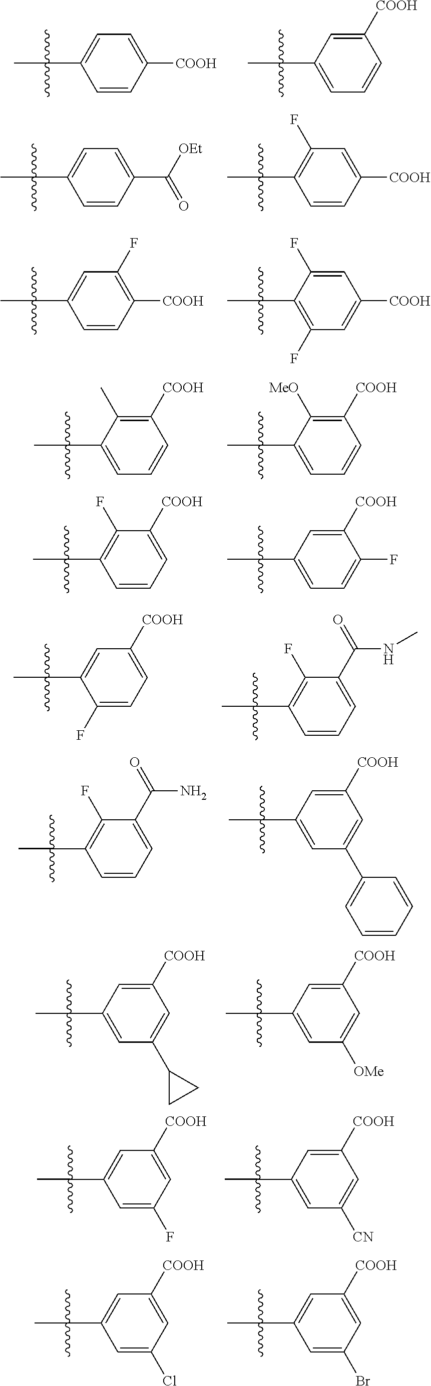

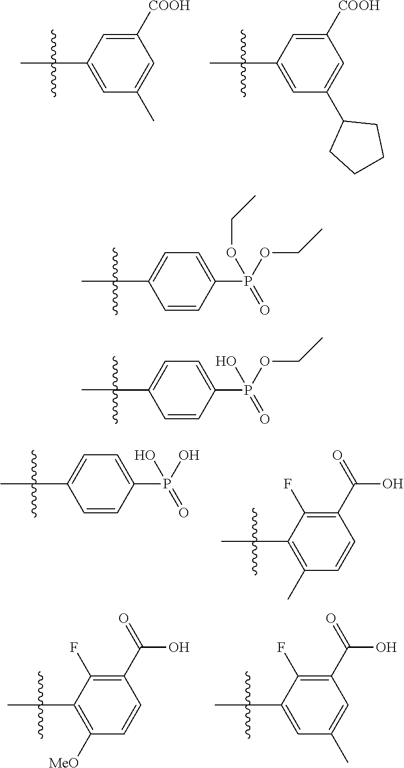

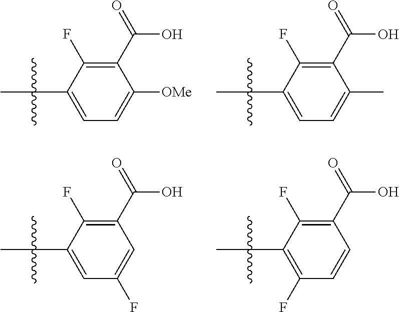

In certain embodiments, the present disclosure provides a compound of Formula (I), or a pharmaceutical acceptable salt thereof, wherein Ring B is selected from:

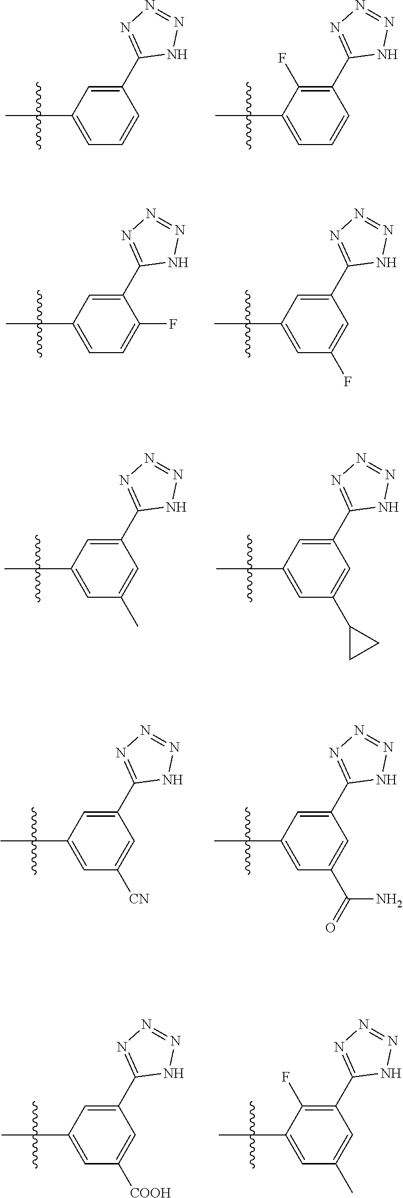

In certain embodiments, the present disclosure provides a compound of Formula (II), or a pharmaceutically acceptable salt thereof, wherein each of R¹, R², R³, R⁴, R⁵, R⁶, R⁷, R¹⁰, n, and X is independently as described herein.

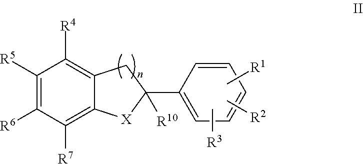

In certain embodiments, the present disclosure provides a compound of Formula (III), or a pharmaceutically acceptable salt thereof, wherein each of R¹, R², R³, R⁴, R⁵, R⁶, R⁷, Z, and X is independently as described herein.

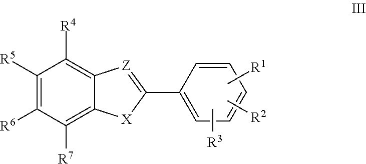

In certain embodiments, the present disclosure provides a compound of Formula (IV-1 to IV-6), or a pharmaceutically acceptable salt thereof, wherein each of R¹, R², R³, R⁴, R⁵, R⁶, R⁷, R⁸, and R¹⁰ is independently as described herein.

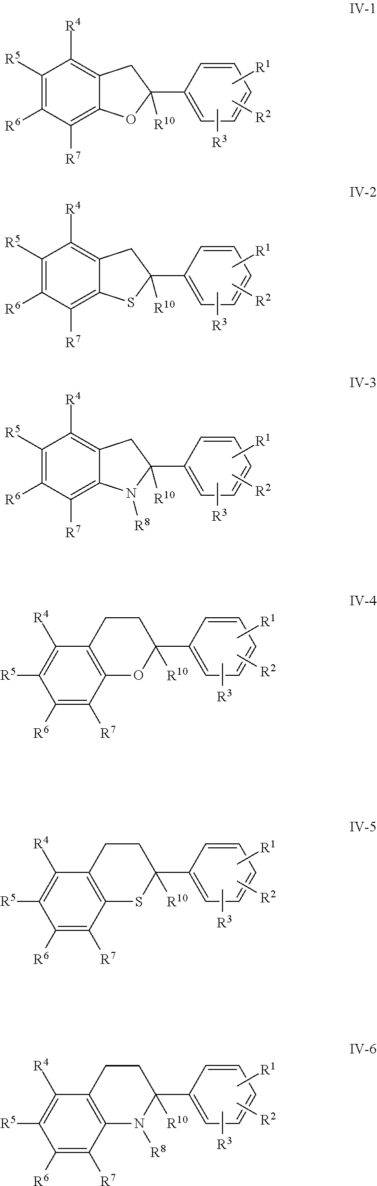

In certain embodiments, the present disclosure provides a compound of Formula (V-1 to V-6), or a pharmaceutically acceptable salt thereof, wherein each of R¹, R², R³, R⁴, R⁵, R⁶, R⁷, R⁸ and R⁹ is independently as described herein.

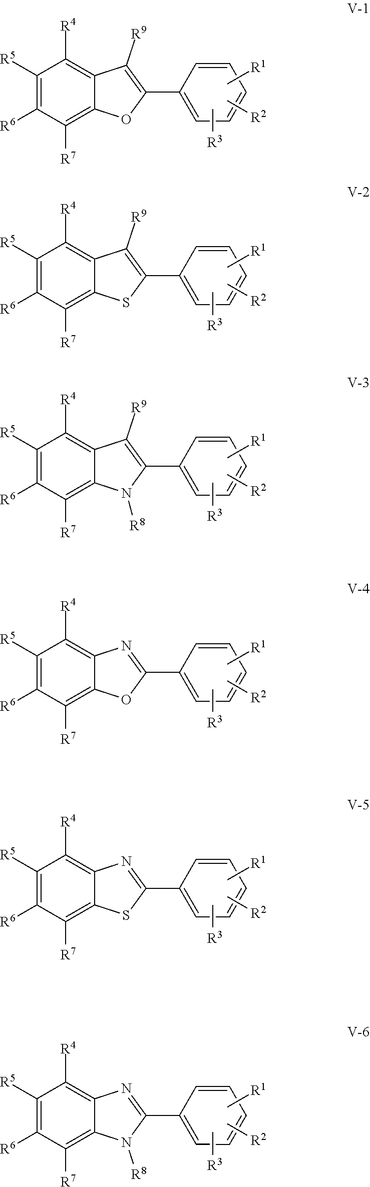

In certain embodiments, the present disclosure provides a compound of the following formulas, or a pharmaceutically acceptable salt thereof, wherein each of R¹, R², R³, R⁴, R⁵, R⁶, R⁷, R⁸, R⁹, R¹⁰, n, and X is independently as described herein and each of the ring is independently optionally substituted. In some embodiments, the present disclosure provides a compound of the following formulas, or a pharmaceutically acceptable salt thereof, wherein R¹ is

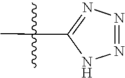 [SMILES: *c1nnn[nH]1]

and each of R², R³, R⁴, R⁵, R⁶, R⁷, R⁸, R⁹, R¹⁰, n, and X is independently as described herein and each of the ring is independently optionally substituted.

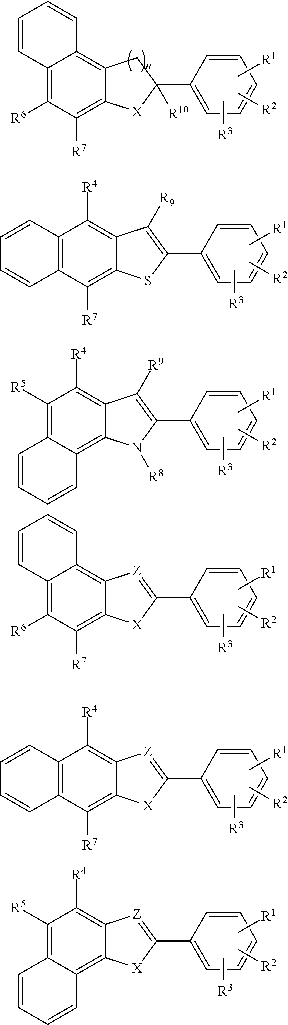

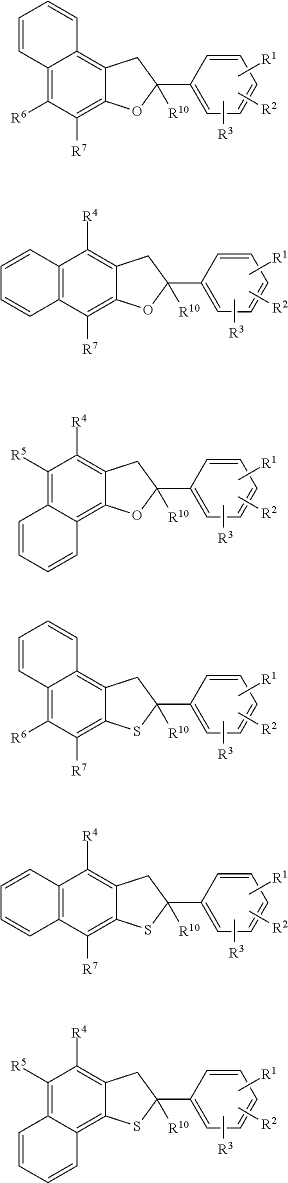

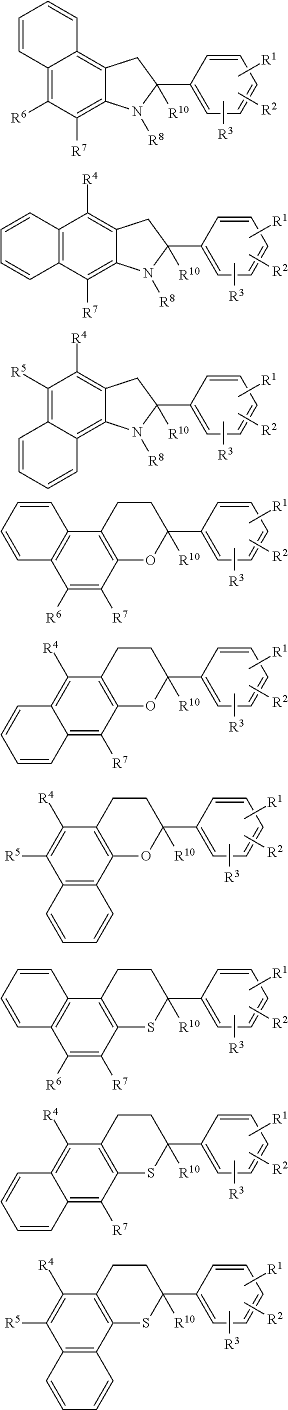

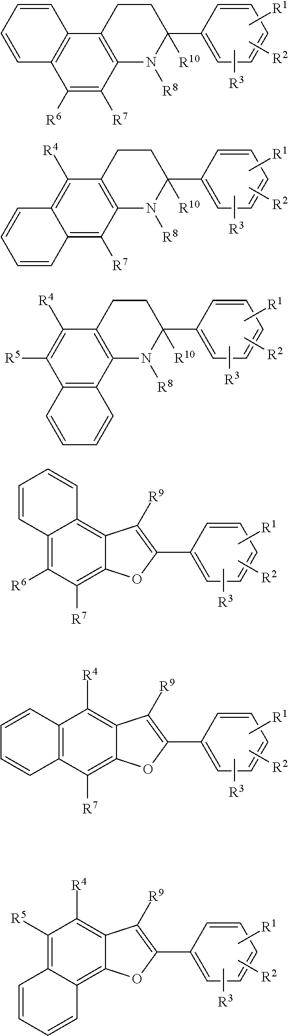

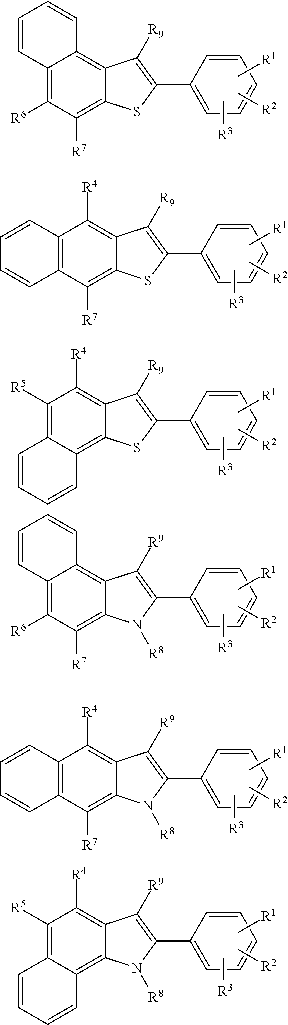

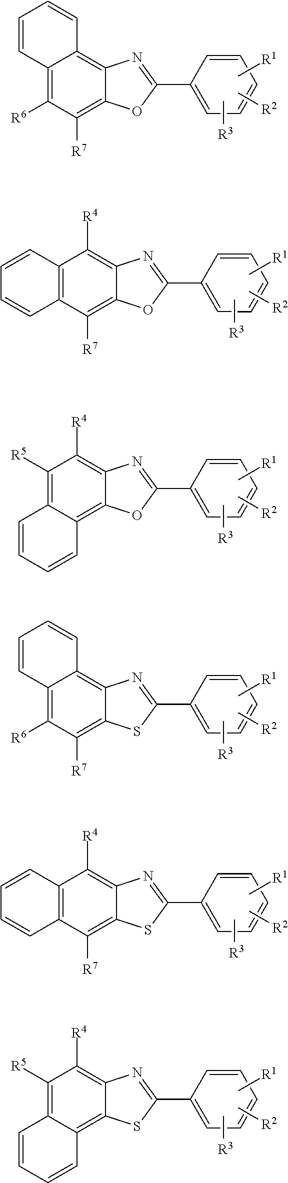

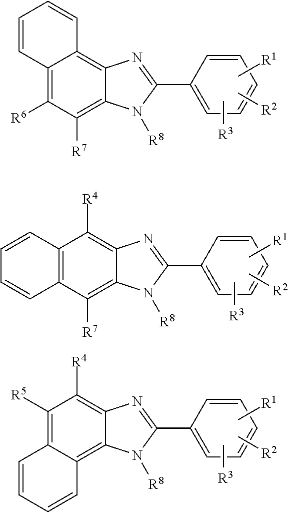

In some embodiments, a compound of Formula (I) or a pharmaceutical acceptable salt thereof has a structure of:

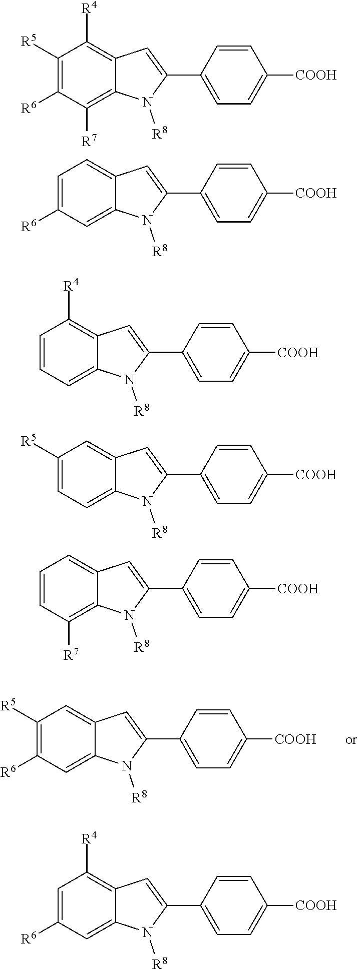

or a pharmaceutical acceptable salt thereof, wherein each variable is independently as described herein.

In some embodiments, a compound of Formula (I) or a pharmaceutical acceptable salt thereof has a structure of

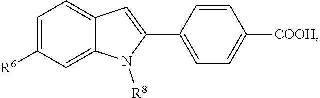

or a pharmaceutically acceptable salt thereof, wherein each variable is independently as described herein.

In some embodiments, a compound of Formula (I) or a pharmaceutical acceptable salt thereof has a structure of

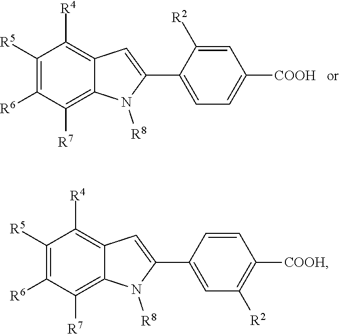

or a pharmaceutically acceptable salt thereof, wherein each variable is independently as described herein.

In some embodiments, a compound of Formula (I) or a pharmaceutical acceptable salt thereof has a structure of

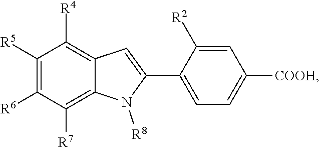

or a pharmaceutically acceptable salt thereof, wherein each variable is independently as described herein. In some embodiments, R² is halogen. In some embodiments, R² is F. In some embodiments, R⁸ is H or optionally substituted C₁-C₆ alkyl. In some embodiments, R⁸ is H. In some embodiments, R⁸ optionally substituted C₁-C₆ alkyl. In some embodiments, R⁸ is methyl. In some embodiments, R⁸ is propyl. In some embodiments, R⁸ is isopropyl. In some embodiments, R⁸ is isobutyl. In some embodiments, R⁸ is butyl. In some embodiments, R⁸ is optionally substituted C₃-C₈ cycloalkyl. In some embodiments, R⁸ is optionally substituted C₃-C₈ cycloalkyl. In some embodiments, R⁸ is

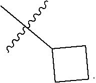 [SMILES: *C1CCC1]

In some embodiments, R⁸ is

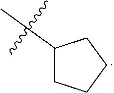 [SMILES: *C1CCCC1]

In some embodiments, R⁸ is

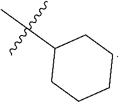 [SMILES: *C1CCCCC1]

In some embodiments, R⁸ is optionally substituted 6-10 membered aryl. In some embodiments, R⁸ is phenyl. In some embodiments, R⁶ is —CN. In some embodiments, R⁶ is halogen. In some embodiments, R⁶ is F. In some embodiments, R⁶ is C₁. In some embodiments, R⁶ is optionally substituted C₁-C₆ alkyl. In some embodiments, R⁶ is —CF₃.

In some embodiments, a compound of Formula (I) or a pharmaceutical acceptable salt thereof has a structure of

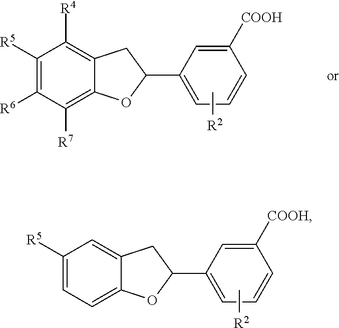

or a pharmaceutically acceptable salt thereof, wherein each variable is independently as described herein.

In some embodiments, a compound of Formula (I) or a pharmaceutical acceptable salt thereof has a structure of

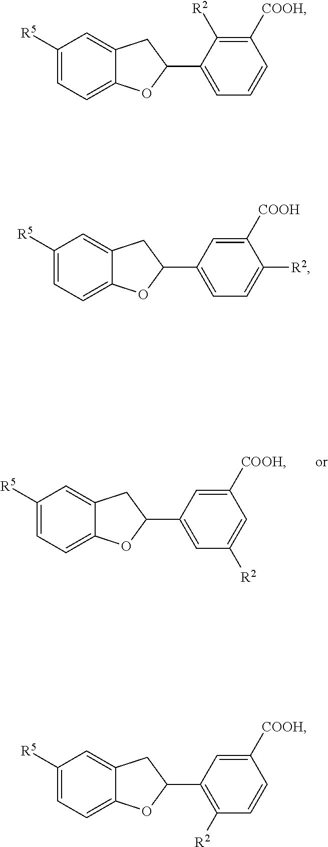

or a pharmaceutically acceptable salt thereof, wherein each variable is independently as described herein. In some embodiments, R⁵ is H. In some embodiments, R⁵ is halogen. In some embodiments, R⁵ is F. In some embodiments, R⁵ is C₁. In some embodiments, R⁵ is Br. In some embodiments, R⁵ is optionally substituted C₁-C₆ alkyl. In some embodiments, R⁵ is —CF₃. In some embodiments, R² is H. In some embodiments, R² is halogen. In some embodiments, R² is F. In some embodiments, R² is C₁. In some embodiments, R² is Br. In some embodiments, R² is optionally substituted C₁-C₆ alkyl. In some embodiments, R² is methyl. In some embodiments, R² is optionally substituted C₁-C₆ alkyl. In some embodiments, R² is optionally substituted C₃-C₈ cycloalkyl. In some embodiments, R² is

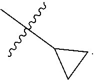 [SMILES: *C1CC1]

In some embodiments, R² is

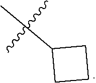 [SMILES: *C1CCC1]

In some embodiments, R² is

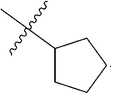 [SMILES: *C1CCCC1]

In some embodiments, R² is —OR wherein R is optionally substituted C₁-C₆ alkyl. In some embodiments, R² is —OMe. In some embodiments, R² is optionally substituted 6-10 membered aryl. In some embodiments, R² is phenyl. In some embodiments, R² is optionally substituted 5-10 membered heteroaryl having 1-3 heteroatoms. In some embodiments, R² is

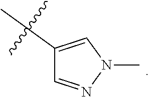 [SMILES: *c1cnn(C)c1]

In some embodiments, a compound of Formula (I) or a pharmaceutical acceptable salt thereof has a structure of

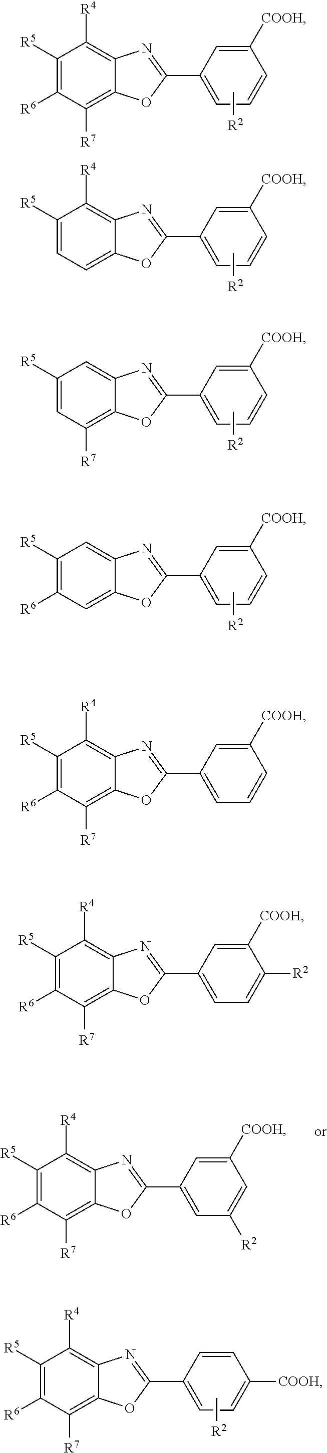

or a pharmaceutically acceptable salt thereof, wherein each variable is independently as described herein. In some embodiments, R² is H. In some embodiments, R² is halogen. In some embodiments, R² is F. In some embodiments, R² is C₁. In some embodiments, R² is Br. In some embodiments, R⁴ is optionally substituted C₁-C₆ alkyl. In some embodiments, R⁴ is optionally substituted C₁-C₆ alkyl. In some embodiments, R⁴ is —CF₃. In some embodiments, R⁵ is halogen. In some embodiments, R⁵ is F. In some embodiments, R⁵ is C₁. In some embodiments, R⁵ is Br. In some embodiments, R⁵ is optionally substituted C₁-C₆ alkyl. In some embodiments, R⁵ is —CF₃. In some embodiments, R⁶ is halogen. In some embodiments, R⁶ is F. In some embodiments, R⁶ is C₁. In some embodiments, R⁶ is Br. In some embodiments, R⁷ is halogen. In some embodiments, R⁷ is F. In some embodiments, R⁷ is C₁. In some embodiments, R⁷ is Br.

In some embodiments, a compound of Formula (I) or a pharmaceutical acceptable salt thereof has a structure of

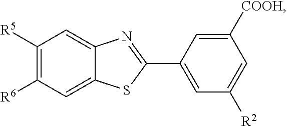

or a pharmaceutically acceptable salt thereof, wherein each variable is independently as described herein. In some embodiments, R² is halogen. In some embodiments, R² is F. In some embodiments, R² is C₁. In some embodiments, R² is Br. In some embodiments, R⁵ is optionally substituted C₁-C₆ alkyl. In some embodiments, R⁵ is —CF₃. In some embodiments, R⁶ is halogen. In some embodiments, R⁶ is F. In some embodiments, R⁶ is C₁. In some embodiments, R⁶ is Br.

In some embodiments, a compound of Formula (I) or a pharmaceutical acceptable salt thereof has a structure of

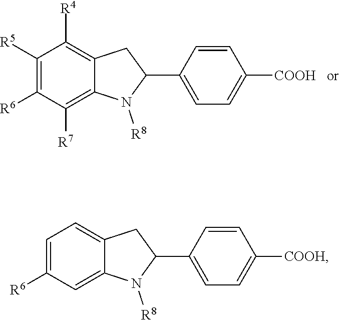

or a pharmaceutically acceptable salt thereof, wherein each variable is independently as described herein. In some embodiments, R⁸ is H. In some embodiments, R⁸ is optionally substituted C₁-C₆ alkyl. In some embodiments, R⁸ is methyl. In some embodiments, R⁶ is optionally substituted C₁-C₆ alkyl. In some embodiments, R⁶ is —CF₃.

In some embodiments, a compound of Formula (I) or a pharmaceutical acceptable salt thereof has a structure of

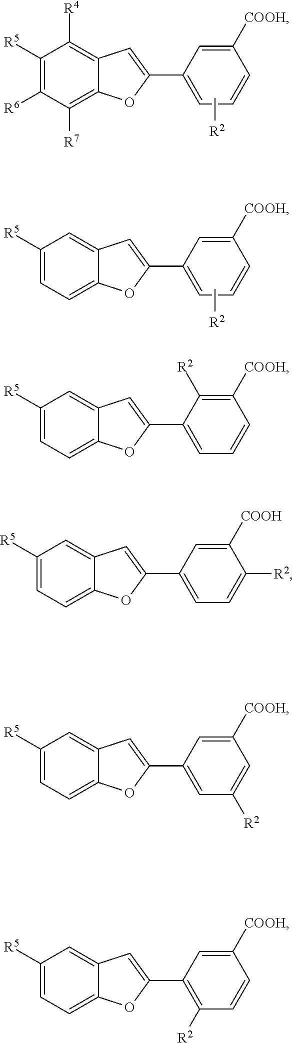

or a pharmaceutically acceptable salt thereof, wherein each variable is independently as described herein. In some embodiments, R² is H. In some embodiments, R² is halogen. In some embodiments, R² is F. In some embodiments, R² is C₁. In some embodiments, R² is Br. In some embodiments, R⁵ is optionally substituted C₁-C₆ alkyl. In some embodiments, R⁵ is —CF₃.

In some embodiments, a compound of Formula (I) or a pharmaceutical acceptable salt thereof has a structure of or a

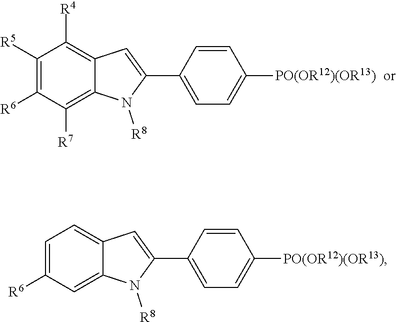

or a pharmaceutically acceptable salt thereof, wherein each variable is independently as described herein. In some embodiments, R⁸ is optionally substituted C₁-C₆ alkyl. In some embodiments, R⁸ is methyl. In some embodiments, R⁶ is optionally substituted C₁-C₆ alkyl. In some embodiments, R⁶ is —CF₃. In some embodiments, each of R¹² and R¹³ is independently H or optionally substituted C₁-C₆ alkyl. In some embodiments, each of R¹² and R¹³ is independently H. In some embodiments, each of R¹² and R¹³ is independently ethyl. In some embodiments, R¹² is H and R¹³ is ethyl.

In some embodiments, a compound of Formula (I) or a pharmaceutical acceptable salt thereof has a structure of

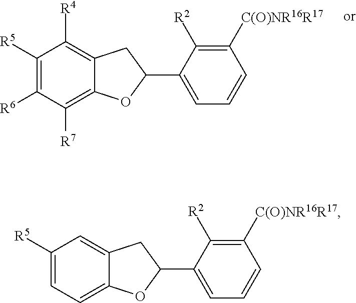

or a pharmaceutically acceptable salt thereof, wherein each variable is independently as described herein. In some embodiments, R² is halogen. In some embodiments, R² is F. In some embodiments, R² is C₁. In some embodiments, R² is Br. In some embodiments, R⁵ is optionally substituted C₁-C₆ alkyl. In some embodiments, R⁵ is —CF₃. In some embodiments, each of R¹⁶ and R¹⁷ is independently H or optionally substituted C₁-C₆ alkyl. In some embodiments, each of R¹⁶ and R¹⁷ is independently optionally substituted C₁-C₆ alkyl. In some embodiments, each of R¹⁶ and R¹⁷ is methyl. In some embodiments, R¹⁶ is H and R¹⁷ is methyl. In some embodiments, R¹⁶ and R¹⁷ are taken together with the nitrogen to form an optionally substituted 3-10 membered ring having, in addition to nitrogen, 0-4 heteroatoms.

In some embodiments, a compound of Formula (I) or a pharmaceutical acceptable salt thereof has a structure of

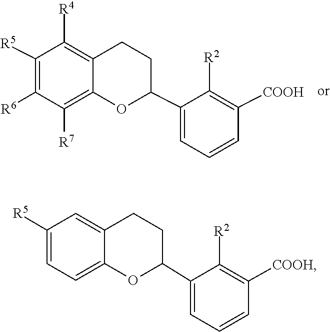

or a pharmaceutically acceptable salt thereof, wherein each variable is independently as described herein. In some embodiments, R² is halogen. In some embodiments, R² is F. In some embodiments, R² is C₁. In some embodiments, R² is Br. In some embodiments, R² is optionally substituted C₁-C₆ alkyl. In some embodiments, R² is —CF₃.

### R′

Various variables can be R′ as described herein. In some embodiments, R¹ is hydrogen.

In some embodiments, R¹ is R as described herein. In some embodiments, R¹ is —OR wherein R is as described herein. In some embodiments, R¹ is —C(O)R wherein R is as described herein. In some embodiments, R¹ is —C(O)OR wherein R is as described herein. In some embodiments, R¹ is —S(O)₂R wherein R is as described herein.

### R

Various variables can be R as described herein. Various embodiments for R are extensively described herein, including in various sections for other variables that can be R (e.g., R¹, R², R′, etc.).

In some embodiments, R is —H. In some embodiments, R is not —H.

In some embodiments, each R is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic.

In some embodiments, R is optionally substituted C₁₋₆ aliphatic. In some embodiments, R is optionally substituted C₁₋₆ alkyl. In some embodiments, R is optionally substituted methyl. In some embodiments, R is optionally substituted ethyl. In some embodiments, R is optionally substituted n-propyl. In some embodiments, R is optionally substituted isopropyl. In some embodiments, R is n-butyl. In some embodiments, R is t-butyl. In some embodiments, R is pentyl. In some embodiments, R is hexyl.

In some embodiments, R is optionally substituted C₁₋₆ heteroaliphatic having 1-3 (e.g., 1, 2, or 3) heteroatoms independently selected from oxygen, nitrogen, sulfur, phosphorus and silicon. In some embodiments, R is optionally substituted C₁₋₆ heteroaliphatic having 1-3 (e.g., 1, 2, or 3) heteroatoms independently selected from nitrogen, oxygen and sulfur. In some embodiments, a heteroatom is nitrogen. In some embodiments, a heteroatom is oxygen. In some embodiments, a heteroatom is sulfur.

In some embodiments, R is optionally substituted C₃₋₁₀ (e.g., C₄₋₁₀, C₃₋₉, C₃₋₇, or 3, 4, 5, 6, 7, 8, 9, or 10-membered)cycloaliphatic. In some embodiments, a cycloaliphatic group is a cycloalkyl group. In some embodiments, a cycloaliphatic group is monocyclic. In some embodiments, it is bicyclic. In some embodiments, it is polycyclic. In some embodiments, each monocyclic unit is independently a 3-10 (e.g., C₄₋₁₀, C₃₋₉, C₃₋₇, or 3, 4, 5, 6, 7, 8, 9, or 10, etc.) membered cycloaliphatic ring. In some embodiments, a cycloaliphatic group is saturated. In some embodiments, it is partially unsaturated. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted cycloheptyl. In some embodiments, R is cyclopropyl. In some embodiments, R is cyclobutyl. In some embodiments, R is cyclopentyl. In some embodiments, R is cyclohexyl. In some embodiments, R is cycloheptyl.

In some embodiments, R is optionally substituted 3-10 (e.g., 3-9, 3-6, 3-5, or 3, 4, 5, 6, 7, 8, 9, or 10, etc.) membered heterocyclyl having 1-4 (e.g., 1, 2, 3, or 4, etc.) heteroatoms independently selected from oxygen, nitrogen, sulfur, phosphorus and silicon. In some embodiments, R is optionally substituted 3-10 (e.g., 3-9, 3-6, 3-5, or 3, 4, 5, 6, 7, 8, 9, or 10, etc.) membered heterocyclyl having 1-4 (e.g., 1, 2, 3, or 4, etc.) heteroatoms independently selected from oxygen, nitrogen and sulfur. In some embodiments, a heterocyclyl group is monocyclic. In some embodiments, it is bicyclic. In some embodiments, it is polycyclic. In some embodiments, each monocyclic unit is independently a 3-10 (e.g., 3, 4, 5, 6, 7, 8, 9, or 10, etc.) membered heterocyclyl ring having 1-4 (e.g., 1, 2, 3, or 4 etc.) heteroatoms independently selected from nitrogen, oxygen and sulfur. In some embodiments, a heterocyclyl group is saturated. In some embodiments, it is partially unsaturated. In some embodiments, a heterocyclyl ring has one heteroatom. In some embodiments, a heterocyclyl ring has two or more heteroatoms. In some embodiments, a heterocyclyl ring has three or more heteroatoms. In some embodiments, a heterocyclyl ring has four or more heteroatoms. In some embodiments, a heteroatom is nitrogen. In some embodiments, a heteroatom is oxygen. In some embodiments, a heteroatom is sulfur.

In some embodiments, R is optionally substituted C₆₋₁₀ (e.g., C₆, C₁₀, etc.) aryl. In some embodiments, R is optionally substituted C6-10 aryl. In some embodiments, an aryl ring is monocyclic. In some embodiments, an aryl ring is bicyclic. In some embodiments, an aryl ring is polycyclic. In some embodiments, each monocyclic unit is independently a 6-membered aromatic ring. In some embodiments, R is optionally substituted phenyl. In some embodiments, R is phenyl. In some embodiments, R is optionally substituted 10-membered aryl. In some embodiments, R is optionally substituted naphthyl. In some embodiments, R is naphthyl.

In some embodiments, R is optionally substituted 5-10 (e.g., 5-9, or 5, 6, 7, 8, 9, or 10 etc.) membered heteroaryl having 1-6 (e.g., 1-6, 1-5, 1-4, or 1, 2, 3, 4, 5, or 6 etc.) heteroatoms independently selected from oxygen, nitrogen, sulfur, phosphorus and silicon. In some embodiments, R is 5-10 (e.g., 5-9, or 5, 6, 9, 10 etc.) membered heteroaryl having 1-4 (e.g., 1, 2, 3, or 4, etc.) heteroatoms independently selected from nitrogen, oxygen and sulfur. In some embodiments, a heteroaryl ring is monocyclic. In some embodiments, a heteroaryl ring is bicyclic. In some embodiments, a heteroaryl ring is polycyclic. In some embodiments, each monocyclic unit is independently a 5- or 6-membered aromatic ring having 0-4 heteroatoms, e.g., independently selected from nitrogen, oxygen and sulfur, wherein at least one monocyclic unit contains 1-4 heteroatoms. In some embodiments, R is optionally substituted 5-membered monocyclic heteroaryl having 1-4 heteroatoms independently selected from nitrogen, oxygen and sulfur. In some embodiments, R is optionally substituted 6-membered monocyclic heteroaryl having 1-4 heteroatoms independently selected from nitrogen, oxygen and sulfur. In some embodiments, R is optionally substituted 9-membered bicyclic heteroaryl having 1-4 heteroatoms independently selected from nitrogen, oxygen and sulfur. In some embodiments, R is optionally substituted 10-membered bicyclic heteroaryl having 1-4 heteroatoms independently selected from nitrogen, oxygen and sulfur. In some embodiments, a heteroaryl ring has one heteroatom. In some embodiments, a heteroaryl ring has two or more heteroatoms. In some embodiments, a heteroaryl ring has three or more heteroatoms. In some embodiments, a heteroaryl ring has four or more heteroatoms. In some embodiments, a heteroatom is nitrogen. In some embodiments, a heteroatom is oxygen. In some embodiments, a heteroatom is sulfur.

In some embodiments, R is optionally substituted C₆₋₁₀ aryl-C₁₋₆ aliphatic, wherein the aryl and aliphatic are independently as described herein. In some embodiments, R is optionally substituted C6-10 aryl-C₁₋₆ alkyl.

In some embodiments, R is optionally substituted 5-10 membered heteroaryl having 1-6 (e.g., 1, 2, 3, 4, 5, or 6) heteroatoms-C₁₋₆ aliphatic wherein the heteroaryl and aliphatic are independently as described herein. In some embodiments, R is optionally substituted 5-10 membered heteroaryl having 1-5 heteroatoms-C₁₋₆ aliphatic. In some embodiments, R is optionally substituted 5-6 membered heteroaryl having 1-4 heteroatoms-C₁₋₆ aliphatic. In some embodiments, R is optionally substituted 5-10 membered heteroaryl having 1-6 (e.g., 1, 2, 3, 4, 5, or 6) heteroatoms-C₁₋₆ alkyl wherein the heteroaryl and aliphatic are independently as described herein. In some embodiments, R is optionally substituted 5-10 membered heteroaryl having 1-5 heteroatoms-C₁₋₆ alkyl. In some embodiments, R is optionally substituted 5-6 membered heteroaryl having 1-4 heteroatoms-C₁₋₆ alkyl. Various suitable heteroaryl and aliphatic groups are as described herein.

In some embodiments, two R groups on the same atom are optionally and independently taken together with the atom to form an optionally substituted 3-10 (e.g., 3, 4, 5, 6, 7, 8, 9, or 10, etc.) membered ring having, in addition to the atom, 0-4 (e.g., 0, 1, 2, 3, or 4) heteroatoms. In some embodiments, two R groups on two atoms are optionally and independently taken together with their intervening atoms to form an optionally substituted 3-10 (e.g., 3, 4, 5, 6, 7, 8, 9, or 10, etc.) membered ring having, in addition to the intervening atoms, 0-4 (e.g., 0, 1, 2, 3, or 4) heteroatoms

As described herein, in various instances, two R groups, or two groups that are or can be R (e.g., R⁴, R⁵, R⁶ R⁷, etc.), can be taken together with their intervening atom(s) to form an optionally substituted ring as described herein. In some embodiments, a formed ring is substituted (in addition to groups attached to the intervening atom(s)). In some embodiments, a formed ring is unsubstituted. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is 9-membered. In some embodiments, a formed ring is 10-membered. In some embodiments, a formed ring is saturated. In some embodiments, a formed ring is partially unsaturated. In some embodiments, a formed ring is aromatic. In some embodiments, a formed ring is monocyclic. In some embodiments, it is bicyclic. In some embodiments, it is polycyclic. In some embodiments, each monocyclic unit is independently a 3-10 (e.g., 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4) heteroatoms. In some embodiments, each monocyclic unit is independently a 3-10 (e.g., 3-10, 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4, etc.) heteroatoms independently selected from nitrogen, oxygen and sulfur. In some embodiments, each monocyclic ring unit is independently 3-7 membered. In some embodiments, each monocyclic ring unit is independently 3-6 membered. In some embodiments, each monocyclic ring unit is independently 5-7 membered. In some embodiments, each monocyclic unit is independently saturated or partially unsaturated. In some embodiments, at least one monocyclic unit is saturated. In some embodiments, at least one monocyclic unit is partially unsaturated. In some embodiments, at least one monocyclic unit is aromatic. In some embodiments, a formed ring has, in addition to the intervening atom(s), 0-4 (e.g., 0, 1, 2, 3, or 4, etc.) heteroatoms independently selected from oxygen, nitrogen, sulfur, phosphorus and silicon. In some embodiments, there are no additional heteroatoms. In some embodiments, there is one additional heteroatom. In some embodiments, there are 2 additional heteroatoms. In some embodiments, there are 3 additional heteroatoms. In some embodiments, there are 4 additional heteroatoms. In some embodiments, there are 5 additional heteroatoms. In some embodiments, there are 6 or more additional heteroatoms. In some embodiments, an additional heteroatom is nitrogen. In some embodiments, an additional heteroatom is oxygen. In some embodiments, an additional heteroatom is sulfur. For example, in some embodiments, R⁴ and R⁵ are taken together with their intervening atoms to form a ring as described herein; in some embodiments, R⁴ and R⁵ are taken together with their intervening atoms to form an optionally substituted phenyl ring; in some embodiments, R⁴ and R⁵ are taken together with their intervening atoms to form an optionally substituted 5- or 6-membered heteroaryl ring having 1-4 (e.g., 1, 2, 3, or 4, etc.) heteroatoms independently selected from nitrogen, oxygen and sulfur.

As described herein, various groups may be optionally substituted. Substituents are routinely utilized in chemistry including in development of various therapeutics. Many substituents can be utilized in accordance with the present disclosure. In some embodiments, an optionally substituted group is unsubstituted. In some embodiments, an optionally substituted group is substituted. Substituents are preferably those that result in the formation of compounds for a desired property, activity, use, etc., as described herein. In some embodiments, compounds are stable for therapeutic use as described herein. The term “stable,” as used herein, refers to compounds that are not substantially altered when subjected to conditions to allow for their production, detection, and, in certain embodiments, their recovery, purification, and use for one or more of the purposes disclosed herein. In some embodiments, a substituent is a hydrocarbon group. In some embodiments, a substituent comprises a heteroatom. In some embodiments, a substituent comprises multiple heteroatoms. In some embodiments, each atom in a substituent is independently selected from hydrogen, carbon, halogen, nitrogen, oxygen, sulfur, phosphorus and silicon. In some embodiments, each atom in a substituent is independently selected from hydrogen, carbon, halogen, nitrogen, oxygen, and sulfur. In some embodiments, each atom in a substituent is independently selected from hydrogen, carbon, fluorine, chlorine, bromine, iodine, nitrogen, oxygen, and sulfur. In some embodiments, the total number of carbon and non-halogen heteroatom(s) in a substituent is about or no more than about 1; in some embodiments, it is no more than about 2; in some embodiments, it is no more than about 3; in some embodiments, it is no more than about 4; in some embodiments, it is no more than about 5; in some embodiments, it is no more than about 6; in some embodiments, it is no more than about 7; in some embodiments, it is no more than about 8; in some embodiments, it is no more than about 9; in some embodiments, it is no more than about 10; in some embodiments, it is no more than about 11; in some embodiments, it is no more than about 12; in some embodiments, it is no more than about 13; in some embodiments, it is no more than about 14; in some embodiments, it is no more than about 15; in some embodiments, it is no more than about 20. In some embodiments, the total number of carbon and non-halogen heteroatom(s) in each substituent is independently no more than about 20. In some embodiments, the total number of carbon and non-halogen heteroatom(s) in each substituent is independently no more than about 15. In some embodiments, the total number of carbon and non-halogen heteroatom(s) in each substituent is independently no more than about 10. In some embodiments, the total number of carbon and non-halogen heteroatom(s) in each substituent is independently no more than about 6. In some embodiments, each optional substituent on a substitutable group (e.g., Ring A, Ring B, R, etc.) is independently halogen, C1-4 alkyl, —OH, —CN, —NO₂, C1-4 haloalkyl (e.g., —CF₃), —OR^{SB}, —N(R^{SB})₂, —C(O)OR^{SB}, —C(O)N(R^{SB})₂, or —S(O)₂N(R^{SB})₂, wherein each R^{SB} is independently —H, C₁₋₄ alkyl or C₁₋₄ haloalkyl, or is phenyl optionally substituted with halogen, C₁₋₄ alkyl, OH, CN, —NO₂, C₁₋₄ haloalkyl (e.g., —CF₃), —OR^{SB}, —N(R^{SB})₂, —C(O)OR^{SB}, —C(O)N(R^{SB})₂, or —S(O)₂N(R^{SB})₂. In some embodiments, each optional substituent on a substitutable group (e.g., Ring A, Ring B, R, etc.) is independently halogen, C₁₋₄ alkyl, C₁₋₄ haloalkyl, or —OH. In some embodiments, each optional substituent on a substitutable group (e.g., Ring A, Ring B, R, etc.) is independently halogen, C₁₋₄ alkyl or C₁₋₄ haloalkyl. In some embodiments, each halogen is —F.

In some embodiments, a provided compound is a compound selected from compounds 1-101 in the Examples or a salt thereof.

In some embodiments, each heteroatom is independently selected from oxygen, nitrogen, sulfur, phosphorus and silicon. In some embodiments, each heteroatom is independently selected from oxygen, nitrogen and sulfur.

In some embodiments, one or more isotopes may be utilized or enriched in compounds of the present disclosure at one or more locations. For example, in some embodiments, deuterium is utilized or enriched at one or more positions. In some embodiments, an enrichment is about or at least about 5%, 10%, 15%, 20%, 25%, 30%, 35%, 40%, 45%, 50%, 60%, 70%, 80%, 85%, 90%, 95%, or 99% more than a natural abundance as applicable. In some embodiments, a level of an isotope at a position is about or at least about 5%, 10%, 15%, 20%, 25%, 30%, 35%, 40%, 45%, 50%, 60%, 70%, 80%, 85%, 90%, 95%, or 99% of all compound molecules. For example, in some embodiments, L^{ra} is -(CD₂)ₙ-; in some embodiments, L^{ra} is -CD₂-.

### Methods of Preparing

In some embodiments, the present disclosure provides various technologies, e.g., reagents, intermediates, conditions, etc. for preparing compounds and compositions as described herein. Those skilled in the art appreciate that many technologies are available and can be utilized in accordance with the present disclosure.

As appreciated by those skilled in the art, in chemical reactions various groups, e.g., hydroxyl, amino, carboxyl, etc. may be protected to avoid undesired reactions. Many technologies for protection/deprotection are available to those skilled in the art and may be utilized in accordance with the present disclosure. Certain such technologies are described herein including exemplified in the Examples.

Various chemical reactions are typically performed in a solvent. In some embodiments, a reaction is performed in a single solvent, e.g., DCM, THF, Et₂O, EtOH, toluene, etc. In some embodiments, a reaction is performed in a mixture of two or more solvents. In some embodiments, a solvent is polar. In some embodiments, a solvent is non-polar. In some embodiments, a solvent is protic. In some embodiments, a solvent is non-protic. In some embodiments, a solvent is polar but is not protic. Suitable solvents for various reactions are available to those skilled in the art and can be utilized in accordance with the present disclosure.

In some embodiments, a reaction is conducted under an inert atmosphere, e.g., N2, Ar, etc. In some embodiments, a reaction is conducted with exposure to air. In some embodiments, a reaction is conducted under anhydrous conditions, e.g., with reagents, solvents, vessels, etc., properly dried. In some embodiments, a reaction is conducted in the presence of significant of water (e.g., about or more than about 0.1, 0.5, or 1 equivalent).

In some embodiments, reactions are performed, or are performed for periods of time, at temperatures that are higher or lower than or about a standard ambient temperature (25° C.). In some embodiments, a reaction temperature is lower than a standard ambient temperature. In some embodiments, a temperature is about or no more than about −78, −60, −50, −40, −30, −20, −10, 0 or 10° C. In some embodiments, a temperature is about or no more than about 10° C. In some embodiments, a temperature is about or no more than about 15° C. In some embodiments, a temperature is about or no more than about 20° C. In some embodiments, a reaction temperature is about a standard ambient temperature. In some embodiments, a reaction temperature is higher than a standard ambient temperature. In some embodiments, a reaction temperature is about or at least about 35, 40, 50, 60, 70, 80, 90, 100, or 100° C. In some embodiments, a reaction comprises refluxing in a boiling solvent system, e.g., in ether, toluene, etc. In some embodiments, temperature changes during a reaction process, e.g., increasing from a lower temperature to a higher temperature, decreasing from a higher temperature to a lower temperature, or both.

In some embodiments, a product is selectively produced over another potential product. In some embodiments, a product is produced with chemoselectivity, stereoselectivity and/or regioselectivity. In some embodiments, a selectivity is presented as a ratio, e.g., of one product over another. In some embodiments, a ratio is about or at least about 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 40:1, 50:1, 60:1, 70:1, 80:1, 90:1, 100:1, 200:1, 500:1 or more.

Reactions may be performed for a variety of time lengths. In some embodiments, reactions complete instantly. In some embodiments, reaction times varies from minutes to hours to days, e.g., 5, 10, 15, 20, 30, 45 minutes, or 1, 1.5, 2, 3, 4, 5, 6, 7, 8, 9, 10, 12, 15, 18, 20 or 22 hours, or one or two days or longer. Those skilled in the art can use various technologies to determine when to terminate reactions, e.g., based on consumption of starting materials, products formation, by-products formation, etc.

In some embodiments, the present disclosure provides compounds of high purity. In some embodiments, purity of a compound is or greater than about 85%, 90%, 91%, 92%, 93%, 94%, 95%, 96%, 97%, 98%, 99%, 99.7%, or 99.9%. In some embodiments, purity of a compound is or greater than about 80%. In some embodiments, purity of a compound is or greater than about 85%. In some embodiments, purity of a compound is or greater than about 90%. In some embodiments, purity of a compound is or greater than about 95%. In some embodiments, purity of a compound is or greater than about 96%. In some embodiments, purity of a compound is or greater than about 97%. In some embodiments, purity of a compound is or greater than about 98%. In some embodiments, purity of a compound is or greater than about 99%. In some embodiments, purity of a compound is or greater than about 99.5%. In some embodiments, purity of a compound is or greater than about 99.7%. In some embodiments, purity of a compound is or greater than about 99.9%.

In some embodiments, the present disclosure provides compounds of high stereochemical purity. In some embodiments, stereochemical purity of a compound is or greater than about 85%, 90%, 91%, 92%, 93%, 94%, 95%, 96%, 97%, 98%, 99%, 99.7%, or 99.9%. In some embodiments, stereochemical purity of a compound is or greater than about 80%. In some embodiments, stereochemical purity of a compound is or greater than about 85%. In some embodiments, stereochemical purity of a compound is or greater than about 90%. In some embodiments, stereochemical purity of a compound is or greater than about 95%. In some embodiments, stereochemical purity of a compound is or greater than about 96%. In some embodiments, stereochemical purity of a compound is or greater than about 97%. In some embodiments, stereochemical purity of a compound is or greater than about 98%. In some embodiments, stereochemical purity of a compound is or greater than about 99%. In some embodiments, stereochemical purity of a compound is or greater than about 99.5%. In some embodiments, stereochemical purity of a compound is or greater than about 99.7%. In some embodiments, stereochemical purity of a compound is or greater than about 99.9%.

In some embodiments, the present disclosure provides compounds of high enantiomeric purity. In some embodiments, enantiomeric purity of a compound is or greater than about 85%, 90%, 91%, 92%, 93%, 94%, 95%, 96%, 97%, 98%, 99%, 99.7%, or 99.9%. In some embodiments, enantiomeric purity of a compound is or greater than about 80%. In some embodiments, enantiomeric purity of a compound is or greater than about 85%. In some embodiments, enantiomeric purity of a compound is or greater than about 90%. In some embodiments, enantiomeric purity of a compound is or greater than about 95%. In some embodiments, enantiomeric purity of a compound is or greater than about 96%. In some embodiments, enantiomeric purity of a compound is or greater than about 97%. In some embodiments, enantiomeric purity of a compound is or greater than about 98%. In some embodiments, enantiomeric purity of a compound is or greater than about 99%. In some embodiments, enantiomeric purity of a compound is or greater than about 99.5%. In some embodiments, enantiomeric purity of a compound is or greater than about 99.7%. In some embodiments, enantiomeric purity of a compound is or greater than about 99.9%.

Stereochemically pure, e.g., enantiomerically pure, compounds and compositions can be prepared utilizing various technologies in accordance with the present disclosure. For example, in some embodiments, they can be prepared through separation including chiral separation; in some embodiments, they can be prepared through stereoselective synthesis.

For example, in some embodiments, the present disclosure provides a method, comprising:
reacting a compound of formula B-4

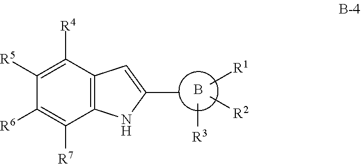

or a salt thereof with a compound having the structure of R⁸-LG or a salt thereof, wherein LG is a leaving group to provide a compound having the structure of formula B:

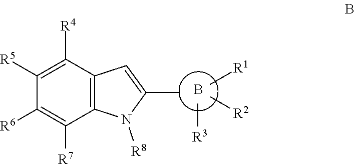

or a salt thereof, wherein each variable is independently as described herein.

Various leaving groups may be utilized in accordance with the present disclosure. For example, in some embodiments, a leaving group is a halogen. In some embodiments, LG is Cl. In some embodiments, LG is Br. In some embodiments, LG is I. In some embodiments, LG is —S(O)₂R wherein R is as described herein and is not H. In some embodiments, R is optionally substituted C₁₋₆ aliphatic. In some embodiments, R is optionally substituted phenyl. Many suitable conditions may be utilized in accordance with the present disclosure. For example, in some embodiments, a condition is an alkylation condition. In some embodiments, a reaction is performed in the presence of a base. In some embodiments, a base is NaH.

In some embodiments, a compound having the structure of formula B-4 or a salt thereof is a compound having the structure of

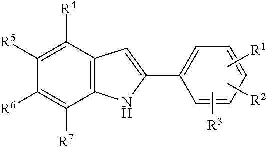

or a salt thereof, wherein each variable is independently as described herein. In some embodiments, a compound having the structure of formula B or a salt thereof is a compound having the structure of

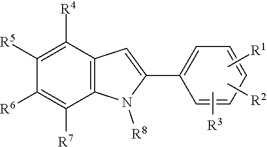

or a salt thereof, wherein each variable is independently as described herein.

In some embodiments, the present disclosure provides a method, comprising:
reacting a compound having the structure of formula B-3:

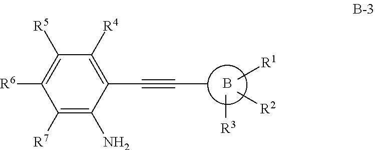

or a salt thereof to provide a compound having the structure of formula B-4:

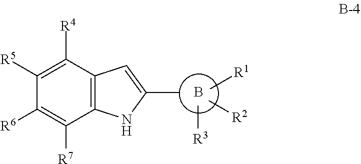

or a salt thereof, wherein each variable is independently as described herein.

In some embodiments, a compound having the structure of B-3 or a salt thereof is a compound having the structure of

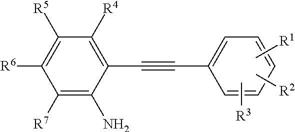

or a salt thereof, wherein each variable is independently as described herein. In some embodiments, a compound having the structure of B-4 or a salt thereof is a compound having the structure of

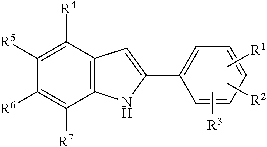

or a salt thereof, wherein each variable is independently as described herein. In some embodiments, a reaction is performed in the presence of a metal. In some embodiments, a metal is in a metal complex. In some embodiments, a metal complex is a Pd complex. In some embodiments, a metal complex is PdCl₂. In some embodiments, a suitable solvent is CH₃CN.

In some embodiments, the present disclosure provides a method, comprising:
reacting a compound having the structure of formula B-1:

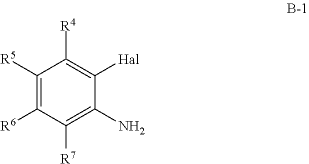

or a salt thereof and a compound having the structure of formula B-2:

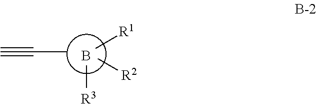

or a salt thereof, to provide a compound having the structure of formula B-3:

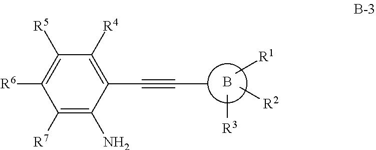

or a salt thereof, wherein Hal is halogen, and each variable is independently as described herein.

In some embodiments, Hal is Cl. In some embodiments, Hal is Br. In some embodiments, Hal is I. In some embodiments, a compound having the structure of B-3 or a salt thereof is a compound having the structure of

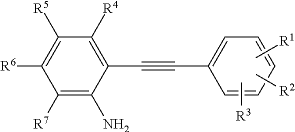

or a salt thereof, wherein each variable is independently as described herein. In some embodiments, a compound having the structure of B-2 or a salt thereof is a compound having the structure of

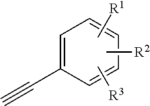

or a salt thereof, wherein each variable is independently as described herein. In some embodiments, a reaction is performed in the presence of a metal. In some embodiments, a metal is in a metal complex. In some embodiments, a metal is Pd. In some embodiments, Pd is in a complex. In some embodiments, a Pd complex is PdCl₂(PPh₃)₂. In some embodiments, a metal is Cu. In some embodiments, a metal is Cu(I). In some embodiments, a Cu(I) complex is CuI. In some embodiments, a reaction is performed in the presence of Pd and Cu. In some embodiments, a reaction is performed in the presence of Pd and Cu(I). In some embodiments, a reaction is performed before a base. In some embodiments, a base is N(R)₃ wherein each R is independently as described herein. In some embodiments, a base is NEt₃.

In some embodiments, a method is described in Scheme 1 as an example:

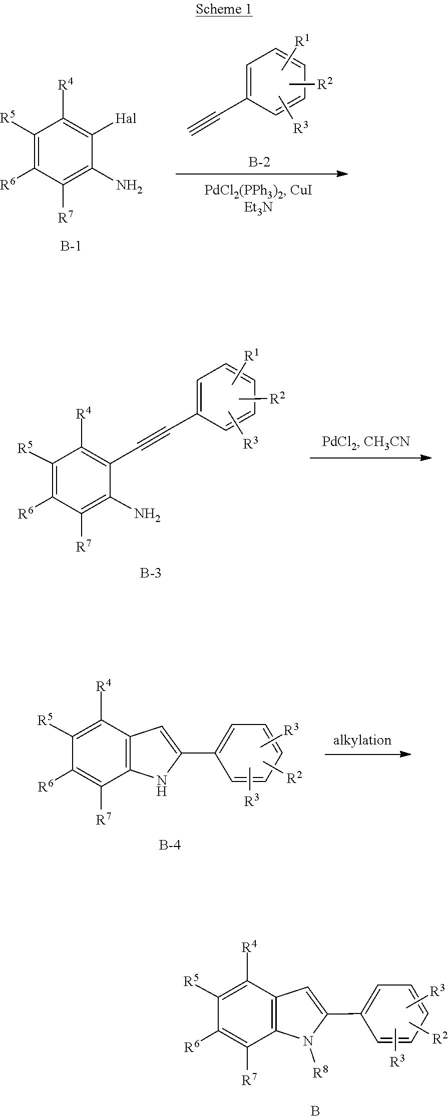

A method for preparing provided compounds, e.g., compounds of formula I or salts thereof such as compounds of formula B or salts thereof, is illustrated in Scheme 1 as an example, wherein each variable is independently as described herein. In some embodiments, treatment of aniline compound of formula B-1 or a salt thereof, wherein Hal is Cl, Br, or I, and alkyne compound of formula B-2 or a salt thereof under Sonogashira coupling conditions such as PdCl₂ (PPh₃)₂Cl₂/CuI/Et₃N provides alkyne compound of formula B-3 or a salt thereof. In some embodiments, cyclization of a compound of formula B-3 or a salt thereof using transition metal catalysts such as PdCl₂ affords indole compound of formula B-4 or a salt thereof. In some embodiments, treatment of a compound of formula B-4 or a salt thereof under alkylation conditions such as NaH/R⁸-Hal furnishes compound of formula I or a salt thereof, e.g., a compound of formula B or a salt thereof.

In some embodiments, the present disclosure provides a method, comprising:
reacting a compound having the structure of formula B-5:

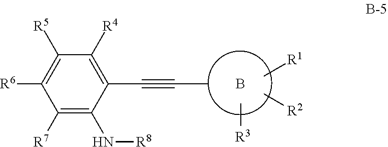

or a salt thereof to provide a compound having the structure of formula B:

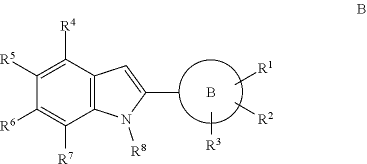

or a salt thereof, wherein each variable is independently as described herein.

In some embodiments, a compound having the structure of B-5 or a salt thereof is a compound having the structure of

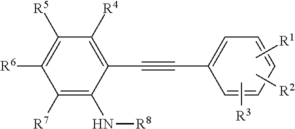

or a salt thereof, wherein each variable is independently as described herein. In some embodiments, a compound having the structure of B or a salt thereof is a compound having the structure of

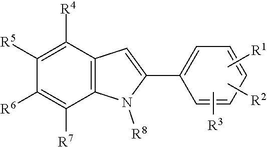

or a salt thereof, wherein each variable is independently as described herein. In some embodiments, a reaction is performed in the presence of a metal. In some embodiments, a metal is in a metal complex. In some embodiments, a metal complex is a Pd complex. In some embodiments, a metal complex is PdCl₂. In some embodiments, a suitable solvent is CH₃CN.

In some embodiments, the present disclosure provides a method, comprising:
reacting a compound having the structure of formula B-3:

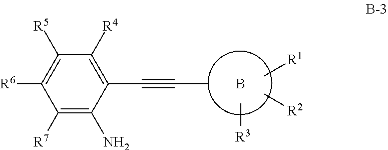

or a salt thereof to provide a compound having the structure of formula B-5:

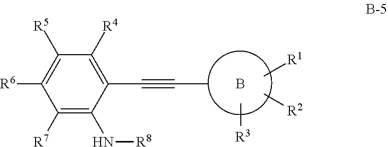

or a salt thereof wherein each variable is independently as described herein.

In some embodiments, a compound having the structure of B-5 or a salt thereof is a compound having the structure of

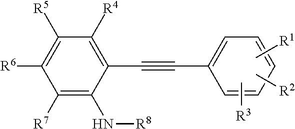

or a salt thereof, wherein each variable is independently as described herein. In some embodiments, a compound having the structure of B-3 or a salt thereof is a compound having the structure of

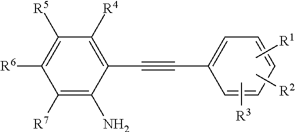

or a salt thereof, wherein each variable is independently as described herein.

In some embodiments, a method comprises reacting a compound of formula B-3 or a salt thereof with a compound having the structure of R⁸-LG or a salt thereof, wherein LG is a leaving group. Various leaving groups may be utilized in accordance with the present disclosure. For example, in some embodiments, a leaving group is a halogen. In some embodiments, LG is Cl. In some embodiments, LG is Br. In some embodiments, LG is I. In some embodiments, LG is —S(O)₂R wherein R is as described herein and is not H. In some embodiments, R is optionally substituted C₁₋₆ aliphatic. In some embodiments, R is optionally substituted phenyl. Many suitable conditions may be utilized in accordance with the present disclosure. For example, in some embodiments, a condition is an alkylation condition. In some embodiments, a reaction is performed in the presence of a base. In some embodiments, a base is NaH.

In some embodiments, a method comprises reacting a compound of formula B-3 or a salt thereof with a compound having the structure of R⁸′—CHO or a salt thereof, wherein R⁸ is bonded to the —NH-through-CH₂—, and R⁸′ is of such a structure that R⁸′—CH₂— is R⁸. For example, in some embodiments, R⁸ is —CH₂CH₃, and R⁸′ is —CH₃. In some embodiments, a reaction is a reductive amination reaction. In some embodiments, a reaction is performed in the presence of a reducing agent. In some embodiments, a reducing agent is a boron hydride agent, e.g., NaBH₃CN. In some embodiments, a reaction is performed in the presence of a protic solvent, e.g., an alcohol such as methanol.

In some embodiments, a method is described in Scheme 2 as an example:

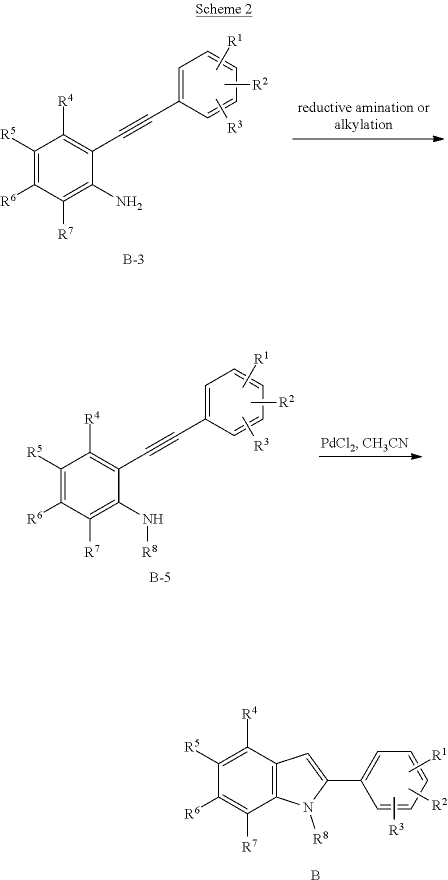

A method for preparing provided compounds, e.g., compounds of formula I or salts thereof such as compounds of formula B or salts thereof, is illustrated in Scheme 2 as an example, wherein each variable is independently as described herein. In some embodiments, treatment of a compound of formula B-3 or a salt thereof, wherein each variable is independently as described herein, under a reductive amination condition such as NaBH₃CN/MeOH or a alkylation condition such as NaH/R⁸-Hal affords a compound of formula B-5 or a salt thereof (Scheme 2). in some embodiments, cyclization of a compound of formula B-5 or a salt thereof using transition metal catalysts such as PdCl₂ affords a compound having the structure of formula I or a salt thereof, e.g., an indole compound of formula B or a salt thereof.

In some embodiments, a method comprises reacting a compound having the structure of formula B-3 or a salt thereof with a compound having the structure of R⁸—B(OH)₂ or a salt thereof to provide a compound having the structure of formula B or a salt thereof.

In some embodiments, a method is described in Scheme 3 as an example:

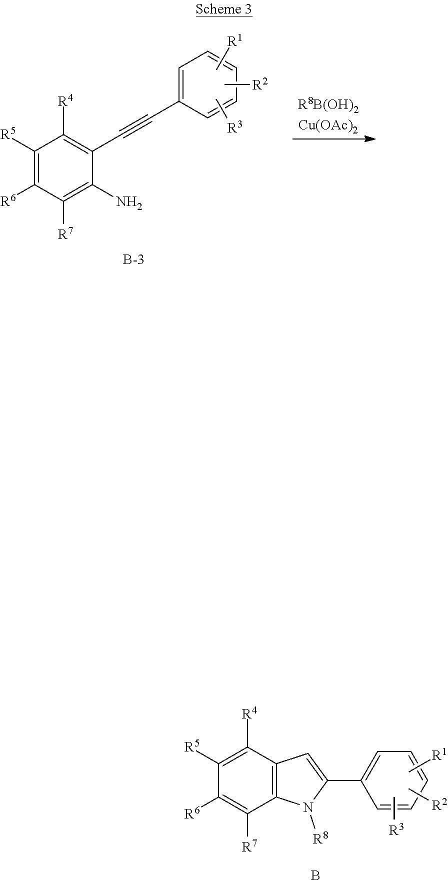

A method for preparing provided compounds, e.g., compounds of formula I or salts thereof such as compounds of formula B or salts thereof, is illustrated in Scheme 3 as an example, wherein each variable is independently as described herein. In some embodiments, treatment of a compound of formula B-3 or a salt thereof under Chan-Lam coupling conditions such as R⁸B(OH)₂/Cu (OAc)₂ provides a compound of formula I or a salt thereof, e.g., a compound having the structure of formula B or a salt thereof, in one step (Scheme 3).

In some embodiments, the present disclosure provides a method, comprising converting a first compound of formula I or a salt thereof wherein R¹ is —C(O)OR¹¹, wherein R¹¹ is not —H (e.g., optionally substituted C₁₋₆ aliphatic) to a second compound of formula I or a salt thereof, wherein R¹ is —C(O)OH. In some embodiments, a first compound of formula I or a salt thereof has the structure of

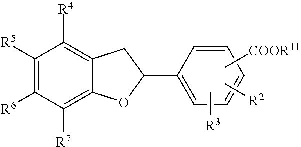

or a salt thereof, wherein each variable is independently as described herein. In some embodiments, a second compound of formula I or a salt thereof has the structure of

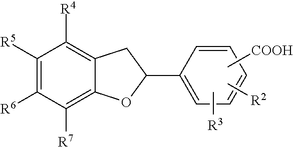

or a salt thereof, wherein each variable is independently as described herein. In some embodiments, a reaction is performed in the presence of a base. In some embodiments, a base is LiOH. In some embodiments, a reaction is performed in the presence of water, e.g., in THF/H₂O.

In some embodiments, the present disclosure provides a method, comprising:
reacting a compound having the structure of formula C-4:

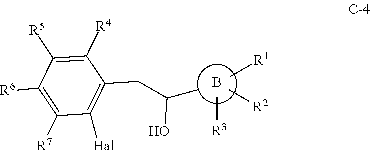

or a salt thereof to provide a compound having the structure of formula C-5:

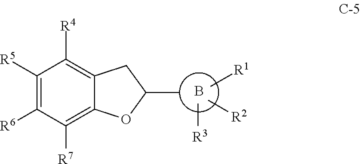

or a salt thereof, wherein Hal is a halogen, and each other variable is independently as described herein.

In some embodiments, a compound having the structure of formula C-4 or a salt of has the structure of

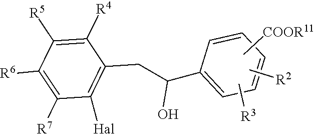

or a salt thereof, wherein each variable is independently as described herein. In some embodiments, a compound having the structure of formula C-5 or a salt thereof has the structure

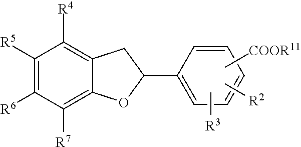

of or a salt thereof, wherein each variable is independently as described herein. In some embodiments, R¹¹ is not —H. In some embodiments, R¹ is optionally substituted C₁₋₆ aliphatic. In some embodiments, Hal is Cl. In some embodiments, Hal is Br. In some embodiments, Hal is I.

In some embodiments, a reaction is performed in the presence of a metal. In some embodiments, a metal is in a metal complex. In some embodiments, a metal is Pd. In some embodiments, Pd is in a complex. In some embodiments, a Pd complex is Pd(OAc)₂. In some embodiments, a reaction is performed in the presence of a phosphine compound, e.g., having the structure of formula P(R)₃ or a salt thereof wherein each R is independently as described herein and is not H. In some embodiments, a compound is

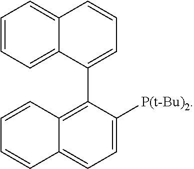

In some embodiments, a metal is Cu. In some embodiments, a metal is Cu(I). In some embodiments, a Cu(I) complex is CuCl. In some embodiments, a reaction is performed in the presence of Pd and Cu. In some embodiments, a reaction is performed in the presence of Pd and Cu(I). In some embodiments, a reaction is performed before a base. In some embodiments, a base is Cs₂CO₃. In some embodiments, a base is NaH.

In some embodiments, the present disclosure provides a method, comprising:
reacting a compound having the structure of formula C-2:

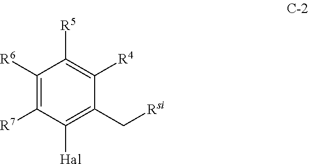

or a salt thereof with a compound having the structure of formula C-3:

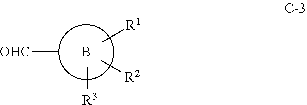

to provide a compound having the structure of formula C-4:

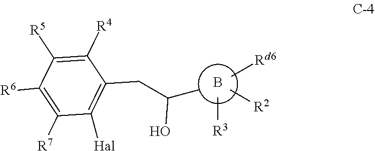

or a salt thereof, wherein Hal is a halogen, R^{si} is —Si(R)₃, and each variable is independently as described herein.

As described herein, in some embodiments, Hal is Cl; in some embodiments, Hal is Br; and in some embodiments, Hal is I. In some embodiments, R^{si} is —Si(R)₃ wherein each R is independently as described herein and is not —H. In some embodiments, each R is independently an optionally substituted group selected from C₁₋₆ aliphatic and C₆₋₁₀ aryl. In some embodiments, each R is independently an optionally substituted group selected from C₁₋₆ aliphatic and phenyl. In some embodiments, a compound having the structure of formula C-4 or a salt thereof has the structure of

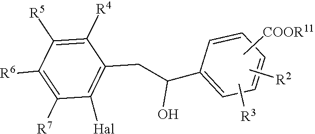

or a salt thereof, wherein each variable is independently as described herein. In some embodiments, a compound having the structure of formula C-3 or a salt thereof has the structure of

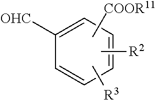

or a salt thereof, wherein each variable is independently as described herein. In some embodiments, a compound having the structure of C-2 or salt thereof has the structure of

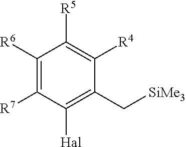

or a salt thereof, wherein each variable is independently as described herein. In some embodiments, a reaction is performed under a desilylation condition. In some embodiments, a reaction is performed in the presence of a fluoride agent. In some embodiments, a fluoride agent is TBAF.

In some embodiments, the present disclosure provides a method, comprising:
reacting a compound having the structure of formula C-1:

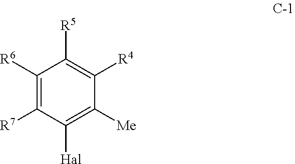

or a salt thereof to provide a compound having the structure of formula C-2:

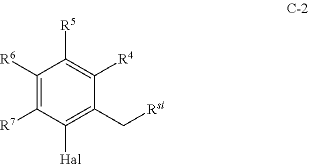

or a salt thereof, wherein Hal is a halogen, R^{si} is —Si(R)₃, and each variable is independently as described herein.

As described herein, in some embodiments, Hal is Cl; in some embodiments, Hal is Br; and in some embodiments, Hal is I. In some embodiments, R^{si} is —Si(R)₃ wherein each R is independently as described herein and is not —H. In some embodiments, each R is independently an optionally substituted group selected from C₁₋₆ aliphatic and C₆₋₁₀ aryl. In some embodiments, each R is independently an optionally substituted group selected from C₁₋₆ aliphatic and phenyl. In some embodiments, a compound having the structure of C-2 or salt thereof has the structure of

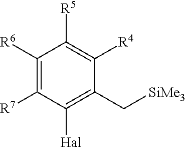

or a salt thereof, wherein each variable is independently as described herein. In some embodiments, a reaction is performed in the presence of a base. In some embodiments, a base is LDA. In some embodiments, a reaction is performed in the presence of a silylating agent, e.g. a compound having the structure of formula R^{si}-LG or a salt thereof, wherein LG is a leaving group, e.g., Cl, OTf, etc. In some embodiments, a reaction is performed at a reduced temperature, e.g., −100° C.

In some embodiments, a method is described in Scheme 4 as an example:

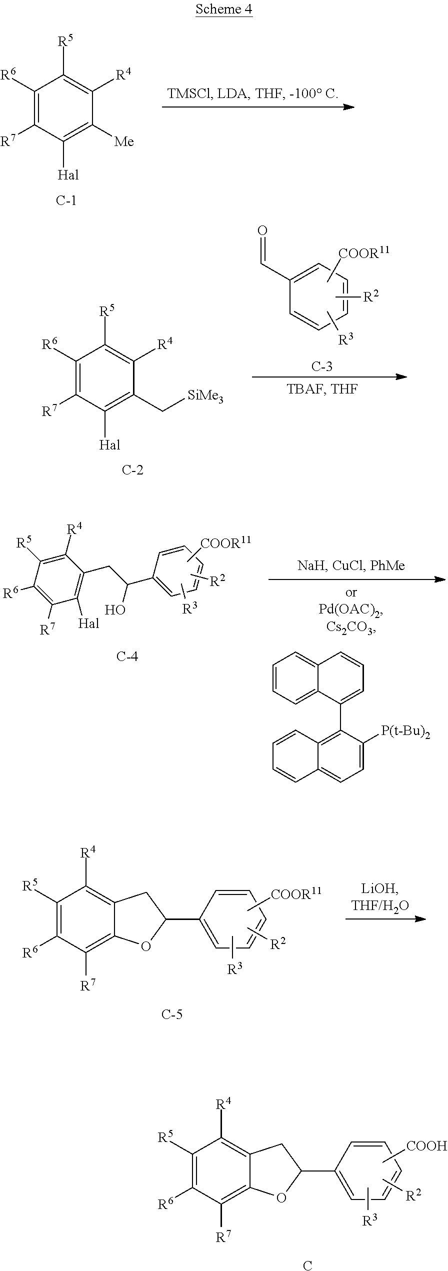

A method for preparing provided compounds, e.g., compounds of formula I or salts thereof such as compounds of formula C or salts thereof, is illustrated in Scheme 4 as an example, wherein each variable is independently as described herein. In some embodiments, silylation of a compound having the structure of formula C-1 or a salt thereof under conditions such as LDA/TMSCl/THF provides a compound having the structure of formula C-2 or a salt thereof. In some embodiments, treatment of a compound having the structure of formula C-2 or a salt thereof and an aldehyde compound having the structure of formula C-3 or a salt thereof under desilylation conditions such as TBAF/THF provides an alcohol compound having the structure of formula C-4 or a salt thereof. In some embodiments, cyclization of a compound having the structure of C-4 or a salt thereof under transition metal catalyzed intramolecular C—O coupling conditions such as NaH/CuCl/PhMe or Pd(OAc)₂/TrixiePhos/Cs₂CO₃ affords a compound having the structure of formula C-5 or a salt thereof. Subjection of a compound having the structure of formula C-5 or a salt thereof to hydrolysis conditions such as LiOH/THF/H₂O furnishes a compound having the structure of formula I or a salt thereof, e.g., a compound having the structure of formula C or a salt thereof.

In some embodiments, the present disclosure provides a method, comprising:
reacting a compound having the structure of formula D-7:

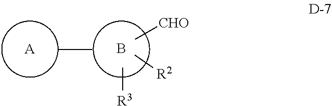

or a salt thereof to provide a compound having the structure of formula I or a salt thereof, wherein each variable is independently as described herein.

In some embodiments, a compound having the structure of formula D-7 or a salt thereof has the structure of

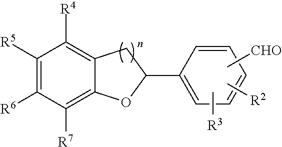

or a salt thereof, wherein each variable is independently as described herein. In some embodiments, a compound having the structure of formula I or a salt thereof has the structure of

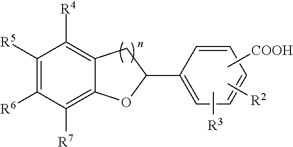

or a salt thereof, wherein each variable is independently as described herein. In some embodiments, a reaction is performed under an oxidation condition (e.g., a Pinnick oxidation condition). Various technologies for converting an aldehyde to a carboxylic acid may be utilized in accordance with the present disclosure.

In some embodiments, the present disclosure provides a method, comprising:
reacting a compound having the structure of formula D-6:

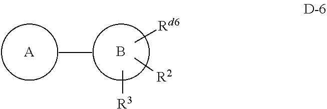

or a salt thereof to provide a compound having the structure of formula D-7:

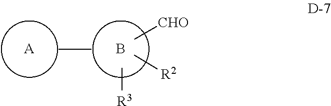

or a salt thereof, wherein R^{d6} is —CH(OR)₂, and each variable is independently as described herein.

In some embodiments, a compound having the structure of formula D-6 or a salt thereof has the structure of

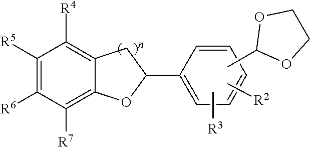

or a salt thereof, wherein each variable is independently as described herein. In some embodiments, a compound having the structure of formula D-7 or a salt thereof has the structure of

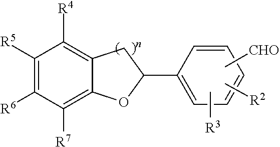

or a salt thereof, wherein each variable is independently as described herein.

In some embodiments, R^{d6} is —CH(OR)₂ wherein each R is independently as described herein and is not —H. In some embodiments, each R is independently C₁₋₆ aliphatic. In some embodiments, the two R are taken together with their intervening atoms to form an optionally substituted 4-10, e.g., 5-10, 5-6, 4, 5, 6, 7, 8, 9, or 10 membered ring having 0-3 heteroatoms in addition to the intervening atoms. In some embodiments, there are no heteroatoms in addition to the intervening atoms. In some embodiments, a ring is 4-membered. In some embodiments, a ring is 5-membered. In some embodiments, a ring is 6-membered. In some embodiments, a ring is substituted. In some embodiments, a ring is unsubstituted. In some embodiments, a ring is saturated. In some embodiments, a ring is monocyclic. In some embodiments, R^{d6} is optionally substituted

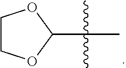 [SMILES: *C1OCCO1]

In some embodiments, R^{d6} is

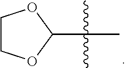 [SMILES: *C1OCCO1]

Various technologies can be utilized to convert a compound having the structure of formula D-6 or a salt thereof into a compound having the structure of formula D-7 or a salt thereof and can be utilized in accordance with the present disclosure. In some embodiments, a useful condition is an acidic condition. In some embodiments, a reaction is performed in the presence of an acid.

In some embodiments, the present disclosure provides a method, comprising:
reacting a compound having the structure of formula D-5:

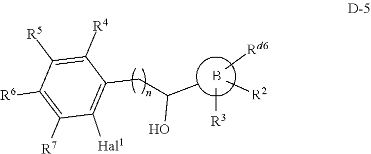

or a salt thereof to provide a compound having the structure of formula D-6:

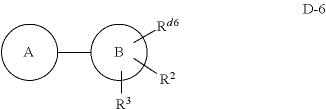

or a salt thereof, wherein Hall is Hal as described herein, and each other variable is independently as described herein.

In some embodiments, a compound having the structure of formula D-5 or a salt of has the structure of

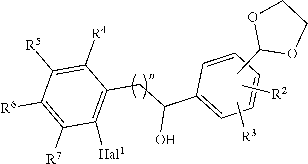

or a salt thereof, wherein each variable is independently as described herein. In some embodiments, a compound having the structure of formula D-6 or a salt thereof has the structure of

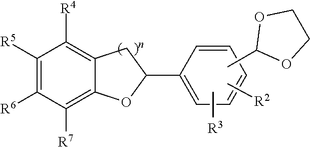

or a salt thereof, wherein each variable is independently as described herein. In some embodiments, Hal¹ is Cl. In some embodiments, Hal¹ is Br. In some embodiments, Hall is I.

In some embodiments, a reaction is performed in the presence of a metal. In some embodiments, a metal is in a metal complex. In some embodiments, a metal is Pd. In some embodiments, Pd is in a complex. In some embodiments, a Pd complex is Pd(OAc)₂. In some embodiments, a reaction is performed in the presence of a phosphine compound, e.g., having the structure of formula P(R)₃ or a salt thereof wherein each R is independently as described herein and is not H. In some embodiments, a compound is

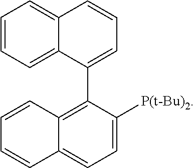

In some embodiments, a metal is Cu. In some embodiments, a metal is Cu(I). In some embodiments, a Cu(I) complex is CuCl. In some embodiments, a reaction is performed in the presence of Pd and Cu. In some embodiments, a reaction is performed in the presence of Pd and Cu(I). In some embodiments, a reaction is performed before a base. In some embodiments, a base is Cs₂CO₃. In some embodiments, a base is NaH.

In some embodiments, the present disclosure provides a method, comprising:
reacting a compound having the structure of formula D-4:

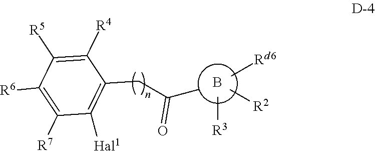

or a salt thereof to provide a compound having the structure of formula D-5:

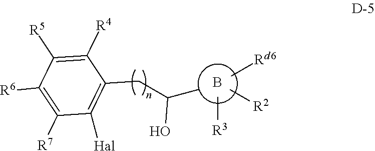

or a salt thereof, wherein each variable is independently as described herein.

In some embodiments, a compound having the structure of formula D-5 or a salt of has the structure of

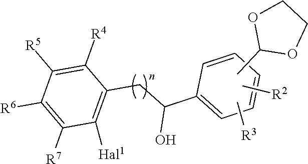

or a salt thereof, wherein each variable is independently as described herein. In some embodiments, a compound having the structure of formula D-4 or a salt thereof has the structure of

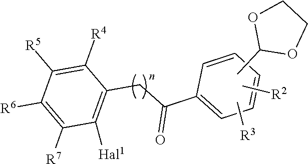

or a salt thereof, wherein each variable is independently as described herein.

In some embodiments, a useful reaction condition is a reduction condition. Various technologies for reducing a ketone to an alcohol can be utilized in accordance with the present disclosure. In some embodiments, a reaction is performed in the presence of a reducing agent. In some embodiments, a reducing agent is a boron hydride. In some embodiments, a reducing agent is NaBH₄.

In some embodiments, the present disclosure provides a method, comprising:
reacting a compound having the structure of formula D-2:

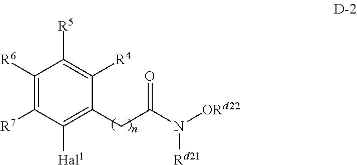

or a salt thereof with a compound having the structure of formula D-3:

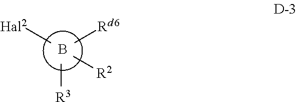

or a salt thereof to provide a compound having the structure of formula D-4:

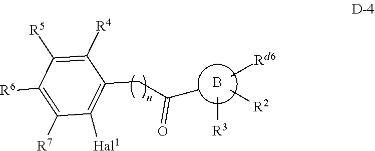

or a salt thereof, wherein each Hal² is Hal as described herein, each of Rd²¹ and Rd²² is independently R as described herein, and each other variable is independently as described herein.

In some embodiments, Rd²¹ is optionally substituted C₁₋₆ aliphatic. In some embodiments, Rd²¹ is optionally substituted C₁₋₆ alkyl. In some embodiments, R⁴²¹ is methyl. In some embodiments, R⁴²² is optionally substituted C₁₋₆ aliphatic. In some embodiments, R⁴²² is optionally substituted C₁₋₆ alkyl. In some embodiments, Rd²² is methyl.

In some embodiments, a compound having the structure of formula D-2 or a salt thereof has the structure of

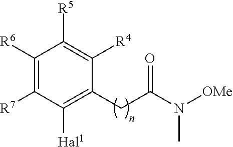

or a salt thereof, wherein each variable is independently as described herein. In some embodiments, a compound having the structure of formula D-3 or a salt thereof has the structure of

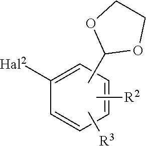

or a salt thereof, wherein each variable is independently as described herein. In some embodiments, a compound having the structure of formula D-4 or a salt thereof has the structure of

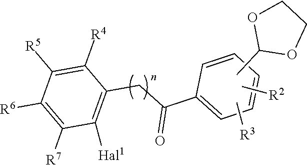

or a salt thereof, wherein each variable is independently as described herein.

In some embodiments, Hal² is Cl. In some embodiments, Hal² is Br. In some embodiments, Hal² is I.

In some embodiments, a reaction is performed in the presence of an organometallic agent. In some embodiments, an agent is a Li agent, e.g., n-BuLi. In some embodiments, an agent is a Mg agent, e.g., i-PrMgBr. In some embodiments, a compound having the structure of formula D-3 or a salt thereof is contacted with an organometallic agent, and the resulting agent is contacted with a compound having the structure of formula D-2 or a salt thereof.

In some embodiments, the present disclosure provides a method, comprising:
reacting a compound having the structure of formula D-1:

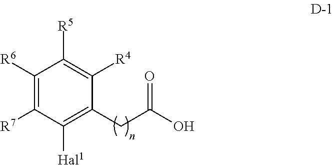

or a salt thereof with a compound having the structure of formula NH(R⁴²¹) OR^{d22} or a salt thereof to provide a compound having the structure of formula D-2:

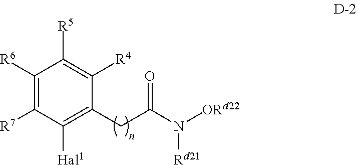

or a salt thereof, wherein each variable is independently as described herein.

In some embodiments, a compound having the structure of formula D-2 or a salt thereof has the structure of

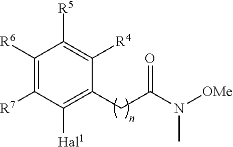

or a salt thereof, wherein each variable is independently as described herein. In some embodiments, a compound having the structure of formula NH(R^{d21}) OR^{d22} or a salt thereof is MeNHOMe or a salt thereof, e.g., MeNHOMe-HCl.

In some embodiments, a reaction is performed under a coupling condition. Various coupling technologies are available and can be utilized in accordance with the present disclosure.

In some embodiments, a method is described in Scheme 5 as an example:

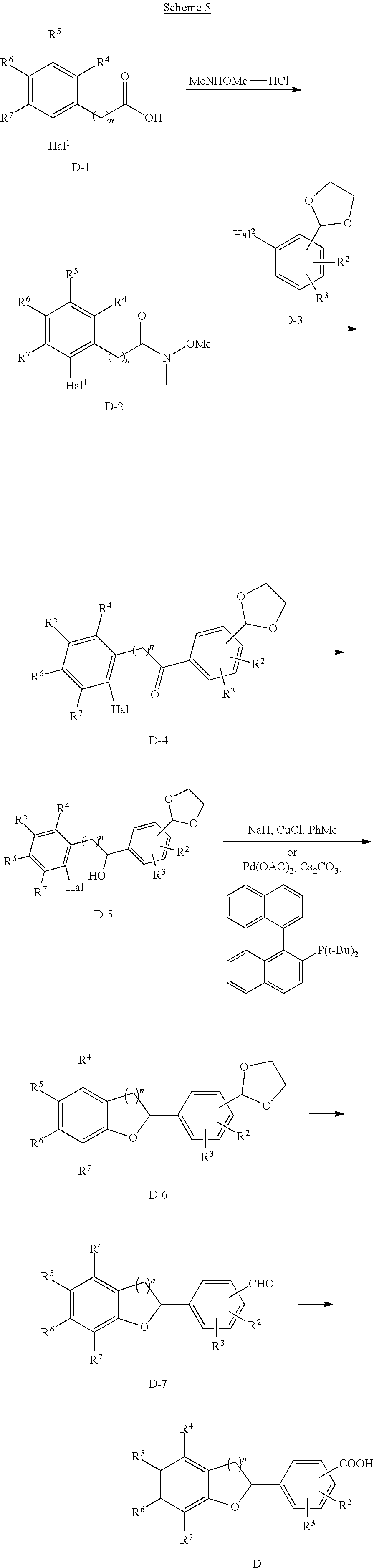

A method for preparing provided compounds, e.g., compounds of formula I or salts thereof such as compounds of formula D or salts thereof, is illustrated in Scheme 5 as an example, wherein each variable is independently as described herein. In some embodiments, treatment of an acid compound of formula D-1 or a salt thereof (in some embodiments, Hal¹ is Cl, Br, or I) with MeNHOMe-HCl under amide coupling conditions such as HATU/DIPEA affords a compound of formula D-2 or a salt thereof. In some embodiments, halide-metal exchange of a compound of formula D-3 or a salt thereof with a protected aldehyde (in some embodiments, Hal² is Br or I) using organometallic reagents such as n-BuLi or i-PrMgBr, and addition of the resulting arylmetal intermediate to a compound of formula D-2 or a salt thereof provides a ketone compound of formula D-4 or a salt thereof. In some embodiments, reduction of a ketone of formula D-4 or a salt thereof with a reducing reagent such as NaBH₄ affords an alcohol compound of formula D-5 as described herein. In some embodiments, cyclization of a compound of formula D-5 as described herein under a transition metal catalyzed intramolecular C—O coupling condition such as NaH/CuCl/PhMe or Pd(OAc)₂/TrixiePhos/Cs₂CO₃ provides a compound of formula D-6 or a salt thereof. In some embodiments, subjection of a compound of formula D-6 or a salt thereof to acid hydrolysis conditions such as HCl, followed by oxidation of the resulting aldehyde compound of formula D-7 or a salt thereof under a condition such as Pinnick oxidation condition furnishes a compound of formula I or a salt thereof, e.g., a compound of formula D or a salt thereof.

In some embodiments, the present disclosure provides a method, comprising:
reacting a compound having the structure of formula D′-5:

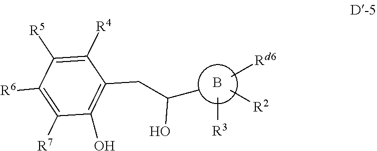

or a salt thereof to provide a compound having the structure of formula D′-6:

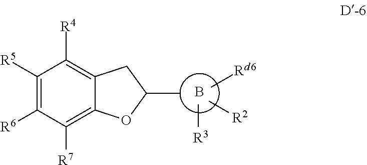

or a salt thereof, wherein each variable is independently as described herein.

In some embodiments, a compound having the structure of formula D′-5 or a salt of has the structure of

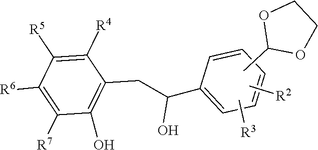

or a salt thereof, wherein each variable is independently as described herein. In some embodiments, a compound having the structure of formula D′-6 or a salt thereof has the structure of

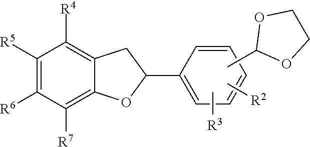

or a salt thereof, wherein each variable is independently as described herein.

Various technologies can be utilized to convert a compound having the structure of formula D′-5 or a salt thereof into a compound having the structure of formula D-6 or a salt thereof. In some embodiments, a condition is a Mitsunobu condition. In some embodiments, a reaction is performed in the presence of a phosphine compound. In some embodiments, a reaction is performed in the presence of an azodicarboxylate compound. In some embodiments, a phosphine compound has the structure of P(R)₃ wherein each R is independently as described herein and is not —H. In some embodiments, each R is independently optionally substituted phenyl. In some embodiments, a phosphine compound is PPh₃. In some embodiments, an azodicarboxylate compound has the structure of R^{a1}O₂C—N═N—CO₂R^{a2} or a salt thereof, wherein each of R^{a1} and R^{a2} is independently R. In some embodiments, each of R^{a1} and R^{a2} is not —H. In some embodiments, R^{a1} is C1-6 aliphatic. In some embodiments, R^{a1} is ethyl. In some embodiments, R^{a1} is isopropyl. In some embodiments, R^{a2} is C₁₋₆ aliphatic. In some embodiments, R³² is ethyl. In some embodiments, R^{a2} is isopropyl. In some embodiments, an azodicarboxylate compound is DIAD. In some embodiments, an azodicarboxylate compound is DEAD.

In some embodiments, the present disclosure provides a method, comprising:
reacting a compound having the structure of formula D′-4:

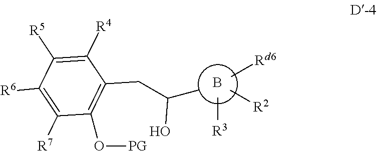

or a salt thereof to provide a compound having the structure of formula D′-5:

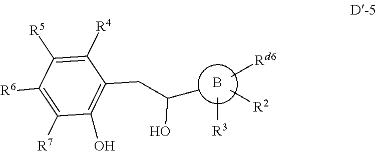

or a salt thereof, wherein PG is a protecting group, and each other variable is independently as described herein.

In some embodiments, a compound having the structure of formula D′-4 or a salt of has the structure of

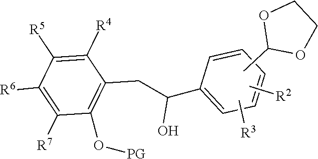

or a salt thereof, wherein each variable is independently as described herein. In some embodiments, a compound having the structure of formula D′-5 or a salt of has the structure of

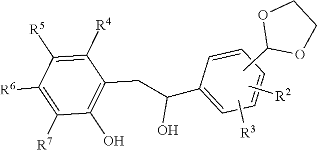

or a salt thereof, wherein each variable is independently as described herein. Those skilled in the art appreciate that many technologies are available for protecting and de-protecting a phenol group and can be utilized in accordance with the present disclosure. For example, in some embodiments, PG is a suitable protecting group such as Bn, MEM, allyl, etc.

In some embodiments, the present disclosure provides a method, comprising:
reacting a compound having the structure of formula D′-3:

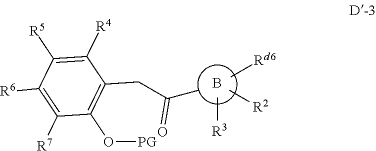

or a salt thereof to provide a compound having the structure of formula D′-4:

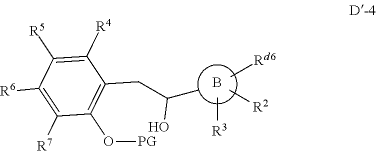

or a salt thereof, wherein each variable is independently as described herein.

In some embodiments, a compound having the structure of formula D′-3 or a salt of has the structure of

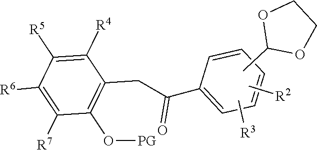

or a salt thereof, wherein each variable is independently as described herein. In some embodiments, a compound having the structure of formula D′-4 or a salt thereof has the structure of

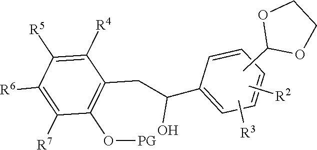

or a salt thereof, wherein each variable is independently as described herein.

In some embodiments, a useful reaction condition is a reduction condition. Various technologies for reducing a ketone to an alcohol can be utilized in accordance with the present disclosure.

In some embodiments, the present disclosure provides a method, comprising:
reacting a compound having the structure of formula D′-1:

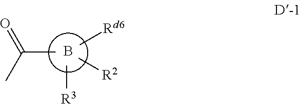

or a salt thereof with a compound having the structure of formula D′-2:

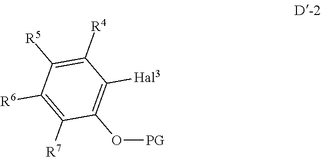

or a salt thereof to provide a compound having the structure of formula D′-3:

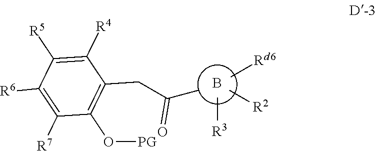

or a salt thereof, wherein each Hal³ is Hal as described herein, and each other variable is independently as described herein.

In some embodiments, a compound having the structure of formula D′-1 or a salt thereof has the structure

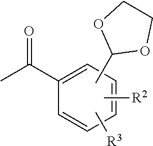

or a salt thereof, wherein each variable is independently as described herein. In some embodiments, a compound having the structure of formula D′-2 or a salt thereof has the structure of

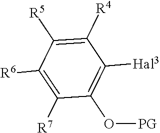

or a salt thereof, wherein each variable is independently as described herein. In some embodiments, a compound having the structure of formula D′-3 or a salt thereof has the structure of

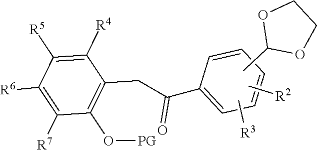

or a salt thereof, wherein each variable is independently as described herein.

In some embodiments, Hal³ is C₁. In some embodiments, Hal³ is Br. In some embodiments, Hal³ is I.

In some embodiments, a reaction is performed in the presence of a metal. In some embodiments, a metal is in a metal complex. In some embodiments, a metal complex is a Pd complex. In some embodiments, a metal complex is XPhos Pd G3. In some embodiments, a suitable solvent is dioxane. In some embodiments, a reaction is performed in the presence of a base. In some embodiments, a base is Cs₂CO₃.

In some embodiments, the present disclosure provides a method, comprising:
reacting a compound having the structure of formula D′-0:

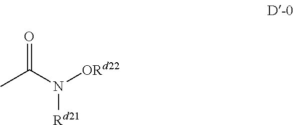

or a salt thereof with a compound having the structure of formula D-3:

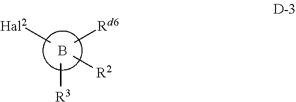

or a salt thereof to provide a compound having the structure of formula D′-1:

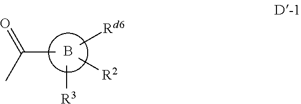

or a salt thereof, wherein each variable is independently as described herein.

In some embodiments, a compound having the structure of formula D′-0 a salt thereof has the structure of

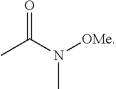

In some embodiments, a compound having the structure of formula D-3 or a salt thereof has the structure of

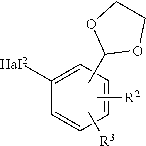

or a salt thereof, wherein each variable is independently as described herein. In some embodiments, a compound having the structure of formula D′-1 or a salt thereof has the structure of

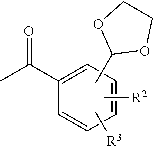

or a salt thereof, wherein each variable is independently as described herein.

In some embodiments, a reaction is performed in the presence of an organometallic agent. In some embodiments, an agent is a Li agent, e.g., n-BuLi. In some embodiments, an agent is a Mg agent, e.g., i-PrMgBr. In some embodiments, a compound having the structure of formula D-3 or a salt thereof is contacted with an organometallic agent, and the resulting agent is contacted with a compound having the structure of formula D′-0 or a salt thereof.

In some embodiments, a method is described in Scheme 6 as an example:

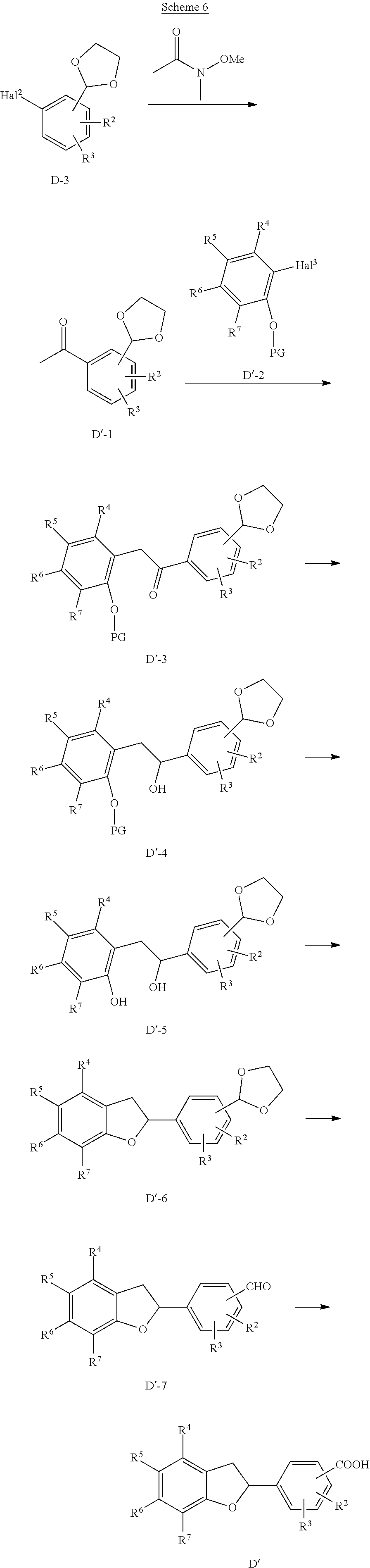

A method for preparing provided compounds, e.g., compounds of formula I or salts thereof such as compounds of formula D′ or salts thereof, is illustrated in Scheme 6 as an example, wherein each variable is independently as described herein. In some embodiments, halide-metal exchange of a compound of formula D-3 or a salt thereof, using a organometallic reagent such as n-BuLi or i-PrMgBr, and addition of the resulting arylmetal intermediate to N-methoxy-N-methylacetamide provides a ketone compound of formula D′-1 or a salt thereof. In some embodiments, treatment of a compound of formula D′-1 or a salt thereof and an aryl halide of formula D′-2 or a salt thereof under a suitable condition, e.g., Pd-catalyzed ketone α-arylation condition such as XPhos Pd G3/Cs₂CO₃/dioxane, affords a-aryl a ketone of formula D′-3 or a salt thereof. In some embodiments, reduction of a ketone of formula D′-3 or a salt thereof with a reducing reagent such as NaBH₄ affords an alcohol compound of formula D′-4 or a salt thereof. In some embodiments, removal of a protecting group of phenol provides a compound of formula D′-5 or a salt thereof. In some embodiments, cyclization of a compound of D′-5 or a salt thereof under a suitable condition, e.g., a Mitsunobu conditions such as DIAD/PPh₃, provides a compound of formula D′-6 or a salt thereof. In some embodiments, subjection of a compound of D′-6 or a salt thereof to an acid hydrolysis condition such as HCl and oxidation of a resulting aldehyde compound of formula D′-7 or a salt thereof (e.g. Pinnick oxidation) furnishes a compound of formula I or a salt thereof, e.g., a compound of formula D′ or a salt thereof.

In some embodiments, a method is described in Scheme 7 as an example:

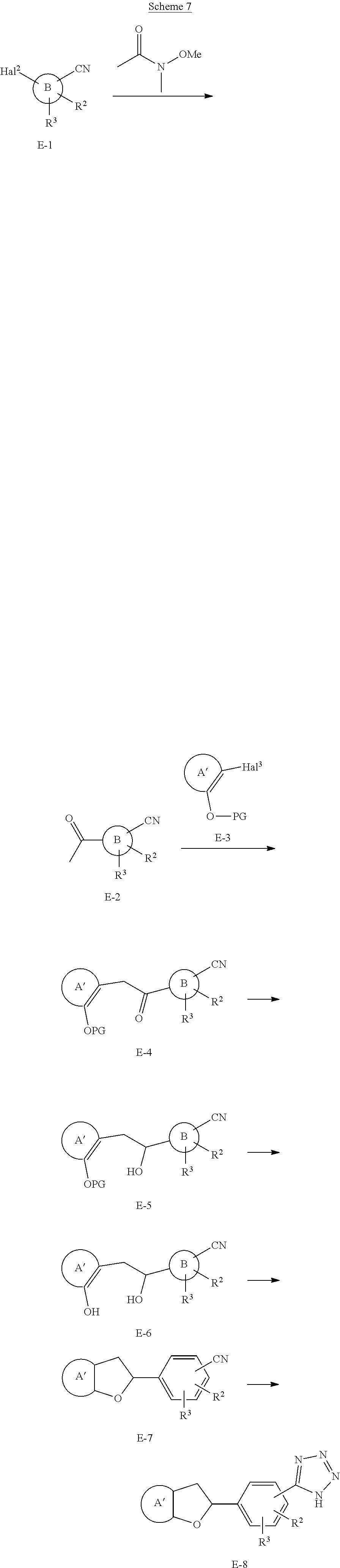

A method for preparing provided compounds, e.g., compounds of formula I or salts thereof such as compounds of formula E (e.g., formula E-8) or salts thereof, is illustrated in Scheme 7 as an example, wherein each variable is independently as described herein. In some embodiments, a provided method comprises halide-metal exchange of a compound of formula E-1 or a salt thereof, using an organometallic reagent such as n-BuLi or i-PrMgBr to provide an arylmetal compound. In some embodiments, a provided comprises addition of an arylmetal compound to N-methoxy-N-methylacetamide to provide a ketone compound of formula E-2 or a salt thereof. In some embodiments, a method comprises treatment of a compound of formula E-2 or a salt thereof and an aryl halide of formula E-3 or a salt thereof under a suitable condition, e.g., Pd-catalyzed ketone α-arylation condition such as XPhos Pd G3/Cs₂CO₃/dioxane, to provide α-aryl a ketone of formula E-4 or a salt thereof. In some embodiments, a method comprises reduction of a ketone of formula E-4 or a salt thereof with a reducing reagent such as NaBH₄ to provide an alcohol compound of formula E-5 or a salt thereof. In some embodiments, a method comprises removal of a protecting group of phenol to provide a compound of formula E-6 or a salt thereof. In some embodiments, a method comprises cyclization of a compound of E-6 or a salt thereof under a suitable condition, e.g., a Mitsunobu conditions such as DIAD/PPh₃, to provide a compound of formula E-7 or a salt thereof. In some embodiments, a provided method comprises contacting a compound of E-7 or a salt thereof with an azide compound (e.g., TMSN₃) to provide a compound of formula I or a salt thereof, e.g., a compound of formula E-8 or a salt thereof.

In some embodiments, a method is described in Scheme 8 as an example:

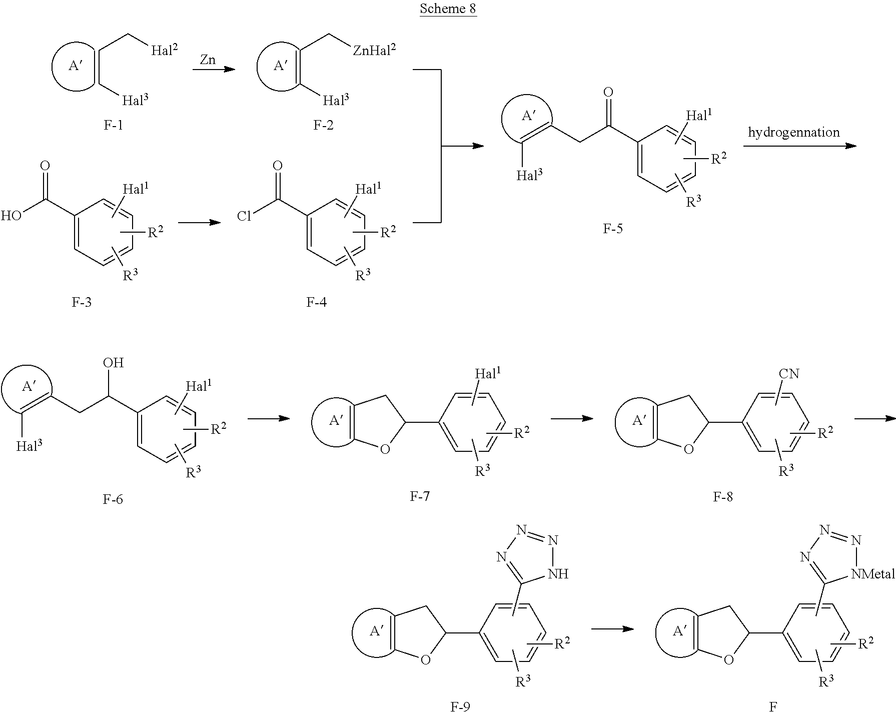

A method for preparing provided compounds, e.g., compounds of formula I or salts thereof such as compounds of formula F-7, formula F-8, formula F-9 or formula F, or salts thereof, is illustrated in Scheme 8 as an example, wherein each variable is independently as described herein. In some embodiments, each of Hal¹, Hal², and Hal³ is independently F, Cl, Br, or I. In some embodiments, Hall is —Br. In some embodiments, Hal² is —Br. In some embodiments, Hal³ is —F. In some embodiments, a metal is Na. In some embodiments, a method comprises reacting a compound of formula F-1 or a salt thereof with a Zn reagent to provide a compound of formula F-2 or a salt thereof. In some embodiments, a Zn reagent is or comprise Zn. In some embodiments, a reaction is performed in the presence of a salt, e.g., a lithium salt like LiCl. In some embodiments, a method comprises reacting a compound of formula F-3 or a salt thereof to provide a compound of formula F-4 or a salt thereof under a suitable condition, e.g., using SOCl₂. In some embodiments, a provided method comprises reacting a compound of formula F-2 or a salt thereof with a compound of formula F-4 or a salt thereof (e.g., transition coupling) to form a compound of formula F-5 or a salt thereof. In some embodiments, a coupling of a compound of formula F-2 or a salt thereof and a compound of formula F-4 or a salt thereof utilizes a metal agent, e.g., CuCN. In some embodiments, such a reaction is performed in the presence of a salt, e.g., a lithium salt like LiCl. In some embodiments, a method comprises reducing a compound of formula F-5 or a salt thereof to provide a compound of formula F-6 or a salt thereof. In some embodiments, reducing is or comprises hydrogenation. In some embodiments, a hydrogenation utilizes H₂. In some embodiments, a hydrogenation is an in situ transhydrogenation. In some embodiments, a hydrogenation utilizes a metal catalyst. In some embodiments, a hydrogenation utilizes a metal catalyst and HCOOH. In some embodiments, a metal catalyst is or comprises a transition metal complex. In some embodiments, a metal catalyst is or comprises a Ru complex. In some embodiments, it is RuCl(p-cymene) [(S,S)-Ts-DPEN]. In some embodiments, a hydrogenation utilizes RuCl (p-cymene) [(S,S)-Ts-DPEN] and HCOOH. Various reduction technologies are available and can be utilized in accordance with the present disclosure. In some embodiments, a method comprises reacting a compound of formula F-6 or a salt thereof to provide a compound of formula F-7 or a salt thereof under suitable conditions, e.g., using a base. In some embodiments, a base is t-BuOK. In some embodiments, a base is t-BuONa. In some embodiments, a base is a metal alkoxide. In some embodiments, a method comprises reacting a compound of formula F-7 or a salt thereof with a cyanide reagent to provide a compound of formula F-8 or a salt thereof. In some embodiments, a cyanide reagent is CuCN. In some embodiments, a method comprises reacting a compound of formula F-8 or a salt thereof with an azide reagent to provide a compound of formula F-9 or a salt thereof. In some embodiments, an azide reagent is TMSN₃. In some embodiments, an azide reagent is NaN₃. In some embodiments, such a reaction is performed in the presence of a catalyst. In some embodiments, a catalyst is Bu₂SnO. In some embodiments, a method comprises reacting a compound of formula F-8 or a salt thereof with TMSN₃ and Bu₂SnO to provide a compound of formula F-9 or a salt thereof. In some embodiments, a reaction is performed in the presence of a base and/or a salt of a base (e.g., TEA-HCl). In some embodiments, a base is an amine base. In some embodiments, a method comprises reacting a compound of formula F-8 or a salt thereof with NaN₃, TEA. HCl, and NMP to provide a compound of formula F-9 or a salt thereof. In some embodiments, a method comprises reacting a compound of formula F-8 or a salt thereof with an azide reagent to provide a compound of formula F-9 or a salt thereof in a scale at or greater than about 1 mmol, 5 mmol, 10 mmol, 20 mmol, 50 mmol, 100 mmol, 250 mmol, 500 mmol, 1 mol, 5 mol, 10 mol, 100 mol, 1000 mol, or 2000 mmol. In some embodiments, a method comprises reacting a compound of formula F-8 or a salt thereof with an azide reagent to provide a compound of formula F-9 or a salt thereof in a scale less than about 1 mmol, 5 mmol, 10 mmol, 20 mmol, 50 mmol, 100 mmol, 250 mmol, 500 mmol, 1 mol, 5 mol, 10 mol, 100 mol, 1000 mol, or 2000 mmol. In some embodiments, a method comprises reacting a compound of formula F-9 or a salt thereof with a base to provide a compound of formula F or a salt thereof. In some embodiments, a base is NaOH. In some embodiments, a base is KOH.

In some embodiments, the present disclosure provides a method, comprising contacting a compound of formula I or a salt thereof, wherein R¹ is —C(O)OR¹¹, —P(O)(OR¹²)(OR¹³), or

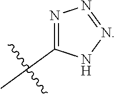

wherein R¹¹ is hydrogen, and at least one of R¹² and R¹³ is hydrogen, with a base to prepare a salt of such a compound of formula I. In some embodiments, a base is an alkaline hydroxide. In some embodiments, a base is NaOH. In some embodiments, a base is an amine base. Various bases are useful for preparing salts including pharmaceutically acceptable salts and can be utilized in accordance with the present disclosure.

Those skilled in the art reading the present disclosure will appreciate that, in some embodiments, a compound of formula F-1 or a salt thereof may be replaced with a compound of formula F-1′

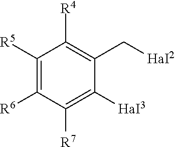

or a salt thereof wherein each variable is as described herein. In some embodiments, Hal³ is F. In some embodiments, Hal² is Br. In some embodiments, a compound of formula F-1 or F-1′ is

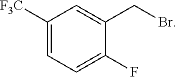 [SMILES: Fc1ccc(C(F)(F)F)cc1CBr]

In some embodiments, a compound of formula F-2 or a salt thereof may be replaced with a compound of formula F-2′

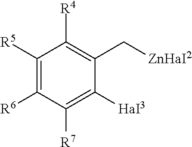

or a salt thereof wherein each variable is as described herein. In some embodiments, a compound of F-2 or F-2′ is

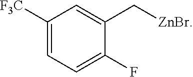

In some embodiments, a compound of F-2 or F-2′ is

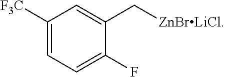

In some embodiments, a compound of formula F-3 is

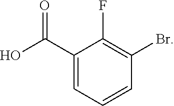 [SMILES: O=C(O)c1cccc(Br)c1F]

In some embodiments, a compound of formula F-4

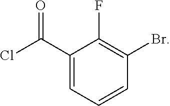 [SMILES: O=C(Cl)c1cccc(Br)c1F]

In some embodiments, a compound of formula F-5 or a salt thereof may be replaced with a compound of formula F-5′

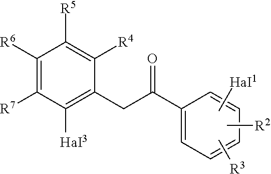

or a salt thereof wherein each variable is as described herein. In some embodiments, Hall is Br. In some embodiments, a compound of formula F-5 or F-5′ is

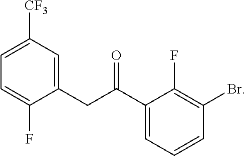 [SMILES: O=C(Cc1cc(C(F)(F)F)ccc1F)c1cccc(Br)c1F]

In some embodiments, a compound of formula F-6 or a salt thereof may be replaced with a compound of formula F-6′

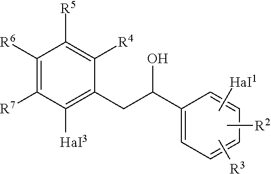

or a salt thereof wherein each variable is as described herein. In some embodiments, a compound of formula F-6 or F-6′ is

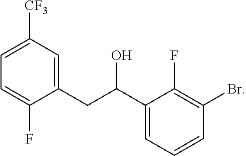 [SMILES: OC(Cc1cc(C(F)(F)F)ccc1F)c1cccc(Br)c1F]

In some embodiments, a compound of formula F-6 or F-6′ is

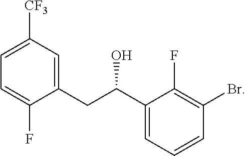 [SMILES: O[C@@H](Cc1cc(C(F)(F)F)ccc1F)c1cccc(Br)c1F]

In some embodiments, a compound of formula F-7 or a salt thereof may be replaced with a compound of

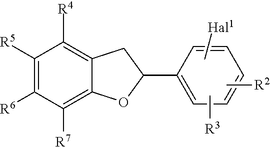

formula F-7′ or a salt thereof wherein each variable is as described herein. In some embodiments, a compound of formula F-7 or F-7′ is

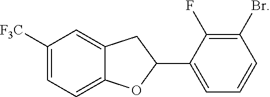 [SMILES: Fc1c(Br)cccc1C1Cc2cc(C(F)(F)F)ccc2O1]

In some embodiments, a compound of formula F-7 or F-7′ is

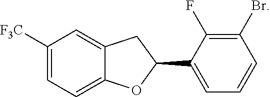 [SMILES: Fc1c(Br)cccc1[C@@H]1Cc2cc(C(F)(F)F)ccc2O1]

In some embodiments, a compound of formula F-8 or a salt thereof may be replaced with a compound of formula F-8′

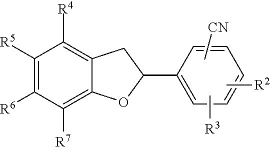

or a salt thereof wherein each variable is as described herein. In some embodiments, a compound of formula F-8 or F-8′ is

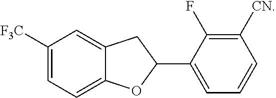 [SMILES: N#Cc1cccc(C2Cc3cc(C(F)(F)F)ccc3O2)c1F]

In some embodiments, a compound of formula F-8 or F-8′ is

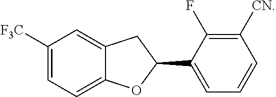 [SMILES: N#Cc1cccc([C@@H]2Cc3cc(C(F)(F)F)ccc3O2)c1F]

In some embodiments, a compound of formula F-9 or a salt thereof may be replaced with a compound of formula F-9′

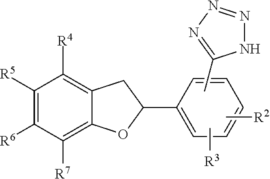

or a salt thereof wherein each variable is as described herein. In some embodiments, a compound of formula F-9 or F-9′ or a salt thereof is

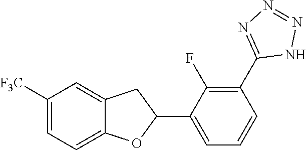 [SMILES: Fc1c(-c2nnn[nH]2)cccc1C1Cc2cc(C(F)(F)F)ccc2O1]

or a salt thereof. In some embodiments, a compound of formula F-9 or F-9′ or a salt thereof is

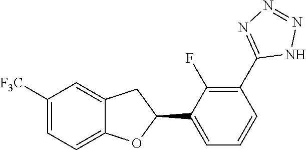 [SMILES: Fc1c(-c2nnn[nH]2)cccc1[C@@H]1Cc2cc(C(F)(F)F)ccc2O1]

or a salt thereof. In some embodiments, a compound of formula For a salt thereof is a compound of formula F′

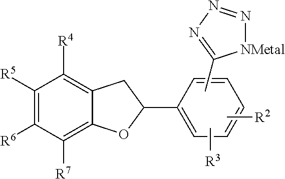

or a salt thereof wherein each variable is as described herein. In some embodiments, a compound of formula F or F′ or a salt thereof is

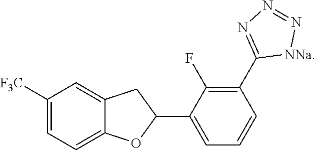

In some embodiments, a compound of formula F or F′ or a salt thereof is

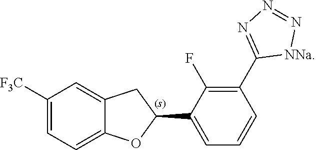

In some embodiments, Ring A′ in formula F-1, F-2, F-5, F-6, F-7, F-8, F-9, or F is independently optionally substituted

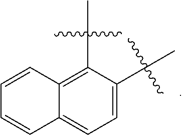 [SMILES: *c1ccc2ccccc2c1*]

In some embodiments, Ring A′ in formula F-1, F-2, F-5, F-6, F-7, F-8, F-9, or F is independently

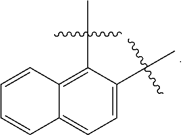 [SMILES: *c1ccc2ccccc2c1*]

In some embodiments, Ring A′ in formula F-1, F-2, F-5, F-6, F-7, F-8, F-9, or F is independently optionally substituted

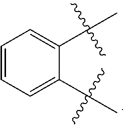 [SMILES: *c1ccccc1*]

In some embodiments, Ring A′ in formula F-1, F-2, F-5, F-6, F-7, F-8, F-9, or F is independently

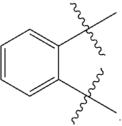 [SMILES: *c1ccccc1*]

In some embodiments, Ring A′ in formula F-1, F-2, F-5, F-6, F-7, F-8, F-9, or F is independently

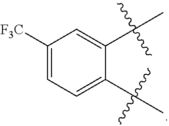 [SMILES: *c1ccc(C(F)(F)F)cc1*]

In some embodiments, the present disclosure provides a method, comprising reacting a compound having the structure of formula I wherein R¹ is —CN or a salt thereof to provide a compound having the structure of formula I wherein R¹ is

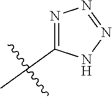 [SMILES: *c1nnn[nH]1]

or a salt thereof. Various technologies are available for such reacting and can be utilized in accordance with the present disclosure. For example, in some embodiments, a method comprising contacting a compound having the structure of formula I wherein R¹ is —CN or a salt thereof with an azide and a tin oxide. In some embodiments, an azide is TMSN₃. In some embodiments, a tin oxide has the structure of R²Sn(O) or a salt thereof. In some embodiments, each R is optionally substituted C₁₋₆ aliphatic. In some embodiments, each R is independently C₁₋₆ alkyl. In some embodiments, each R is independently C₁₋₆ alkyl. In some embodiments, the two R the same. In some embodiments, a tin oxide is dibutyltin oxide. In some embodiments, a reaction is performed at a temperature higher than an ambient temperature, e.g., at about 100° C., 110° C. or higher temperature. In some embodiments, a reaction is performed in a solvent, e.g., toluene. In some embodiments, a reaction time is about 1, 2, 3, 4, 5, 6, 7, 8, 9, 10, 11, 12, or more hours.

In some embodiments, the present disclosure provides a method, comprising reacting a compound having the structure of formula I wherein R¹ is —C(O)NH₂ or a salt thereof to provide a compound having the structure of formula I wherein R¹ is —CN. Various technologies are available for such reacting and can be utilized in accordance with the present disclosure. For example, in some embodiments, a compound having the structure of formula I wherein R¹ is —C(O)NH₂ or a salt thereof is contacted with an anhydride. In some embodiments, a compound having the structure of formula I wherein R¹ is —C(O)NH₂ or a salt thereof is contacted with TFAA. In some embodiments, a contact is performed in the presence of a base, e.g., Et₃N. In some embodiments, a reaction is performed in a suitable solvent, e.g., DCM. In some embodiments, a reaction is performed at a temperature lower than an ambient temperature, e.g., at about 0° C.

In some embodiments, the present disclosure provides a method, comprising reacting a compound having the structure of formula I wherein R¹ is —C(O)OH or a salt thereof to provide a compound having the structure of formula I wherein R¹ is —C(O)NH₂. Various technologies, e.g., amidation technologies, are available for such reacting and can be utilized in accordance with the present disclosure. For example, in some embodiments, a compound having the structure of formula I wherein R¹ is —C(O)OH or a salt thereof is activated. In some embodiments, a compound having the structure of formula I wherein R¹ is —C(O)OH or a salt thereof is contacted with SOCl₂. In some embodiments, a compound having the structure of formula I wherein R¹ is —C(O)OH or a salt thereof or an activated form thereof is contacted with NH₃ (e.g., NH₃ in MeOH). In some embodiments, the present disclosure provides a method, comprising reacting a compound having the structure of formula I wherein R¹ is —C(O)OH or a salt thereof to provide a compound having the structure of formula I wherein R¹ is —CN.

In some embodiments, a preparation or composition is enriched for a stereoisomer. In some embodiments, a preparation or composition is enriched for a diastereomer. In some embodiments, a preparation or composition is enriched for an enantiomer. In some embodiments, a preparation or composition is diastereomerically pure. In some embodiments, a preparation of composition is enantiomerically pure. Stereochemically enriched or pure preparations and compositions may be prepared utilizing various stereoselective technologies, e.g., chiral auxiliaries, stereoselective reactions, stereoselective catalysis, etc., in accordance with the present disclosure. For example, in some embodiments, a compound of formula F-5 or F-5′ or a salt thereof may be stereoselectively reduced to provide a compound of formula F-6 or F-6′ or a salt thereof. In some embodiments, the formed stereogenic carbon from the reduction is R. In some embodiments, it is S. In some embodiments, a reduction is preformed in the presence of a chiral metal catalyst, e.g., RuCl (p-cymene) [(S,S)-Ts-DPEN].

Certain technologies for preparing provided compounds are illustrated in the Examples.

### Use

In certain embodiments, the present disclosure provides a method for modulating MRGPRX4 activity by contacting MRGPRX4 with an effective amount of a compound or a pharmaceutical composition as described herein.

In certain embodiments, the present disclosure provides a method for modulating MRGPRX4 activity in a system comprising MRGPRX4, comprising administering or delivering to the system an effective amount of a compound or a pharmaceutical composition as described herein. In some embodiments, a system is or comprises a cell. In some embodiments, a system is or comprises a tissue. In some embodiments, a system is or comprises an organ. In some embodiments, a system is or comprises an organism. In some embodiments, a system is a subject. In some embodiments, a system is an animal. In some embodiments, a system is a human. In some embodiments, a system expresses MRGPRX4. In some embodiments, a method reduces MRGPRX4 activity level compared absence of a provided compound. In some embodiments, a reduction is about or at least about 20%, 25%, 30%, 40%, 50%, 60%, 70%, 80%, 90%, or 95%.

In certain embodiments, the present disclosure provides a method for preventing a condition, disorder or disease, comprising administering to a subject susceptible thereto an effective amount of a provided compound or composition. In certain embodiments, the present disclosure provides a method for treating a condition, disorder or disease, comprising administering to a subject suffering therefrom an effective amount of a provided compound or composition. In certain embodiments, the present disclosure provides a method for preventing a condition, disorder or disease, comprising delivering to a subject susceptible thereto an effective amount of a provided compound or composition. In certain embodiments, the present disclosure provides a method for treating a condition, disorder or disease, comprising delivering to a subject suffering therefrom an effective amount of a provided compound or composition. In some embodiments, a compound is administered or delivered as a pharmaceutically acceptable salt form. In some embodiments, a composition is a pharmaceutical composition. In some embodiments, to deliver a provided compound, a prodrug thereof may be administered.

In some embodiments, a compound is utilized in a racemic form. In some embodiments, a composition is a stereorandom mixture of multiple stereoisomers. For example, in some embodiments, a composition is a stereorandom mixture of two enantiomers. In some embodiments, a compound is utilized in a stereochemically pure form as described herein. In some embodiments, a compound is utilized in an enantiomerically pure form. In some embodiments, a composition is enriched for one or more stereoisomers over the others as described herein. In some embodiments, a composition is enriched for an enantiomer as described herein. In some embodiments, a composition is stereochemically pure. In some embodiments, a composition is enantiomerically pure.

In some embodiments, a condition, disorder or disease is or comprises itch. In some embodiments, a condition, disorder or disease is itch. In some embodiments, a condition, disorder or disease is a MRGPRX4-associated condition, disorder or disease. In some embodiments, a condition, disorder or disease is associated with MRGPRX4 activation.

In some embodiments, a condition, disorder or disease is chronic itch, cholestatic pruritus, contact dermatitis, allergic blepharitis, anemia, atopic dermatitis, bullous pemphigoid, candidiasis, chicken pox, cholestasis, end-stage renal failure, hemodialysis, contact dermatitis, dermatitis herpetiformis, diabetes, drug allergy, dry skin, dyshidrotic dermatitis, ectopic eczema, eczema, erythrasma, folliculitis, fungal skin infection, hemorrhoids, herpes, HIV infection, Hodgkin's disease, hyperthyroidism, iron deficiency anemia, kidney disease, leukemia, liver disease, lymphoma, malignancy, multiple myeloma, neurodermatitis, onchocerciasis, Paget's disease, pediculosis, polycythemia rubra vera, pruritus ani, pseudorabies, psoriasis, rectal prolapse, scabies, schistosomiasis, scleroderma, severe stress, stasis dermatitis, swimmer's itch, thyroid disease, tinea cruris, uremic pruritus, or urticaria.

In some embodiments, a condition, disorder or disease is MRGPRX4-associated pruritus. In some embodiments, a condition, disorder or disease is MRGPRX4-associated acute or chronic pruritus associated a liver condition, disorder or disease.

In certain embodiments, an MRGPRX4-associated pruritus is acute or chronic pruritus associated with a hepatobiliary condition, disorder or disease. In some embodiments, a hepatobiliary condition, disorder or disease is intrahepatic cholestasis of pregnancy (ICP), estrogen-, progesterone- or testosterone-induced cholestasis, toxin- or other drug induced hepatocellular cholestasis, benign recurrent intrahepatic cholestasis (BRIC), progressive familial intrahepatic cholestasis (PFIC), chronic viral hepatitis C, chronic hepatitis B, alcoholic or nonalcoholic fatty liver disease (NAFLD), nonalcoholic steatohepatitis (NASH), primary biliary cholangitis (PBC), primary sclerosing cholangitis (PSC), secondary sclerosing cholangitis (SSC), sarcoidosis, ABCB4 deficiency, alagille syndrome, drug-induce small duct cholangiopathies, gallstone disease, IgG4-associated cholangitis, biliary atresia, cholangiocellular carcinoma, benign bile duct adenoma, or other obstructive cholestasis.

In some embodiments, a condition, disorder or disease is a liver condition, disorder or disease. In some embodiments, a liver condition, disorder or disease is NASH. In some embodiments, a liver condition, disorder or disease is NAFLD. In some embodiments, a liver condition, disorder or disease is ICP. In some embodiments, a liver condition, disorder or disease is PBC. In some embodiments, a liver condition, disorder or disease is PFIC. In some embodiments, a liver condition, disorder or disease is PSC. In some embodiments, a liver condition, disorder or disease is BRIC. In some embodiments, a liver condition, disorder or disease is chronic hepatitis B.

In some embodiments, a condition, disorder or disease is nonalcoholic steatohepatitis (NASH). In some embodiments, a condition, disorder or disease is bile acid synthesis condition, disorder or disease. In some embodiments, a bile acid synthesis condition, disorder or disease is due to single enzyme defects (SEDs). In some embodiments, a condition, disorder or disease is a peroxisomal condition, disorder or disease, e.g., a Zellweger spectrum disorder. In some embodiments, a condition, disorder or disease is a liver condition, disorder or disease, steatorrhea or complications from decreased fat-soluble vitamin absorption.

In some embodiments, a condition, disorder or disease is cardiovascular disease, atherosclerosis, arteriosclerosis, hypercholesteremia, hyperlipidemia, chronic liver disease, gastrointestinal disease, renal disease, metabolic disease, cancer (i.e., colorectal cancer), or neurological indications such as stroke. In certain embodiments, a condition, disorder or disease is primary biliary cirrhosis (PBC), cerebrotendinous xanthomatosis (CTX), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, chronic viral hepatitis, alcoholic liver disease, nonalcoholic fatty liver disease (NAFLD), nonalcoholic steatohepatitis (NASH), liver transplant associated graft versus host disease, living donor transplant liver regeneration, congenital hepatic fibrosis, choledocholithiasis, granulomatous liver disease, intra- or extrahepatic malignancy, Sjogren's syndrome, Sarcoidosis, Wilson's disease, Gaucher's disease, hemochromatosis, or alpha 1-antitrypsin deficiency. In certain embodiments, a gastrointestinal disease is inflammatory bowel disease (IBD)(including Crohn's disease and ulcerative colitis), irritable bowel syndrome (IBS), bacterial overgrowth, malabsorption, postradiation colitis, or microscopic colitis. In certain embodiments, the renal disease is diabetic nephropathy, focal segmental glomerulosclerosis (FSGS), hypertensive nephrosclerosis, chronic glomerulonephritis, chronic transplant glomerulopathy, chronic interstitial nephritis, or poly cystic kidney disease. In certain embodiments, a cardiovascular disease is atherosclerosis, arteriosclerosis, dyslipidemia, hypercholesterolemia, or hypertriglyceridemia. In certain embodiments, a metabolic disease is insulin resistance, Type I and Type II diabetes, or obesity. In some embodiments, a condition, disorder or disease is an inflammatory condition, disorder or disease, e.g., allergy, osteoarthritis, appendicitis, bronchial asthma, pancreatitis, allergic rash, psoriasis, etc. In some embodiments, a condition, disorder or disease is an autoimmune condition, disorder or disease. In some embodiments, a condition, disorder or disease is rheumatoid arthritis, multiple sclerosis, and type I diabetes. In some embodiments, a condition, disorder or disease is a gastrointestinal disease, e.g., inflammatory bowel disease (Crohn's disease, ulcerative colitis), short bowel syndrome (post-radiation colitis), microscopic colitis, irritable bowel syndrome (malabsorption), and bacterial overgrowth. In some embodiments, a condition, disorder or disease is cancer. In some embodiments, a cancer is colorectal cancer, liver cancer, hepatocellular carcinoma, cholangiocarcinoma, renal cancer, gastric cancer, pancreatic cancer, prostate cancer, or insulanoma. In some embodiments, a condition, disorder or disease is FXR-mediated. In some embodiments, a condition, disorder or disease is TGR5-mediated. In some embodiments, subjects susceptible thereto and/or suffering therefrom benefit from increased levels of FXR and/or TGR5 activity. In some embodiments, a condition, disorder or disease is a chronic kidney disease. In some embodiments, a condition, disorder or disease is uremic pruritus.

In some embodiments, a condition, disorder or disease is associated with administration or delivery of an agent that can activate MRGPRX4. Many technologies for assessing MRGPRX4 activation, e.g., in vivo, in vitro, etc. can be utilized to assess if an agent can activate MRGPRX4. Certain useful technologies are described in, e.g., Meixiong et al. MRGPRX4 is a G protein-coupled receptor activated by bile acids or analogs or derivatives thereof that may contribute to cholestatic pruritus, PNAS, 2019, 116 (21), 10525-10530; Yu et al. MRGPRX4 is bile acid receptor for human cholestatic itch, eLife, 2019, 8, e48431.

In some embodiments, a condition, disorder or disease is associated with administration or delivery of an agent that can activate MRGPRX4 but can also provide another activity or can be utilized as a therapeutic agent for treating a condition, disorder or disease. In some embodiments, the present disclosure provides methods for preventing or treating a condition, disorder or disease associated with administration of an agent, comprising administering or delivering to a subject an effective amount of a provided compound or composition. In some embodiments, the present disclosure provides methods for preventing a condition, disorder or disease associated with administration of an agent, comprising administering to a subject an effective amount of a provided compound or composition. In some embodiments, the present disclosure provides methods for preventing a condition, disorder or disease associated with administration of an agent, comprising delivering to a subject an effective amount of a provided compound or composition. In some embodiments, the present disclosure provides methods for treating a condition, disorder or disease associated with administration of an agent, comprising administering to a subject an effective amount of a provided compound or composition. In some embodiments, the present disclosure provides methods for treating a condition, disorder or disease associated with administration of an agent, comprising delivering to a subject an effective amount of a provided compound or composition. In some embodiments, an agent can activate MRGPRX4. In some embodiments, an agent is a bile acid or a salt (e.g., a pharmaceutically acceptable salt) thereof. In some embodiments, an agent is a bile acid analog or a salt (e.g., a pharmaceutically acceptable salt) thereof. In some embodiments, an agent is a bile acid derivative or a salt (e.g., a pharmaceutically acceptable salt) thereof. In some embodiments, an agent is a bile acid conjugate, e.g., a taurine conjugate, or a salt (e.g., a pharmaceutically acceptable salt) thereof. In some embodiments, a bile acid is cholic acid. In some embodiments, a bile acid is ursodeoxycholic acid (UDCA). In some embodiments, a bile acid is ursocholic acid. In some embodiments, a bile acid is chenodeoxycholic acid. In some embodiments, a bile acid or an analog or derivative thereof is obeticholic acid. In some embodiments, a bile acid or an analog or derivative thereof is taurursodiol or a salt (e.g., a pharmaceutically acceptable salt) thereof; in some embodiments, it is taurursodiol; in some embodiments, it is sodium taurursodiol. In some embodiments, an agent is a FXR agonist. In some embodiments, an agent is a TGR5 agonist. In some embodiments, an agent is a therapeutic agent. In some embodiments, an agent is an approved therapeutic agent, e.g., by the U.S. Food and Drug Administration (e.g., cholic acid, obeticholic acid, taurursodiol, ursodeoxycholic acid, etc.), either individually or in combination with another therapeutic agent. In some embodiments, an agent is obeticholic acid and it is utilized in combination with ursodeoxycholic acid. In some embodiments, an agent is taurursodiol and it is utilized with sodium phenylbutyrate. In some embodiments, a condition, disorder or disease associated with administration of agent is or comprises itch. In some embodiments, provided methods can increase patient adherence of the agent. In some embodiments, provided methods can increase single doses, total doses, dose frequency, and/or length of dosage regimen of an agent. In some embodiments, provided compounds can reduce severity of a side effect, e.g., itch. In some embodiments, a provided compound is administered or delivered about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months before administration of an agent. In some embodiments, a provided compound is administered or delivered about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months after administration of an agent. In some embodiments, a provided compound is administered or delivered together with administration of an agent.

In certain embodiments, a compound as described herein can be used together with another therapeutic agent as a combination therapy to prevent or treat a condition, disorder or disease. In some embodiments, a condition, disorder or disease is associated with MRGPRX4 activation. In some embodiments, a therapeutic agent administered or delivered to a subject can activate MRGPRX4. In some embodiments, a provided compound can reduce a condition, disorder or disease associated with MRGPRX4 activation. In some embodiments, a condition, disorder or disease is or comprises concurring MRGPRX4 related pruritus. In certain embodiments, a therapeutic agent is a Farnesoid X receptor (FXR) agonist, such as obeticholic acid (OCA), cilofexor (GS-9674), tropifexor (LJN452), EDP-305, EDP-297, nidufexor, TERN-101 (LY2562175), MET-409, BAR704, BAR502, EYP-001, RDX-023, AGN-242266, HPG-1860, AGN-242256, IoT-022, M-480, INV-33, etc. In certain embodiments, a therapeutic agent is an ileal bile acid transport (IBAT) inhibitor, such as odevixibat, maralixibat, etc. In certain embodiments, a therapeutic agent is ursocholic acid. In certain embodiments, a therapeutic agent is a thyroid hormone receptor β (THR-β) agonist, such as resmetirom (MGL-3196), GC-24, MGL-3745, VK-2809, KB141 [3,5-dichloro-4-(4-hydroxy-3-isopropylphenoxy)phenylacetic acid], MB07811 (2R,4S)-4-(3-chlorophenyl)-2-((3,5-dimethyl-4-(4′-hydroxy-3′-isopropylbenzyl)phenoxy)methyl]-2-oxido-[1,3,2]-dioxaphosphonane), etc. In certain embodiments, a therapeutic agent is a peroxisome proliferator-activated receptors (PPAR) agonist, such as elafibranor, lanifibranor, saroglitazar, pioglitazone, rosiglitazone etc. In certain embodiments, a therapeutic agent is a glucagon-like peptide 1 (GLP-1) agonist, such as semaglutide, exenatide, dulaglutide, liraglutide, lixisenatide, danuglipron (PF-06882961) PF-07081532 etc. In certain embodiments, a therapeutic agent is a glucose-dependent insulinotropic polypeptide (GIP) receptor agonist such as tirzepatide. In certain embodiments, a therapeutic agent is an acetyl CoA-carboxylase (ACC) inhibitor such as firsocostat, PF-05221304, WZ66, etc. In certain embodiments, a therapeutic agent is a diacylglycerol O-acyltransferase 2 (DGAT2) inhibitor such as PF-06865571. In certain embodiments, the other therapeutic agent is a ketohexokinase (KHK) inhibitor such as PF-06835919. In some embodiments, a therapeutic agent is an approved agent (e.g., by U.S. Food and Drug Administration), either individually or as a combination, for treating a condition, disorder or disease. Various approved agents and their uses are publicly available and can be utilized with provided compounds in accordance with the present disclosure. For example, in some embodiments, an agent is obeticholic acid approved for treatment of adult patients with primary biliary cholangitis (PBC) without cirrhosis or with compensated cirrhosis who do not have evidence of portal hypertension, either in combination with ursodeoxycholic acid (UDCA) with an inadequate response to UDCA or as monotherapy in patients unable to tolerate UDCA. In some embodiments, an agent is cholic acid approved for treatment of bile acid synthesis disorders due to single enzyme defects (SEDs) or adjunctive treatment of peroxisomal disorders (PDs) including Zellweger spectrum disorders in patients who exhibit manifestations of liver disease, steatorrhea or complications from decreased fat-soluble vitamin absorption. In some embodiments, an agent is taurursodiol, in combination with sodium phenylbutyrate (Relyvrio) approved for the treatment of amyotrophic lateral sclerosis (ALS).

In some embodiments, a provided compound is administered or delivered concurrently with another therapeutic agent. In some embodiments, a provided compound is administered or delivered in a single composition with another therapeutic agent. In some embodiments, a provided compound is administered or delivered concurrently with another therapeutic agent but in different compositions. In some embodiments, a provided compound is administered or delivered prior to another therapeutic agent (e.g., about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months prior to another therapeutic agent). In some embodiments, a provided compound is administered or delivered after another therapeutic agent (e.g., about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months after another therapeutic agent). In some embodiments, a provided compound is administered or delivered when a subject is under the therapeutic effect of another therapeutic agent.

In some embodiments, a bile acid or an analog or derivative thereof is reported in Meixiong et al. MRGPRX4 is a G protein-coupled receptor activated by bile acids that may contribute to cholestatic pruritus, PNAS, 2019, 116 (21), 10525-10530; Yu et al. MRGPRX4 is bile acid receptor for human cholestatic itch, eLife, 2019, 8, e48431. In some embodiments, a bile acid or an analog or derivative thereof is reported in WO 2016086169, US 10519191, WO 2016130809, US 10246483, WO 2017147174, WO 2017147159, WO 2017147137, US 10364267, US 10323061, US 10323060, WO 2017189663, WO 2017189652, WO 2017189651, US 10080743, US 10080742, US 10080741, WO 2017201155, WO 2017201152, WO 2017201150, US 10144729, US 10138228, WO 2018067704, US 10450306, WO 2018081285, US 10597391, WO 2018102418, US 10584145, WO 2018152171, US 10472386, WO 2018187804, US 10676500, WO 2019118571, US 10689391, WO 2020231917, WO 2016073767, US 10266560, WO 2016086134, US 10208081, WO 2016086218, US 10696713, US 10968249, WO 2016086115, WO 2016161003, US 10457703, US 11040998, US 11034684, US 10947264, US 10961272, WO 2019160813, or US 10829486, the bile acids and analogs or derivatives thereof of each of which are incorporated herein by reference. In some embodiments, a FXR agonist is reported in WO 2016086169, US 10519191, WO 2016130809, US 10246483, WO 2017147174, WO 2017147159, WO 2017147137, US 10364267, US 10323061, US 10323060, WO 2017189663, WO 2017189652, WO 2017189651, US 10080743, US 10080742, US 10080741, WO 2017201155, WO 2017201152, WO 2017201150, US 10144729, US 10138228, WO 2018067704, US 10450306, WO 2018081285, US 10597391, WO 2018102418, US 10584145, WO 2018152171, US 10472386, WO 2018187804, US 10676500, WO 2019118571, US 10689391, WO 2020231917, WO 2016073767, US 10266560, WO 2016086134, US 10208081, WO 2016086218, US 10696713, US 10968249, WO 2016086115, WO 2016161003, US US 10457703, US 11040998, US 11034684, US 10947264, US 10961272, WO 2019160813, or US 10829486, the FXR agonists of each of which are incorporated herein by reference.

In some embodiments, a condition, disorder or disease is primary biliary cholangitis (PBC). In some embodiments, a condition, disorder or disease is primary biliary cholangitis (PBC) without cirrhosis or with compensated cirrhosis which does not have evidence of portal hypertension. In some embodiments, another therapeutic agent is or delivers obeticholic acid or a pharmaceutically acceptable salt thereof. In some embodiments, another therapeutic agent is or delivers ursodeoxycholic acid (UDCA) or a pharmaceutically acceptable salt thereof. In some embodiments, the present disclosure provides methods for treating primary biliary cholangitis (PBC) comprising administering or delivering to a subject suffering therefrom an effective amount of a provided compound, wherein the subject is receiving or is exposed to or is under the effect of another therapeutic agent (e.g., UDCA or a salt thereof). In some embodiments, another therapeutic agent is administered or delivered concurrently with a provide compound. In some embodiments, another therapeutic agent is administered or delivered through the same pharmaceutical composition with a provide compound. In some embodiments, a provided compound is administered prior to or subsequent to another therapeutic agent. In some embodiments, a subject is exposed to therapeutically relevant levels of a provided compound and another therapeutic agent at the same time. In some embodiments, a subject is exposed to therapeutically relevant effects of a provided compound and another therapeutic agent at the same time.

In some embodiments, a condition, disorder or disease is a bile acid synthesis disorder. In some embodiments, a condition, disorder or disease is a bile acid synthesis disorder due to single enzyme defects (SEDs). In some embodiments, a condition, disorder or disease is peroxisomal disorders (PDs). In some embodiments, a condition, disorder or disease is peroxisomal disorders (PDs) including Zellweger spectrum disorders. In some embodiments, a subject exhibits manifestations of liver disease, steatorrhea or complications. In some embodiments, another therapeutic agent is cholic acid.

In some embodiments, a condition, disorder or disease is a neurodegenerative condition, disorder or disease. In some embodiments, a condition, disorder or disease is ALS. In some embodiments, another therapeutic agent is taurursodiol in combination with sodium phenylbutyrate (Relyvrio).

In some embodiments, a subject is an adult patient. In some embodiments, a subject is a pediatric patient.

### Pharmaceutical Compositions and Administration

In some embodiments, the present disclosure provides a pharmaceutical composition that comprise a provided compound or a pharmaceutically acceptable salt thereof, and a pharmaceutically acceptable carrier. In some embodiments, the present disclosure provides a pharmaceutical composition that can deliver a provided compound or a pharmaceutically acceptable salt thereof, and a pharmaceutically acceptable carrier. In some embodiments, a compound is provided in a pharmaceutically acceptable salt form.

Various technologies, e.g., routes, modes, dosage regimens, etc. may be utilized to administer and/or deliver provided compounds and compositions in accordance with the present disclosure. In some embodiments, a route and/or mode of administration can vary depending upon desired results. One with skill in the art, i.e., a physician, is aware that dosage regimens can be adjusted to provide a desired response, e.g., a therapeutic response. Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. In some embodiments, a compound is administered or delivered topically. In some embodiments, a composition is or comprises a topical composition. In some embodiments, a composition is or comprises a solution. In some embodiments, a composition is or comprises an emulsion. In some embodiments, a composition is or comprises a lotion. In some embodiments, a composition is or comprises an ointment. In some embodiments, a composition is or comprises a cream. In some embodiments, a composition is or comprises a gel. In some embodiments, a mode of administration is left to discretion of a practitioner.

In some embodiments, compounds can be incorporated into and administered as pharmaceutical compositions. Such pharmaceutical compositions are useful for, among other things, administration and delivery to a subject in vivo or ex vivo. In some embodiments, pharmaceutical compositions also contain a pharmaceutically acceptable carrier. In some embodiments, a pharmaceutically acceptable carrier is a pharmaceutical agent that does not itself induce an immune response harmful to the individual receiving a composition, and which may be administered without undue toxicity. Pharmaceutically acceptable carriers (or excipients) include, but are not limited to, liquids such as water, saline, glycerol, sugars and ethanol. Additionally, auxiliary substances, such as wetting or emulsifying agents, pH buffering substances, and the like, may be present in such vehicles.

Compounds in pharmaceutical compositions may be provided as pharmaceutically acceptable salts. In some embodiments, salts can be formed with many acids, including but not limited to, hydrochloric, sulfuric, acetic, lactic, tartaric, malic, succinic, benzenesulfonic, etc. In some embodiments, salts can be formed with bases. In some embodiments, salts are alkali, alkaline earth metal, or ammonium salts, e.g., sodium, calcium, diethanolamine, ethanolamine, trialkylamine salts, etc.

In some embodiments, salts are more soluble in aqueous or other protonic solvents than corresponding, free acid or base forms. In some embodiments, a pharmaceutical composition may be a lyophilized powder. In some embodiments, a pharmaceutical composition comprises a provided compound, e.g., a compound of formula I or a pharmaceutically acceptable salt thereof dissolved in a pharmaceutically acceptable buffer. In some embodiments, a buffer is a saline buffer. In some embodiments, a buffer has a pH around 7.4.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. In some embodiments, pharmaceutical compositions or formulations are tablets (coated or uncoated), capsules (hard or soft), microbeads, powder, granules and/or crystals. Supplementary active compounds (e.g., preservatives, antibacterial, antiviral and antifungal agents) can also be incorporated into pharmaceutical compositions.

Pharmaceutical compositions can be formulated to be compatible with a particular route of administration or delivery as set forth herein or known to one of skill in the art.

In some embodiments, provided compositions are suitable for parenteral administration. In some embodiments, such compositions comprise aqueous and non-aqueous solutions, suspensions or emulsions of active compounds, which preparations are typically sterile and can be isotonic with blood of intended recipients. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, a suspension may also contain suitable stabilizers or agents which increase solubility to allow for the preparation of highly concentrated solutions.

Co-solvents and adjuvants may be added to compositions and formulations. Non-limiting examples of co-solvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Various pharmaceutical compositions and delivery systems appropriate for compositions, methods and uses of the present disclosure are known in the art (see, e.g., Remington: The Science and Practice of Pharmacy. 21st Edition. Philadelphia, PA. Lippincott Williams & Wilkins, 2005) and can be utilized in accordance with the present disclosure.

In some embodiments, the present disclosure provides methods for delivering provided compounds and compositions into cells, animals or subjects. In some embodiments, such methods include contacting a subject (e.g., a cell or tissue of a subject) with, or administering or delivering to a subject (e.g., a subject such as a mammal or human) a provided compound, e.g., a compound of formula I or a salt thereof, or a composition thereof.

A compound or composition described herein can be administered in a sufficient or effective amount to a subject (or a cell, tissue or organ thereof) in need thereof. Doses can vary and may depend upon the type, onset, progression, severity, frequency, duration, or probability of a condition, disorder or disease to which treatment is directed, a clinical endpoint desired, previous or simultaneous treatments, general health, age, gender, race or immunological competency of a subject and other factors that will be appreciated by a skilled artisan. Dose amount, number, frequency or duration may be proportionally increased or reduced, as indicated by efficacy, any adverse side effects, complications or other risk factors of a treatment or therapy and the status of a subject. A skilled artisan will appreciate factors that may influence dosage and timing required to provide an amount sufficient for providing a therapeutic or prophylactic benefit.

A dose to achieve a therapeutic effect will vary based on several factors including route of administration, amount to achieve a therapeutic effect, specific condition, disorder or disease treated, any host immune response to administered compound or composition, stability of administered compound or composition, etc.

An effective amount or a sufficient amount can be provided in a single administration, may require multiple administrations, and, can be, administered alone or in combination with another composition (e.g., comprising or delivering another therapeutic agent). For example, an amount may be proportionally increased as indicated by the need of a subject, type, status and severity of a condition, disorder or disease treated and/or side effects (if any) of treatment. Amounts considered effective also include amounts that result in a reduction of the use of another treatment, therapeutic regimen or protocol.

In some embodiments, pharmaceutical compositions comprise or deliver active ingredients, e.g., compounds of formula I or pharmaceutically acceptable salts thereof, in effective amounts to achieve intended purposes e.g., therapeutic purposes. Various technologies may be utilized to determine therapeutically effective amounts in accordance with the present disclosure. Therapeutic doses can depend on, among other factors, ages and general conditions of subjects, severity of conditions, disorders or diseases, etc. In some embodiments, therapeutically effective amounts in humans may fall in a relatively broad range that may be determined by medical practitioners based on responses of individual patients.

In some embodiments, methods and uses of the present disclosure include delivery and administration systemically, regionally or locally, or by any route, for example, by injection or infusion or orally. In some embodiments, delivery of a pharmaceutical composition in vivo may generally be accomplished via injection using a conventional syringe, although other delivery methods such as convection-enhanced delivery can also be used In some embodiments, compounds and compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. In some embodiments, modes of administration include oral and pulmonary administration, suppositories, and transdermal applications. Clinicians specializing in treating patients may determine optimal routes for administration of compounds and compositions as described herein.

Among other things, the present disclosure provides the following Embodiments:

1. A compound having the structure of Formula I:

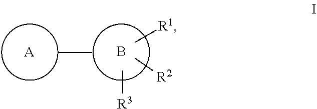

or a pharmaceutically acceptable salt thereof, wherein:
R¹ is —C(O)OR¹¹, —P(O)(OR¹²)(OR¹³), —C(O)N(R¹⁴)SO₂R¹⁵, —C(O)NR¹⁶R¹⁷, —CN,

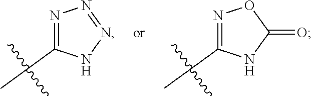 [SMILES: *c1nnn[nH]1 ; *c1noc(=O)[nH]1]

each of R² and R³ is independently R′, —OR′, halogen, CN, —NO₂, or —N(R′)₂;
Ring A is

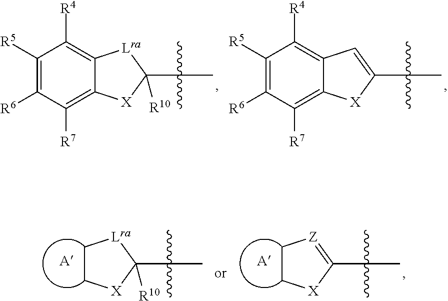

wherein Ring A′ is an optionally substituted 5-10 membered aromatic ring having 0-4 heteroatoms;
L^{ra} is optionally substituted —(CH₂)ₙ—;
n is 1, 2 or 3;
X is —O—, —S—, —N(R⁸)- or optionally substituted —CH₂—;
Z is —N═ or —C(R⁹)═;
each of R⁴, R⁵, R⁶, R⁷ and R⁹ is independently R′, —OR′, halogen, CN, —NO₂, or —N(R′)₂;
Ring B is an optionally substituted ring selected from a 6-10 membered aryl ring and a 5-10 membered heteroaryl ring having 1-6 heteroatoms;
each of R⁸, R¹⁰, R¹¹, R¹², R¹³, R¹⁴, R¹⁵, R¹⁶, and R¹⁷ is independently R′;
each R¹ is independently R, —OR, —C(O)R, —C(O) OR, or —S(O)₂R;
each R is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

2. A compound having the structure of Formula I:

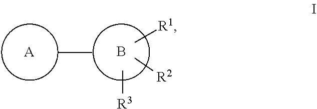

or a pharmaceutically acceptable salt thereof, wherein:
R′ is —C(O)OR¹¹, —P(O)(OR¹²)(OR¹³), —C(O)N(R¹⁴)SO₂R¹⁵, —C(O)NR¹⁶R¹⁷, —CN,

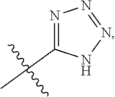 [SMILES: *c1nnn[nH]1]

halogen, or

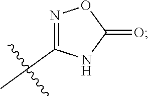 [SMILES: *c1noc(=O)[nH]1]

each of R² and R³ is independently R′, —OR′, halogen, —CN, —NO₂, or —N(R′)₂;
Ring A is

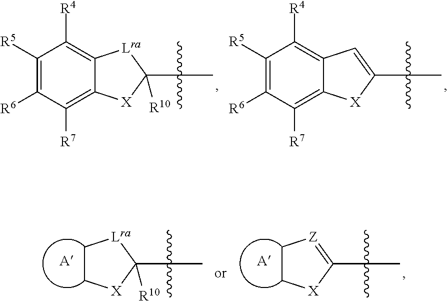

wherein Ring A′ is an optionally substituted 5-10 membered aromatic ring having 0-4 heteroatoms;
L^{ra} is optionally substituted —(CH₂)ₙ—;
n is 1, 2 or 3;
X is —O—, —S—, —N(R⁸)—or optionally substituted —CH₂—;
Z is —N═ or —C(R⁹)═;
each of R⁴, R⁵, R⁶, R⁷ and R⁹ is independently R′, —OR′, halogen, —CN, —NO₂, or —N(R′)₂;
Ring B is an optionally substituted ring selected from a 6-10 membered aryl ring and a 5-10 membered heteroaryl ring having 1-6 heteroatoms;
each of R⁸, R¹⁰, R¹, R¹², R¹³, R¹⁴, R¹⁵, R¹⁶, and R¹⁷ is independently R′;
each R¹ is independently R, OR, C(O)R, C(O)OR, or —S(O)₂R;
each R is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

3. A compound having the structure of Formula I:

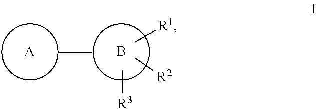

or a pharmaceutically acceptable salt thereof, wherein:
R¹ is —C(O)OH or an isostere thereof, optionally protected-CHO or R^{d6};
R^{d6} is —CH(OR)₂;
each of R² and R³ is independently R′, OR′, halogen, —CN, —NO₂, or —N(R′)₂;
Ring A is

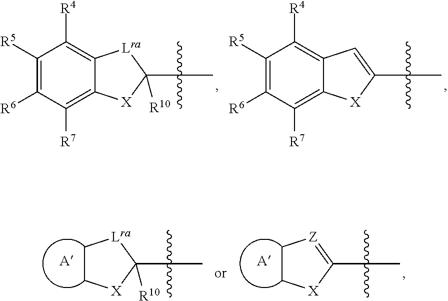

wherein Ring A′ is an optionally substituted 5-10 membered aromatic ring having 0-4 heteroatoms;
L^{ra} is optionally substituted —(CH₂)ₙ;
n is 1, 2 or 3;
X is —O—, —S—, —N(R⁸)- or optionally substituted —CH₂—;
Z is —N═ or —C(R⁹)═;
each of R⁴, R⁵, R⁶, R⁷ and R⁹ is independently R′, —OR′, halogen, —CN, —NO₂, or —N(R′)₂;
Ring B is an optionally substituted ring selected from a 6-10 membered aryl ring and a 5-10 membered heteroaryl ring having 1-6 heteroatoms;
each of R⁸, R¹⁰, R¹¹, R¹², R¹³, R¹⁴, R¹⁵, R¹⁶, and R¹⁷ is independently R′;
each R¹ is independently R, —OR, —C(O)R, —C(O) OR, or —S(O)₂R;
each R is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

4. The compound of any one of the preceding Embodiments, wherein Ring A′ is an optionally substituted 5-6 membered aromatic ring having 0-4 heteroatoms.

5. The compound of any one of Embodiments 1-3, wherein Ring A is

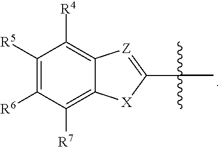

6. The compound of any one of Embodiments 1-4, wherein Ring A is

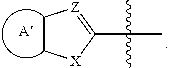

7. The compound of Embodiment 6, wherein Ring A′ is an optionally substituted 5-6 membered aromatic ring having 1, 2, 3 or 4 heteroatoms.

8. The compound of Embodiment 6, wherein Ring A′ is an optionally substituted 5 membered aromatic ring having 1 or 2 heteroatoms.

9 The compound of Embodiment 6, wherein Ring A′ is an optionally substituted phenyl ring.

10. The compound of Embodiment 6, wherein Ring A′ is an optionally substituted 6 membered aromatic ring having 1 or 2 heteroatoms.

11. The compound of Embodiment 6, wherein Ring A′ is an optionally substituted 9 membered aromatic ring having 1, 2, 3, or 4 heteroatoms.

12. The compound of Embodiment 6, wherein Ring A′ is an optionally substituted 9 membered aromatic ring having 1 or 2 heteroatoms.

13. The compound of Embodiment 6, wherein Ring A′ is an optionally substituted 10 membered aromatic ring having 1, 2, 3, or 4 heteroatoms.

14. The compound of Embodiment 6, wherein Ring A′ is an optionally substituted 10 membered aromatic ring having 1 or 2 heteroatoms.

15. The compound of Embodiment 6, wherein Ring A′ is an optionally substituted bivalent naphthyl ring.

16. The compound of Embodiment 6, wherein Ring A′ is optionally substituted

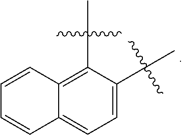 [SMILES: *c1ccc2ccccc2c1*]

17. The compound of Embodiment 6, wherein Ring A′ is

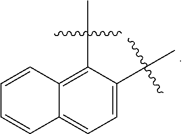 [SMILES: *c1ccc2ccccc2c1*]

18. The compound of any one of the preceding Embodiments, wherein Z is —C(R⁹)═.

19. The compound of any one of Embodiments 1-11, wherein R⁹ is H.

20. The compound of any one of Embodiments 1-11, wherein R⁹ is halogen.

21. The compound of any one of Embodiments 1-11, wherein R⁹ is F.

22. The compound of any one of Embodiments 1-11, wherein R⁹ is optionally substituted C₁-C₆ aliphatic.

23. The compound of any one of Embodiments 1-11, wherein R⁹ is optionally substituted C₁-C₆ alkyl.

24. The compound of any one of Embodiments 1-11, wherein R⁹ is methyl.

25. The compound of any one of Embodiments 1-4, wherein Ring A is

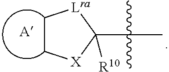

26. The compound of any one of Embodiments 1-4, wherein Ring A is

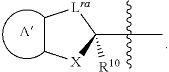

27. The compound of any one of Embodiments 1-4, wherein Ring A is

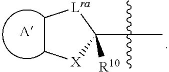

28. The compound of any one of Embodiments 1-4, wherein Ring A is

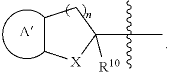

29. The compound of any one of Embodiments 1-4, wherein Ring A is

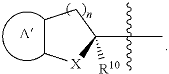

30. The compound of any one of Embodiments 1-4, wherein Ring A is

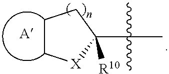

31. The compound of any one of Embodiments 25-30, wherein Ring A′ is an optionally substituted 5-6 membered aromatic ring having 1, 2, 3 or 4 heteroatoms.

32. The compound of any one of Embodiments 25-30, wherein Ring A′ is an optionally substituted 5 membered aromatic ring having 1 or 2 heteroatoms.

33. The compound of any one of Embodiments 25-30, wherein Ring A′ is an optionally substituted phenyl ring.

34. The compound of any one of Embodiments 25-30, wherein Ring A′ is an optionally substituted 6 membered aromatic ring having 1 or 2 heteroatoms.

35. The compound of any one of Embodiments 25-30, wherein Ring A′ is an optionally substituted 9 membered aromatic ring having 1, 2, 3, or 4 heteroatoms.

36. The compound of any one of Embodiments 25-30, wherein Ring A′ is an optionally substituted 9 membered aromatic ring having 1 or 2 heteroatoms.

37. The compound of any one of Embodiments 25-30, wherein Ring A′ is an optionally substituted 10 membered aromatic ring having 1, 2, 3, or 4 heteroatoms.

38. The compound of any one of Embodiments 25-30, wherein Ring A′ is an optionally substituted 10 membered aromatic ring having 1 or 2 heteroatoms.

39. The compound of any one of Embodiments 25-30, wherein Ring A′ is an optionally substituted bivalent naphthyl ring.

40. The compound of any one of Embodiments 25-30, wherein Ring A′ is optionally substituted

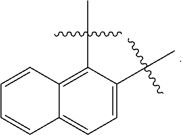 [SMILES: *c1ccc2ccccc2c1*]

41. The compound of any one of Embodiments 25-30, wherein Ring A′ is

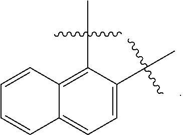 [SMILES: *c1ccc2ccccc2c1*]

42. The compound of Embodiment 1 or 2, wherein Ring A is

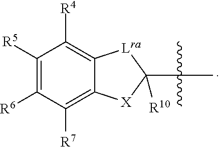

43. The compound of Embodiment 1 or 2, wherein Ring A is

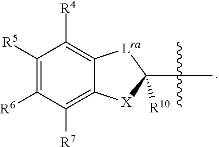

44. The compound of Embodiment 1 or 2, wherein Ring A is

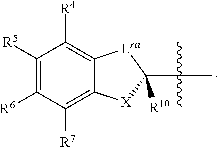

45. The compound of Embodiment 1 or 2, wherein Ring A is

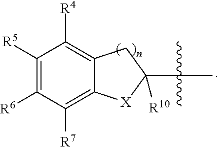

46. The compound of Embodiment 1 or 2, wherein Ring A is

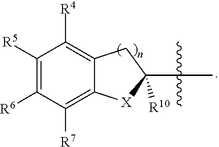

47. The compound of Embodiment 1 or 2, wherein Ring A is

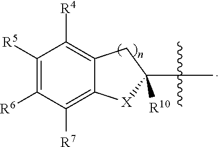

48. The compound of any one of Embodiments 25-197, wherein n is 1.

49. The compound of any one of Embodiments 25-197, wherein n is 2.

50. The compound of any one of Embodiments 25-197, wherein n is 3.

51. The compound of any one of Embodiments 25-50, wherein R¹⁰ is H.

52. The compound of any one of the preceding Embodiments, wherein X is —N(R⁸)—

53. A compound, wherein the compound has the structure of formula B-5:

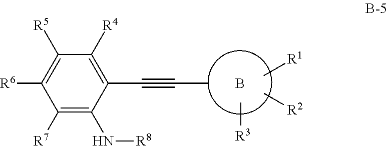

or a salt thereof, wherein each variable is independently as described in Embodiment 1 or 2.

54. The compound of any one of the preceding Embodiments, wherein R⁸ is H.

55. The compound of any one of Embodiments 1-53, wherein R⁸ is not H.

56. The compound of any one of Embodiments 1-53, wherein R⁸ is optionally substituted C₁-C₆ alkyl.

57. The compound of Embodiment 56, wherein R⁸ is methyl.

58. The compound of Embodiment 56, wherein R⁸ is propyl.

59. The compound of Embodiment 56, wherein R⁸ is isopropyl.

60. The compound of Embodiment 56, wherein R⁸ is isobutyl.

61. The compound of any one of Embodiments 1-53, wherein R⁸ is optionally substituted C₃-C₈ cycloalkyl.

62. The compound of Embodiment 61, wherein R⁸ is

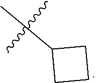 [SMILES: *C1CCC1]

63. The compound of Embodiment 61, wherein R⁸ is

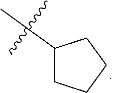 [SMILES: *C1CCCC1]

64. The compound of Embodiment 61, wherein R⁸ is

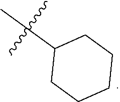 [SMILES: *C1CCCCC1]

65. The compound of any one of Embodiments 1-53, wherein R⁸ is optionally substituted 6-10 membered aryl.

66. The compound of Embodiment 65, wherein R⁸ is phenyl.

67. The compound of any one of Embodiments 1-53, wherein R⁸ is optionally substituted 6-10 membered aryl-C₁-C₆ alkyl.

68. The compound of Embodiment 67, wherein R⁸ is

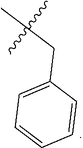 [SMILES: *Cc1ccccc1]

69. The compound of any one of Embodiments 1-51, wherein X is —O—.

70. The compound of any one of Embodiments 1-51, wherein X is optionally substituted —CH₂—.

71. The compound of any one of Embodiments 1-51, wherein X is —CH₂—.

72. The compound of any one of Embodiments 1-51, wherein X is —S—.

73. The compound of any one of Embodiments 1-10 and 53-72, wherein Z is —N═.

74. A compound, wherein the compound has the structure of formula B-1:

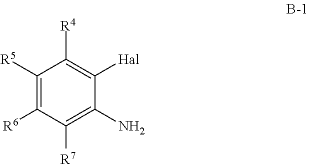

or a salt thereof, wherein Hal is halogen, and each other variable is independently as described in Embodiment 1 or 2.

75. A compound, wherein the compound has the structure of formula C-1:

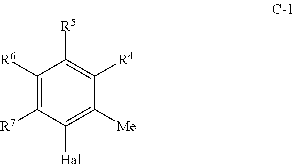

or a salt thereof, wherein Hal is halogen and each other variable is independently as described in Embodiment 1 or 2.

76. A compound, wherein the compound has the structure of formula C-2:

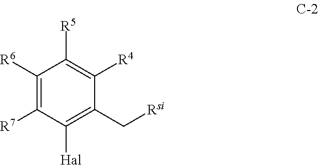

or a salt thereof, wherein Hal is halogen, R^{si} is —Si(R)₃, and each other variable is independently as described in Embodiment 1 or 2.

77. The compound of Embodiment 76, wherein R^{si} is —Si(R)₃ wherein in each R is independently not —H.

78. The compound of Embodiment 76, wherein R^{si} is —Si(R)₃ wherein in each R is independently an optionally substituted selected from C₁₋₆ aliphatic and C₆₋₁₀ aryl.

79. The compound of Embodiment 76, wherein R^{si} is —Si(R)₃ wherein in each R is independently an optionally substituted selected from C₁₋₆ aliphatic and phenyl.

80. The compound of Embodiment 76, wherein R^{si} is —Si(R)₃ wherein in each R is independently optionally substituted C₁₋₆ aliphatic.

81. The compound of Embodiment 76, wherein R^{si} is —Si(Me)₃.

82. A compound, wherein the compound has the structure of formula C-4:

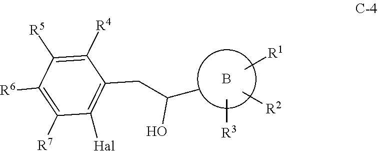

or a salt thereof, wherein Hal is halogen, and each other variable is independently as described in Embodiment 1 or 2.

83. The compound of any one of Embodiments 74-82, wherein Hal is Cl.

84. The compound of any one of Embodiments 74-82, wherein Hal is Br.

85. The compound of any one of Embodiments 74-82, wherein Hal is I.

86. A compound, wherein the compound has the structure of formula D-2:

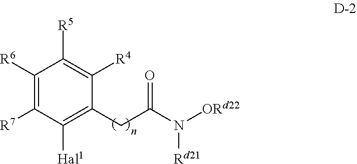

or a salt thereof, wherein Hal is halogen, and each other variable is independently as described in Embodiment 1 or 2.

87. The compound of Embodiment 86, wherein R^{d21} is optionally substituted C₁₋₆ aliphatic.

88. The compound of Embodiment 86, wherein R^{d21} is methyl.

89. The compound of any one of Embodiments 86-88, wherein R^{d22} is optionally substituted C₁₋₆ aliphatic.

90. The compound of any one of Embodiments 86-88, wherein R^{d22} is methyl.

91. A compound, wherein the compound has the structure of formula D-2:

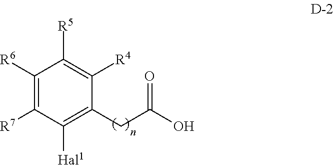

or a salt thereof, wherein Hall is halogen, and each other variable is independently as described in Embodiment 1 or 2.

92. A compound, wherein the compound has the structure of formula D-4:

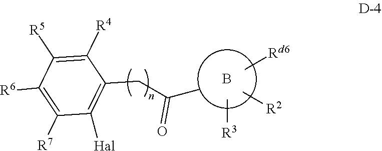

or a salt thereof, wherein Hall is halogen, and each other variable is independently as described in Embodiment 1 or 2.

93. A compound, wherein the compound has the structure of formula D-5:

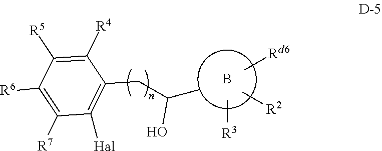

or a salt thereof, wherein Hal¹ is halogen, and each other variable is independently as described in Embodiment 1 or 2.

94. The compound of any one of Embodiments 86-93, wherein Hall is Cl.

95. The compound of any one of Embodiments 86-93, wherein Hall is Br.

96. The compound of any one of Embodiments 86-93, wherein Hall is Br.

97. The compound of any one of Embodiments 86-96, wherein n is 1.

98. The compound of any one of Embodiments 86-96, wherein n is 2.

99. The compound of any one of Embodiments 86-96, wherein n is 3.

100. A compound, wherein the compound has the structure of formula D′-2:

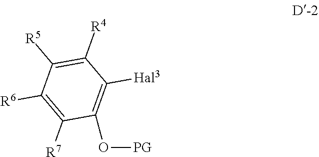

or a salt thereof, wherein Hal³ is halogen, PG is R′ or a protecting group, and each other variable is independently as described in Embodiment 1 or 2.

101. The compound of Embodiment 100, wherein Hal³ is Cl.

102. The compound of Embodiment 100, wherein Hal³ is Br.

103 The compound of Embodiment 100, wherein Hal³ is I.

104. A compound, wherein the compound has the structure of formula D′-3:

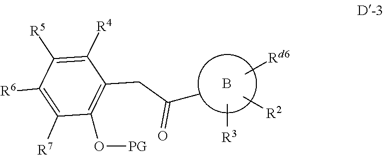

or a salt thereof, wherein PG is R′ or a protecting group, and each other variable is independently as described in Embodiment 1 or 2.

105. A compound, wherein the compound has the structure of formula D′-4:

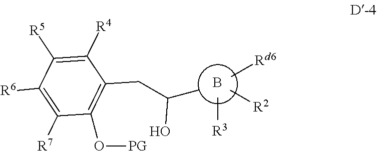

or a salt thereof, wherein PG is R′ or a protecting group, and each other variable is independently as described in Embodiment 1 or 2.

106. The compound of any one of Embodiments 100-105, wherein PG is R′ wherein R¹ is not H.

107. The compound of any one of Embodiments 100-105, wherein PG is a protecting group.

108 The compound of any one of Embodiments 100-105, wherein PG is selected from Bn, MEM, and allyl.

109. A compound, wherein the compound has the structure of formula D′-5:

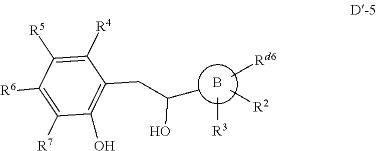

or a salt thereof, wherein each variable is independently as described in Embodiment 1 or 2.

110. A compound, wherein the compound has the structure of formula B-3:

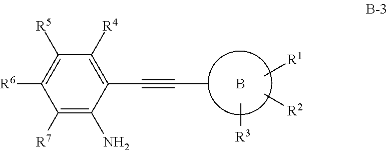

111. or a salt thereof, wherein each variable is independently as described in Embodiment 1 or 2. The compound of any one of the preceding Embodiments, wherein R⁴ is R.

112. The compound of any one of the preceding Embodiments, wherein R⁴ is optionally substituted C₁-C₆ alkyl.

113. The compound of any one of the preceding Embodiments, wherein R⁴ is —CF₃.

114. The compound of any one of Embodiments 1-110, wherein R⁴ is —H.

115. The compound of any one of Embodiments 1-110, wherein R⁴ is halogen.

116. The compound of any one of Embodiments 1-110, wherein R⁴ is —F.

117. The compound of any one of Embodiments 1-110, wherein R⁴ is —Cl.

118. The compound of any one of Embodiments 1-110, wherein R⁴ is R.

119. The compound of any one of Embodiments 1-110, wherein R⁴ is optionally substituted phenyl.

120. The compound of any one of Embodiments 1-110, wherein R⁴ is phenyl.

121. The compound of any one of the preceding Embodiments, wherein R⁵ is —H.

122. The compound of any one of Embodiments 1-120, wherein R⁵ is optionally substituted C₁₋₆ aliphatic.

123. The compound of any one of Embodiments 1-120, wherein R⁵ is optionally substituted C₁₋₆ alkyl.

124. The compound of any one of Embodiments 1-120, wherein R⁵ is —CF₃.

125. The compound of any one of Embodiments 1-120, wherein R⁵ is halogen.

126. The compound of any one of Embodiments 1-120, wherein R⁵ is —F.

127. The compound of any one of Embodiments 1-120, wherein R⁵ is —Cl.

128. The compound of any one of Embodiments 1-120, wherein R⁵ is —CN.

129 The compound of Embodiment 1 or 2, wherein Ring A is

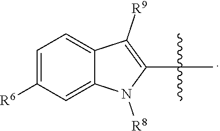

130. The compound of any one of the preceding Embodiments, wherein R⁶ is —H.

131 The compound of nay one of Embodiments 1-128, wherein R⁶ is R.

132. The compound of nay one of Embodiments 1-128, wherein R⁶ is halogen, —CN, optionally substituted C₁-C₆ alkyl, —OR wherein R is optionally substituted C₁-C₆ alkyl, optionally substituted 6-10 membered aryl, C(O)OR wherein R is H or optionally substituted C₁-C₆ alkyl, or —S(O)₂R wherein R is H or optionally substituted C₁-C₆ alkyl.

133. The compound of nay one of Embodiments 1-128, wherein R⁶ is optionally substituted C₁₋₆ aliphatic.

134. The compound of nay one of Embodiments 1-128, wherein R⁶ is optionally substituted C₁₋₆ alkyl.

135. The compound of nay one of Embodiments 1-128, wherein R⁶ is —CF₃.

136. The compound of nay one of Embodiments 1-128, wherein R⁶ is optionally substituted phenyl.

137 The compound of nay one of Embodiments 1-128, wherein R⁶ is —OR.

138. The compound of nay one of Embodiments 1-128, wherein R⁶ is —OR wherein R is optionally substituted C₁₋₆ aliphatic.

139. The compound of nay one of Embodiments 1-128, wherein R⁶ is —OMe.

140. The compound of nay one of Embodiments 1-128, wherein R⁶ is —OCF₃.

141. The compound of nay one of Embodiments 1-128, wherein R⁶ is halogen.

142. The compound of nay one of Embodiments 1-128, wherein R⁶ is —F.

143. The compound of nay one of Embodiments 1-128, wherein R⁶ is —Cl.

144. The compound of nay one of Embodiments 1-128, wherein R⁶ is —CN.

145. The compound of nay one of Embodiments 1-128, wherein R⁶ is —C(O)OR.

146. The compound of nay one of Embodiments 1-128, wherein R⁶ is —C(O)OH.

147 The compound of nay one of Embodiments 1-128, wherein R⁶ is —S(O)₂R.

148. The compound of nay one of Embodiments 1-128, wherein R⁶ is —S(O)₂R wherein R is C₁₋₆ aliphatic.

149. The compound of nay one of Embodiments 1-128, wherein R⁶ is —S(O)₂Me.

150. The compound of any one of the preceding Embodiments, wherein R⁷ is —H.

151. The compound of any one of Embodiments 1-149, wherein R⁷ is R.

152. The compound of any one of Embodiments 1-149, wherein R⁷ is optionally substituted C₁₋₆ aliphatic.

153. The compound of any one of Embodiments 1-149, wherein R⁷ is halogen.

154. The compound of any one of Embodiments 1-149, wherein R⁷ is —F.

155. The compound of any one of Embodiments 1-149, wherein R⁷ is —Cl.

156. The compound of Embodiment 1 or 2, wherein Ring A is

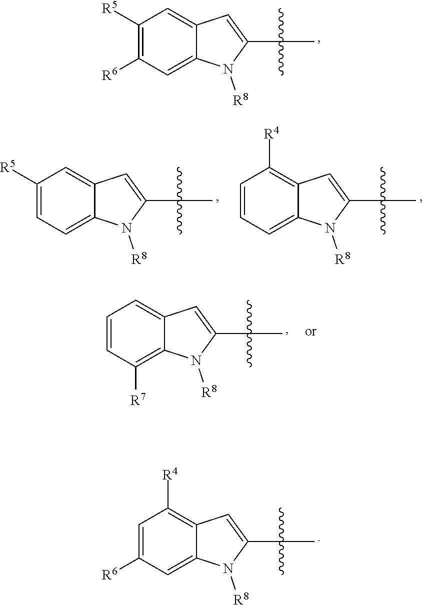

157. The compound of Embodiment 1 or 2, wherein Ring A is

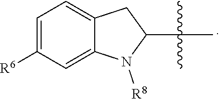

158. The compound of Embodiment 157, wherein R⁶ is —CF₃.

159. The compound of Embodiment 157 or 158, wherein R⁸ is H or methyl.

160. The compound of Embodiment 1 or 2, wherein Ring A is

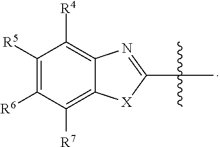

161. The compound of Embodiment 160, wherein Ring A is

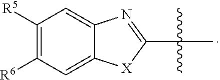

162. The compound of Embodiment 160 or 161, wherein X is O or S.

163. The compound of any one of Embodiments 160-162, wherein R⁵ is optionally substituted C₁-C₆ alkyl.

164. The compound of any one of Embodiments 160-163, wherein R⁵ is —CF₃.

165. The compound of any one of Embodiments 160-164, wherein R⁶ is halogen.

166. The compound of any one of Embodiments 160-165, wherein R⁶ is Cl.

167 The compound of Embodiment 1 or 2, wherein Ring A is

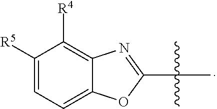

168. The compound of Embodiment 167, wherein R⁴ is optionally substituted C₁-C₆ alkyl.

169. The compound of Embodiment 167 or 168, wherein R⁴ is —CF₃.

170. The compound of any one of Embodiments 167-169, wherein R⁵ is halogen.

171. The compound of any one of Embodiments 167-170, wherein R⁵ is F or C1.

172. The compound of Embodiment 1 or 2, wherein Ring A is

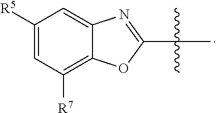

173. The compound of Embodiment 172, wherein R⁵ is optionally substituted C₁-C₆ alkyl.

174. The compound of Embodiment 172 or 173, wherein R⁵ is —CF₃.

175. The compound of any one of Embodiments 172-174, wherein R⁷ is halogen.

176. The compound of any one of Embodiments 172-175, wherein R⁷ is Cl.

177. The compound of Embodiment 1 or 2, wherein Ring A is

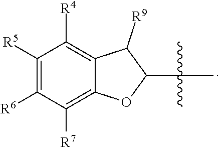

178. The compound of Embodiment 177, wherein Ring A is

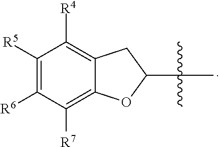

179 The compound of Embodiment 177 or 178, wherein Ring A is

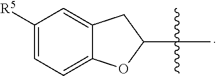

180. The compound of any one of Embodiments 177-179, wherein R⁵ is halogen or optionally substituted C₁-C₆ alkyl.

181. The compound of any one of Embodiments 177-180, wherein R⁵ is Cl.

182. The compound of any one of Embodiments 177-180, wherein R⁵ is —CF₃.

183. The compound of any one of Embodiments 177-179, wherein Ring A is

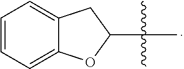 [SMILES: *C1Cc2ccccc2O1]

184. The compound of Embodiment 1 or 2, wherein Ring A is

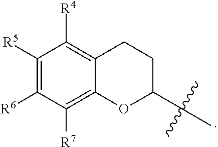

185. The compound of Embodiment 184, wherein Ring A is

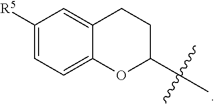

186. The compound of Embodiment 184 or 185, wherein R⁵ is optionally substituted C₁-C₆ alkyl.

187. The compound of any one of Embodiments 184-186, wherein R⁵ is —CF₃.

188. The compound of Embodiment 1 or 2, wherein Ring A is

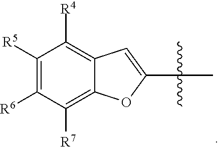

189. The compound of Embodiment 188, wherein Ring A is

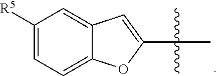

190. The compound of Embodiment 188 or 189, wherein R⁵ is optionally substituted C₁-C₆ alkyl.

191. The compound of any one of Embodiments 188-190, wherein R⁵ is —CF₃.

192. The compound of Embodiment 188, wherein Ring A is

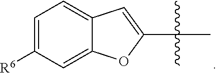

193. The compound of Embodiment 192, wherein R⁶ is optionally substituted C₁-C₆ alkyl.

194. The compound of Embodiment 192 or 193, wherein R⁶ is —CF₃.

195. The compound of any one of the preceding Embodiments, wherein R⁴ and R⁵ are taken together with their intervening atoms to form an optionally substituted 5- or 6-membered aromatic ring having 0-4 heteroatoms independently selected from nitrogen, oxygen and sulfur.

196. The compound of any one of the preceding Embodiments, wherein R⁴ and R⁵ are taken together with their intervening atoms to form an optionally substituted phenyl ring.

197. The compound of any one of the preceding Embodiments, wherein the formed ring is unsubstituted.

198. A compound, wherein the compound has the structure of formula B-2:

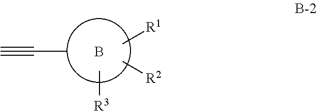

or a salt thereof, wherein each variable is independently as described in Embodiment 1 or 2.

199. A compound, wherein the compound has the structure of formula C-3:

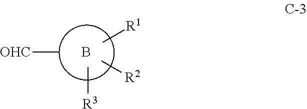

or a salt thereof, wherein each variable is independently as described in Embodiment 1 or 2.

200. A compound, wherein the compound has the structure of formula D′-1:

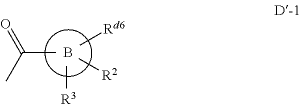

or a salt thereof, wherein each variable is independently as described in Embodiment 1 or 2.

201. A compound, wherein the compound has the structure of formula D-3:

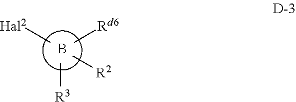

or a salt thereof, wherein Hal² is halogen, and each other variable is independently as described in Embodiment 1 or 2.

202. The compound of Embodiment 201, wherein Hal² is Cl.

203 The compound of Embodiment 201, wherein Hal² is Br.

204. The compound of Embodiment 201, wherein Hal² is I.

205. A compound, wherein the compound has the structure of formula B-2:

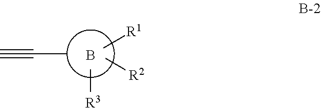

or a salt thereof, wherein each variable is independently as described in Embodiment 1 or 2.

206. The compound of any one of the preceding Embodiments, wherein Ring B is an optionally substituted phenyl ring.

207. The compound of any one of the preceding Embodiments, wherein Ring B is

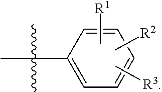

208. The compound of any one of Embodiments 206-207, wherein R¹ is at an o position (relative to the carbon bonded to Ring A).

209. The compound of Embodiment 208, wherein R² is at an o position.

210. The compound of Embodiment 209, wherein R³ is at an m position next to R².

211. The compound of Embodiment 209, wherein R³ is at an m position next to R¹.

212. The compound of Embodiment 209, wherein R³ is at a p position.

213 The compound of Embodiment 208, wherein R² is at an m position next to R¹.

214 The compound of Embodiment 213, wherein R³ is at an o position.

215. The compound of Embodiment 213, wherein R³ is at an m position.

216. The compound of Embodiment 213, wherein R³ is at a p position.

217 The compound of Embodiment 208, wherein R³ is at an m position next to R¹.

218 The compound of Embodiment 217, wherein R² is at an o position.

219 The compound of Embodiment 217, wherein R² is at an m position.

220. The compound of Embodiment 217, wherein R² is at a p position.

221. The compound of Embodiment 208, wherein R² is at a p position.

222. The compound of Embodiment 221, wherein R³ is at an o position.

223. The compound of Embodiment 221, wherein R³ is at an m position.

224. The compound of any one of Embodiments 206-207, wherein R¹ is at an m position (relative to the carbon bonded to Ring A).

225. The compound of Embodiment 224, wherein R² is at an o position next to R¹.

226. The compound of Embodiment 225, wherein R³ is at an o position.

227. The compound of Embodiment 225, wherein R³ is at an m position.

228 The compound of Embodiment 225, wherein R³ is at a p position.

229. The compound of Embodiment 224, wherein R³ is at an o position next to R¹.

230 The compound of Embodiment 229, wherein R² is at an o position.

231. The compound of Embodiment 229, wherein R² is at an m position.

232 The compound of Embodiment 229, wherein R² is at a p position.

233. The compound of Embodiment 224, wherein R² is at an m position.

234. The compound of Embodiment 233, wherein R³ is at an o position next to R².

235. The compound of Embodiment 233, wherein R³ is at a p position.

236. The compound of Embodiment 224, wherein R² is at a p position.

237. The compound of Embodiment 236, wherein R³ is at an o position next to R¹.

238. The compound of Embodiment 236, wherein R³ is at an o position not next to R¹.

239. The compound of Embodiment 236, wherein R³ is at an m position.

240. The compound of any one of Embodiments 206-207, wherein R¹ is at a p position (relative to the carbon bonded to Ring A).

241. The compound of Embodiment 240, wherein R² is at an o position.

242. The compound of Embodiment 241, wherein R³ is at an o position.

243. The compound of Embodiment 241, wherein R³ is at an m position between R¹ and R².

244. The compound of Embodiment 241, wherein R³ is at an m position not between R¹ and R².

245. The compound of Embodiment 240, wherein R² is at an m position.

246. The compound of Embodiment 245, wherein R³ is at an o position next to R².

247. The compound of Embodiment 245, wherein R³ is at an o position not next to R².

248. The compound of Embodiment 245, wherein R³ is at an m position.

249. The compound of any one of Embodiments 1-207, wherein Ring B is

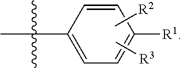

250. The compound of any one of Embodiments 1-207, wherein Ring B is

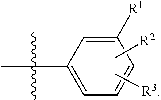

251. The compound of any one of Embodiments 1-207, wherein Ring B is

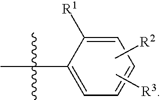

252. The compound of any one of Embodiments 1-205, wherein Ring B is an optionally substituted 10-membered bicyclic aryl ring.

253. The compound of any one of Embodiments 1-205, wherein Ring B is an optionally substituted 5-membered heteroaryl ring having 1-4 heteroatoms.

254. The compound of any one of Embodiments 1-205, wherein Ring B is an optionally substituted 6-membered heteroaryl ring having 1-4 heteroatoms.

255. The compound of any one of Embodiments 1-205, wherein Ring B is an optionally substituted 9-membered bicyclic heteroaryl ring having 1-4 heteroatoms.

256. The compound of any one of Embodiments 1-205, wherein Ring B is an optionally substituted 10-membered bicyclic heteroaryl ring having 1-4 heteroatoms.

257. The compound of any one of the preceding Embodiments, wherein R¹ is —C(O)OH.

258. The compound of any one of Embodiments 1-256, wherein R¹ is an isostere of —C(O)OH.

259. The compound of any one of Embodiments 1-256, wherein R¹ is —N(OH)C(O)R¹¹.

260 The compound of any one of Embodiments 1-256, wherein R¹ is —C(O)NHOR¹¹.

261. The compound of any one of Embodiments 1-256, wherein R¹ is —C(O)NR¹¹CN.

262. The compound of any one of Embodiments 1-256, wherein R¹ is —S(0)₂OR¹¹.

263. The compound of any one of Embodiments 259-262, wherein R¹¹ is R.

264. The compound of any one of Embodiments 259-262, wherein R¹¹ is H.

265. The compound of any one of Embodiments 259-262, wherein R¹¹ is optionally substituted C₁₋₆ aliphatic.

266. The compound of any one of Embodiments 1-256, wherein R¹ is —C(O)NHOH.

267. The compound of any one of Embodiments 1-256, wherein R¹ is —C(O)NHCN.

268 The compound of any one of Embodiments 1-256, wherein R¹ is —P(O)H(OR¹²).

269 The compound of Embodiment 268, wherein R¹² is R.

270 The compound of Embodiment 268, wherein R¹² is H.

271. The compound of Embodiment 268, wherein R¹² is optionally substituted C₁₋₆ aliphatic.

272. The compound of any one of Embodiments 1-256, wherein R¹ is —C(O)N(R¹⁴) OH.

273. The compound of any one of Embodiments 1-256, wherein R¹ is —N(R¹⁴)S(O)₂R¹⁵.

274. The compound of any one of Embodiments 1-256, wherein R¹ is —S(O)₂N(R¹⁴)C(O)R¹⁵.

275 The compound of any one of Embodiments 1-256, wherein R¹ is —N(R¹⁶)C(O)(R¹⁴)S(O)₂R¹⁵.

276. The compound of any one of Embodiments 273-275, wherein R¹⁵ is R.

277 The compound of any one of Embodiments 273-275, wherein R¹⁵ is H.

278. The compound of any one of Embodiments 273-275, wherein R¹⁵ is optionally substituted C₁₋₆ aliphatic.

279. The compound of any one of Embodiments 1-256, wherein R¹ is —S(O)₂N(R¹⁴)C(O)NR¹⁶R¹⁷.

280. The compound of any one of Embodiments 272-279, wherein R¹⁴ is R.

281. The compound of any one of Embodiments 272-279, wherein R¹⁴ is H.

282. The compound of any one of Embodiments 272-279, wherein R¹⁴ is optionally substituted C₁₋₆ aliphatic.

283. The compound of any one of Embodiments 1-256, wherein R¹ is —S(O)₂NR¹⁶R¹⁷.

284. The compound of any one of Embodiments 279-283, wherein R¹⁶ is R.

285. The compound of any one of Embodiments 279-283, wherein R¹⁶ is H.

286. The compound of any one of Embodiments 279-283, wherein R¹⁶ is optionally substituted C₁₋₆ aliphatic.

287. The compound of any one of Embodiments 279-286, wherein R¹⁷ is R.

288. The compound of any one of Embodiments 279-286, wherein R¹⁷ is H.

289. The compound of any one of Embodiments 279-286, wherein R¹⁷ is optionally substituted C₁₋₆ aliphatic.

290. The compound of any one of Embodiments 1-256, wherein R¹ is optionally substituted

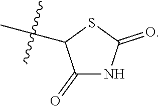 [SMILES: *C1SC(=O)NC1=O]

291. The compound of any one of Embodiments 1-256, wherein R¹ is optionally substituted

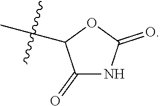 [SMILES: *C1OC(=O)NC1=O]

292. The compound of any one of Embodiments 1-256, wherein R¹ is optionally substituted

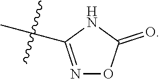 [SMILES: *c1noc(=O)[nH]1]

293. The compound of any one of Embodiments 1-256, wherein R¹ is optionally substituted

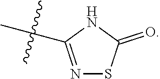 [SMILES: *c1nsc(=O)[nH]1]

294. The compound of any one of Embodiments 1-256, wherein R¹ is optionally substituted

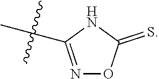 [SMILES: *c1noc(=S)[nH]1]

295. The compound of any one of Embodiments 1-256, wherein R¹ is optionally substituted

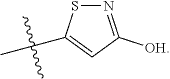 [SMILES: *c1cc(O)ns1]

296. The compound of any one of Embodiments 1-256, wherein R¹ is optionally substituted

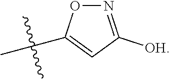 [SMILES: *c1cc(O)no1]

297. The compound of any one of Embodiments 1-256, wherein R¹ is substituted phenyl wherein a substituent is —OH.

298. The compound of any one of Embodiments 1-256, wherein R¹ is 4-hydroxylphenyl.

299. The compound of any one of Embodiments 1-256, wherein R¹ is 3-methyl-4-hydroxylphenyl.

300. The compound of any one of Embodiments 1-256, wherein R¹ is phenyl substituted with one or more fluoro and —OH.

301. The compound of any one of Embodiments 1-256, wherein R¹ is phenyl substituted with two or more fluoro and —OH.

302. The compound of any one of Embodiments 1-256, wherein R¹ is 3,5-difluoro-4-hydroxylphenyl.

303. The compound of any one of Embodiments 1-256, wherein R¹ is optionally substituted

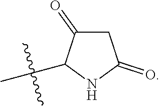 [SMILES: *C1NC(=O)CC1=O]

304. The compound of any one of Embodiments 1-256, wherein R¹ is optionally substituted

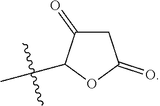 [SMILES: *C1OC(=O)CC1=O]

305. The compound of any one of Embodiments 1-256, wherein R¹ is optionally substituted

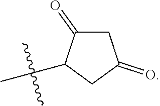 [SMILES: *C1CC(=O)CC1=O]

306. The compound of any one of Embodiments 1-256, wherein R¹ is

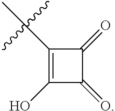 [SMILES: *c1c(O)c(=O)c1=O]

307. The compound of any one of Embodiments 1-256, wherein R¹ is

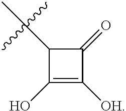 [SMILES: *C1C(=O)C(O)=C1O]

308. The compound of any one of Embodiments 1-256, wherein R¹ is optionally substituted

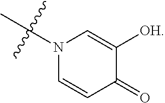 [SMILES: *n1ccc(=O)c(O)c1]

309. The compound of any one of Embodiments 1-256, wherein R¹ is optionally substituted

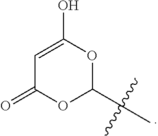 [SMILES: *C1OC(=O)C=C(O)O1]

310. The compound of any one of Embodiments 1-256, wherein R¹ is optionally substituted

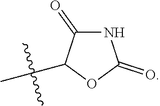 [SMILES: *C1OC(=O)NC1=O]

311. The compound of any one of Embodiments 1-256, wherein R¹ is optionally substituted

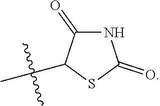 [SMILES: *C1SC(=O)NC1=O]

312. The compound of any one of Embodiments 1-256, wherein R¹ is optionally substituted

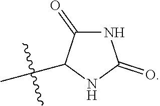 [SMILES: *C1NC(=O)NC1=O]

313. The compound of any one of Embodiments 1-256, wherein R¹ is optionally substituted

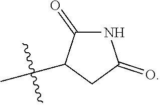 [SMILES: *C1CC(=O)NC1=O]

314. The compound of any one of Embodiments 1-256, wherein R¹ is optionally substituted

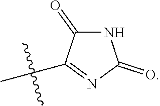 [SMILES: *C1=NC(=O)NC1=O]

315. The compound of any one of Embodiments 1-256, wherein R¹ is optionally substituted

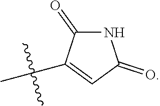 [SMILES: *C1=CC(=O)NC1=O]

316. The compound of any one of Embodiments 290-315, wherein R¹ is substituted.

317. The compound of any one of Embodiments 290-315, wherein R¹ is unsubstituted.

318 The compound of any one of Embodiments 2-256, wherein R¹ is halogen.

319. The compound of any one of Embodiments 2-256, wherein R¹ is —Br.

320. The compound of any one of Embodiments 1-256, wherein R¹ is

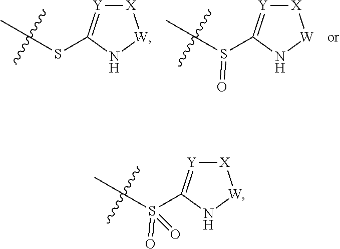

wherein each of X and W is independently-O—, —S—, —N(R⁸), or optionally substituted —CH₂—, and Y is —N═ or —C(R⁹)═, and the —NH— is optionally substituted.

321. The compound of Embodiment 320, wherein R¹ is

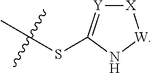

322. The compound of Embodiment 320, wherein R¹ is

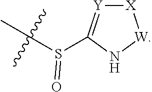

323. The compound of Embodiment 320, wherein R¹ is

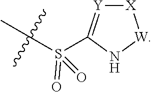

324. The compound of any one of Embodiments 320-323, wherein X is —O—.

325. The compound of any one of Embodiments 320-323, wherein X is —S—.

326. The compound of any one of Embodiments 320-323, wherein X is —N(R⁸)—.

327. The compound of any one of Embodiments 320-323, wherein X is —N(R⁸)— wherein R⁸ is R.

328. The compound of any one of Embodiments 320-323, wherein X is —N(R⁸)— wherein R⁸ is H.

329. The compound of any one of Embodiments 320-323, wherein X is —N(R⁸)— wherein R⁸ is optionally substituted C₁₋₆ aliphatic.

330. The compound of any one of Embodiments 320-323, wherein X is optionally substituted —CH₂—.

331 The compound of any one of Embodiments 320-323, wherein X is —CH₂—.

332. The compound of any one of Embodiments 320-331, wherein W is —O—.

333. The compound of any one of Embodiments 320-331, wherein W is —S—.

334. The compound of any one of Embodiments 320-331, wherein W is —N(R⁸)—.

335 The compound of any one of Embodiments 320-331, wherein W is —N(R⁸)— wherein R⁸ is R.

336. The compound of any one of Embodiments 320-331, wherein W is —N(R⁸)— wherein R⁸ is H.

337. The compound of any one of Embodiments 320-331, wherein W is —N(R⁸)— wherein R⁸ is optionally substituted C₁₋₆ aliphatic.

338. The compound of any one of Embodiments 320-331, wherein W is optionally substituted —CH₂—.

339. The compound of any one of Embodiments 320-331, wherein W is —CH₂—.

340. The compound of any one of Embodiments 1-256, wherein R¹ is

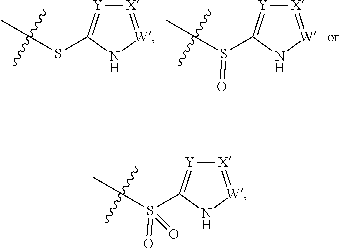

wherein each of X′, Y and W′ is independently-N═ or —C(R⁹)═, and the —NH—is optionally substituted.

341. The compound of Embodiment 340, wherein R¹ is

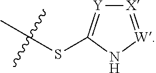

342. The compound of Embodiment 340, wherein R¹ is

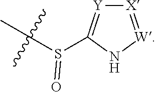

343. The compound of Embodiment 340, wherein R¹ is

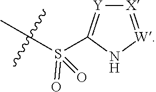

344. The compound of any one of Embodiments 340-343, wherein X′ is —N═.

345. The compound of any one of Embodiments 340-343, wherein W′ is —N═.

346 The compound of any one of Embodiments 340-343, wherein Y is —N═.

347. The compound of any one of Embodiments 340-343 and 345-346, wherein X′ is —C(R⁹)═.

348. The compound of any one of Embodiments 340-343 and 345-346, wherein X′ is —C(R⁹)═ wherein R⁹ is R.

349. The compound of any one of Embodiments 340-343 and 345-346, wherein X′ is —CH═.

350. The compound of any one of Embodiments 340-344 and 346-349, wherein W′ is —C(R⁹)═.

351. The compound of any one of Embodiments 340-344 and 346-349, wherein W′ is —C(R⁹)═ wherein R⁹ is R.

352. The compound of any one of Embodiments 340-344 and 346-349, wherein W′ is —CH═.

353. The compound of any one of Embodiments 340-345 and 347-352, wherein Y is —C(R⁹)═.

354. The compound of any one of Embodiments 340-345 and 347-352, wherein Y is —C(R⁹)═ wherein R⁹ is R.

355. The compound of any one of Embodiments 340-345 and 347-352, wherein Y is —H═.

356. The compound of any one of Embodiments 320-355, wherein the —NH— is not substituted.

357 The compound of any one of Embodiments 320-355, wherein the —NH— is substituted.

358 The compound of any one of Embodiments 1-256, wherein R¹ is —C(O)OR¹¹.

359. The compound of any one of Embodiments 1-256, wherein R¹ is —C(O)OR¹¹ wherein R¹¹ is optionally substituted C₁₋₆ aliphatic.

360. The compound of any one of Embodiments 1-256, wherein R¹ is —C(O)OR¹¹ wherein R¹¹ is optionally substituted C₁₋₆ alkyl.

361. The compound of any one of Embodiments 1-256, wherein R¹ is —P(O)(OR¹²)(OR¹³).

362. The compound of Embodiment 361, wherein R¹² is R.

363. The compound of Embodiment 361, wherein R¹² is H.

364. The compound of Embodiment 361, wherein R¹² is optionally substituted C₁₋₆ aliphatic.

365. The compound of Embodiment 361, wherein R¹² is optionally substituted C₁₋₆ alkyl.

366. The compound of Embodiment 361, wherein R¹² is methyl.

367 The compound of Embodiment 361, wherein R¹² is ethyl.

368 The compound of any one of Embodiments 361-367, wherein R¹³ is R.

369. The compound of Embodiment 368, wherein R¹² is H.

370. The compound of Embodiment 368, wherein R¹² is optionally substituted C₁₋₆ aliphatic.

371. The compound of Embodiment 368, wherein R¹² is optionally substituted C₁₋₆ alkyl.

372. The compound of Embodiment 368, wherein R¹² is methyl.

373. The compound of Embodiment 368, wherein R¹² is ethyl.

374. The compound of any one of Embodiments 1-256, wherein R¹ is —C(O)N(R¹⁴)SO₂R¹⁵.

375. The compound of Embodiment 374, wherein R¹⁴ is R.

376. The compound of Embodiment 374, wherein R¹⁴ is H.

377. The compound of Embodiment 374, wherein R¹⁴ is optionally substituted C₁₋₆ aliphatic.

378. The compound of any one of Embodiments 374-377, wherein R¹⁵ is R.

379. The compound of Embodiment 378, wherein R¹⁵ is optionally substituted C₁₋₆ aliphatic.

380 The compound of any one of Embodiments 1-256, wherein R¹ is —C(O)NR¹⁶R¹⁷.

381. The compound of Embodiment 380, wherein R¹⁶ is R.

382. The compound of Embodiment 380, wherein R¹⁶ is H.

383. The compound of Embodiment 380, wherein R¹⁶ is C₁₋₆ aliphatic.

384. The compound of Embodiment 380, wherein R¹⁶ is C₁₋₆ alkyl.

385 The compound of Embodiment 380, wherein R¹⁶ is methyl.

386. The compound of any one of Embodiments 380-385, wherein R¹⁷ is R.

387. The compound of Embodiment 386, wherein R¹⁷ is H.

388. The compound of Embodiment 386, wherein R¹⁷ is C₁₋₆ aliphatic.

389 The compound of Embodiment 386, wherein R¹⁷ is C₁₋₆ alkyl.

390 The compound of Embodiment 386, wherein R¹⁷ is methyl.

391. The compound of any one of Embodiments 1-256, wherein R¹ is —CN.

392. The compound of any one of Embodiments 1-256, wherein R¹ is

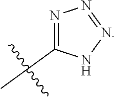 [SMILES: *c1nnn[nH]1]

393. The compound of any one of Embodiments 1-256, wherein R¹ is R^{d6}.

394. The compound of any one of the preceding Embodiments, wherein R^{d6} is —CH(OR)₂ wherein each R is independent not —H.

395. The compound of any one of the preceding Embodiments, wherein R^{d6} is —CH(OR)₂ wherein the two R are taken together with their intervening atoms to form an optionally substituted 4-10, e.g., 5-10, 5-6, 4, 5, 6, 7, 8, 9, or 10 membered ring having 0-3 heteroatoms in addition to the intervening atoms.

396. The compound of any one of the preceding Embodiments, wherein R^{d6} is optionally substituted

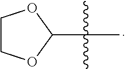 [SMILES: *C1OCCO1]

397. The compound of any one of the preceding Embodiments, wherein R^{d6} is

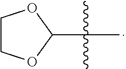 [SMILES: *C1OCCO1]

398. The compound of any one of Embodiments 1-393, wherein R^{d6} is —CH(OR)₂ wherein each R is independently optionally substituted C₁₋₆ aliphatic.

399. The compound of any one of Embodiments 1-256, wherein R¹ is —CHO.

400. The compound of any one of Embodiments 1-256, wherein R¹ is halogen.

401. The compound of any one of Embodiments 1-256, wherein R¹ is F.

402. The compound of any one of Embodiments 1-256, wherein R¹ is Cl.

403. The compound of any one of Embodiments 1-256, wherein R¹ is Br.

404 The compound of any one of Embodiments 1-256, wherein R¹ is I.

405. The compound of any one of Embodiments 1-256, wherein R¹ is

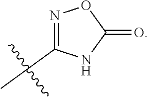 [SMILES: *c1noc(=O)[nH]1]

406. The compound of any one of the preceding Embodiments, wherein R² is halogen.

407. The compound of Embodiment 406, wherein R² is F.

408. The compound of Embodiment 406, wherein R² is Cl.

409. The compound of Embodiment 406, wherein R² is Br.

410 The compound of any one of Embodiments 1-399, wherein R² is R.

411. The compound of any one of Embodiments 1-399, wherein R² is H.

412. The compound of any one of Embodiments 1-399, wherein R² is —C(O)OR.

413. The compound of any one of Embodiments 1-399, wherein R² is —C(O)OH.

414. The compound of any one of Embodiments 1-399, wherein R² is —C(O)OR wherein R is optionally substituted C₁₋₆ aliphatic.

415. The compound of any one of Embodiments 1-399, wherein R² is —OR.

416. The compound of any one of Embodiments 1-399, wherein R² is —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms.

417. The compound of any one of Embodiments 1-399, wherein R² is —OR wherein R is optionally substituted C₁₋₆ aliphatic.

418. The compound of any one of Embodiments 1-399, wherein R² is —OMe.

419. The compound of any one of Embodiments 1-399, wherein R² is optionally substituted phenyl.

420. The compound of any one of Embodiments 1-399, wherein R² is phenyl.

421. The compound of any one of Embodiments 1-399, wherein R² is optionally substituted C₁₋₆ aliphatic.

422. The compound of any one of Embodiments 1-399, wherein R² is optionally substituted C₁₋₆ alkyl.

423. The compound of any one of Embodiments 1-399, wherein R² is methyl.

424. The compound of any one of Embodiments 1-399, wherein R² is optionally substituted C₃₋₆ cycloalkyl.

425. The compound of any one of Embodiments 1-399, wherein R² is

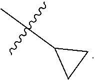 [SMILES: *C1CC1]

426. The compound of any one of Embodiments 1-399, wherein R² is

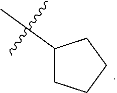 [SMILES: *C1CCCC1]

427. The compound of any one of Embodiments 1-399, wherein R² is optionally substituted 5-6 membered heteroaryl having 1-4 heteroatoms.

428. The compound of any one of Embodiments 1-399, wherein R² is

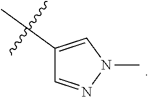 [SMILES: *c1cnn(C)c1]

429. The compound of any one of the preceding Embodiments, wherein R³ is R.

430. The compound of any one of the preceding Embodiments, wherein R³ is H.

431. The compound of any one of Embodiments 1-428, wherein R³ is —C(O)OR.

432. The compound of any one of Embodiments 1-428, wherein R³ is —C(O)OH.

433. The compound of any one of Embodiments 1-428, wherein R³ is —C(O)OR wherein R is optionally substituted C₁₋₆ aliphatic.

434. The compound of any one of Embodiments 1-428, wherein R³ is —OR.

435. The compound of any one of Embodiments 1-428, wherein R³ is —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms.

436. The compound of any one of Embodiments 1-428, wherein R³ is —OR wherein R is optionally substituted C₁₋₆ aliphatic.

437. The compound of any one of Embodiments 1-428, wherein R³ is —OMe.

438. The compound of any one of Embodiments 1-428, wherein R³ is optionally substituted phenyl.

439 The compound of any one of Embodiments 1-428, wherein R³ is phenyl.

440. The compound of any one of Embodiments 1-428, wherein R³ is optionally substituted C₁₋₆ aliphatic.

441. The compound of any one of Embodiments 1-428, wherein R³ is optionally substituted C₁₋₆ alkyl.

442. The compound of any one of Embodiments 1-428, wherein R³ is methyl.

443. The compound of any one of Embodiments 1-428, wherein R³ is optionally substituted C₃₋₆ cycloalkyl.

444. The compound of any one of Embodiments 1-428, wherein R³ is

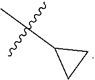 [SMILES: *C1CC1]

445. The compound of any one of Embodiments 1-428, wherein R³ is

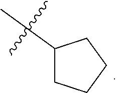 [SMILES: *C1CCCC1]

446. The compound of any one of Embodiments 1-428, wherein R³ is optionally substituted 5-6 membered heteroaryl having 1-4 heteroatoms.

447. The compound of any one of Embodiments 1-428, wherein R³ is

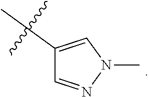 [SMILES: *c1cnn(C)c1]

448. The compound of any one of Embodiments 1-428, wherein R³ is halogen.

449. The compound of any one of Embodiments 1-428, wherein R³ is F.

450. The compound of any one of Embodiments 1-428, wherein R³ is Cl.

451. The compound of any one of Embodiments 1-428, wherein R³ is Br.

452. The compound of any one of Embodiments 1-207, wherein Ring B is

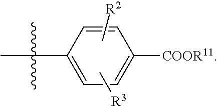

453. The compound of any one of Embodiments 1-207, wherein Ring B is

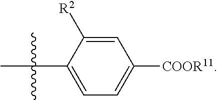

454. The compound of Embodiment 453, wherein R² is halogen.

455. The compound of Embodiment 454, wherein R² is F.

456. The compound of any one of Embodiments 1-207, wherein Ring B is

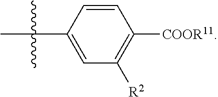

457. The compound of Embodiment 456, wherein R² is halogen.

458. The compound of Embodiment 456 or 457, wherein R² is F.

459. The compound of any one of Embodiments 1-207, wherein Ring B is

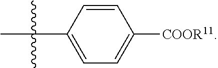

460. The compound of any one of Embodiments 1-207, wherein Ring B is

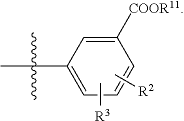

461. The compound of Embodiment 460, wherein Ring B is

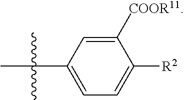

462. The compound of Embodiment 461, wherein R² is F.

463. The compound of Embodiment 460, wherein Ring B is

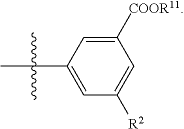

464. The compound of Embodiment 463, wherein R² is halogen, optionally substituted C₁-C₆ alkyl, —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms.

465. The compound of Embodiment 463 or 464, wherein R² is F, Cl, or Br.

466. The compound of Embodiment 463 or 464, wherein R² is —OMe.

467. The compound of Embodiment 463 or 464, wherein R² is phenyl.

468. The compound of Embodiment 463 or 464, wherein R² is

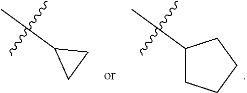

469. The compound of Embodiment 463 or 464, wherein R² is methyl.

470. The compound of Embodiment 463 or 464, wherein R² is

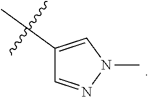 [SMILES: *c1cnn(C)c1]

471. The compound of Embodiment 460, wherein Ring B is

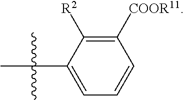

472. The compound of Embodiment 471, wherein R² is halogen, optionally substituted C₁-C₆ alkyl, or —OR wherein R is optionally substituted C₁-C₆ alkyl.

473. The compound of Embodiment 471 or 472, wherein R² is F.

474. The compound of Embodiment 471 or 472, wherein R² is methyl.

475. The compound of Embodiment 471 or 472, wherein R² is —OMe.

476. The compound of Embodiment 460, wherein Ring B is

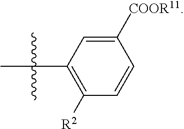

477. The compound of Embodiment 476, wherein R² is halogen.

478. The compound of Embodiment 476 or 477, wherein R² is F.

479. The compound of any one of Embodiments 207-478, wherein R¹¹ is H.

480. The compound of any one of Embodiments 1-207, wherein Ring B is

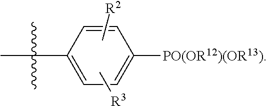

481. The compound of Embodiment 480, wherein Ring B is

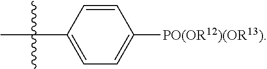

482. The compound of Embodiment 480 or 481, wherein R¹² and R¹³ are each independently H or optionally substituted C₁-C₆ alkyl.

483. The compound of any one of Embodiments 480-482, wherein R¹² and R¹³ are each H.

484. The compound of any one of Embodiments 480-482, wherein R¹² is H and R¹³ is optionally substituted C₁-C₆ alkyl.

485. The compound of any one of Embodiments 480-482, wherein R¹² is H and R¹³ is ethyl.

486. The compound of any one of Embodiments 480-482, wherein R¹² and R¹³ are each independently an optionally substituted C₁-C₆ alkyl.

487. The compound of any one of Embodiments 480-482, wherein R¹² is ethyl and R¹³ is ethyl.

488. The compound of any one of Embodiments 1-207, wherein Ring B is

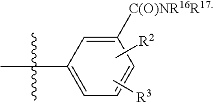

489. The compound of Embodiment 488, wherein Ring B is

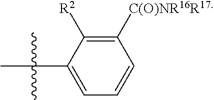

490. The compound of Embodiment 488 or 489, wherein R² is H or halogen.

491. The compound of any one of Embodiments 488-490, wherein R² is F.

492. The compound of any one of Embodiments 488-491, wherein R¹⁶ and R¹⁷ are each independently H or optionally substituted C₁-C₆ alkyl.

493. The compound of any one of Embodiments 488-492, wherein R¹⁶ and R¹⁷ are each H.

494. The compound of any one of Embodiments 488-492, wherein R¹⁶ is H and R¹⁷ is optionally substituted C₁-C₆ alkyl.

495. The compound of any one of Embodiments 488-492, wherein R¹⁶ is H and R¹⁷ is methyl.

496. The compound of any one of Embodiments 488-492, wherein R¹⁶ and R¹⁷ are each independently an optionally substituted C₁-C₆ alkyl.

497. The compound of any one of Embodiments 488-492, wherein R¹⁶ is methyl and R¹⁷ is methyl.

498. The compound of any one of Embodiments 1-207, wherein Ring B is

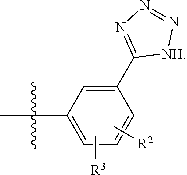

499. The compound of Embodiment 498, wherein Ring B is

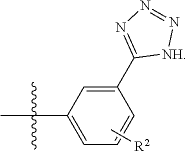

500. The compound of any one of Embodiments 498-499, wherein Ring B is

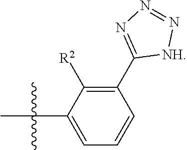

501. The compound of any one of Embodiments 498-500, wherein Ring B is

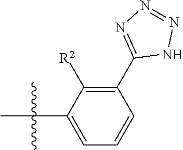

### and R² is H.

502. The compound of any one of Embodiments 498-500, wherein Ring B is

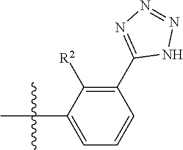

and R² is halogen.

503. The compound of any one of Embodiments 498-500, wherein Ring B is

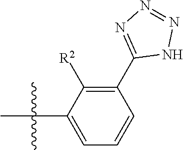

### and R² is F.

504. The compound of any one of Embodiments 498-499, wherein Ring B is

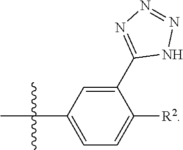

505. The compound of Embodiment 504, wherein Ring B is

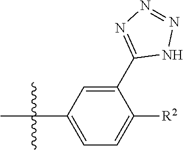

and R² is halogen.

506. The compound of Embodiment 504, wherein Ring B is

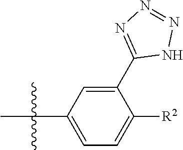

### and R² is F.

507. The compound of any one of Embodiments 498-499, wherein Ring B is

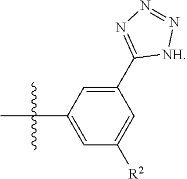

508. The compound of Embodiment 507, wherein Ring B is

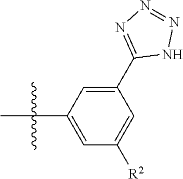

and R² is halogen.

509. The compound of Embodiment 508, wherein Ring B is

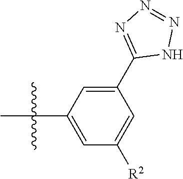

### and R² is F.

510. The compound of Embodiment 507, wherein Ring B is

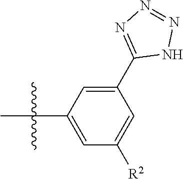

and R² is optionally substituted C₁-C₆ alkyl.

511. The compound of Embodiment 510, wherein Ring B is

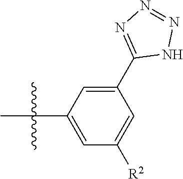

and R² is methyl.

512. The compound of Embodiment 507, wherein Ring B is

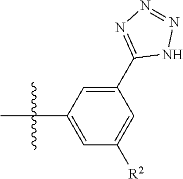

and R² is optionally substituted C₃-C₈ cycloalkyl.

513. The compound of Embodiment 512, wherein Ring B is

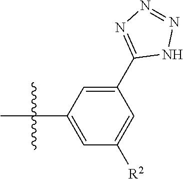

### and R² is

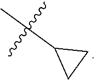 [SMILES: *C1CC1]

514. The compound of Embodiment 507, wherein Ring B is

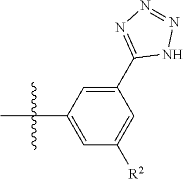

### and R² is —C(O)OR.

515. The compound of Embodiment 514, wherein Ring B is

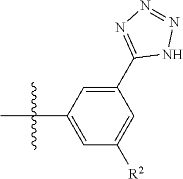

and R² is —C(O)OR, wherein R is hydrogen.

516. The compound of Embodiment 514, wherein Ring B is

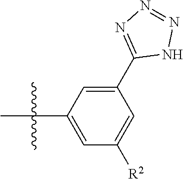

and R² is —C(O)OR, wherein R is an optionally substituted C₁-C₆ alkyl group.

517. The compound of Embodiment 507, wherein Ring B is

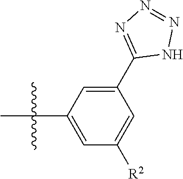

### and R² is —CN.

518. The compound of any one of Embodiments 1-207, wherein Ring B is

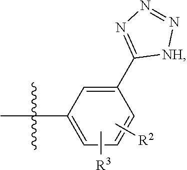

wherein each of R² and R³ is independently halogen, —CN, optionally substituted C₁-C₆ alkyl, —OR wherein R is optionally substituted C₁-C₆ alkyl, optionally substituted 6-10 membered aryl, optionally substituted C₃—Cs cycloalkyl, or optionally substituted 5-10 membered heteroaryl having 1-6 heteroatoms.

519. The compound of Embodiment 518, wherein Ring B is

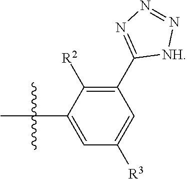

520. The compound of any one of Embodiment 518-519, wherein each of R² and R³ is independently halogen and optionally substituted C₁-C₆ alkyl.

521. The compound of any one of Embodiment 518-520, wherein Ring B is

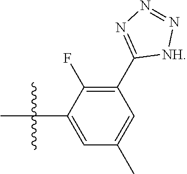 [SMILES: *c1cc(C)cc(-c2nnn[nH]2)c1F]

522. The compound of Embodiment 1 or 2, wherein the compound has a structure of:

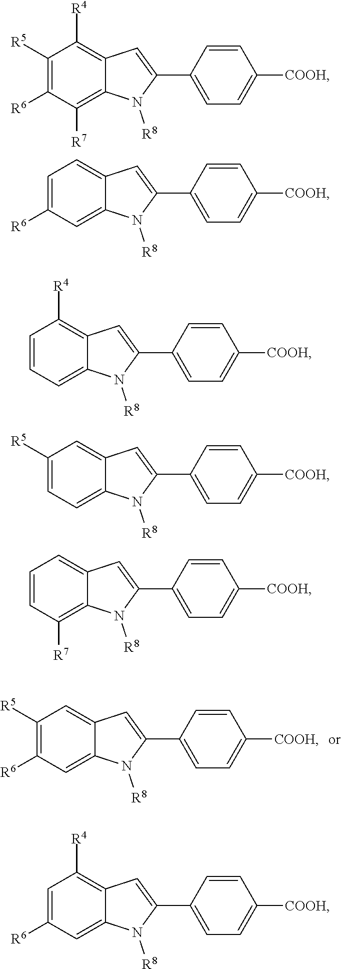

or a pharmaceutically acceptable salt thereof.

523. The compound of Embodiment 498, wherein the compound has a structure of:

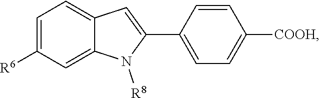

or a pharmaceutically acceptable salt thereof.

524. The compound of Embodiment 1 or 2, wherein the compound has a structure of:

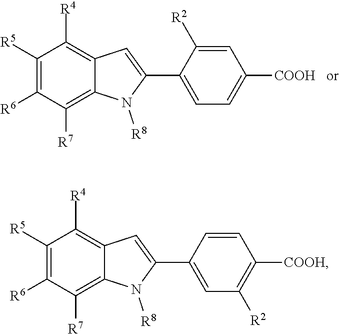

or a pharmaceutically acceptable salt thereof.

525. The compound of Embodiment 524, wherein the compound has a structure of:

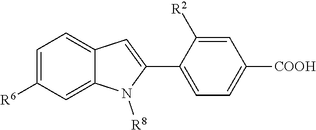

or a pharmaceutically acceptable salt thereof.

526. The compound of Embodiment 524 or 525, wherein R² is halogen.

527. The compound of any one of Embodiments 524-526, wherein R² is F.

528. The compound of any one of Embodiments 498-527, wherein R⁸ is H or optionally substituted C₁-C₆ alkyl.

529 The compound of any one of Embodiments 498-528, wherein R⁸ is H.

530. The compound of any one of Embodiments 498-528, wherein R⁸ optionally substituted C₁-C₆ alkyl.

531. The compound of Embodiment 530, wherein R⁸ is methyl.

532. The compound of Embodiment 530, wherein R⁸ is propyl.

533. The compound of Embodiment 530, wherein R⁸ is isopropyl.

534. The compound of Embodiment 530, wherein R⁸ is isobutyl.

535. The compound of Embodiment 530, wherein R⁸ is butyl.

536. The compound of any one of Embodiments 498-527, wherein R⁸ is optionally substituted C₃-C₈ cycloalkyl.

537. The compound of Embodiment 536, wherein R⁸ is

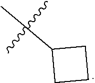 [SMILES: *C1CCC1]

538. The compound of a of Embodiment 536, wherein R⁸ is

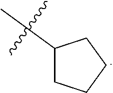 [SMILES: *C1CCCC1]

539 The compound of a of Embodiment 536, wherein R⁸ is

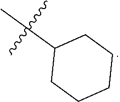 [SMILES: *C1CCCCC1]

540. The compound of any one of Embodiments 498-527, wherein R⁸ is optionally substituted 6-10 membered aryl.

541. The compound of Embodiment 540, wherein R⁸ is phenyl.

542. The compound of any one of Embodiments 498-541, wherein R⁶ is —CN.

543. The compound of any one of Embodiments 498-541, wherein R⁶ is halogen.

544. The compound of any one of Embodiments 498-541, wherein R⁶ is F or Cl.

545. The compound of any one of Embodiments 498-541, wherein R⁶ is optionally substituted C₁-C₆ alkyl.

546. The compound of any one of Embodiments 498-541, wherein R⁶ is —CF₃.

547. The compound of Embodiment 1 or 2, wherein the compound has a structure of:

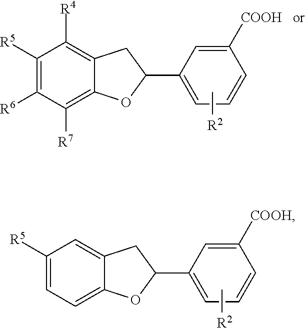

or a pharmaceutically acceptable salt

548. The compound of Embodiment 547, wherein the compound has a structure of:

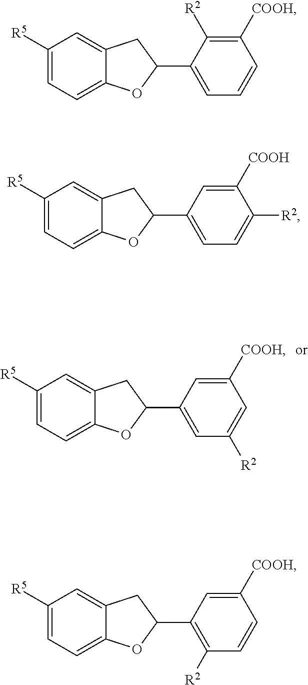

or a pharmaceutically acceptable salt thereof.

549. The compound of Embodiment 547 or 548, wherein R⁵ is H.

550. The compound of Embodiment 547 or 548, wherein R⁵ is optionally substituted C₁-C₆ alkyl.

551. The compound of Embodiment 550, wherein R⁵ is —CF₃.

552. The compound of Embodiment 547 or 548, wherein R⁵ is halogen.

553. The compound of Embodiment 547 or 548, wherein R⁵ is F or Cl.

554. The compound of any one of Embodiments 547-553, wherein R² is H.

555. The compound of any one of Embodiments 547-553, wherein R² is halogen.

556. The compound of Embodiment 555, wherein R² is F, Cl, or Br.

557 The compound of any one of Embodiments 547-553, wherein R² is optionally substituted C₁-C₆ alkyl.

558. The compound of Embodiment 557, wherein R² is methyl.

559. The compound of any one of Embodiments 547-553, wherein R² is optionally substituted C₃-C₈ cycloalkyl.

560. The compound of Embodiment 559, wherein R² is

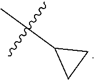 [SMILES: *C1CC1]

561. The compound of Embodiment 559, wherein R² is

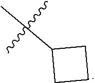 [SMILES: *C1CCC1]

562. The compound of Embodiment 559, wherein R² is

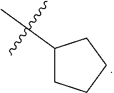 [SMILES: *C1CCCC1]

563. The compound of any one of Embodiments 547-553, wherein R² is —OR wherein R is optionally substituted C₁-C₆ alkyl.

564. The compound of Embodiment 563, wherein R² is —OMe.

565. The compound of any one of Embodiments 547-553, wherein R² is optionally substituted 6-10 membered aryl.

566. The compound of Embodiment 565, wherein R² is phenyl.

567. The compound of any one of Embodiments 547-553, wherein R² is optionally substituted 5-10 membered heteroaryl having 1-3 heteroatoms.

568. The compound of Embodiment 567, wherein R² is

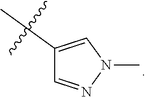 [SMILES: *c1cnn(C)c1]

569. The compound of Embodiment 1 or 2, wherein the compound has a structure of:

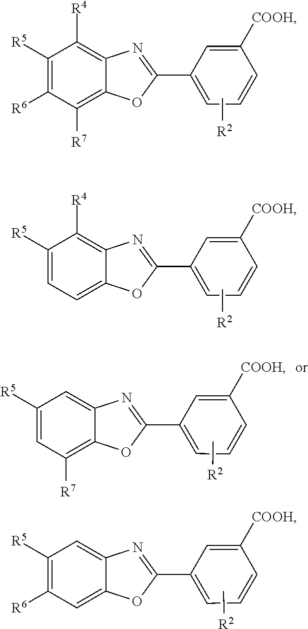

or a pharmaceutically acceptable salt thereof.

570. The compound of Embodiment 569, wherein the compound has a structure of:

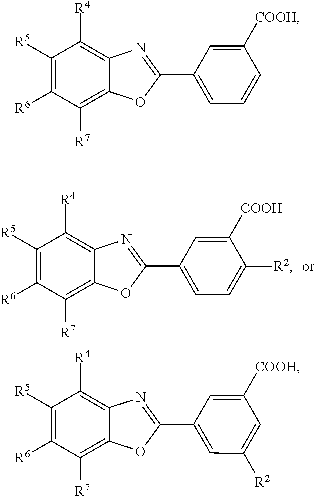

or a pharmaceutically acceptable salt thereof.

571. The compound of Embodiment 1 or 2, wherein the compound has a structure of:

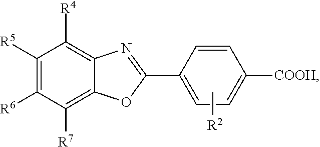

or a pharmaceutically acceptable salt thereof.

572. The compound of any one of Embodiments 569-571, wherein R² is H.

573. The compound of any one of Embodiments 569-571, wherein R² is halogen.

574. The compound of Embodiment 573, wherein R² is F, Cl, or Br.

575. The compound of any one of Embodiments 569-574, wherein R⁴ is optionally substituted C₁-C₆ alkyl.

576. The compound of Embodiment 575, wherein R⁴ is —CF₃.

577 The compound of any one of Embodiments 569-576, wherein R⁵ is halogen.

578 The compound of Embodiment 577, wherein R⁵ is F or Cl.

579. The compound of any one of Embodiments 569-576, wherein R⁵ is optionally substituted C₁-C₆ alkyl.

580 The compound of Embodiment 579, wherein R⁵ is —CF₃.

581. The compound of any one of Embodiments 569-580, wherein R⁶ is halogen.

582 The compound of Embodiment 581, wherein R⁶ is F or Cl.

583. The compound of any one of Embodiments 569-582, wherein R⁷ is halogen.

584 The compound of Embodiment 583, wherein R⁷ is F or Cl.

585. The compound of Embodiment 1 or 2, wherein the compound has a structure of:

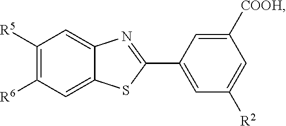

or a pharmaceutically acceptable salt thereof.

586. The compound of Embodiment 585, wherein R² is halogen.

587. The compound of Embodiment 586, wherein R² is F, Cl, or Br.

588. The compound of any one of Embodiments 585-587, wherein R⁵ is optionally substituted C₁-C₆ alkyl.

589 The compound of Embodiment 588, wherein R⁵ is —CF₃.

590. The compound of any one of Embodiments 585-589, wherein R⁶ is halogen.

591 The compound of Embodiment 590, wherein R⁶ is Cl.

592. The compound of Embodiment 1 or 2, wherein the compound has a structure of:

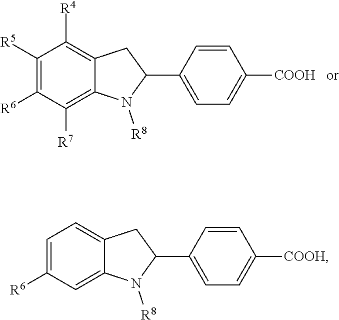

or a pharmaceutically acceptable salt thereof.

593. The compound of Embodiment 592, wherein R⁸ is H or methyl.

594. The compound of Embodiment 592 or 593, wherein R⁶ is —CF₃.

595. The compound of Embodiment 1 or 2, wherein the compound has a structure of:

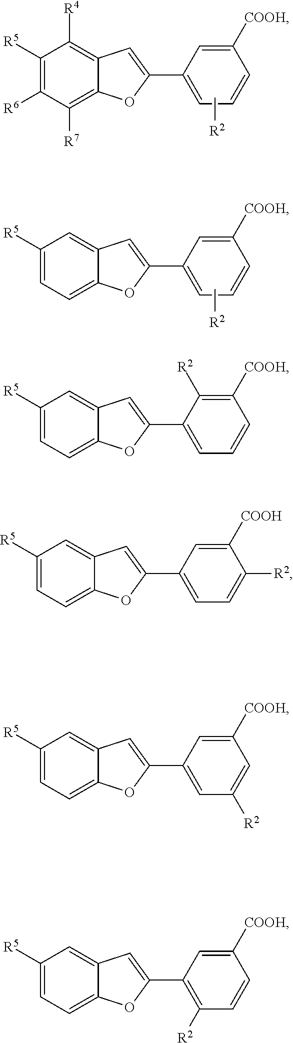

or a pharmaceutically acceptable salt thereof.

596. The compound of Embodiment 595, wherein R² is H.

597 The compound of Embodiment 595, wherein R² is halogen.

598. The compound of Embodiment 597, wherein R² is F, Cl, or Br.

599. The compound of any one of Embodiment 595-598, wherein R⁵ is optionally substituted C₁-C₆ alkyl.

600. The compound of Embodiment 599, wherein R⁵ is —CF₃.

601. The compound of Embodiment 1 or 2, wherein the compound has a structure of:

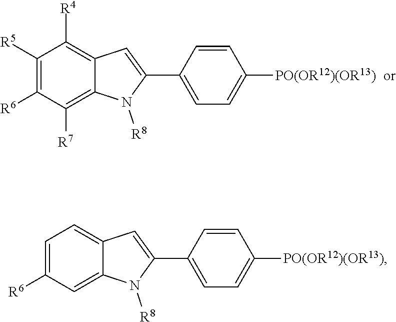

or a pharmaceutically acceptable salt thereof.

602. The compound of Embodiment 601, wherein R⁸ is optionally substituted C₁-C₆ alkyl.

603. The compound of Embodiment 601 or 602, wherein R⁸ is methyl.

604. The compound of any one of Embodiments 601-603, wherein R⁶ is optionally substituted C₁-C₆ alkyl.

605. The compound of Embodiment 604, wherein R⁶ is —CF₃.

606. The compound of any one of Embodiments 601-605, wherein R¹² and R¹³ are each independently

H or optionally substituted C₁-C₆ alkyl.

607. The compound of Embodiment 606, wherein R¹² and R¹³ are each independently H.

608. The compound of Embodiment 606, wherein R¹² and R¹³ are each independently ethyl.

609. The compound of Embodiment 606, wherein R¹² is H and R¹³ is ethyl.

610. The compound of Embodiment 1 or 2, wherein the compound has a structure of:

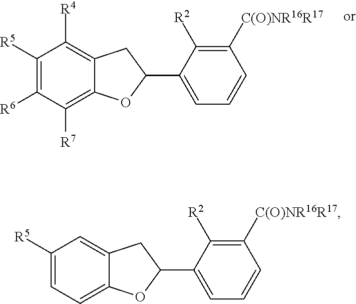

or a pharmaceutically acceptable salt thereof.

611. The compound of Embodiment 610, wherein R² is halogen.

612. The compound of Embodiment 610 or 611, wherein R² is F.

613. The compound of any one of Embodiments 610-612, wherein R⁵ is optionally substituted C₁-C₆ alkyl.

614. The compound of Embodiment 613, wherein R⁵ is —CF₃.

615. The compound of any one of Embodiments 610-614, wherein R¹⁶ and R¹⁷ are each independently

H or optionally substituted C₁-C₆ alkyl.

616. The compound of Embodiment 615, wherein R¹⁶ is methyl and R¹⁷ is methyl.

617. The compound of Embodiment 1 or 2, wherein the compound has a structure of:

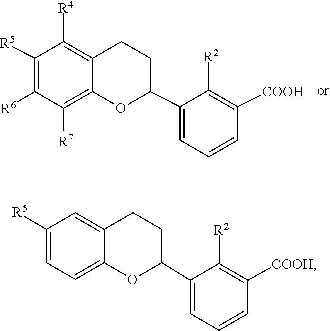

or a pharmaceutically acceptable salt thereof.

618. The compound of Embodiment 617, wherein R² is halogen.

619. The compound of Embodiment 617 or 618, wherein R² is F.

620. The compound of any one of Embodiment 617-619, wherein R² is optionally substituted C₁-C₆ alkyl.

621. The compound of Embodiment 620, wherein R² is —CF₃.

622. The compound of any one of the preceding Embodiments, wherein each heteroatom is independently selected from nitrogen, oxygen and sulfur.

623. A compound, wherein the compound is a compound selected from Table 1 or a salt thereof.

624. A compound, wherein the compound is a compound selected from compounds 88-105 or a salt thereof:

| Compound | Structure |
|---|---|
| 88 | 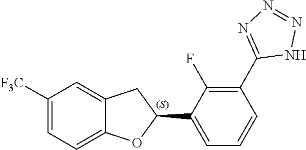 |
| 89 | 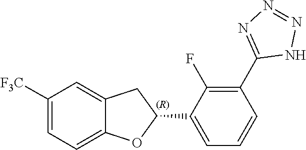 |
| 90 | 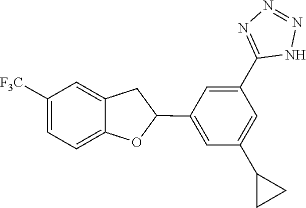 [SMILES: FC(F)(F)c1ccc2c(c1)CC(c1cc(-c3nnn[nH]3)cc(C3CC3)c1)O2] |
| 91 | 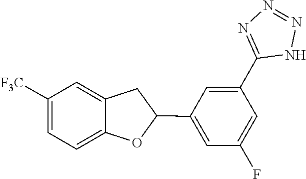 [SMILES: Fc1cc(-c2nnn[nH]2)cc(C2Cc3cc(C(F)(F)F)ccc3O2)c1] |
| 92 | 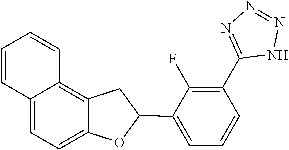 [SMILES: Fc1c(-c2nnn[nH]2)cccc1C1Cc2c(ccc3ccccc23)O1] |
| 93 | 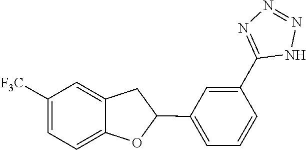 [SMILES: FC(F)(F)c1ccc2c(c1)CC(c1cccc(-c3nnn[nH]3)c1)O2] |
| 94 | 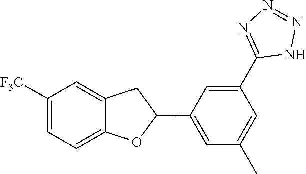 [SMILES: Cc1cc(-c2nnn[nH]2)cc(C2Cc3cc(C(F)(F)F)ccc3O2)c1] |
| 95 | 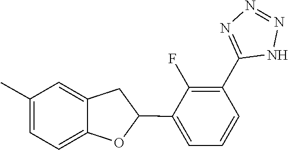 [SMILES: Cc1ccc2c(c1)CC(c1cccc(-c3nnn[nH]3)c1F)O2] |
| 96 | 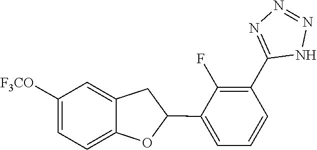 [SMILES: Fc1c(-c2nnn[nH]2)cccc1C1Cc2cc(OC(F)(F)F)ccc2O1] |
| 97 | 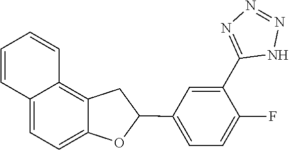 [SMILES: Fc1ccc(C2Cc3c(ccc4ccccc34)O2)cc1-c1nnn[nH]1] |
| 98 | 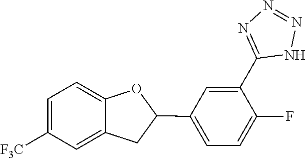 [SMILES: Fc1ccc(C2Cc3cc(C(F)(F)F)ccc3O2)cc1-c1nnn[nH]1] |
| 99 | 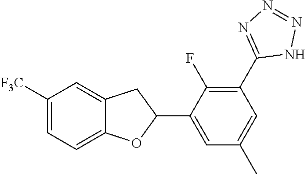 [SMILES: Cc1cc(-c2nnn[nH]2)c(F)c(C2Cc3cc(C(F)(F)F)ccc3O2)c1] |
| 100 | 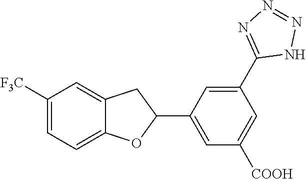 [SMILES: O=C(O)c1cc(-c2nnn[nH]2)cc(C2Cc3cc(C(F)(F)F)ccc3O2)c1] |
| 101 | 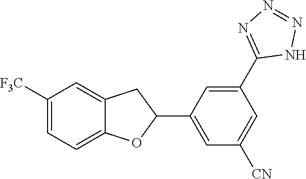 [SMILES: N#Cc1cc(-c2nnn[nH]2)cc(C2Cc3cc(C(F)(F)F)ccc3O2)c1] |
| 102 | 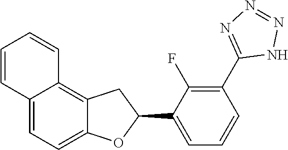 [SMILES: Fc1c(-c2nnn[nH]2)cccc1[C@@H]1Cc2c(ccc3ccccc23)O1] |
| 103 | 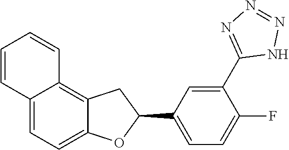 [SMILES: Fc1ccc([C@@H]2Cc3c(ccc4ccccc34)O2)cc1-c1nnn[nH]1] |
| 104 | 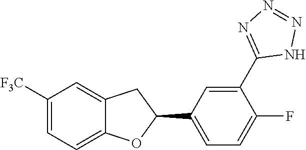 [SMILES: Fc1ccc([C@@H]2Cc3cc(C(F)(F)F)ccc3O2)cc1-c1nnn[nH]1] |
| 105 | 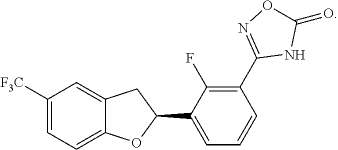 [SMILES: O=c1[nH]c(-c2cccc([C@@H]3Cc4cc(C(F)(F)F)ccc4O3)c2F)no1] |

625. A compound, wherein the compound has the structure of

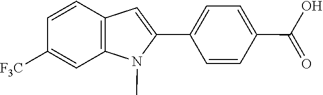 [SMILES: Cn1c(-c2ccc(C(=O)O)cc2)cc2ccc(C(F)(F)F)cc21]

or a salt thereof.

626. A compound, wherein the compound has the structure of

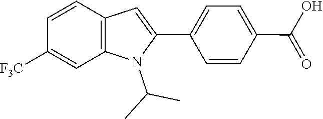 [SMILES: CC(C)n1c(-c2ccc(C(=O)O)cc2)cc2ccc(C(F)(F)F)cc21]

or a salt thereof.

627. A compound, wherein the compound has the structure of

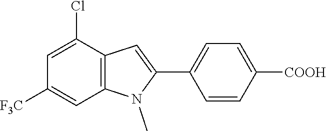 [SMILES: Cn1c(-c2ccc(C(=O)O)cc2)cc2c(Cl)cc(C(F)(F)F)cc21]

or a salt thereof.

628. A compound, wherein the compound has the structure of

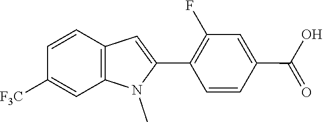 [SMILES: Cn1c(-c2ccc(C(=O)O)cc2F)cc2ccc(C(F)(F)F)cc21]

or a salt thereof.

629. A compound, wherein the compound has the structure of

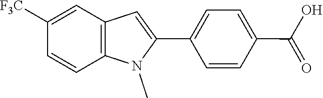 [SMILES: Cn1c(-c2ccc(C(=O)O)cc2)cc2cc(C(F)(F)F)ccc21]

or a salt thereof.

630. A compound, wherein the compound has the structure of

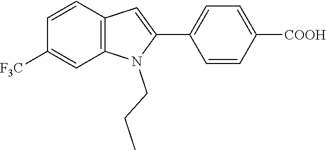 [SMILES: CCCn1c(-c2ccc(C(=O)O)cc2)cc2ccc(C(F)(F)F)cc21]

or a salt thereof.

631. A compound, wherein the compound has the structure of

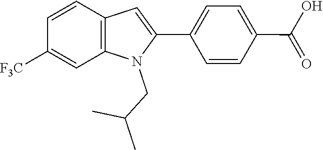 [SMILES: CC(C)Cn1c(-c2ccc(C(=O)O)cc2)cc2ccc(C(F)(F)F)cc21]

or a salt thereof.

632. A compound, wherein the compound has the structure of

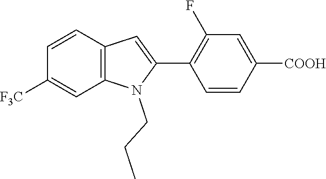 [SMILES: CCCn1c(-c2ccc(C(=O)O)cc2F)cc2ccc(C(F)(F)F)cc21]

or a salt thereof.

633. A compound, wherein the compound has the structure of

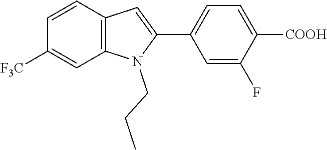 [SMILES: CCCn1c(-c2ccc(C(=O)O)c(F)c2)cc2ccc(C(F)(F)F)cc21]

or a salt thereof.

634. A compound, wherein the compound has the structure of

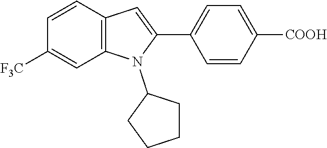 [SMILES: O=C(O)c1ccc(-c2cc3ccc(C(F)(F)F)cc3n2C2CCCC2)cc1]

or a salt thereof.

635. A compound, wherein the compound has the structure of

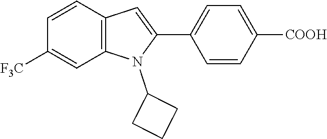 [SMILES: O=C(O)c1ccc(-c2cc3ccc(C(F)(F)F)cc3n2C2CCC2)cc1]

or a salt thereof.

636. A compound, wherein the compound has the structure of

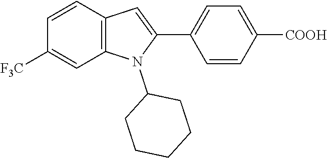 [SMILES: O=C(O)c1ccc(-c2cc3ccc(C(F)(F)F)cc3n2C2CCCCC2)cc1]

or a salt thereof.

637. A compound, wherein the compound has the structure of

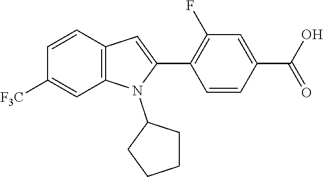 [SMILES: O=C(O)c1ccc(-c2cc3ccc(C(F)(F)F)cc3n2C2CCCC2)c(F)c1]

or a salt thereof.

638. A compound, wherein the compound has the structure of

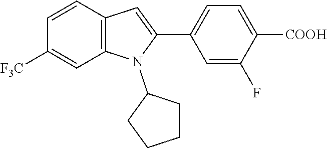 [SMILES: O=C(O)c1ccc(-c2cc3ccc(C(F)(F)F)cc3n2C2CCCC2)cc1F]

or a salt thereof.

639. A compound, wherein the compound has the structure of

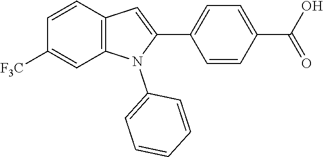 [SMILES: O=C(O)c1ccc(-c2cc3ccc(C(F)(F)F)cc3n2-c2ccccc2)cc1]

or a salt thereof.

640. A compound, wherein the compound has the structure of

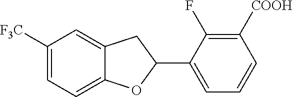 [SMILES: O=C(O)c1cccc(C2Cc3cc(C(F)(F)F)ccc3O2)c1F]

or a salt thereof.

641. A compound, wherein the compound has the structure of

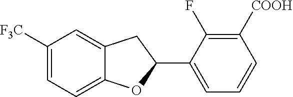 [SMILES: O=C(O)c1cccc([C@@H]2Cc3cc(C(F)(F)F)ccc3O2)c1F]

or a salt thereof.

642. A compound, wherein the compound has the structure of

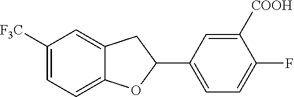 [SMILES: O=C(O)c1cc(C2Cc3cc(C(F)(F)F)ccc3O2)ccc1F]

or a salt thereof.

643. A compound, wherein the compound has the structure of

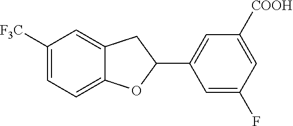 [SMILES: O=C(O)c1cc(F)cc(C2Cc3cc(C(F)(F)F)ccc3O2)c1]

or a salt thereof.

644. A compound, wherein the compound has the structure of

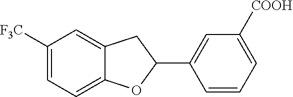 [SMILES: O=C(O)c1cccc(C2Cc3cc(C(F)(F)F)ccc3O2)c1]

or a salt thereof.

645. A compound, wherein the compound has the structure of

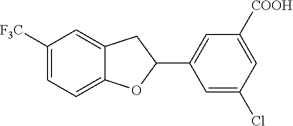 [SMILES: O=C(O)c1cc(Cl)cc(C2Cc3cc(C(F)(F)F)ccc3O2)c1]

or a salt thereof.

646. A compound, wherein the compound has the structure of

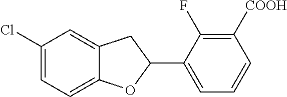 [SMILES: O=C(O)c1cccc(C2Cc3cc(Cl)ccc3O2)c1F]

or a salt thereof.

647. A compound, wherein the compound has the structure of

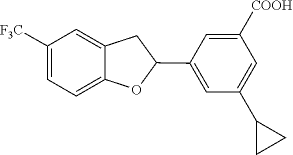 [SMILES: O=C(O)c1cc(C2CC2)cc(C2Cc3cc(C(F)(F)F)ccc3O2)c1]

or a salt thereof.

648. A compound, wherein the compound has the structure of

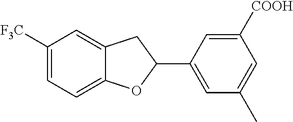 [SMILES: Cc1cc(C(=O)O)cc(C2Cc3cc(C(F)(F)F)ccc3O2)c1]

or a salt thereof.

649. A compound, wherein the compound has the structure of

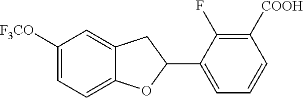 [SMILES: O=C(O)c1cccc(C2Cc3cc(OC(F)(F)F)ccc3O2)c1F]

or a salt thereof.

650. A compound, wherein the compound has the structure of

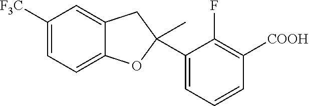 [SMILES: CC1(c2cccc(C(=O)O)c2F)Cc2cc(C(F)(F)F)ccc2O1]

or a salt thereof.

651. A compound, wherein the compound has the structure of

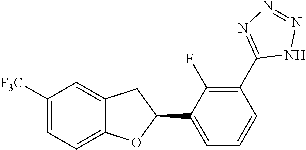 [SMILES: Fc1c(-c2nnn[nH]2)cccc1[C@@H]1Cc2cc(C(F)(F)F)ccc2O1]

or a salt thereof.

652. A compound, wherein the compound has the structure of

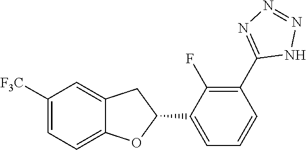 [SMILES: Fc1c(-c2nnn[nH]2)cccc1[C@H]1Cc2cc(C(F)(F)F)ccc2O1]

or a salt thereof.

653. A compound, wherein the compound has the structure of

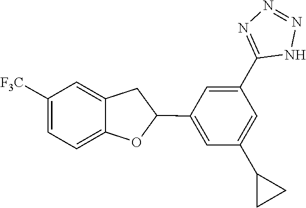 [SMILES: FC(F)(F)c1ccc2c(c1)CC(c1cc(-c3nnn[nH]3)cc(C3CC3)c1)O2]

or a salt thereof.

654. A compound, wherein the compound has the structure of

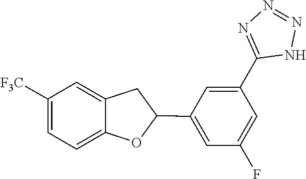 [SMILES: Fc1cc(-c2nnn[nH]2)cc(C2Cc3cc(C(F)(F)F)ccc3O2)c1]

or a salt thereof.

655. A compound, wherein the compound has the structure of

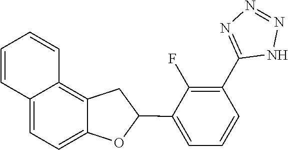 [SMILES: Fc1c(-c2nnn[nH]2)cccc1C1Cc2c(ccc3ccccc23)O1]

or a salt thereof.

656. A compound, wherein the compound has the structure of

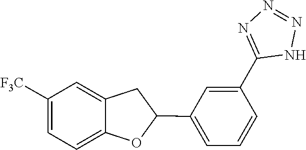 [SMILES: FC(F)(F)c1ccc2c(c1)CC(c1cccc(-c3nnn[nH]3)c1)O2]

or a salt thereof.

657. A compound, wherein the compound has the structure of

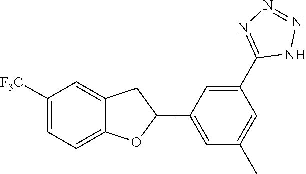 [SMILES: Cc1cc(-c2nnn[nH]2)cc(C2Cc3cc(C(F)(F)F)ccc3O2)c1]

or a salt thereof.

658. A compound, wherein the compound has the structure of

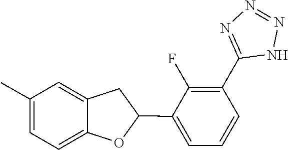 [SMILES: Cc1ccc2c(c1)CC(c1cccc(-c3nnn[nH]3)c1F)O2]

or a salt thereof.

659. A compound, wherein the compound has the structure of

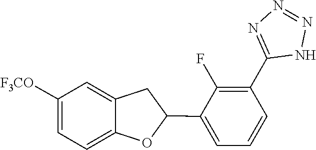 [SMILES: Fc1c(-c2nnn[nH]2)cccc1C1Cc2cc(OC(F)(F)F)ccc2O1]

or a salt thereof.

660. A compound, wherein the compound has the structure of

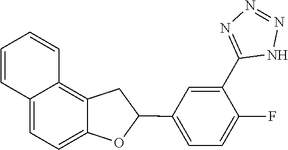 [SMILES: Fc1ccc(C2Cc3c(ccc4ccccc34)O2)cc1-c1nnn[nH]1]

or a salt thereof.

661. A compound, wherein the compound has the structure of

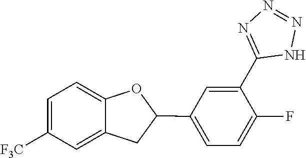 [SMILES: Fc1ccc(C2Cc3cc(C(F)(F)F)ccc3O2)cc1-c1nnn[nH]1]

or a salt thereof.

662. A compound, wherein the compound has the structure of

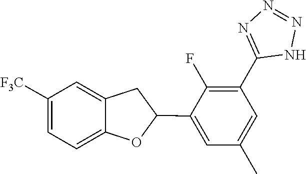 [SMILES: Cc1cc(-c2nnn[nH]2)c(F)c(C2Cc3cc(C(F)(F)F)ccc3O2)c1]

or a salt thereof.

663. A compound, wherein the compound has the structure of

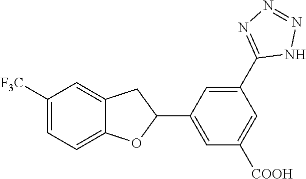 [SMILES: O=C(O)c1cc(-c2nnn[nH]2)cc(C2Cc3cc(C(F)(F)F)ccc3O2)c1]

or a salt thereof.

664. A compound, wherein the compound has the structure of

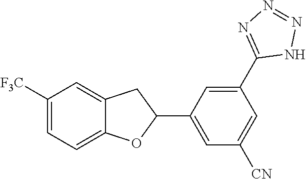 [SMILES: N#Cc1cc(-c2nnn[nH]2)cc(C2Cc3cc(C(F)(F)F)ccc3O2)c1]

or a salt thereof.

665. A compound, wherein the compound has the structure of

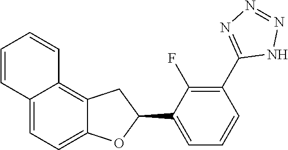 [SMILES: Fc1c(-c2nnn[nH]2)cccc1[C@@H]1Cc2c(ccc3ccccc23)O1]

or a salt thereof.

666. A compound, wherein the compound has the structure of

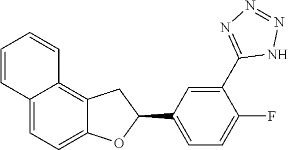 [SMILES: Fc1ccc([C@@H]2Cc3c(ccc4ccccc34)O2)cc1-c1nnn[nH]1]

or a salt thereof.

667. A compound, wherein the compound has the structure of

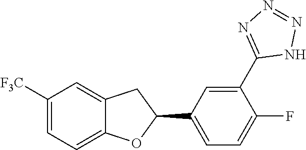 [SMILES: Fc1ccc([C@@H]2Cc3cc(C(F)(F)F)ccc3O2)cc1-c1nnn[nH]1]

or a salt thereof.

668. A compound, wherein the compound has the structure of

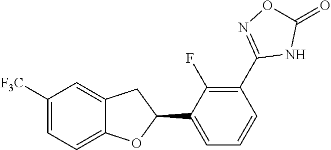 [SMILES: O=c1[nH]c(-c2cccc([C@@H]3Cc4cc(C(F)(F)F)ccc4O3)c2F)no1]

or a salt thereof.

669. The compound of any one of the preceding Embodiments, wherein the compound is a pharmaceutically acceptable salt.

670. The compound of any one of the preceding Embodiments, wherein the compound is a sodium salt.

671. The compound of any one of the preceding Embodiments, wherein the compound has an enantiomeric purity of about or at least about 80%.

672. The compound of any one of the preceding Embodiments, wherein the compound has an enantiomeric purity of about or at least about 90%.

673. The compound of any one of the preceding Embodiments, wherein the compound has an enantiomeric purity of about or at least about 95%.

674. The compound of any one of the preceding Embodiments, wherein the compound has an enantiomeric purity of about or at least about 98%.

675. The compound of any one of the preceding Embodiments, wherein the compound has a purity of about or at least about 90%.

676. The compound of any one of the preceding Embodiments, wherein the compound has a purity of about or at least about 95%.

677. The compound of any one of the preceding Embodiments, wherein the compound has a purity of about or at least about 98%.

678. The compound of any one of the preceding Embodiments, wherein the compound has a purity of about or at least about 98%.

679. A pharmaceutical composition comprising a compound of any one of the preceding Embodiments and a pharmaceutically acceptable carrier.

680. The composition of claim 679, wherein the composition is a topical composition.

681. The composition of claim 679, wherein the composition is a cream, oil or lotion.

682. The composition of claim 679, wherein the composition is a cream, ointment, lotion or gel.

683. A method for modulating a Mas-related G-protein coupled receptor X4 (MRGPRX4) activity by contacting MRGPRX4 with an effective amount of the compound or pharmaceutical composition of any one of Embodiments 1-682.

684. A method for modulating MRGPRX4 activity in a system comprising MRGPRX4, comprising administering or delivering to the system an effective amount of a compound or a pharmaceutical composition of any one of the preceding Embodiments.

685. The method of Embodiment 684, wherein a system is or comprises a cell.

686. The method of Embodiment 684, wherein a system is or comprises a tissue.

687 The method of Embodiment 684, wherein a system is or comprises an organ.

688. The method of Embodiment 684, wherein a system is or comprises an organism.

689. The method of Embodiment 684, wherein a system is a subject.

690. The method of Embodiment 684, wherein a system is an animal.

691. The method of Embodiment 684, wherein a system is a human.

692. The method of any one of Embodiments 683-691, wherein level of MRGPRX4 activity is reduced.

693. The method of any one of Embodiments 683-691, wherein level of MRGPRX4 activity is reduced by at least about 50% compared to absence of the compound or composition.

694. A method for treating a condition, disorder or disease, comprising administering to a subject suffering therefrom an effective amount of the compound or pharmaceutical composition of any one of Embodiments 1-682.

695. A method for treating a condition, disorder or disease, comprising delivering to a subject suffering therefrom an effective amount of the compound or pharmaceutical composition of any one of Embodiments 1-682.

696. A method for preventing a condition, disorder or disease, comprising administering to a subject susceptible thereto an effective amount of the compound or pharmaceutical composition of any one of Embodiments 1-682.

697. A method for preventing a condition, disorder or disease, comprising delivering to a subject susceptible thereto an effective amount of the compound or pharmaceutical composition of any one of Embodiments 1-682.

698. The method of any one of Embodiment 694-697, wherein the condition, disorder or disease is associated with MRGPRX4.

699. The method of any one of Embodiment 694-698, wherein the condition, disorder or disease is associated with MRGPRX4 activation by administration or delivery another therapeutic agent. 700. The method of any one of Embodiment 694-699, comprising administering or delivering to the subject an effective amount of another therapeutic agent.

701. A method for improving adherence of an agent, comprising administering or delivering to a subject receiving the agent an effective amount of the compound or pharmaceutical composition of any one of Embodiments 1-682.

702. A method for improving a single dose, total dose, dose frequency, and/or treatment duration of an agent, comprising administering or delivering to a subject receiving the agent an effective amount of the compound or pharmaceutical composition of any one of Embodiments 1-682.

703. The method of any one of Embodiments 701-702, wherein the agent can activate MRGPRX4.

704. The method of any one of Embodiments 699-703, wherein the agent is a bile acid or a pharmaceutically acceptable salt thereof.

705. The method of any one of Embodiments 699-703, wherein the agent is a bile acid analog or a pharmaceutically acceptable salt thereof.

706. The method of any one of Embodiments 699-703, wherein the agent is a bile acid derivative or a pharmaceutically acceptable salt thereof.

707. The method of any one of Embodiments 699-703, wherein the agent is a bile acid conjugate or a pharmaceutically acceptable salt thereof.

708. The method of any one of Embodiments 699-703, wherein the agent is a bile acid taurine conjugate or a pharmaceutically acceptable salt thereof.

709. The method of any one of Embodiments 704-708, wherein the bile acid is cholic acid.

710. The method of any one of Embodiments 704-708, wherein the bile acid is chenodeoxycholic acid.

711. The method of any one of Embodiments 704-708, wherein the bile acid is ursocholic acid.

712. The method of any one of Embodiments 704-708, wherein the bile acid is ursodeoxycholic acid.

713. The method of any one of Embodiments 699-703, wherein the agent is taurursodiol or a pharmaceutically acceptable salt thereof.

714. The method of any one of Embodiments 699-703, wherein the agent is taurursodiol or a pharmaceutically acceptable salt thereof and it is in combination with phenylbutyric acid or a pharmaceutically acceptable salt thereof.

715. The method of any one of Embodiments 699-703, wherein the agent is taurursodiol and it is in combination with sodium phenylbutyrate.

716. The method of any one of Embodiments 699-703, wherein the agent is obeticholic acid.

717. The method of any one of Embodiments 699-703, wherein the agent is obeticholic acid and it is in combination with ursodeoxycholic acid.

718. The method of any one of Embodiments 699-717, wherein the agent is a FXR agonist.

719. The method of any one of Embodiments 699-717 and 718, wherein the agent is obeticholic acid (OCA), cilofexor (GS-9674), tropifexor (LJN452), EDP-305, EDP-297, nidufexor, TERN-101 (LY2562175), MET-409, BAR704, BAR502, EYP-001, RDX-023, AGN-242266, HPG-1860, AGN-242256, IoT-022, M-480, or INV-33.

720. The method of any one of Embodiments 699-703, wherein the agent is an ileal bile acid transport (IBAT) inhibitor.

721. The method of any one of Embodiments 699-703 and 720, wherein the agent is odevixibat, or maralixibat.

722. The method of any one of Embodiments 699-703, wherein the agent is a glucagon-like peptide 1 (GLP-1) agonist.

723. The method of any one of Embodiments 699-703 and 722, wherein the agent is semaglutide, exenatide, dulaglutide, liraglutide, lixisenatide, danuglipron (PF-06882961), or PF-0708153.

724. The method of any one of Embodiments 699-703, wherein the agent is a glucose-dependent insulinotropic polypeptide (GIP) receptor agonist.

725. The method of any one of Embodiments 699-703 and 724, wherein the agent is tirzepatide.

726. The method of any one of Embodiments 699-703, wherein the agent is a peroxisome proliferator-activated receptors (PPAR) agonist, 727. The method of any one of Embodiments 699-703 and 726, wherein the agent is elafibranor, lanifibranor, saroglitazar, pioglitazone, or rosiglitazone.

728. The method of any one of Embodiments 699-703, wherein the agent is a thyroid hormone receptor β (THR-β) agonist.

729. The method of any one of Embodiments 699-703 and 728, wherein the agent is resmetirom (MGL-3196), GC-24, MGL-3745, VK-2809, KB141 [3,5-dichloro-4-(4-hydroxy-3-isopropylphenoxy)phenylacetic acid], or MB07811 (2R,4S)-4-(3-chlorophenyl)-2-((3,5-dimethyl-4-(4′-hydroxy-3′-isopropylbenzyl) phenoxy)methyl]-2-oxido-[1,3,2]-dioxaphosphonane).

730.

The method of any one of Embodiments 699-703, wherein the agent is an acetyl CoA-carboxylase (ACC) inhibitor.

731. The method of any one of Embodiments 699-703 and 730, wherein the agent is firsocostat, PF-05221304, or WZ66.

732. The method of any one of Embodiments 699-703, wherein the agent is a diacylglycerol O-acyltransferase 2 (DGAT2) inhibitor.

733. The method of any one of Embodiments 699-703 and 732, wherein the agent is PF-06865571. 734. The method of any one of Embodiments 699-703, wherein the agent is a ketohexokinase (KHK) inhibitor.

735. The method of any one of Embodiments 699-703 and 734, wherein the agent is PF-06835919.

736. The method of any one of Embodiments 699-717, wherein the agent is a TGR5 agonist.

737. The method of any one of Embodiments 699-736, wherein the compound or composition is administered or delivered concurrently with the agent.

738. The method of any one of Embodiments 699-736, wherein the compound or composition is administered or delivered in the same composition as the agent.

739. The method of any one of Embodiments 699-736, wherein the compound or composition is administered or delivered about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months before administration of the agent.

740. The method of any one of Embodiments 699-736, wherein the compound or composition is administered or delivered about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months after administration of the agent. 741. The method of any one of Embodiments 694-740, wherein the condition, disorder or disease is or comprises itch.

742. The method of any one of Embodiments 694-740, wherein the condition, disorder or disease is or comprises chronic itch, cholestatic pruritus, contact dermatitis, allergic blepharitis, anemia, atopic dermatitis, bullous pemphigoid, candidiasis, chicken pox, cholestasis, end-stage renal failure, hemodialysis, contact dermatitis, dermatitis herpetiformis, diabetes, drug allergy, dry skin, dyshidrotic dermatitis, ectopic eczema, eczema, erythrasma, folliculitis, fungal skin infection, hemorrhoids, herpes, HIV infection, Hodgkin's disease, hyperthyroidism, iron deficiency anemia, kidney disease, leukemia, liver disease, lymphoma, malignancy, multiple myeloma, neurodermatitis, onchocerciasis, Paget's disease, pediculosis, polycythemia rubra vera, pruritus ani, pseudorabies, psoriasis, rectal prolapse, scabies, schistosomiasis, scleroderma, severe stress, stasis dermatitis, swimmer's itch, thyroid disease, tinea cruris, uremic pruritus, or urticaria.

743. The method of any one of Embodiments 694-740, wherein the condition, disorder or disease is pruritus, atopic dermatitis, dry skin, psoriasis, contact dermatitis, or eczema.

744. The method of any one of Embodiments 694-740, wherein the condition, disorder or disease is pruritus.

745. The method of any one of Embodiments 744, wherein pruritus is an acute or chronic pruritus associated a liver condition, disorder or disease.

746. The method of any one of Embodiments 694-740, wherein the condition, disorder or disease is a liver condition, disorder or disease.

747. The method of Embodiment 745 or 746, wherein the liver condition, disorder or disease is intrahepatic cholestasis of pregnancy (ICP), estrogen-, progesterone- or testosterone-induced cholestasis, toxin- or other drug induced hepatocellular cholestasis, benign recurrent intrahepatic cholestasis (BRIC), progressive familial intrahepatic cholestasis (PFIC), chronic viral hepatitis C, chronic hepatitis B, alcoholic or nonalcoholic fatty liver disease (NAFLD), nonalcoholic steatohepatitis (NASH), primary biliary cholangitis (PBC), primary sclerosing cholangitis (PSC), secondary sclerosing cholangitis (SSC), sarcoidosis, ABCB4 deficiency, alagille syndrome, drug-induce small duct cholangiopathies, gallstone disease, IgG4-associated cholangitis, biliary atresia, cholangiocellular carcinoma, benign bile duct adenoma, or other obstructive cholestasis.

748. The method of any one of Embodiments 694-740, wherein the condition, disorder or disease is primary biliary cirrhosis.

749. The method of any one of Embodiments 694-740, wherein the condition, disorder or disease is primary biliary cholangitis.

750. The method of any one of Embodiments 694-740, wherein the condition, disorder or disease is primary biliary cholangitis without cirrhosis.

751. The method of any one of Embodiments 694-740, wherein the condition, disorder or disease is primary biliary cholangitis with compensated cirrhosis.

752. The method of any one of Embodiments 694-740, wherein the condition, disorder or disease is primary biliary cholangitis with compensated cirrhosis who do not have evidence of portal hypertension.

753. The method of any one of Embodiments 694-740, wherein the condition, disorder or disease is a bile acid synthesis disorder.

754. The method of any one of Embodiments 694-740, wherein the condition, disorder or disease is a bile acid synthesis disorder due to a single enzyme defect (SED).

755. The method of any one of Embodiments 694-740, wherein the condition, disorder or disease is a peroxisomal disorder.

756. The method of any one of Embodiments 694-740, wherein the condition, disorder or disease is Zellweger spectrum disorder.

757. The method of any one of Embodiments 694-740, wherein the condition, disorder or disease is Zellweger spectrum disorder, wherein the subject exhibits manifestations of liver disease, steatorrhea or complications.

758. The method of any one of Embodiments 694-740, wherein the condition, disorder or disease is a neurodegenerative condition, disorder or disease.

759. The method of any one of Embodiments 694-740, wherein the condition, disorder or disease is amyotrophic lateral sclerosis.

760. The method of any one of Embodiments 694-740, wherein the condition, disorder or disease is NASH.

761. The method of any one of Embodiments 694-740, wherein the condition, disorder or disease is NAFLD.

762. The method of any one of Embodiments 694-740, wherein the condition, disorder or disease is ICP.

763. The method of any one of Embodiments 694-740, wherein the condition, disorder or disease is a chronic kidney disease.

764. The method of any one of Embodiments 694-740, wherein the condition, disorder or disease is uremic pruritus.

765. The method of any one of Embodiments 694-740, wherein the condition, disorder or disease is PFIC.

766. The method of any one of Embodiments 694-740, wherein the condition, disorder or disease is PSC.

767. The method of any one of Embodiments 694-740, wherein the condition, disorder or disease is BRIC.

768. The method of any one of Embodiments 694-740, wherein the condition, disorder or disease is chronic hepatitis B.

769. The method of any one of Embodiments 694-768, wherein a compound or composition is administered or delivered topically.

770. The method of any one of Embodiments 694-768, wherein a compound or composition is administered or delivered as a cream.

771. The method of any one of Embodiments 694-768, wherein a compound or composition is administered or delivered as a lotion.

772. The method of any one of Embodiments 694-768, wherein a compound or composition is administered or delivered as an ointment.

773. The method of any one of Embodiments 694-768, wherein the subject is an adult patient.

774. The method of any one of Embodiments 694-768, wherein the subject is a pediatric patient.

775. A method, comprising:
reacting a compound having the structure of formula B-4

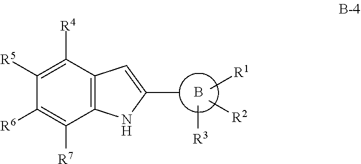

or a salt thereof with a compound having the structure of R⁸-LG or a salt thereof, wherein LG is a leaving group to provide a compound having the structure of formula B:

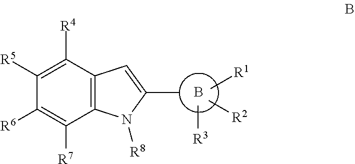

or a salt thereof, wherein each variable is independently as described in any one of the preceding Embodiments.

776. The method of Embodiment 775, wherein the compound having the structure of formula B-4 or a salt thereof is a compound having the structure of

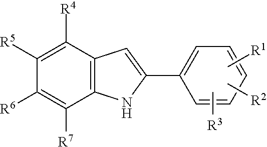

or a salt thereof.

777. The method of any one of Embodiment 775-776, wherein the compound having the structure of formula B or a salt thereof is a compound having the structure of

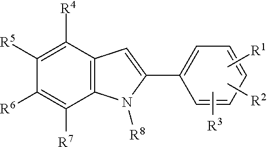

or a salt thereof.

778. The method of any one of Embodiments 775-777, wherein the reaction is performed in the presence of a base.

779. The method of any one of Embodiment 775-778, wherein LG is Cl, Br or I.

780. A method, comprising:
reacting a compound having the structure of formula B-3:

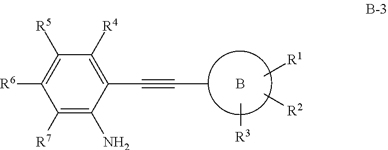

or a salt thereof to provide a compound having the structure of formula B-4:

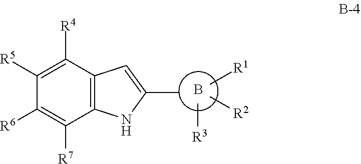

or a salt thereof, wherein each variable is independently as described in any one of the preceding Embodiments.

781. The method of any one of Embodiments 775-779, comprising a method of Embodiment 780.

782. The method of Embodiment 780 or 781, wherein the compound having the structure of B-3 or a salt thereof is a compound having the structure of

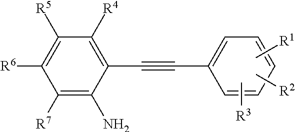

or a salt thereof.

783. The method of any one of Embodiments 780-782, wherein the compound having the structure of B-4 or a salt thereof is a compound having the structure of

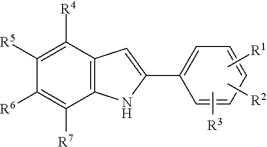

or a salt thereof.

784. The method of any one of Embodiments 780-783, wherein the reaction is performed in the presence of a metal.

785. The method of Embodiment 784, wherein the reaction is performed in the presence of Pd.

786. The method of Embodiment 784, wherein the reaction is performed in the presence of a Pd complex.

787. The method of Embodiment 786, wherein the complex is PdCl₂.

788. A method, comprising:
reacting a compound having the structure of formula B-1:

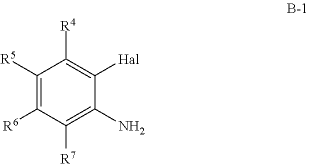

or a salt thereof and a compound having the structure of formula B-2:

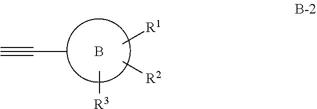

or a salt thereof, to provide a compound having the structure of formula B-3:

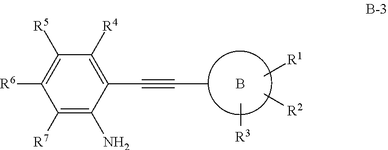

or a salt thereof, wherein Hal is halogen, and each variable is independently as described in any one of the preceding Embodiments.

789. The method of any one of Embodiments 775-787, comprising a method of Embodiment 788.

790. The method of Embodiment 788 or 789, wherein the compound having the structure of B-3 or a salt thereof is a compound having the structure of

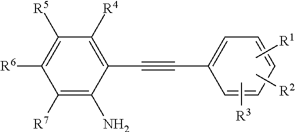

or a salt thereof.

791. The method of any one of Embodiments 788-790, wherein the compound having the structure of B-2 or a salt thereof is a compound having the structure of

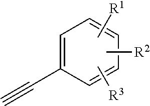

or a salt thereof.

792. The method of any one of Embodiments 788-791, wherein Hal is Cl.

793. The method of any one of Embodiments 788-791, wherein Hal is Br.

794. The method of any one of Embodiments 788-791, wherein Hal is I.

795. The method of any one of Embodiments 788-794, wherein the reaction is performed in the presence of a metal.

796. The method of Embodiment 795, wherein the reaction is performed in the presence of Pd.

797. The method of Embodiment 796, wherein reaction is performed in the presence of a Pd complex.

798. The method of Embodiment 797, wherein the complex is PdCl₂(PPh₃)₂.

799. The method of any one of Embodiments 795-798, wherein the reaction is performed in the presence of Cu.

800. The method of any one of Embodiments 795-798, wherein the reaction is performed in the presence of Cu(I).

801. The method of any one of Embodiments 799-800, wherein the reaction is performed in the presence of a Cu(I) complex.

802. The method of Embodiment 801, wherein the complex is CuI.

803. The method of any one of Embodiments 788-802, wherein the reaction is performed in the presence of a base.

804. The method of Embodiment 803, wherein the base is Et₃N.

805. A method, comprising:
reacting a compound having the structure of formula B-5:

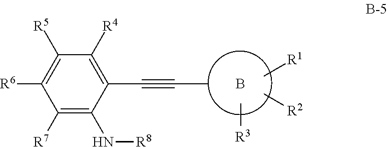

or a salt thereof to provide a compound having the structure of formula B:

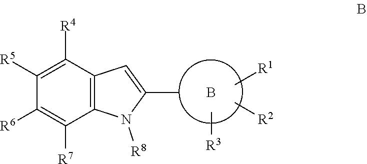

or a salt thereof, wherein each variable is independently as described in any one of the preceding Embodiments.

806. The method of Embodiment 805, wherein the compound having the structure of B-5 or a salt thereof is a compound having the structure of

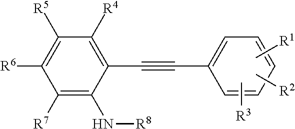

or a salt thereof.

807. The method of any one of Embodiments 780-782, wherein the compound having the structure of B or a salt thereof is a compound having the structure of

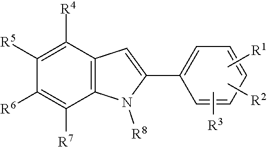

or a salt thereof.

808. The method of any one of Embodiments 805-807, wherein the reaction is performed in the presence of a metal.

809. The method of Embodiment 808, wherein the reaction is performed in the presence of Pd.

810. The method of Embodiment 808, wherein the reaction is performed in the presence of a Pd complex.

811. The method of Embodiment 809, wherein the complex is PdCl₂.

812. A method, comprising:
reacting a compound having the structure of formula B-3:

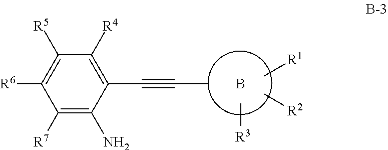

or a salt thereof to provide a compound having the structure of formula B-5:

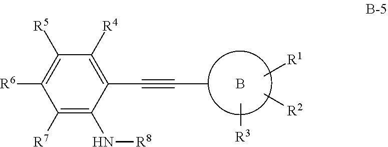

or a salt thereof wherein each variable is independently as described in any one of the preceding Embodiments.

813. The method of any one of Embodiments 805-811, comprising a method of Embodiment 812.

814. The method of Embodiment 812 or 813, wherein the compound having the structure of B-5 or a salt thereof is a compound having the structure of

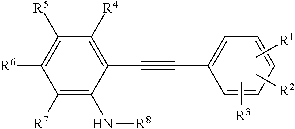

or a salt thereof.

815. The method of any one of Embodiments 812-814, wherein the compound having the structure of B-3 or a salt thereof is a compound having the structure of

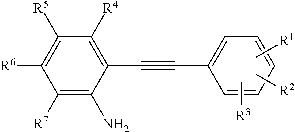

or a salt thereof.

816. The method of any one of Embodiments 812-815, comprising reacting a compound of formula B-3 or a salt thereof with a compound having the structure of R⁸-LG or a salt thereof.

817. The method of Embodiment 816, wherein the method is performed in the presence of a base.

818. The method of any one of Embodiments 816-817, wherein LG is Cl, Br or I.

819 The method of any one of Embodiments 812-815, comprising reacting a compound of formula B-3 or a salt thereof with a compound having the structure of R⁸′—CHO or a salt thereof, wherein R⁸ is bonded to the —NH-through-CH₂—, and R⁸′ is of such a structure that R⁸′—CH₂—is R⁸.

820. The method of Embodiment 819, wherein the reaction is performed in the presence of a reducing agent.

821. The method of Embodiment 820, wherein the reducing agent is a boron hydride agent.

822. A method, comprising reacting a compound of formula B-3:

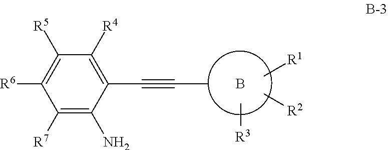

or a salt thereof with a compound having the structure of R⁸—B(OH)₂ or a salt thereof to provide a compound having the structure of formula B:

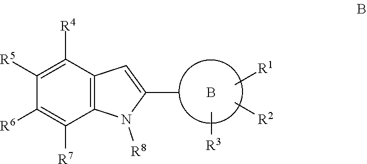

or a salt thereof wherein each variable is independently as described in any one of the preceding Embodiments.

823. The method of Embodiment 822, wherein the compound having the structure of B-3 or a salt thereof is a compound having the structure of

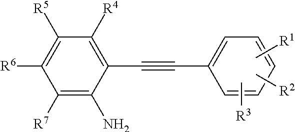

or a salt thereof.

824. The method of any one of Embodiments 822-823, wherein the compound having the structure of B or a salt thereof is a compound having the structure of

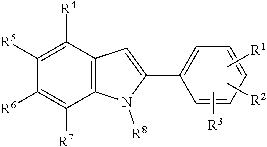

or a salt thereof.

825. The method of any one of Embodiments 822-824, wherein the reaction is performed in the presence of a metal.

826. The method of Embodiment 823, wherein the reaction is performed in the presence of Cu.

827. The method of Embodiment 823, wherein the reaction is performed in the presence of Cu(II).

828. The method of any one of Embodiments 822-827, wherein the reaction is performed in the presence of a Cu complex.

829. The method of Embodiment 828, wherein the complex is Cu(OAc)₂.

830. A method, comprising reacting a compound of formula I or a salt thereof of any one of the preceding Embodiments wherein R¹¹ is not —H to a compound of formula I or a salt thereof of any one of the preceding Embodiments wherein R¹¹ is —H.

831. The method of Embodiment 830, wherein in a compound of formula I or a salt thereof wherein R¹¹ is not —H, R¹¹ is optionally substituted C₁₋₆ aliphatic.

832. The method of any one of Embodiments 830-831, wherein a compound of formula I or a salt thereof wherein R¹¹ is not —H has the structure of formula C-5:

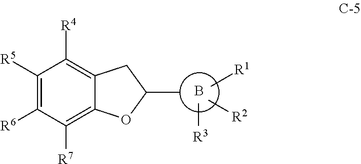

or a salt thereof, wherein each variable is independently described in any one of the preceding Embodiments.

833. The method of any one of Embodiments 830-832, wherein a compound of formula I or a salt thereof wherein R¹¹ is not —H has the structure of

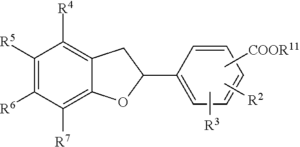

or a salt thereof wherein each variable is independently described in any one of the preceding Embodiments and R¹¹ is not-H.

834. The method of any one of Embodiments 830-832, wherein a compound of formula I or a salt thereof wherein R¹¹ is —H has the structure of formula C:

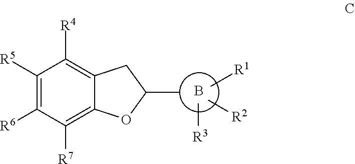

or a salt thereof, wherein R¹ is —C(O)OH.

835. The method of any one of Embodiments 830-834, wherein a compound of formula I or a salt thereof wherein R¹¹ is —H has the structure of

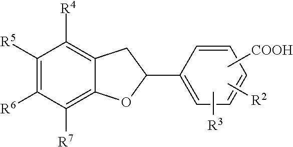

or a salt thereof wherein each variable is independently described in any one of the preceding Embodiments.

836. The method of any one of Embodiments 830-835, wherein the reaction is performed in the presence of a base and water.

837. The method of Embodiment 836, wherein a base is LiOH.

838. A method, comprising:
reacting a compound having the structure of formula C-4:

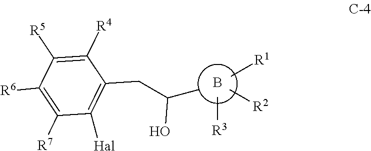

or a salt thereof to provide a compound having the structure of formula C-5:

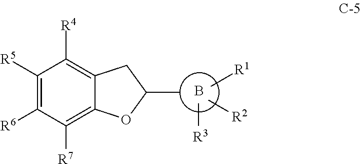

or a salt thereof, wherein Hal is a halogen, and each other variable is independently as described in any one of the preceding Embodiments.

839. The method of any one of Embodiments 830-837, comprising a method of Embodiment 838.

840. The method of Embodiment 838 or 839, wherein R¹ in formula C-4 is —C(O)OR¹¹, wherein R¹¹ is not —H.

841. The method of any one of Embodiments 838-840, wherein R¹ in formula C-5 is —C(O)OR″, wherein R¹¹ is not —H.

842. The method of any one of Embodiments 838-841, wherein the compound having the structure of formula C-4 or a salt of has the structure of

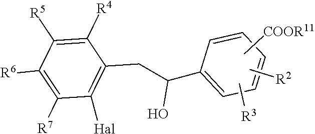

or a salt thereof.

843. The method of any one of Embodiments 838-842, wherein the compound having the structure of formula C-5 or a salt thereof has the structure

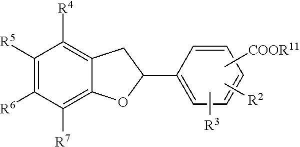

or a salt thereof.

844. The method of any one of Embodiments 838-843, wherein R¹¹ is optionally substituted C₁₋₆ aliphatic.

845. The method of any one of Embodiments 838-844, wherein Hal is Cl.

846. The method of any one of Embodiments 838-844, wherein Hal is Br.

847. The method of any one of Embodiments 838-844, wherein Hal is I.

848. The method of any one of Embodiments 838-847, wherein the reaction is performed in the presence of a metal.

849. The method of Embodiment 848, wherein the reaction is performed in the presence of Pd.

850. The method of Embodiment 849, wherein the reaction is performed in the presence of a Pd complex.

851. The method of Embodiment 850, wherein the complex is Pd(OAc)₂.

852. The method of any one of Embodiments 838-851, wherein the reaction is performed in the presence of a phosphine compound.

853. The method of Embodiment 852, wherein the phosphine compound has the structure of PPh(R)₃ or a salt thereof, wherein each R is independently not —H.

854. The method of Embodiment 852, wherein the phosphine compound is

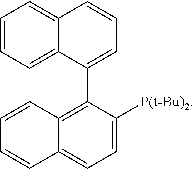

855. The method of any one of Embodiments 838-854, wherein the reaction is performed in the presence of a base.

856. The method of Embodiment 855, wherein the base is Cs₂CO₃.

857. The method of any one of Embodiments 838-856, wherein the reaction is performed in the presence of Cu.

858. The method of any one of Embodiments 838-856, wherein the reaction is performed in the presence of Cu(I).

859. The method of any one of Embodiments 857-858, wherein the reaction is performed in the presence of a Cu complex.

860. The method of Embodiment 859, wherein the complex is CuCl.

861. The method of any one of Embodiments 857-860, wherein the reaction is performed in the presence of a base.

862. The method of Embodiment 861, wherein the base is NaH.

863. A method, comprising:
reacting a compound having the structure of formula C-2:

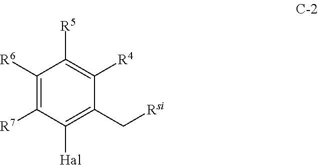

or a salt thereof with a compound having the structure of formula C-3:

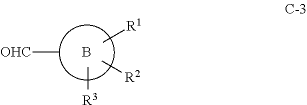

to provide a compound having the structure of formula C-4:

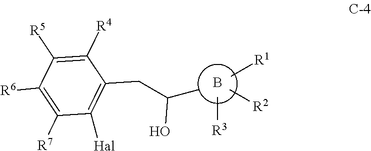

or a salt thereof, wherein Hal is a halogen, R^{si} is —Si(R)₃, and each variable is independently as described in any one of the preceding Embodiments.

864. The method of any one of Embodiments 830-863, comprising a method of Embodiment 863.

865. The method of any one of Embodiments 863-864, wherein Hal is Cl.

866 The method of any one of Embodiments 863-864, wherein Hal is Br.

867 The method of any one of Embodiments 863-864, wherein Hal is I.

868 The method of any one of Embodiments 863-867, wherein R^{si} is —Si(R)₃ wherein each R is not —H.

869. The method of any one of Embodiments 863-867, wherein R^{si} is —Si(R)₃ wherein each R is independently optionally substituted C₁₋₆ aliphatic.

870. The method of any one of Embodiments 863-867, wherein R^{si} is —Si(Me)₃.

871. The method of any one of Embodiments 863-870, wherein compound having the structure of formula C-4 or a salt thereof has the structure of

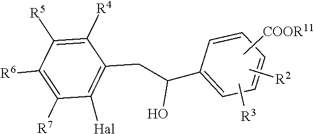

or a salt thereof.

872. The method of any one of Embodiments 863-871, wherein the compound having the structure of formula C-3 or a salt thereof has the structure of

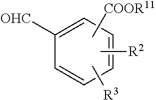

or a salt thereof.

873. The method of any one of Embodiments 863-872, wherein the compound having the structure of C-2 or salt thereof has the structure of

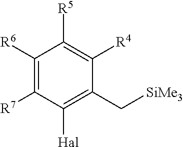

or a salt thereof.

874. The method of any one of Embodiments 863-873, wherein the reaction is performed under a desilylation condition.

875. The method of any one of Embodiments 863-874, wherein the reaction is performed in the presence of a fluoride agent.

876. The method of Embodiment 875, wherein the fluoride agent is TBAF.

877. A method, comprising:
reacting a compound having the structure of formula C-1:

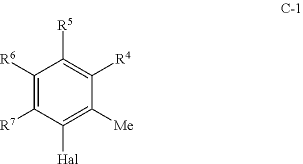

or a salt thereof to provide a compound having the structure of formula C-2:

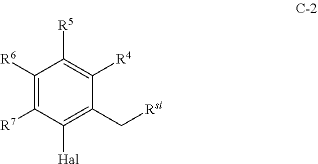

or a salt thereof, wherein each variable is independently as described in any one of the preceding Embodiments.

878. The method of any one of Embodiments 830-876, comprising a method of Embodiment 877.

879 The method of Embodiment 877 or 878, wherein Hal is Cl.

880 The method of Embodiment 877 or 878, wherein Hal is Br.

881. The method of Embodiment 877 or 878, wherein Hal is I.

882. The method of any one of Embodiments 877-881, wherein R^{si} is —Si(R)₃ wherein each R is not —H.

883. The method of any one of Embodiments 877-881, wherein R^{si} is —Si(R)₃ wherein each R is independently optionally substituted C₁₋₆ aliphatic.

884. The method of any one of Embodiments 877-881, wherein R^{si} is —Si(Me)₃.

885. The method of any one of Embodiments 877-881, wherein the compound having the structure of C-2 or salt thereof has the structure of

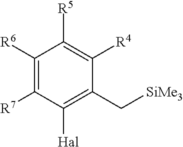

or a salt thereof.

886. The method of any one of Embodiments 877-885, wherein the reaction is performed in the presence of a base.

887. The method of Embodiment 886, wherein the base is LDA.

888. The method of any one of Embodiments 877-887, wherein the reaction is performed in the presence of a silylating agent.

889. The method of Embodiment 888, wherein a silylating agent is a compound having the structure of formula R^{si}-LG or a salt thereof, wherein LG is a leaving group.

890. The method of Embodiment 889, wherein LG is Cl.

891. The method of any one of Embodiments 877-890, wherein the reaction is performed at a reduced temperature.

892. A method, comprising:
reacting a compound having the structure of formula D-7:

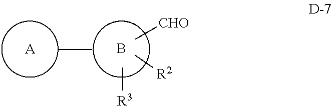

or a salt thereof to provide a compound having the structure of formula I or a salt thereof, wherein each variable is independently as described in any one of the preceding Embodiments.

893. The method of Embodiment 892, wherein the compound having the structure of formula D-7 or a salt thereof has the structure of

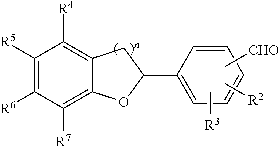

or a salt thereof.

894. The method of any one of Embodiments 892-893, wherein the compound having the structure of formula I or a salt thereof has the structure of

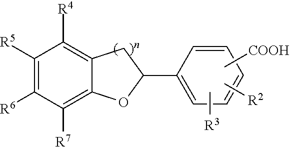

or a salt thereof.

895. The method of any one of Embodiments 892-894, wherein t reaction is performed under an oxidation condition.

896. A method, comprising:
reacting a compound having the structure of formula D-6:

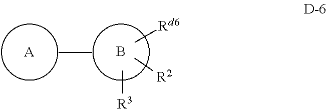

or a salt thereof to provide a compound having the structure of formula D-7:

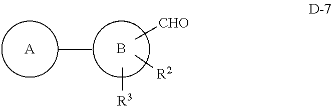

or a salt thereof, wherein R^{d6} is —CH(OR)₂, and each variable is independently as described in any one of the preceding Embodiments.

897. The method of any one of Embodiments 892-895, comprising a method of Embodiment 896.

898. The method of any one of Embodiments 896-897, wherein R^{d6} is —CH(OR)₂ wherein each R is independently not H.

899. The method of any one of Embodiments 896-897, wherein R^{d6} is —CH(OR)₂ wherein each R is independently optionally substituted C₁₋₆ aliphatic.

900. The method of any one of Embodiments 896-897, wherein R^{d6} is —CH(OR)₂ wherein the two R are taken together with their intervening atoms to form an optionally substituted 4-10, e.g., 5-10, 5-6, 4, 5, 6, 7, 8, 9, or 10 membered ring having 0-3 heteroatoms in addition to the intervening atoms.

901. The method of any one of Embodiments 896-897, wherein R^{d6} is optionally substituted

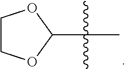 [SMILES: *C1OCCO1]

902. The method of any one of Embodiments 896-897, wherein R^{d6} is

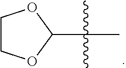 [SMILES: *C1OCCO1]

903. The method of any one of Embodiments 896-902, wherein the compound having the structure of formula D-6 or a salt thereof has the structure of

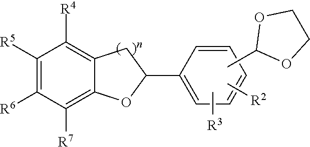

or a salt thereof.

904. The method of any one of Embodiments 896-903, wherein the compound having the structure of formula D-7 or a salt thereof has the structure of

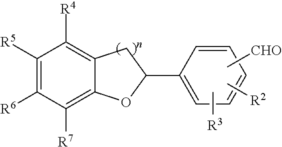

or a salt thereof.

905. The method of any one of Embodiments 896-904, wherein the reaction is performed in the presence of an acid.

906. A method, comprising:
reacting a compound having the structure of formula D-5:

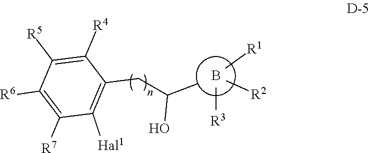

or a salt thereof to provide a compound having the structure of formula D-6:

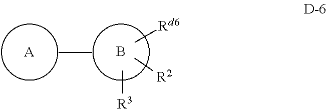

or a salt thereof, wherein Hall is Hal as described herein, and each other variable is independently as described in any one of the preceding Embodiments.

907. The method of any one of Embodiments 892-905, comprising the method of Embodiment 906.

908. The method of any one of Embodiments 906-907, wherein R^{d6} is —CH(OR)₂ wherein each R is independently not H.

909. The method of any one of Embodiments 906-907, wherein R^{d6} is —CH(OR)₂ wherein each R is independently optionally substituted C₁₋₆ aliphatic.

910. The method of any one of Embodiments 906-907, wherein R^{d6} is —CH(OR)₂ wherein the two R are taken together with their intervening atoms to form an optionally substituted 4-10, e.g., 5-10, 5-6, 4, 5, 6, 7, 8, 9, or 10 membered ring having 0-3 heteroatoms in addition to the intervening atoms.

911. The method of any one of Embodiments 906-907, wherein R^{d6} is optionally substituted

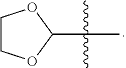 [SMILES: *C1OCCO1]

912. The method of any one of Embodiments 906-907, wherein R^{d6} is

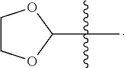 [SMILES: *C1OCCO1]

913. The method of any one of Embodiments 906-912, wherein the compound having the structure of formula D-5 or a salt of has the structure of

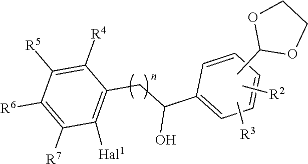

or a salt thereof.

914. The method of any one of Embodiments 906-913, wherein the compound having the structure of formula D-6 or a salt thereof has the structure of

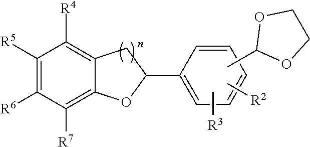

or a salt thereof.

915. The method of any one of Embodiments 906-914, wherein Hall is Cl.

916 The method of any one of Embodiments 906-914, wherein Hall is Br.

917. The method of any one of Embodiments 906-914, wherein Hall is I.

918. The method of any one of Embodiments 906-917, wherein the reaction is performed in the presence of a metal.

919. The method of Embodiment 918, wherein the reaction is performed in the presence of Pd.

920. The method of Embodiment 919, wherein the reaction is performed in the presence of a Pd complex.

921. The method of Embodiment 920, wherein the complex is Pd(OAc)₂.

922. The method of any one of Embodiments 906-921, wherein the reaction is performed in the presence of a phosphine compound.

923. The method of Embodiment 922, wherein the phosphine compound has the structure of PPh (R)₃ or a salt thereof, wherein each R is independently not —H.

924. The method of Embodiment 922, wherein the phosphine compound is

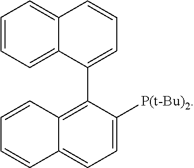

925. The method of any one of Embodiments 906-924, wherein the reaction is performed in the presence of a base.

926. The method of Embodiment 925, wherein the base is Cs₂CO₃.

927. The method of any one of Embodiments 906-926, wherein the reaction is performed in the presence of Cu.

928. The method of any one of Embodiments 906-926, wherein the reaction is performed in the presence of Cu(I).

929. The method of any one of Embodiments 927-928, wherein the reaction is performed in the presence of a Cu complex.

930. The method of Embodiment 929, wherein the complex is CuCl.

931. The method of any one of Embodiments 927-930, wherein the reaction is performed in the presence of a base.

932. The method of Embodiment 931, wherein the base is NaH.

933. A method, comprising:
reacting a compound having the structure of formula D-4:

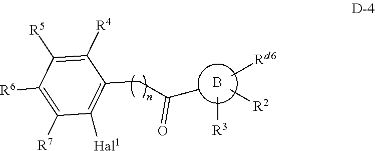

or a salt thereof to provide a compound having the structure of formula D-5:

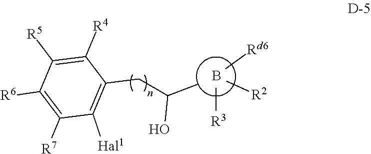

or a salt thereof, wherein each variable is independently as described in any one of the preceding Embodiments.

934. The method of any one of Embodiments 892-932, comprising the method of Embodiment 933.

935. The method of any one of Embodiments 933-934, wherein R^{d6} is —CH(OR)₂ wherein each R is independently not H.

936. The method of any one of Embodiments 933-934, wherein R^{d6} is —CH(OR)₂ wherein each R is independently optionally substituted C₁₋₆ aliphatic.

937. The method of any one of Embodiments 933-934, wherein R^{d6} is —CH(OR)₂ wherein the two R are taken together with their intervening atoms to form an optionally substituted 4-10, e.g., 5-10, 5-6, 4, 5, 6, 7, 8, 9, or 10 membered ring having 0-3 heteroatoms in addition to the intervening atoms.

938. The method of any one of Embodiments 933-934, wherein R^{d6} is optionally substituted

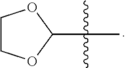 [SMILES: *C1OCCO1]

939. The method of any one of Embodiments 933-934, wherein R^{d6} is

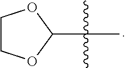 [SMILES: *C1OCCO1]

940. The method of any one of Embodiments 933-939, wherein the compound having the structure of formula D-4 or a salt of has the structure of

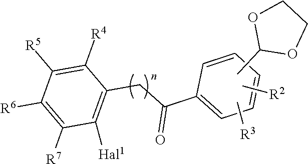

or a salt thereof.

941. The method of any one of Embodiments 933-940, wherein the compound having the structure of formula D-5 or a salt of has the structure of

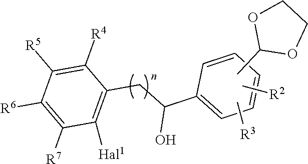

or a salt thereof.

942. The method of any one of Embodiments 933-940, wherein the reaction is performed in the presence of a reducing agent.

943. A method, comprising:
reacting a compound having the structure of formula D-2:

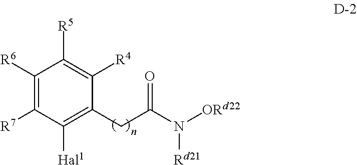

or a salt thereof with a compound having the structure of formula D-3:

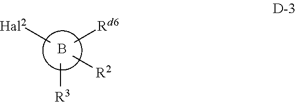

or a salt thereof to provide a compound having the structure of formula D-4:

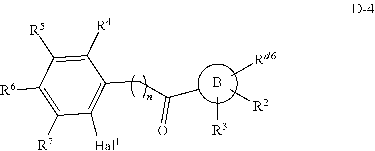

or a salt thereof, wherein each Hal² is Hal, each of R^{d21} and R⁴²² is independently R, and each other variable is independently as described in any one of the preceding Embodiments.

944. The method of any one of Embodiments 892-942, comprising the method of Embodiment 943.

945. The method of any one of Embodiments 943-944, wherein R^{d6} is —CH(OR)₂ wherein each R is independently not H.

946. The method of any one of Embodiments 943-944, wherein R^{d6} is —CH(OR)₂ wherein each R is independently optionally substituted C₁₋₆ aliphatic.

947. The method of any one of Embodiments 943-944, wherein R^{d6} is —CH(OR)₂ wherein the two R are taken together with their intervening atoms to form an optionally substituted 4-10, e.g., 5-10, 5-6, 4, 5, 6, 7, 8, 9, or 10 membered ring having 0-3 heteroatoms in addition to the intervening atoms.

948. The method of any one of Embodiments 943-944, wherein R^{d6} is optionally substituted

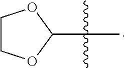 [SMILES: *C1OCCO1]

949. The method of any one of Embodiments 943-944, wherein R^{d6} is

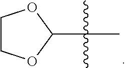 [SMILES: *C1OCCO1]

950. The method of any one of Embodiments 943-949, wherein R^{d21} is optionally substituted C₁₋₆ aliphatic.

951. The method of any one of Embodiments 943-949, wherein R^{d21} is methyl.

952. The method of any one of Embodiments 943-951, wherein R^{d22} is optionally substituted C₁₋₆ aliphatic.

953. The method of any one of Embodiments 943-951, wherein R^{d22} is methyl.

954. The method of any one of Embodiments 943-953, wherein the compound having the structure of formula D-2 or a salt thereof has the structure of

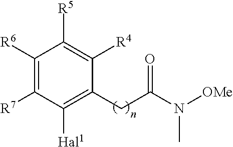

or a salt thereof.

955. The method of any one of Embodiments 943-954, wherein the compound having the structure of formula D-3 or a salt thereof has the structure of

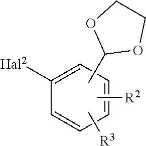

or a salt thereof.

956. The method of any one of Embodiments 943-955, wherein the compound having the structure of formula D-4 or a salt thereof has the structure of

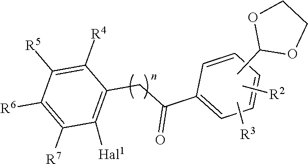

or a salt thereof.

957. The method of any one of Embodiments 943-956, wherein Hal² is Cl.

958 The method of any one of Embodiments 943-956, wherein Hal² is Br.

959. The method of any one of Embodiments 943-956, wherein Hal² is I.

960. The method of any one of Embodiments 943-959, wherein the reaction is performed in the presence of an organometallic agent.

961. The method of Embodiment 960, wherein the agent is a Li agent, e.g., n-BuLi.

962. The method of Embodiment 960, wherein the agent is a Mg agent, e.g., i-PrMgBr.

963. The method of any one of Embodiments 943-962, wherein the compound having the structure of formula D-3 or a salt thereof is contacted with an organometallic agent, and the resulting agent is contacted with a compound having the structure of formula D-2 or a salt thereof.

964. A method, comprising:
reacting a compound having the structure of formula D-1:

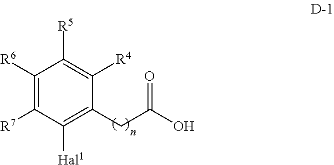

or a salt thereof with a compound having the structure of formula NH(R^{d21}) OR^{d22} or a salt thereof to provide a compound having the structure of formula D-2:

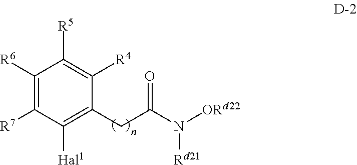

or a salt thereof, wherein each variable is independently as described in any one of the preceding Embodiments.

965. The method of any one of Embodiments 892-963, comprising the method of Embodiment 964.

966. The method of any one of Embodiments 964-965, wherein R^{d21} is optionally substituted C₁₋₆ aliphatic.

967. The method of any one of Embodiments 964-965, wherein R^{d21} is methyl.

968. The method of any one of Embodiments 964-967, wherein R^{d22} is optionally substituted C₁₋₆ aliphatic.

969. The method of any one of Embodiments 964-967, wherein R^{d22} is methyl.

970. The method of any one of Embodiments 964-969, wherein the compound having the structure of formula D-2 or a salt thereof has the structure of

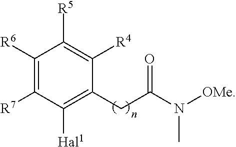

971. The method of any one of Embodiments 964-970, wherein the compound having the structure of formula NH(R^{d21})OR^{d22} or a salt thereof is MeNHOMe or a salt thereof.

972. The method of any one of Embodiments 964-970, wherein the compound having the structure of formula NH R^{d21})OR^{d22} or a salt thereof is MeNHOMe-HCl.

973. The method of any one of Embodiments 964-972, wherein the reaction is performed under a coupling condition.

974. The method of any one of Embodiments 892-973, wherein Hall is Cl.

975. The method of any one of Embodiments 892-973, wherein Hall is Br.

976. The method of any one of Embodiments 892-973, wherein Hall is I.

977. The method of any one of Embodiments 892-976, wherein n is 1.

978 The method of any one of Embodiments 892-976, wherein n is 2.

979. The method of any one of Embodiments 892-976, wherein n is 3.

980. A method, comprising:
reacting a compound having the structure of formula D′-5:

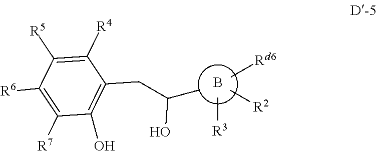

or a salt thereof to provide a compound having the structure of formula D-6:

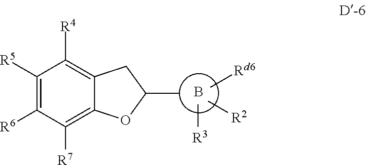

or a salt thereof, wherein each variable is independently as described in any one of the preceding Embodiments.

981. The method of any one of Embodiments 892-905, wherein n is 1, and the method comprises the method of Embodiment 980.

982. The method of any one of Embodiments 980-981, wherein the reaction is performed under a Mitsunobu condition.

983. The method of any one of Embodiments 980-982, wherein the reaction is performed in the presence of a phosphine compound.

984. The method of Embodiment 983, wherein the phosphine compound has the structure of P(R)₃ wherein each R is independently not —H.

985. The method of Embodiment 983, wherein the phosphine compound has the structure of PPh₃.

986. The method of any one of Embodiments 980-985, wherein the reaction is performed in the presence of an azodicarboxylate compound.

987. The method of Embodiment 986, wherein the azodicarboxylate compound has the structure of R^{a1}O₂C—N═N—CO₂R^{a2} or a salt thereof, wherein each of R^{a1} and R^{a2} is independently R.

988. The method of Embodiment 986, wherein the azodicarboxylate compound is DEAD or DIAD.

989. A method, comprising:
reacting a compound having the structure of formula D′-4:

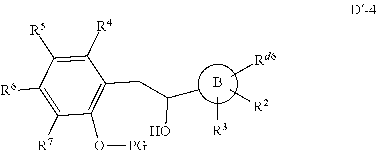

or a salt thereof to provide a compound having the structure of formula D′-5:

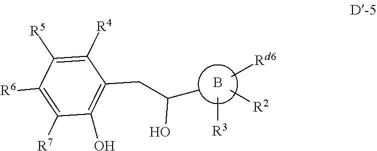

or a salt thereof, wherein PG is a protecting group, and each other variable is independently as described in any one of the preceding Embodiments.

990. The method of any one of Embodiments 892-905 and 980-988, comprising the method of Embodiment 989.

991. A method, comprising:
reacting a compound having the structure of formula D′-3:

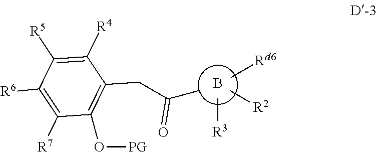

or a salt thereof to provide a compound having the structure of formula D′-4:

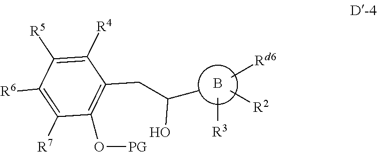

or a salt thereof, wherein each variable is independently as described in any one of the preceding Embodiments.

992. The method of any one of Embodiments 892-905 and 980-990, comprising the method of Embodiment 991.

993. The method of any one of Embodiments 991-992, wherein the reaction is performed in the presence of a reducing agent.

994. A method, comprising:
reacting a compound having the structure of formula D′-1:

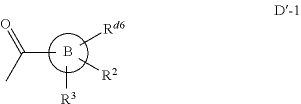

or a salt thereof with a compound having the structure of formula D′-2:

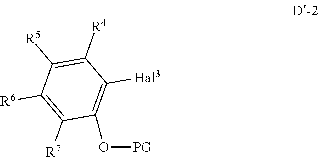

or a salt thereof to provide a compound having the structure of formula D′-3:

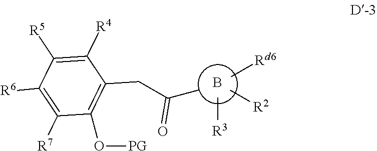

or a salt thereof, wherein each Hal³ is Hal as described herein, and each other variable is independently as described in any one of the preceding Embodiments.

995. The method of any one of Embodiments 892-905 and 980-993, comprising the method of Embodiment 994.

996. The method of any one of Embodiments 994-995, wherein Hal³ is Cl.

997. The method of any one of Embodiments 994-995, wherein Hal³ is Br.

998. The method of any one of Embodiments 994-995, wherein Hal³ is I.

999. The method of any one of Embodiments 994-998, wherein the reaction is performed in the presence of a metal.

1000. The method of Embodiment 999, wherein the reaction is performed in the presence of Pd.

1001. The method of Embodiment 1000, wherein the reaction is performed in the presence of a Pd complex.

1002. The method of Embodiment 1001, wherein the complex is XPhos Pd G3.

1003. The method of any one of Embodiments 994-1002, wherein the reaction is performed in the presence of a base.

1004. The method of Embodiment 1003, wherein the base is Cs₂CO₃.

1005. A method, comprising:
reacting a compound having the structure of formula D′-0:

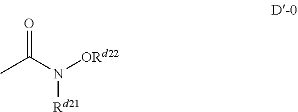

or a salt thereof with a compound having the structure of formula D-3:

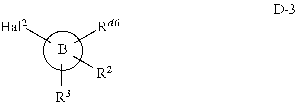

or a salt thereof to provide a compound having the structure of formula D′-1:

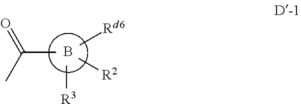

or a salt thereof, wherein each variable is independently as described in any one of the preceding Embodiments.

1006. The method of any one of Embodiments 892-905 and 980-1004, comprising the method of Embodiment 1005.

1007. The method of any one of Embodiments 1005-1006, wherein R^{d21} is optionally substituted C₁₋₆ aliphatic.

1008. The method of any one of Embodiments 1005-1006, wherein R^{d21} is methyl.

1009. The method of any one of Embodiments 1005-1008, wherein R^{d22} is optionally substituted C₁₋₆ aliphatic.

1010. The method of any one of Embodiments 1005-1008, wherein R^{d22} is methyl.

1011. The method of any one of Embodiments 1005-1006, wherein the compound having the structure of formula D′-0 a salt thereof has the structure of

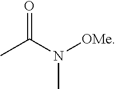

1012. The method of any one of Embodiments 1005-1011, wherein Hal² is Cl.

1013. The method of any one of Embodiments 1005-1011, wherein Hal² is Br.

1014. The method of any one of Embodiments 1005-1011, wherein Hal² is I.

1015. The method of any one of Embodiments 1005-1014, wherein the reaction is performed in the presence of an organometallic agent.

1016. The method of Embodiment 1015, wherein the agent is a Li agent, e.g., n-BuLi.

1017. The method of Embodiment 1015, wherein the agent is a Mg agent, e.g., i-PrMgBr.

1018. The method of any one of Embodiments 1005-1017, wherein the compound having the structure of formula D-3 or a salt thereof is contacted with an organometallic agent, and the resulting agent is contacted with a compound having the structure of formula D′-0 or a salt thereof.

1019. The method of any one of Embodiments 980-1018, wherein R^{d6} is —CH(OR)₂ wherein each R is independently not H.

1020. The method of any one of Embodiments 980-1018, wherein R^{d6} is —CH(OR)₂ wherein each R is independently optionally substituted C₁₋₆ aliphatic.

1021. The method of any one of Embodiments 980-1018, wherein R^{d6} is —CH(OR)₂ wherein the two R are taken together with their intervening atoms to form an optionally substituted 4-10, e.g., 5-10, 5-6, 4, 5, 6, 7, 8, 9, or 10 membered ring having 0-3 heteroatoms in addition to the intervening atoms.

1022. The method of any one of Embodiments 980-1018, wherein R^{d6} is optionally substituted

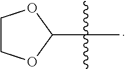 [SMILES: *C1OCCO1]

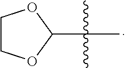 [SMILES: *C1OCCO1]

1023. The method of any one of Embodiments 980-1018, wherein R^{d6} is

1024. The method of any one of Embodiments 980-1023, wherein PG is selected from Bn, MEM, and allyl.

1025. The method of any one of Embodiments 980-1024, wherein the compound having the structure of formula D-3 or a salt thereof has the structure of

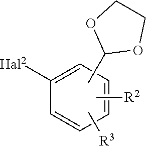

or a salt thereof.

1026. The method of any one of Embodiments 980-1025, wherein the compound having the structure of formula D′-1 or a salt thereof has the structure of

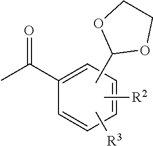

or a salt thereof.

1027. The method of any one of Embodiments 980-1026, wherein the compound having the structure of formula D′-5 or a salt of has the structure of

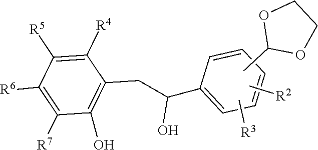

or a salt thereof.

1028. The method of any one of Embodiments 980-1027, wherein the compound having the structure of formula D′-6 or a salt thereof has the structure of

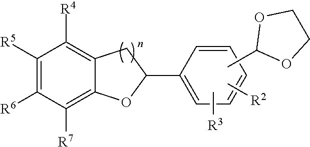

or a salt thereof.

1029. The method of any one of Embodiments 980-1028, wherein the compound having the structure of formula D′-4 or a salt of has the structure of

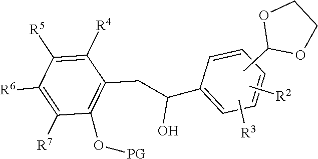

or a salt thereof

1030. The method of any one of Embodiments 980-1029, wherein the compound having the structure of formula D′-5 or a salt of has the structure

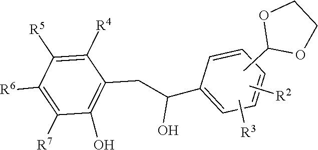

or a salt thereof.

1031. The method of any one of Embodiments 980-1030, wherein the compound having the structure of formula D′-3 or a salt of has the structure of

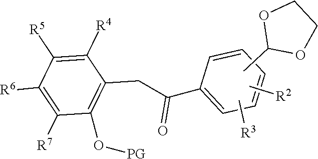

or a salt thereof.

1032. The method of any one of Embodiments 980-1031, wherein the compound having the structure of formula D′-4 or a salt thereof has the structure of

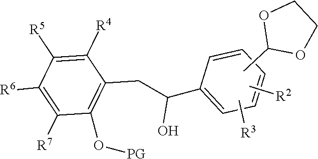

or a salt thereof.

1033. The method of any one of Embodiments 980-1032, wherein the compound having the structure of formula D′-1 or a salt thereof has the structure of

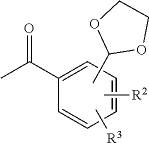

or a salt thereof.

1034. The method of any one of Embodiments 980-1033, wherein the compound having the structure of formula D′-2 or a salt thereof has the structure of

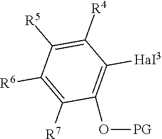

or a salt thereof.

1035. The method of any one of Embodiments 980-1034, wherein the compound having the structure of formula D′-3 or a salt thereof has the structure of

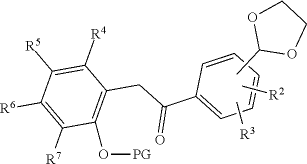

or a salt thereof.

1036. The method of any one of Embodiments 775-1035, comprising separation of two or more stereoisomers.

1037. The method of any one of Embodiments 775-1036, comprising separation of two enantiomers.

1038. A method, comprising:
reacting a compound having the structure of formula E:

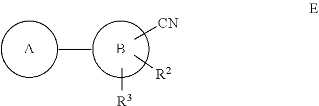

or a salt thereof to provide a compound having the structure of formula I or a salt thereof, wherein each variable is independently as described in any one of the preceding Embodiments.

1039. The method of Embodiment 1038, wherein the compound having the structure of formula E or a salt thereof is a compound of any one of Embodiments 1-678, wherein R¹ is —CN.

1040. The method of Embodiment 1038, wherein the compound having the structure of formula E or a salt thereof has the structure of

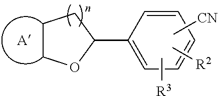

or a salt thereof.

1041. The method of any one of Embodiments 1038-1040, wherein the compound having the structure of formula I or a salt thereof is a compound of any one of Embodiments 1-678, wherein R¹ is

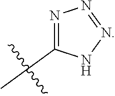 [SMILES: *c1nnn[nH]1]

1042. The method of any one of Embodiments 1038-1040, wherein the compound having the structure of formula I or a salt thereof has the structure of

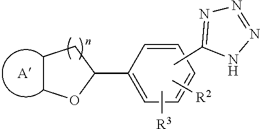

or a salt thereof.

1043. The method of any one of Embodiments 1038-1042, wherein the compound having the structure of formula I or a salt thereof has the structure of

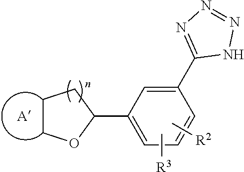

or a salt thereof.

1044. The method of any one of Embodiments 1038-1043, wherein the compound having the structure of formula I or a salt thereof has the structure of

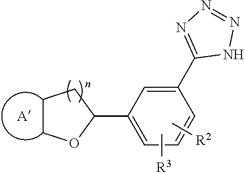

or a salt thereof, wherein Ring A′ is an optionally substituted 10 membered aromatic ring having 0, 1, 2, 3, or 4 heteroatoms.

1045. The method of any one of Embodiments 1038-1044, wherein the compound having the structure of formula I or a salt thereof has the structure of

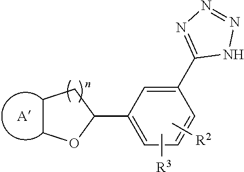

or a salt thereof, wherein Ring A′ is an optionally substituted 10 membered aromatic ring having 0 heteroatoms.

1046. The method of any one of Embodiments 1038-1045, wherein the compound having the structure of formula I or a salt thereof has the structure of

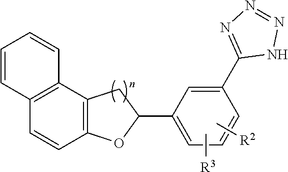

or a salt thereof.

1047. The method of any one of Embodiments 1038-1046, wherein n is 1.

1048. The method of any one of Embodiments 1038-1047, comprising contacting a compound having the structure of formula E or a salt thereof with an azide and an organic tin oxide.

1049. The method of Embodiment 1048, wherein the azide is TMSN₃.

1050. The method of any one of Embodiments 1048-1049, wherein the organic tin oxide has the structure of R²Sn(O).

1051. The method of any one of Embodiments 1048-1049, wherein the organic tin oxide has the structure of R²Sn(O) wherein each R is independently C16 alkyl.

1052. The method of any one of Embodiments 1048-1049, wherein the organic tin oxide is dibutyltin oxide.

1053. The method of any one of Embodiments 775-1037, comprising a method of any one of Embodiments 1038-1052.

1054. A method, comprising:
reacting a compound having the structure of formula I wherein R¹ is —C(O)NH₂ or a salt thereof to provide a compound having the structure of formula I wherein R¹ is —CN or a salt thereof, wherein each variable is independently as described in any one of the preceding Embodiments.

1055. The method of Embodiment 1054, wherein the compound having the structure of formula I wherein R¹ is —C(O)NH₂ or a salt thereof is a compound of any one of Embodiments 1-678, wherein R¹ is —C(O)NH₂.

1056. The method of any one of Embodiments 1054-1055, wherein the compound having the structure of formula I wherein R¹ is —CN or a salt thereof is a compound of any one of Embodiments 1-678, wherein R¹ is —CN.

1057. The method of any one of Embodiments 1054-1056, comprising contacting a compound having the structure of formula I wherein R¹ is —C(O)NH₂ or a salt thereof with TFAA and a base.

1058. The method of Embodiment 1057, wherein the base is Et₃N.

1059. The method of any one of Embodiments 775-1053, comprising a method of any one of Embodiments 1054-1058.

1060. A method, comprising:
reacting a compound having the structure of formula I wherein R¹ is —C(O)OH or a salt thereof to provide a compound having the structure of formula I wherein R¹ is —C(O)NH₂ or a salt thereof, wherein each variable is independently as described in any one of the preceding Embodiments.

1061. The method of Embodiment 1060, wherein the compound having the structure of formula I wherein R¹ is —C(O)OH or a salt thereof is a compound of any one of Embodiments 1-678, wherein R¹ is —C(O)OH.

1062. The method of any one of Embodiments 1060-1061, wherein the compound having the structure of formula I wherein R¹ is —C(O)NH₂ or a salt thereof is a compound of any one of Embodiments 1-678, wherein R¹ is —C(O)NH₂.

1063. The method of any one of Embodiments 1060-1062, wherein the reacting is performed under an amidation condition.

1064. The method of any one of Embodiments 1060-1063, comprising activating-C(O)OH or a salt form thereof of the compound having the structure of formula I wherein R¹ is —C(O)OH or a salt thereof.

1065. The method of any one of Embodiments 1060-1064, comprising contacting the compound having the structure of formula I wherein R¹ is —C(O)OH or a salt thereof with SOCl₂.

1066. The method of any one of Embodiments 1060-1064, comprising contacting the compound having the structure of formula I wherein R¹ is —C(O)OH or a salt thereof or an activated form thereof with NH₃.

1067. The method of any one of Embodiments 775-1053, comprising a method of any one of Embodiments 1060-1066.

1068. A method, comprising:
reacting a compound having the structure of formula E-6:

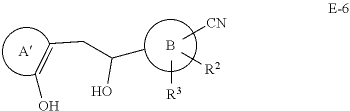

or a salt thereof to provide a compound having the structure of formula E-7:

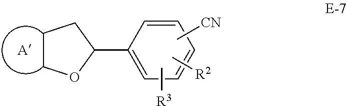

or a salt thereof, wherein each variable is independently as described in any one of the preceding Embodiments.

1069. The method of any one of Embodiments 775-1067, wherein n is 1, and the method comprises the method of Embodiment 1068.

1070. The method of any one of Embodiments 1068-1069, wherein the reaction is performed under a Mitsunobu condition.

1071. The method of any one of Embodiments 1068-1070, wherein the reaction is performed in the presence of a phosphine compound.

1072. The method of Embodiment 1071, wherein the phosphine compound has the structure of P(R)₃ wherein each R is independently not —H.

1073. The method of Embodiment 1071, wherein the phosphine compound has the structure of PPh₃.

1074. The method of any one of Embodiments 1068-1073, wherein the reaction is performed in the presence of an azodicarboxylate compound.

1075. The method of Embodiment 1074, wherein the azodicarboxylate compound has the structure of R^{a1}O₂C—N═N—CO₂R^{a2} or a salt thereof, wherein each of R^{a1} and R^{a2} is independently R.

1076. The method of Embodiment 1074, wherein the azodicarboxylate compound is DEAD or DIAD.

1077. A method, comprising:
reacting a compound having the structure of formula E-5:

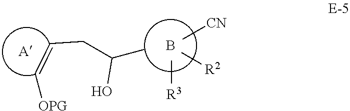

or a salt thereof to provide a compound having the structure of formula E-6:

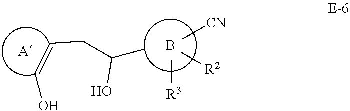

or a salt thereof, wherein PG is a protecting group, and each other variable is independently as described in any one of the preceding Embodiments.

1078. The method of any one of Embodiments 775-1076, comprising the method of Embodiment 1077.

1079. A method, comprising:
reacting a compound having the structure of formula E-4:

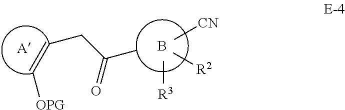

or a salt thereof to provide a compound having the structure of formula E-5:

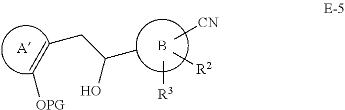

or a salt thereof, wherein each variable is independently as described in any one of the preceding Embodiments.

1080. The method of any one of Embodiments 775-1078, comprising the method of Embodiment 1079.

1081. The method of any one of Embodiments 1079-1080, wherein the reaction is performed in the presence of a reducing agent.

1082. A method, comprising:
reacting a compound having the structure of formula E-2:

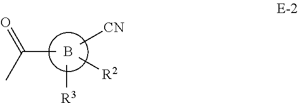

or a salt thereof with a compound having the structure of formula E-3:

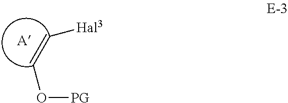

or a salt thereof to provide a compound having the structure of formula E-4:

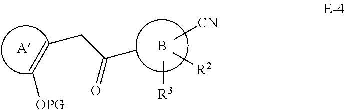

or a salt thereof, wherein each Hal³ is Hal as described herein, and each other variable is independently as described in any one of the preceding Embodiments.

1083. The method of any one of Embodiments 775-1081, comprising the method of Embodiment 1082.

1084. The method of any one of Embodiments 1082-1083, wherein Hal³ is Cl.

1085. The method of any one of Embodiments 1082-1083, wherein Hal³ is Br.

1086. The method of any one of Embodiments 1082-1083, wherein Hal³ is I.

1087. The method of any one of Embodiments 1082-1086, wherein the reaction is performed in the presence of a metal.

1088. The method of Embodiment 1087, wherein the reaction is performed in the presence of Pd.

1089. The method of Embodiment 1088, wherein the reaction is performed in the presence of a Pd complex.

1090. The method of Embodiment 1089, wherein the complex is XPhos Pd G3.

1091. The method of any one of Embodiments 1082-1090, wherein the reaction is performed in the presence of a base.

1092. The method of Embodiment 1091, wherein the base is Cs₂CO₃.

1093. A method, comprising:
reacting a compound having the structure of formula E-0:

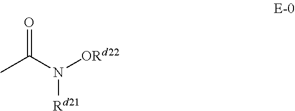

or a salt thereof with a compound having the structure of formula E-1:

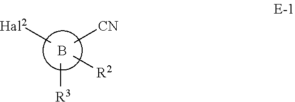

or a salt thereof to provide a compound having the structure of formula E-2:

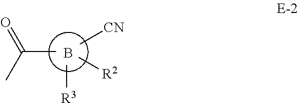

or a salt thereof, wherein each variable is independently as described in any one of the preceding Embodiments.

1094. The method of any one of Embodiments 775-1092, comprising the method of Embodiment 1093.

1095. The method of any one of Embodiments 1093-1094, wherein R^{d21} is optionally substituted C₁₋₆ aliphatic.

1096. The method of any one of Embodiments 1093-1094, wherein R^{d21} is methyl.

1097. The method of any one of Embodiments 1093-1094, wherein R^{d22} is optionally substituted C₁₋₆ aliphatic.

1098. The method of any one of Embodiments 1093-1094, wherein R^{d22} is methyl.

1099. The method of any one of Embodiments 1093-1094, wherein the compound having the structure of formula D′-0 a salt thereof has the structure of

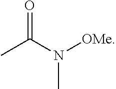

1100. The method of any one of Embodiments 1093-1099, wherein Hal² is Cl.

1101. The method of any one of Embodiments 1093-1099, wherein Hal² is Br.

1102. The method of any one of Embodiments 1093-1099, wherein Hal² is I.

1103. The method of any one of Embodiments 1093-1102, wherein the reaction is performed in the presence of an organometallic agent.

1104. The method of Embodiment 1103, wherein the agent is a Li agent, e.g., n-BuLi.

1105. The method of Embodiment 1103, wherein the agent is a Mg agent, e.g., i-PrMgBr.

1106. The method of any one of Embodiments 1093-1105, wherein the compound having the structure of formula E-1 or a salt thereof is contacted with an organometallic agent, and the resulting agent is contacted with a compound having the structure of formula E-0 or a salt thereof.

1107. A method, comprising:
reacting a compound having the structure of formula F-9:

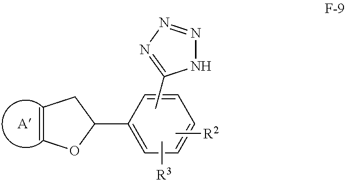

or a salt thereof with a base for form a salt of a compound of formula F-9.

1108. A method, comprising:
reacting a compound having the structure of formula F-9′:

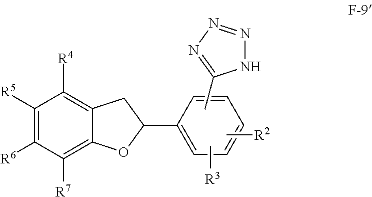

or a salt thereof with a base for form a salt of a compound of formula F-9.

1109. A method, comprising:
reacting a compound having the structure of formula F-9:

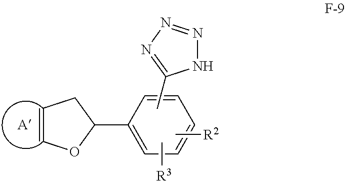

or a salt thereof with a base for form a salt.
to provide a compound having the structure of formula F:

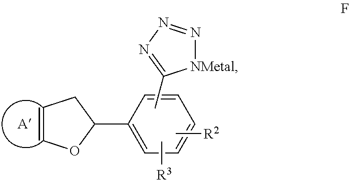

wherein metal is a metal cation, and each variable is independently as described in any one of the preceding Embodiments.

1110. A method, comprising:
reacting a compound having the structure of formula F-9′:

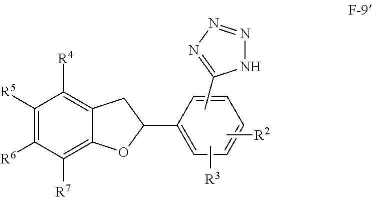

or a salt thereof with a base for form a salt.
to provide a compound having the structure of formula F′:

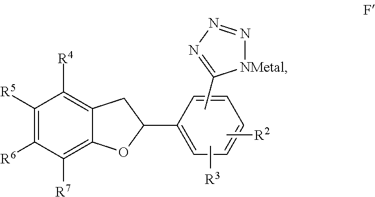

wherein metal is a metal cation, and each variable is independently as described in any one of the preceding Embodiments.

1111. The method of any one of Embodiments 775-1106, comprising a method of any one of Embodiments 1107-1110.

1112. The method of any one of Embodiments 1107-1111, wherein the compound having the structure of F-9 or F-9′ or a salt thereof is a compound having the structure of

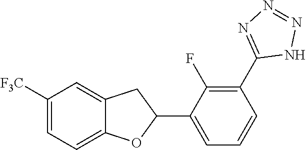 [SMILES: Fc1c(-c2nnn[nH]2)cccc1C1Cc2cc(C(F)(F)F)ccc2O1]

or a salt thereof.

1113. The method of any one of Embodiments 1107-1111, wherein the compound having the structure of F-9 or F-9′ or a salt thereof is a compound having the structure of

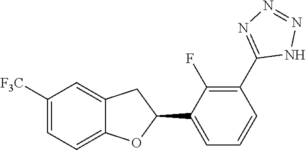 [SMILES: Fc1c(-c2nnn[nH]2)cccc1[C@@H]1Cc2cc(C(F)(F)F)ccc2O1]

or a salt thereof.

1114. The method of any one of Embodiments 1107-1113, wherein the compound having the structure of F or F′ or a salt thereof is a compound having the structure of

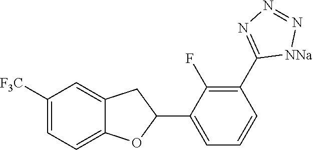

or a salt thereof.

1115. The method of any one of Embodiments 1107-1113, wherein the compound having the structure of F or F′ or a salt thereof is a compound having the structure of

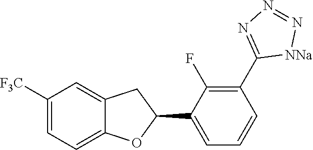

or a salt thereof.

1116. The method of any one of Embodiments 1107-1115, wherein the reaction is performed in the presence of a base.

1117. The method of any one of Embodiments 1107-1115, wherein the reaction is performed in the presence of NaOH.

1118. A method, comprising:
reacting a compound having the structure of formula F-8:

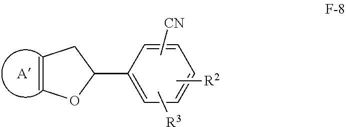

or a salt thereof
to provide a compound having the structure of formula F-9:

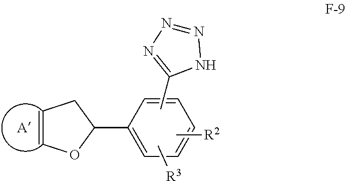

or a salt thereof, wherein each variable is independently as described in any one of the preceding Embodiments.

1119. A method, comprising:
reacting a compound having the structure of formula F-8′:

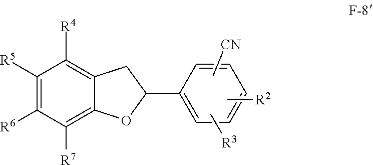

or a salt thereof
to provide a compound having the structure of formula F-9:

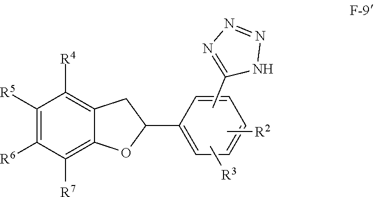

or a salt thereof, wherein each variable is independently as described in any one of the preceding Embodiments.

1120. The method of any one of Embodiments 1118-1119, wherein a compound having the structure of formula F-8 or F-8′ is

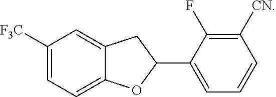 [SMILES: N#Cc1cccc(C2Cc3cc(C(F)(F)F)ccc3O2)c1F]

1121. The method of any one of Embodiments 1118-1119, wherein a compound having the structure of formula F-8 or F-8′ is

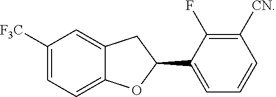 [SMILES: N#Cc1cccc([C@@H]2Cc3cc(C(F)(F)F)ccc3O2)c1F]

1122. The method of any one of Embodiments 1107-1117, comprising a method of any one of Embodiments 1118-1121.

1123. The method of any one of Embodiments 1119-1122, wherein the reaction is performed in the presence of an azide reagent.

1124. The method of any one of Embodiments 1119-1122, wherein the reaction is performed in the presence of an TMSN₃.

1125. The method of any one of Embodiments 1119-1124, wherein the reaction is performed in the presence of Bu₂SnO

1126. The method of any one of Embodiments 1119-1122, wherein the reaction is performed in the presence of an NaN₃.

1127. A method, comprising:
reacting a compound having the structure of formula F-7:

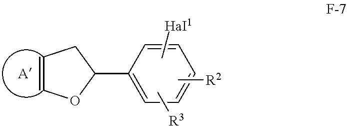

or a salt thereof
to provide a compound having the structure of formula F-8:

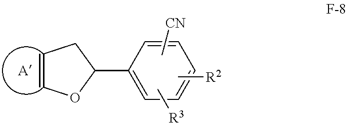

or a salt thereof, wherein each variable is independently as described in any one of the preceding Embodiments.

1128. A method, comprising:
reacting a compound having the structure of formula F-7′:

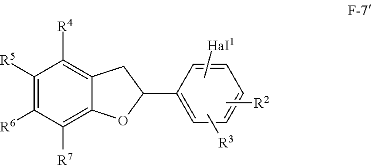

or a salt thereof
to provide a compound having the structure of formula F-8′:

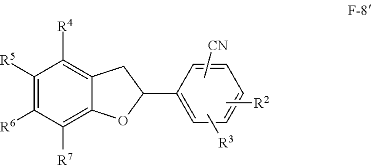

or a salt thereof, wherein each variable is independently as described in any one of the preceding Embodiments.

1129. The method of any one of Embodiments 1127-1128, wherein a compound having the structure of formula F-7 or F-7′ is

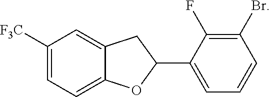 [SMILES: Fc1c(Br)cccc1C1Cc2cc(C(F)(F)F)ccc2O1]

1130. The method of any one of Embodiments 1127-1128, wherein a compound having the structure of formula F-7 or F-7′ is

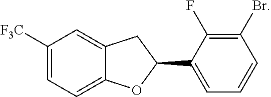 [SMILES: Fc1c(Br)cccc1[C@@H]1Cc2cc(C(F)(F)F)ccc2O1]

1131. The method of any one of Embodiments 1107-1126, comprising a method of any one of Embodiment 1127-1130.

1132. The method of any one of Embodiments 1128-1131, wherein Hall is Br.

1133. The method of any one of Embodiments 1128-1132, wherein the reaction is performed in the presence of a cyanide reagent.

1134. The method of any one of Embodiments 1128-1132, wherein the reaction is performed in the presence of CuCN.

1135. A method, comprising:
reacting a compound having the structure of formula F-6:

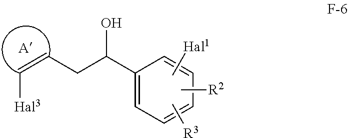

or a salt thereof
to provide a compound having the structure of formula F-7:

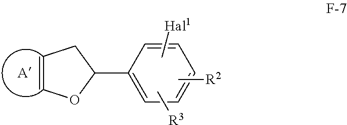

or a salt thereof, wherein each variable is independently as described in any one of the preceding Embodiments.

1136. A method, comprising:
reacting a compound having the structure of formula F-6′:

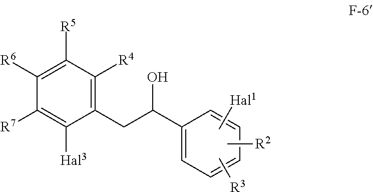

or a salt thereof
to provide a compound having the structure of formula F-7′:

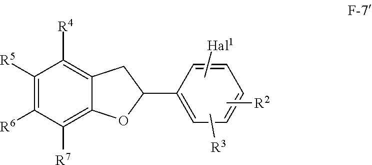

or a salt thereof, wherein each variable is independently as described in any one of the preceding Embodiments.

1137. The method of any one of Embodiments 1135-1136, wherein a compound having the structure of formula F-6 or F-6′ is

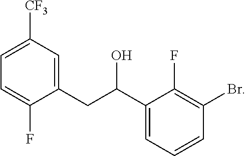 [SMILES: OC(Cc1cc(C(F)(F)F)ccc1F)c1cccc(Br)c1F]

1138. The method of any one of Embodiments 1135-1136, wherein a compound having the structure of formula F-6 or F-6′ is

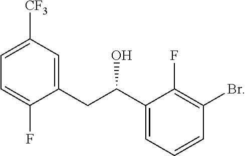 [SMILES: O[C@@H](Cc1cc(C(F)(F)F)ccc1F)c1cccc(Br)c1F]

1139. The method of any one of Embodiments 1107-1134, comprising a method of any one of Embodiments 1135-1138.

1140. The method of any one of Embodiments 1135-1139, wherein Hal³ is F

1141. The method of any one of Embodiments 1136-1140, wherein the reaction is performed in the presence a base.

1142. The method of any one of Embodiments 1136-1140, wherein the reaction is performed in the presence a metal alkoxide.

1143. The method of any one of Embodiments 1136-1140, wherein the reaction is performed in the presence tBuOK.

1144. A method, comprising:
reacting a compound having the structure of formula F-5:

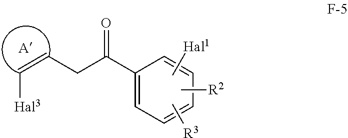

or a salt thereof
to provide a compound having the structure of formula F-6:

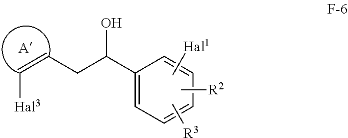

or a salt thereof, wherein each variable is independently as described in any one of the preceding Embodiments.

1145. A method, comprising:
reacting a compound having the structure of formula F-5′:

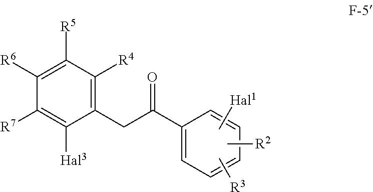

or a salt thereof
to provide a compound having the structure of formula F-6′:

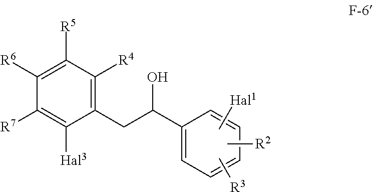

or a salt thereof, wherein each variable is independently as described in any one of the preceding Embodiments.

1146. The method of any one of Embodiments 1144-1145, wherein a compound having the structure of formula F-5 or F-5′ is

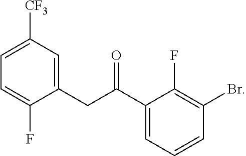 [SMILES: O=C(Cc1cc(C(F)(F)F)ccc1F)c1cccc(Br)c1F]

1147. The method for preparing a compound of any one of Embodiments 1107-1143, comprising a method of any one of Embodiments 1144-1146.

1148. The method of any one of Embodiments 1145-1147, wherein the reaction is performed under a hydrogenation condition.

1149. The method of any one of Embodiments 1145-1148, wherein the reaction is performed in the presence of a metal catalyst.

1150. The method of any one of Embodiments 1145-1149, wherein the reaction is performed stereoselectively.

1151. The method of any one of Embodiments 1145-1150, wherein the reaction is performed in the presence of a chiral metal complex.

1152. The method of any one of Embodiments 1145-1151, wherein the reaction is performed in the presence of a metal catalyst and HCOOH.

1153. The method of any one of Embodiments 1149-1152, wherein the metal catalyst is a transition metal catalyst.

1154. The method of any one of Embodiments 1149-1154, wherein the metal catalyst is a Ru catalyst.

1155. The method of any one of Embodiments 1149-1155, wherein the metal catalyst is or comprises RuCl (p-cymene) [(S,S)-Ts-DPEN].

1156. The method of any one of Embodiments 1145-1155, wherein the reaction is performed in the presence of a Ru catalyst and HCOOH.

1157. The method of any one of Embodiments 1145-1156, wherein the reaction is performed in the presence of RuCl (p-cymene) [(S,S)-Ts-DPEN] and HCOOH.

1158. A method, comprising:
reacting a compound having the structure of formula F-2:

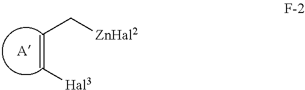

or a salt thereof
and a compound having a structure of formula F-4:

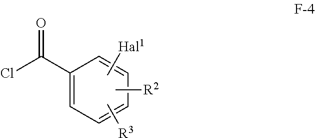

or a salt thereof
to provide a compound having the structure of formula F-5:

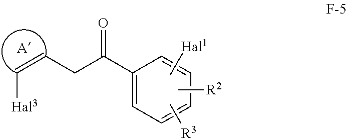

or a salt thereof, wherein variable is independently as described in any one of the preceding Embodiments.

1159. A method, comprising:
reacting a compound having the structure of formula F-2′:

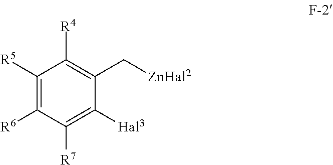

or a salt thereof
and a compound having a structure of formula F-4:

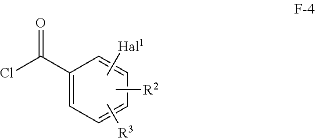

or a salt thereof
to provide a compound having the structure of formula F-5′:

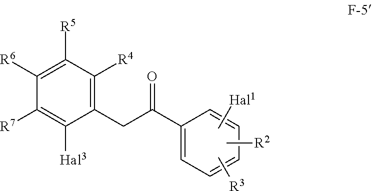

or a salt thereof, wherein variable is independently as described in any one of the preceding Embodiments.

1160. The method of any one of Embodiments 1158-1159, wherein a compound having the structure of formula F-2 or F-2′ is

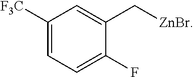

1161. The method of any one of Embodiments 1158-1159, wherein a compound having the structure of formula F-2 or F-2′ is

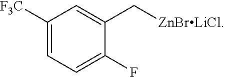

1162. The method of any one of Embodiments 1158-1159, wherein a compound having the structure of formula F-4 is

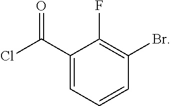 [SMILES: O=C(Cl)c1cccc(Br)c1F]

1163. The method for preparing a compound of any one of Embodiments 1107-1157, comprising a method of any one of Embodiments 1158-1162.

1164. The method of any one of Embodiments 1158-1163, wherein the reaction is performed in the presence of a transition metal catalyst.

1165. The method of any one of Embodiments 1158-1163, wherein the reaction is performed in the presence of CuCN.

1166. A method, comprising:
reacting a compound having the structure of formula F-1:

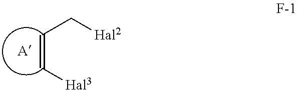

or a salt thereof
to provide a compound having the structure of formula F-2:

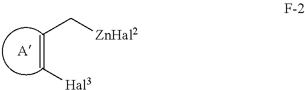

or a salt thereof, wherein each variable is independently as described in any one of the preceding Embodiments.

1167. A method, comprising:
reacting a compound having the structure of formula F-1′:

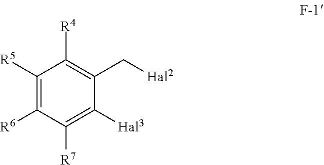

or a salt thereof
to provide a compound having the structure of formula F-2′:

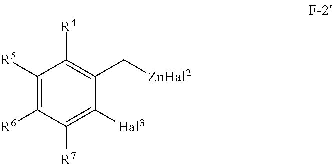

or a salt thereof, wherein each variable is independently as described in any one of the preceding Embodiments.

1168. The method of any one of Embodiments 1166-1167, wherein a compound having the structure of formula F-1 or F-1′ is

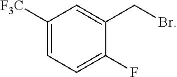 [SMILES: Fc1ccc(C(F)(F)F)cc1CBr]

1169. The method for preparing a compound of any one of Embodiments 1107-1165, comprising a method of any one of Embodiments 1166-1168.

1170. The method of any one of Embodiments 1167-1169, wherein Hal² is Br.

1171. The method of any one of Embodiments 1167-1170, wherein the reaction is performed in the presence of a zinc reagent.

1172. The method of any one of Embodiments 1167-1170, wherein the reaction is performed in the presence of Zn.

1173. A method, comprising:
reacting a compound having the structure of formula F-3:

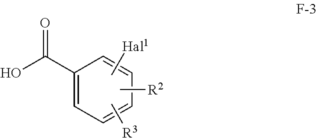

or a salt thereof
to provide a compound having the structure of formula F-4:

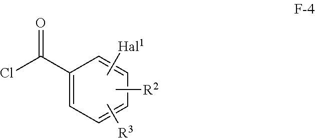

or a salt thereof, wherein each variable is independently as described in any one of the preceding Embodiments.

1174. The method of Embodiment 1173, wherein a compound having the structure of formula F-3 is

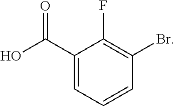 [SMILES: O=C(O)c1cccc(Br)c1F]

1175. The method for preparing a compound of any one of Embodiments 1107-1172, comprising a method of any one of Embodiments 1173-1174.

1176. The method of any one of Embodiments 1173-1175, wherein the reaction is performed in the presence of SOCl₂.

1177. A method for preparing a compound of any one of Embodiments 1-678, comprising a method of any one of Embodiments 775-1176.

1178. A compound, composition, or method described in the specification.

### EXEMPLIFICATION

Certain examples of provided technologies (e.g., compounds, compositions, methods (methods of preparation, use, assessment, etc.), etc.) are described herein. Those skilled in the art reading the present disclosure appreciate that various technologies, including those described below and modifications, variants and derivatives thereof, are available for manufacturing, characterizing and/or assessing provided technologies in accordance with the present disclosure.

### Examples 1-25

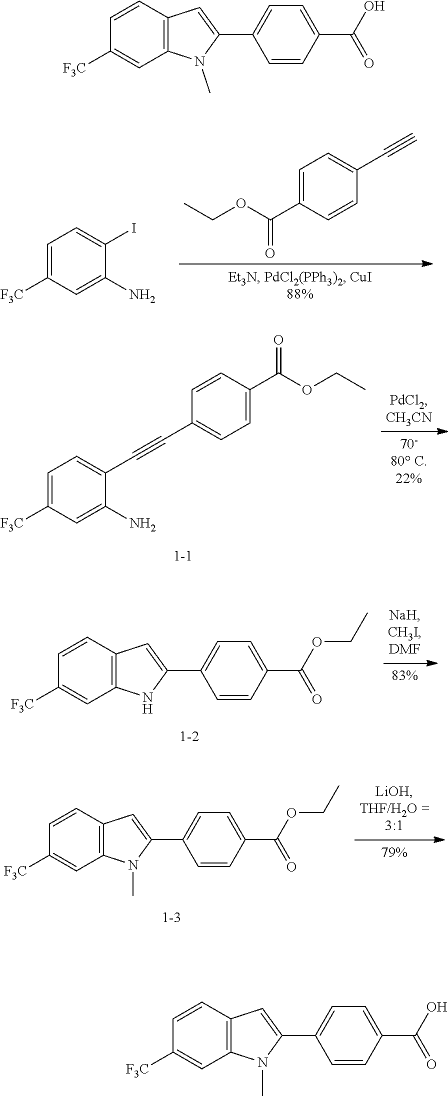

Step 1:2-Iodo-5-(trifluoromethyl) aniline (50.0 mg, 0.174 mmol), PdCl₂ (PPh₃)₂ (2.4 mg, 0.0035 mmol), CuI (1.3 mg, 0.007 mmol) were dissolved in Et₃N(3 mL). The solution was stirred under argon atmosphere at room temperature for 30 minutes. Ethyl 4-ethynylbenzoate (36.0 mg, 0.21 mmol) were added to the above solution at room temperature. The mixture was stirred under argon atmosphere at room temperature overnight. The reaction mixture was quenched by NH₄Cl solution and extracted with EtOAc. The organic phase was dried over anhydrous Na₂SO₄, and the filtrate was evaporated in vacuum. The crude product was purified by silica gel flash chromatography to afford compound 1-1 (51.1 mg, 88% yield) as yellow solid. LC-MS, ES⁻(m/z): 334.10 (M+1). ¹H NMR (400 MHZ, Chloroform-d) δ 8.03 (d, J=8.6 Hz, 2H), 7.58 (d, J=8.6 Hz, 2H), 7.43 (s, 1H), 6.94 (d, J=7.0 Hz, 2H), 4.38 (q, J=7.1 Hz, 4H), 1.40 (t, J=7.1 Hz, 3H).

Step 2: To a solution of 1-1 (48.6 mg, 0.146 mmol) in MeCN(1.0 mL) was added PdCl₂ (2.6 mg, 0.0146 mmol), and the mixture was stirred at 80° C. overnight. TLC showed complete consumption of the starting material. The reaction mixture was concentrated in vacuum and purified by prep-TLC to give the desired product 1-2 (11.0 mg, 22% yield) as yellow solid. LC-MS, ES⁻ (m/z): 334.10 (M+1). ¹H NMR (400 MHZ, Chloroform-d) δ 8.65 (s, 1H), 8.13 (d, J=8.6 Hz, 2H), 7.79-7.65 (m, 4H), 7.41-7.33 (m, 1H), 6.98 (s, 1H), 4.40 (q, J=7.1 Hz, 2H), 1.42 (t, J=7.1 Hz, 3H).

Step 3: To a solution of ethyl 4-(6-(trifluoromethyl)-1H-indol-2-yl)benzoate (11.0 mg, 0.033 mmol) in DMF (1.0 mL) was added NaH (3.3 mg, 0.082 mmol), and the mixture was stirred for 30 minutes at 0° C. CH₃I (9.4 mg, 0.066 mmol) was added to the above solution at 0° C. The mixture was stirred for 1 h at 0° C. TLC showed complete consumption of the starting material. The reaction mixture was quenched by NH₄Cl solution and extracted with EtOAc. The organic phase was dried over anhydrous Na₂SO₄, and the filtrate was evaporated in vacuum to obtain 1-3 (9.6 mg, 83% yield) as yellow solid. LC-MS, ES⁻ (m/z): 348.11 (M+1).

Step 4: To a solution of ethyl 4-(1-methyl-6-(trifluoromethyl)-1H-indol-2-yl)benzoate (9.6 mg, 0.028 mmol) in THF (0.9 mL) and H₂O(0.3 mL) was added LiOH (3.3 mg, 0.138 mmol), and the mixture was stirred for 1 h at room temperature. TLC showed complete consumption of the starting material. The reaction mixture was acidified with 1M HCl and extracted with EtOAc for three times. The combined organic layers were dried over anhydrous Na₂SO₄, filtered, and concentrated in vacuum to give Example 1 (7.0 mg, 79% yield) as white solid. LC-MS, ES⁻ (m/z): 318.08 (M−1). ¹H NMR (400 MHZ, DMSO-d₆) δ 13.09 (s, 1H), 8.05 (d, J=8.2 Hz, 2H), 7.93 (s, 1H), 7.75 (t, J=8.1 Hz, 3H), 7.34 (d, J=8.4 Hz, 1H), 6.79 (s, 1H), 3.83 (s, 3H).

The following examples were prepared employing the same protocol as described in Example 1.

| Example | Structure | NMR | MS |
|---|---|---|---|
| 2 | 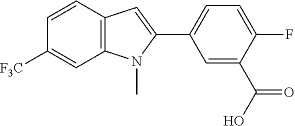 [SMILES: Cn1c(-c2ccc(F)c(C(=O)O)c2)cc2ccc(C(F)(F)F)cc21] | ¹H NMR (400 MHz, CDCl₃) δ 8.13 (s, 1H), 7.71 (s, 1H), 7.66 (s, 1H), 7.38 (s, 2H), 7.32 (s, 1H), 6.72 (s, 1H), 3.84 (s, 3H). | ES⁻ (m/z): 336.01 (M − 1) |
| 3 | 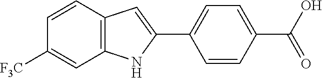 [SMILES: O=C(O)c1ccc(-c2cc3ccc(C(F)(F)F)cc3[nH]2)cc1] | ¹H NMR (400 MHz, DMSO-d₆) δ 12.16 (s, 1H), 8.06-7.95 (m, 4H), 7.74 (d, J = 8.5 Hz, 1H), 7.67 (s, 1H), 7.28 (d, J = 8.8 Hz, 1H), 7.17 (s, 1H). | ES⁻ (m/z): 304.07 (M − 1) |
| 4 | 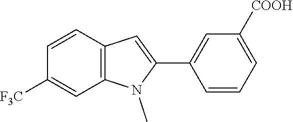 [SMILES: Cn1c(-c2cccc(C(=O)O)c2)cc2ccc(C(F)(F)F)cc21] | 'H NMR (400 MHz, DMSO-d₆) δ 8.09 (t, J = 1.6 Hz, 1 H), 8.01 (dt, J = 7.6, 1.2 Hz, 1 H), 7.91 (s, 1 H), 7.86 (dt, J = 7.6, 1.2 Hz, 1 H), 7.75 (d, J = 8.4 Hz, 1 H), 7.65 (t, J = 7.6 Hz, 1 H), 7.33 (d, J = 8.4 Hz, 1 H), 6.76 (s, 1 H), 3.80 (s, 3 H). | ES⁻ (m/z): 317.89 (M − 1) |
| 5 | 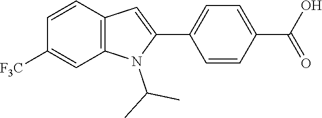 [SMILES: CC(C)n1c(-c2ccc(C(=O)O)cc2)cc2ccc(C(F)(F)F)cc21] | ¹H NMR (400 MHz, Methanol-d₄) δ 8.08 (d, J = 8.1 Hz, 2H), 7.89 (s, 1H), 7.69 (d, J = 8.3 Hz, 1H), 7.52 (d, J = 8.1 Hz, 2H), 7.29 (d, J = 8.3 Hz, 1H), 6.55 (s, 1H), 4.74 (p, J = 7.0 Hz, 1H), 1.60 (d, J = 7.0 Hz, 6H). | ES⁻ (m/z): 345.84 (M − 1) |
| 6 | 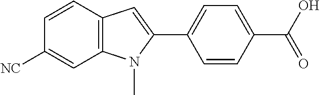 [SMILES: Cn1c(-c2ccc(C(=O)O)cc2)cc2ccc(C#N)cc21] | ¹H NMR (400 MHz, DMSO-d₆) δ 8.15 (s, 1H), 8.04 (d, J = 8.3 Hz, 2H), 7.73 (d, J = 8.2 Hz, 3H), 7.39 (d, J = 9.6 Hz, 1H), 6.81 (s, 1H), 3.81 (s, 3H). | ES⁻ (m/z): 274.85 (M − 1) |
| 7 | 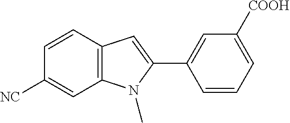 [SMILES: Cn1c(-c2cccc(C(=O)O)c2)cc2ccc(C#N)cc21] | ¹H NMR (400 MHz, DMSO-d₆) δ 8.13 (s, 1H), 8.08 (s, 1H), 8.02 (d, J = 9.0 Hz, 1H), 7.86 (d, J = 9.3 Hz, 1H), 7.77-7.59 (m, 2H), 7.38 (d, J = 9.5 Hz, 1H), 6.78 (s, 1H), 3.78 (s, 3H). | ES⁻ (m/z): 274.86 (M − 1) |
| 8 | 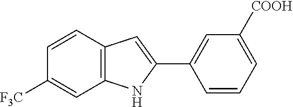 [SMILES: O=C(O)c1cccc(-c2cc3ccc(C(F)(F)F)cc3[nH]2)c1] | ¹H NMR (400 MHz, DMSO-d₆) δ 12.19 (s, 1 H), 8.43 (s, 1 H), 8.13 (d, J = 9.6 Hz, 1 H), 7.90 (d, J = 7.6 Hz, 1 H), 7.72 (d, J = 8.4 Hz, 1 H), 7.66 (s, 1 H), 7.60 (t, J = 7.6 Hz, 1 H), 7.27 (d, J = 8.4 Hz, 1 H), 7.11 (s, 1 H). | ES⁻ (m/z): 303.87 (M − 1) |
| 9 | 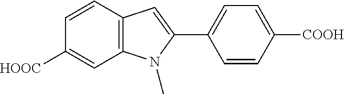 [SMILES: Cn1c(-c2ccc(C(=O)O)cc2)cc2ccc(C(=O)O)cc21] | ¹H NMR (400 MHz, DMSO-d₆) δ 8.11 (s, 1H), 8.04 (d, J = 8.4 Hz, 1H), 7.73 (d, J = 8.4 Hz, 1H), 7.68-7.61 (m, 1H), 6.75 (s, 1H), 3.81 (s, 1H). | ES⁻ (m/z): 293.86 (M − 1) |
| 10 | 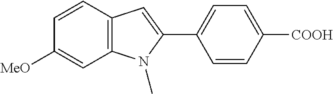 [SMILES: COc1ccc2cc(-c3ccc(C(=O)O)cc3)n(C)c2c1] | ¹H NMR (400 MHz, DMSO-d₆) δ 7.99 (d, J = 8.4 Hz, 2 H), 7.66 (d, J = 8.4 Hz, 2 H), 7.42 (d, J = 8.8 Hz, 1 H), 7.02 (s, 1 H), 6.69 (dd, J = 8.8, 2.4 Hz, 1 H), 6.58 (s, 1 H), 3.79 (s, 3 H), 3.72 (s, 3 H). | ES⁻ (m/z): 279.90 (M − 1) |
| 11 | 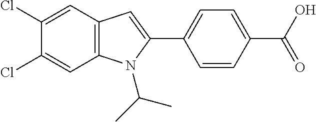 [SMILES: CC(C)n1c(-c2ccc(C(=O)O)cc2)cc2cc(Cl)c(Cl)cc21] | ¹H NMR (400 MHz, Methanol-d₄) δ 8.11-7.99 (m, 2H), 7.90-7.83 (m, 2H), 7.69 (s, 1H), 7.53 (s, 1H), 6.94 (s, 1H), 5.25-5.17 (m, 1H), 1.38 (s, 3H), 1.36 (s, 3H). | ES⁻ (m/z): 345.81 (M + 1) |
| 12 | 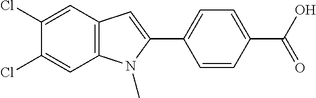 [SMILES: Cn1c(-c2ccc(C(=O)O)cc2)cc2cc(Cl)c(Cl)cc21] | ¹H NMR (400 MHz, Methanol-d₄) δ 8.30 (s, 1H), 8.20 (s, 2H), 8.11 (s, 1H), 7.64 (s, 2H), 6.58 (s, 1H), 3.94 (s, 3H). | ES⁻ (m/z): 317.79 (M − 1) |
| 13 | 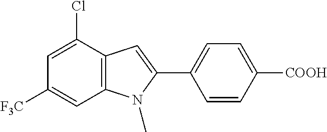 [SMILES: Cn1c(-c2ccc(C(=O)O)cc2)cc2c(Cl)cc(C(F)(F)F)cc21] | ¹H NMR (400 MHz, DMSO-d₆) δ 8.06 (d, J = 8.4 Hz, 2H), 8.01 (s, 1H), 7.78 (d, J = 8.4 Hz, 2H), 7.46 (s, 1H), 6.82 (s, 1H), 3.86 (s, 3H). | ES⁻ (m/z): 351.84 (M − 1) |
| 14 | 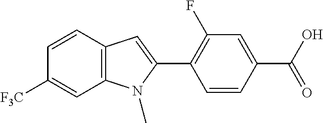 [SMILES: Cn1c(-c2ccc(C(=O)O)cc2F)cc2ccc(C(F)(F)F)cc21] | ¹H NMR (400 MHz, Methanol-d₄) δ 7.96-7.66 (m, 4H), 7.54 (t, J = 6.6 Hz, 1H), 7.32 (d, J = 8.2 Hz, 1H), 6.75-6.58 (m, 1H), 3.71 (s, 3H). | ES⁻ (m/z): 335.82 (M + 1) |
| 15 | 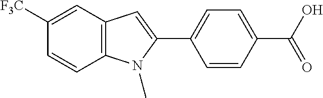 [SMILES: Cn1c(-c2ccc(C(=O)O)cc2)cc2cc(C(F)(F)F)ccc21] | ¹H NMR (400 MHz, DMSO-d₆) δ 8.13-7.91 (m, 3H), 7.72 (t, J = 8.3 Hz, 3H), 7.47 (d, J = 8.6 Hz, 1H), 6.83 (s, 1H), 3.81 (s, 3H). | ES⁻ (m/z): 317.84 (M − 1) |
| 16 | 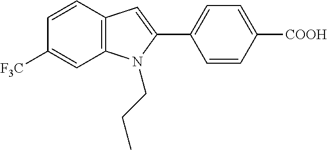 [SMILES: CCCn1c(-c2ccc(C(=O)O)cc2)cc2ccc(C(F)(F)F)cc21] | ¹H NMR (400 MHz, DMSO-d₆) δ 12.85 (s, 1 H), 8.06 (d, J = 8.0 Hz, 2 H), 7.94 (s, 1 H), 7.75 (d, J = 8.4 Hz, 1 H), 7.68 (d, J = 8.4 Hz, 2 H), 7.33 (d, J = 8.4 Hz, 1 H), 6.73 (s, 1 H), 4.28 (t, J = 7.2 Hz, 2 H), 1.57-1.45 (m, 1 H), 0.61 (t, J = 7.6 Hz, 3 H). | ES⁻ (m/z): 345.87 (M − 1) |
| 17 | 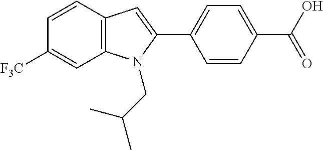 [SMILES: CC(C)Cn1c(-c2ccc(C(=O)O)cc2)cc2ccc(C(F)(F)F)cc21] | ¹H NMR (400 MHz, Methanol-d₄) δ 8.13 (d, J = 8.2 Hz, 2H), 7.77 (s, 1H), 7.71 (d, J = 8.3 Hz, 1H), 7.64 (d, J = 8.2 Hz, 2H), 7.31 (d, J = 9.6 Hz, 1H), 6.67 (s, 1H), 4.18 (d, J = 7.6 Hz, 2H), 1.90-1.78 (m, 1H), 0.59 (d, J = 6.7 Hz, 6H). | ES⁻ (m/z): 359.89 (M − 1) |
| 18 | 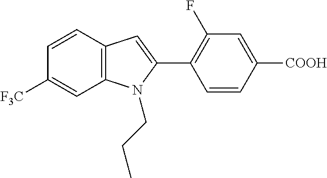 [SMILES: CCCn1c(-c2ccc(C(=O)O)cc2F)cc2ccc(C(F)(F)F)cc21] | ¹H NMR (400 MHz, Methanol-d₄) δ 7.96 (d, J = 9.4 Hz, 1H), 7.89-7.83 (m, 1H), 7.79 (s, 1H), 7.73 (d, J = 8.3 Hz, 1H), 7.61 (t, J = 7.5 Hz, 1H), 7.32 (d, J = 9.3 Hz, 1H), 6.67 (s, 1H), 4.16 (t, J = 7.2 Hz, 2H), 1.59 (q, J = 7.3 Hz, 2H), 0.64 (t, J = 7.4 Hz, 3H). | ES⁻(m/z): 363.86 (M − 1) |
| 19 | 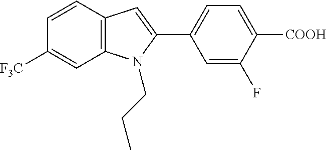 [SMILES: CCCn1c(-c2ccc(C(=O)O)c(F)c2)cc2ccc(C(F)(F)F)cc21] | ¹H NMR (400 MHz, Methanol-d₄) δ 8.06 (t, J = 8.0 Hz, 1 H), 7.79 (s, 1 H), 7.73 (d, J = 8.4 Hz, 1 H), 7.48-7.40 (m, 2 H), 7.32 (d, J = 8.4 Hz, 1 H), 6.73 (s, 1 H), 4.31 (t, J = 7.2 Hz, 2 H), 1.68-1.56 (m, 2 H), 0.69 (t, J = 7.6 Hz, 3 H). | ES⁻ (m/z): 363.87 (M − 1) |
| 20 | 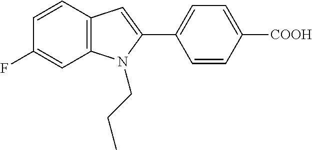 [SMILES: CCCn1c(-c2ccc(C(=O)O)cc2)cc2ccc(F)cc21] | ¹H NMR (400 MHz, Methanol-d₄) δ 8.11 (d, J = 8.4 Hz, 2H), 7.60 (d, J = 8.4 Hz, 2H), 7.51 (dd, J = 8.8, 5.6 Hz, 1H), 7.18 (d, J = 10.4 Hz, 1H), 6.84 (t, J = 9.2 Hz, 1H), 6.56 (s, 1H), 4.22-4.12 (m, 2H), 1.65-1.54 (m, 2H), 0.67 (t, J = 7.6 Hz, 3H). | ES⁻ (m/z): 295.83 (M − 1) |
| 21 | 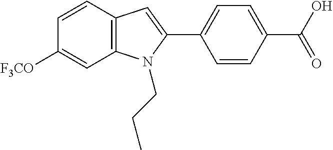 [SMILES: CCCn1c(-c2ccc(C(=O)O)cc2)cc2ccc(OC(F)(F)F)cc21] | ¹H NMR (400 MHz, Methanol-d₄) δ 7.96 (d, J = 9.4 Hz, 1H), 7.89-7.83 (m, 1H), 7.79 (s, 1H), 7.73 (d, J = 8.3 Hz, 1H), 7.61 (t, J = 7.5 Hz, 1H), 7.32 (d, J = 9.3 Hz, 1H), 6.67 (s, 1H), 4.16 (t, J = 7.2 Hz, 2H), 1.59 (q, J = 7.3 Hz, 2H), 0.64 (t, J = 7.4 Hz, 3H). | ES⁻ (m/z): 361.84 (M − 1) |
| 22 | 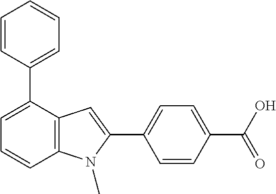 [SMILES: Cn1c(-c2ccc(C(=O)O)cc2)cc2c(-c3ccccc3)cccc21] | ¹H NMR (400 MHz, Methanol-d₄) δ 8.11 (d, J = 8.4 Hz, 2H), 7.71-7.62 (m, 4H), 7.48-7.39 (m, 3H), 7.37-7.26 (m, 2H), 7.15 (d, J = 7.2 Hz, 1H), 6.73 (s, 1H), 3.82 (s, 3H). | ES⁻ (m/z): 325.90 (M − 1) |
| 23 | 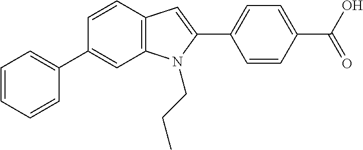 [SMILES: CCCn1c(-c2ccc(C(=O)O)cc2)cc2ccc(-c3ccccc3)cc21] | ¹H NMR (400 MHz, Methanol-d₄) δ 8.13 (d, J = 8.0 Hz, 2H), 7.69-7.61 (m, 6H), 7.42 (t, J = 7.6 Hz, 2H), 7.35 (d, J = 8.8 Hz, 1H), 7.28 (t, J = 7.6 Hz, 1H), 6.59 (s, 1H), 4.28 (t, J = 7.2 Hz, 2H), 1.72-1.61 (m, 2H), 0.70 (t, J = 7.2 Hz, 3H). | ES⁻ (m/z): 353.93 (M − 1) |
| 24 | 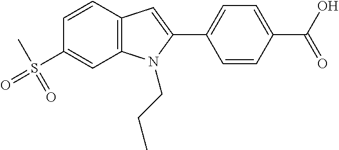 [SMILES: CCCn1c(-c2ccc(C(=O)O)cc2)cc2ccc(S(C)(=O)=O)cc21] | ¹H NMR (400 MHz, Methanol-d₄) δ 8.15 (d, J = 7.6 Hz, 2H), 8.09 (s, 1H), 7.79 (d, J = 8.0 Hz, 1H), 7.67 (d, J = 7.6 Hz, 2H), 7.63-7.59 (m, 1H), 6.73 (s, 1H), 4.35-4.28 (m, 2H), 3.15 (s, 3H), 1.70-1.59 (m, 2H), 0.69 (t, J = 7.6 Hz, 3H). | ES⁻ (m/z): 355.87 (M − 1) |
| 25 | 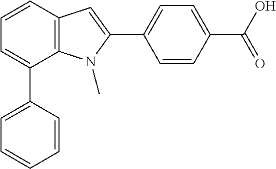 [SMILES: Cn1c(-c2ccc(C(=O)O)cc2)cc2cccc(-c3ccccc3)c21] | ¹H NMR (400 MHz, Methanol-d₄) δ 8.11-8.06 (m, 2H), 7.65-7.60 (m, 2H), 7.57 (dd, J = 8.0, 1.2 Hz, 1H), 7.51-7.42 (m, 4H), 7.41-7.35 (m, 1H), 7.13-7.06 (m, 1H), 7.01 (dd, J = 7.2, 1.2 Hz, 1H), 6.72 (s, 1H), 3.21 (s, 3H). | ES⁻ (m/z): 325.98 (M − 1) |

### Examples 26-32

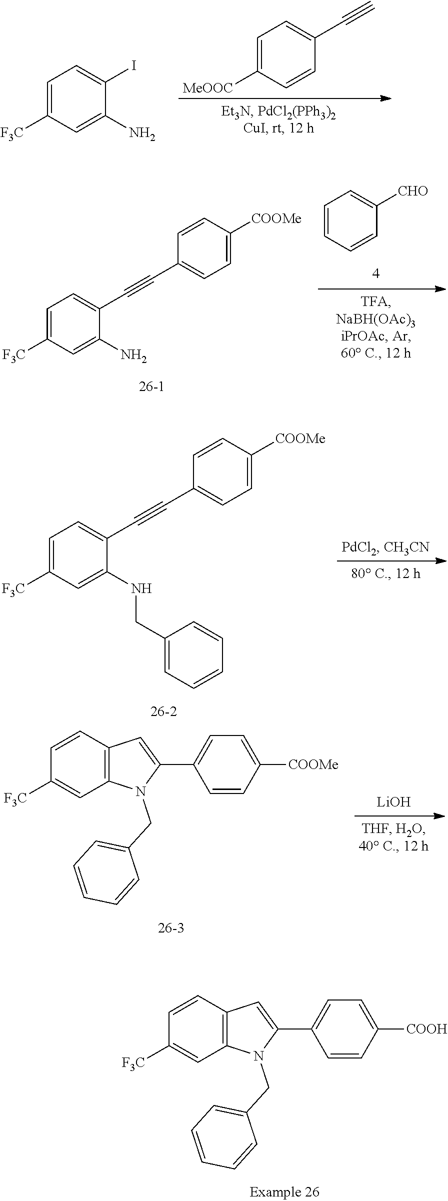

Step 1: A mixture of 2-Iodo-5-(trifluoromethyl) aniline (500 mg, 1.742 mmol), PdCl₂ (PPh₃)₂ (24.6 mg, 0.035 mmol) and CuI (13.2 mg, 0.070 mmol) in Et₃N(10 mL) was stirred at room temperature for 0.5 h under argon atmosphere. A solution of methyl 4-ethynylbenzoate (334.8 mg, 2.090 mmol) in Et₃N (10 mL) was slowly added. The resulting mixture was stirred at room temperature for 12 h under argon atmosphere. The reaction was quenched with saturated NH₄Cl aqueous solution (30 mL) and extracted with EtOAc for three times. The combined organic layers were dried over anhydrous Na₂SO₄, filtered, and concentrated under reduced pressure. The residue was purified by column chromatography to give the 26-1 (540 mg, 97.1% yield) as yellow solid.

Step 2: To a solution of 26-1 (20 mg, 0.063 mmol) and benzaldehyde (8.0 mg, 0.076 mmol) in i-PrOAc (1 mL) was slowly added TFA (14.4 mg, 0.126 mmol). The mixture was stirred at 25° C. for 0.5 h under argon atmosphere. NaBH(OAc)₃ (16.1 mg, 0.076 mmol) was added and the mixture was stirred at 60° C. for 12 h. The reaction mixture was quenched by saturated aq. NaHCO₃ solution (3 mL) and extracted with EtOAc for three times. The combined organic layers were dried over anhydrous Na₂SO₄, filtered, and concentrated under reduced pressure. The residue was purified by column chromatography to give the 26-2 (13 mg, 50.4% yield) as white solid. ¹H NMR (400 MHZ,) δ 8.03-7.98 (m, 2H), 7.53-7.49 (m, 2H), 7.48-7.45 (m, 1H), 7.40-7.35 (m, 4H), 7.34-7.29 (m, 1H), 6.90 (d, J=9.2 Hz, 1H), 6.80 (s, 1H), 5.20 (t, J=5.2 Hz, 1H), 4.47 (d, J=5.6 Hz, 2H), 3.92 (s, 3H).

Step 3: To a solution of 26-2 (12 mg, 0.029 mmol) in CH₃CN(1 mL) was added PdCl₂ (0.5 mg, 0.003 mmol) and the mixture was stirred at 80° C. for 12 h. The reaction mixture was concentrated under reduced pressure. The residue was purified by column chromatography to give the 26-3 (5 mg, 41.7% yield) as yellow solid. ¹H NMR (400 MHZ, CDCl₃) δ 8.08-8.02 (m, 2H), 7.75 (d, J=8.4 Hz, 1H), 7.52-7.45 (m, 3H), 7.39 (d, J=9.2 Hz, 1H), 7.31-7.25 (m, 3H), 6.97 (d, J=6.4 Hz, 2H), 6.76 (s, 1H), 5.40 (s, 2H), 3.92 (s, 3H).

Step 4: To a solution of 26-3 (5 mg, 0.012 mmol) in THF (0.6 mL) and H₂O(0.2 mL) was added LiOH (1.5 mg, 0.061 mmol). The mixture was stirred at 40° C. for 12 h. The reaction mixture was acidified with 1M HCl and extracted with EtOAc for three times. The combined organic layers were dried over anhydrous Na₂SO₄, filtered, and concentrated under reduced pressure. The residue was purified by column chromatography to give the Example 26 (4.5 mg, 97.4% yield) as yellow solid. LC-MS (ESI): m/z=393.91 [M−1]⁻. ¹H NMR (400 MHZ, DMSO-d₆) δ 8.05 (d, J=8.4 Hz, 2H), 7.77 (d, J=8.4 Hz, 1H), 7.61-7.55 (m, 3H), 7.33 (d, J=7.2 Hz, 1H), 7.26-7.16 (m, 3H), 6.90 (d, J=6.8 Hz, 2H), 6.81 (s, 1H), 5.52 (s, 2H).

The following examples were prepared employing the same protocol as described in Example 26.

| Example | Structure | NMR | MS |
|---|---|---|---|
| 27 | 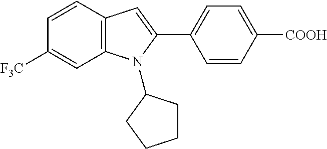 [SMILES: O=C(O)c1ccc(-c2cc3ccc(C(F)(F)F)cc3n2C2CCCC2)cc1] | ¹H NMR (400 MHz, DMSO-d₆) δ 8.06 (d, J = 8.4 Hz, 2 H), 7.80-7.75 (m, 2 H), 7.65-7.59 (m, 2 H), 7.35 (d, J = 8.4 Hz, 1 H), 6.66 (s, 1 H), 4.83-4.76 (m, 1 H), 2.22-2.10 (m, 2 H), 2.09-1.99 (m, 2 H), 1.94-1.85 (m, 2 H), 1.70-1.59 (m, 2 H). | ES⁻ (m/z): 371.90 (M − 1) |
| 28 | 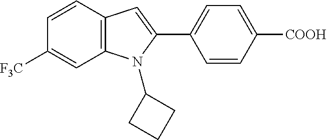 [SMILES: O=C(O)c1ccc(-c2cc3ccc(C(F)(F)F)cc3n2C2CCC2)cc1] | ¹H NMR (400 MHz, Methanol-d₄) δ 8.12 (d, J = 8.0 Hz, 2H), 7.93 (s, 1H), 7.71 (d, J = 8.3 Hz, 1H), 7.59 (d, J = 8.1 Hz, 2H), 7.31 (d, J = 8.3 Hz, 1H), 6.60 (s, 1H), 5.10 (t, J = 8.8 Hz, 1H), 2.70- 2.58 (m, 2H), 2.48-2.27 (m, 2H), 1.93- 1.76 (m, 2H). | ES⁻ (m/z): 357.85 (M − 1) |
| 29 | 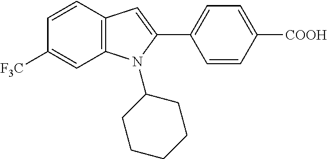 [SMILES: O=C(O)c1ccc(-c2cc3ccc(C(F)(F)F)cc3n2C2CCCCC2)cc1] | ¹H NMR (400 MHz, Methanol-d₄) δ 8.15 (d, J = 8.4 Hz, 2H), 7.91 (s, 1H), 7.71 (d, J = 8.4 Hz, 1H), 7.58 (d, J = 8.4 Hz, 2H), 7.29 (d, J = 8.4 Hz, 1H), 6.60 (s, 1H), 4.32-4.18 (m, 1H), 2.40 2.23 (m, 2H), 1.96-1.87 (m, 4H), 1.36- 1.24 (m, 4H). | ES⁻ (m/z): 385.90 (M − 1) |
| 30 | 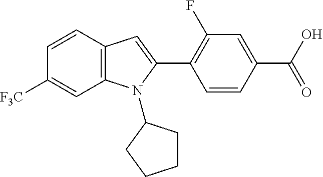 [SMILES: O=C(O)c1ccc(-c2cc3ccc(C(F)(F)F)cc3n2C2CCCC2)c(F)c1] | ¹H NMR (400 MHz, Methanol-d₄) δ 7.96 (d, J = 7.9 Hz, 1H), 7.88-7.81 (m, 1H), 7.79-7.71 (m, 2H), 7.58 (t, J = 7.6 Hz, 1H), 7.32 (d, J = 8.3 Hz, 1H), 6.61 (s, 1H), 4.60 (t, J = 9.0 Hz, 1H), 2.23 (s, 2H), 2.12-1.94 (m, 4H), 1.70 (s, 2H). | ES⁻ (m/z): 389.87 (M − 1) |
| 31 | 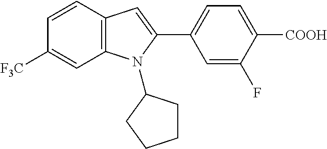 [SMILES: O=C(O)c1ccc(-c2cc3ccc(C(F)(F)F)cc3n2C2CCCC2)cc1F] | ¹H NMR (400 MHz, DMSO-d₆) δ 7.96 (t, J = 8.0 Hz, 1 H), 7.80 (d, J = 8.4 Hz, 1 H), 7.76 (s, 1 H), 7.48-7.39 (m, 2 H), 7.36 (d, J = 8.4 Hz, 1 H), 6.72 (s, 1 H), 4.86-4.74 (m, 1 H), 2.18-2.00 (m, 4 H), 1.94-1.84 (m, 2 H), 1.69-1.59 (m, 2 H). | ES⁻ (m/z): 389.89 (M − 1) |
| 32 | 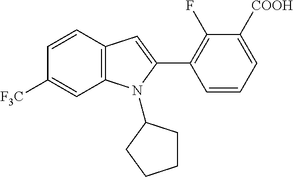 [SMILES: O=C(O)c1cccc(-c2cc3ccc(C(F)(F)F)cc3n2C2CCCC2)c1F] | ¹H NMR (400 MHz, Methanol-d₄) δ 8.28-8.10 (m, 1H), 7.87-7.60 (m, 3H), 7.37 (t, J = 7.3 Hz, 2H), 6.55 (s, 1H), 4.63-4.42 (m, 1H), 2.26 (s, 2H), 2.02 (s, 4H), 1.72 (s, 2H). | ES⁻ (m/z): 389.97 (M − 1) |

### Example 33

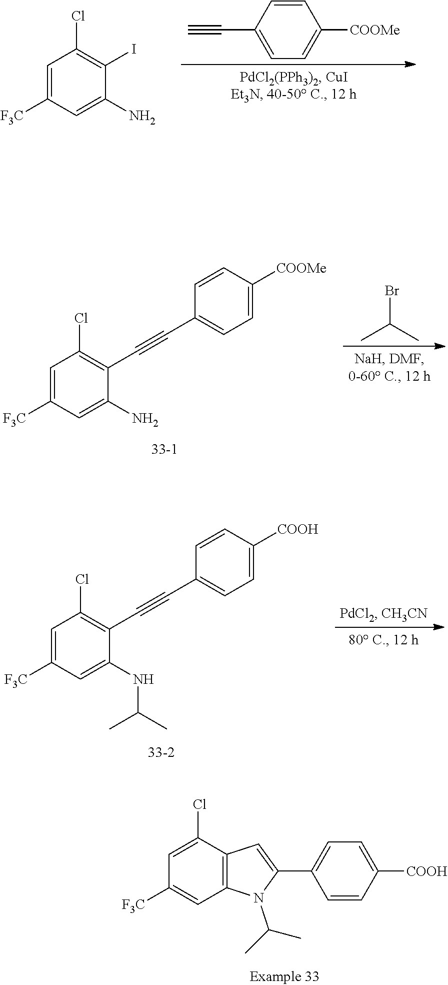

Step 1: A mixture of 3-Chloro-2-iodo-5-(trifluoromethyl) aniline (50 mg, 0.148 mmol), PdCl₂ (PPh₃)₂ (2.1 mg, 0.003 mmol) and CuI (1.1 mg, 0.006 mmol) in Et₃N(2 mL) was stirred at 40° C. for 0.5 h under argon atmosphere. A solution of methyl 4-ethynylbenzoate (71 mg, 0.444 mmol) in Et₃N(1 mL) was slowly added. The resulting mixture was stirred at 50° C. for 12 h under argon atmosphere. The reaction was quenched with saturated aqueous NH₄Cl solution (8 mL) and extracted with EtOAc for three times. The combined organic layers were dried over anhydrous Na₂SO₄, filtered, and concentrated under reduced pressure. The residue was purified by column chromatography to give the 33-1 (40 mg, 76.5% yield) as yellow solid.

Step 2: To a solution of 33-1 (20 mg, 0.057 mmol) in DMF (1 mL) was slowly added NaH (3.4 mg, 0.086 mmol) at 0° C. and the mixture was stirred at 25° C. for 0.5 h. Then 2-bromopropane (10.6 mg, 0.086 mmol) was added at 0° C. The mixture was stirred at 60° C. for 12 h. The reaction mixture was quenched with water (5 mL) and extracted with EtOAc for three times. The combined organic layers were dried over anhydrous Na₂SO₄, filtered, and concentrated under reduced pressure. The residue was purified by column chromatography to give the 33-2 (10.0 mg, 45.9% yield) as yellow solid. ¹H NMR (400 MHZ, CDCl₃) δ 8.93 (s, 1H), 8.13 (d, J=8.4 Hz, 2H), 7.76 (d, J=8.4 Hz, 2H), 7.60 (s, 1H), 7.38 (s, 1H), 7.06 (s, 1H), 5.32-5.23 (m, 1H), 1.39 (d, J=6.4 Hz, 6H).

Step 3: To a solution of 33-2 (10.0 mg, 0.026 mmol) in CH₃CN(1 mL) was added PdCl₂ (0.5 mg, 0.003 mmol) and the mixture was stirred at 80° C. for 12 h. The reaction mixture was concentrated under reduced pressure. The residue was purified by column chromatography to give Example 33 (7.5 mg, 75.0% yield) as white solid. LC-MS (ESI): m/z=379.86 [M−1]⁺. ¹H NMR (400 MHZ, Methanol-d₄) δ 8.13-8.05 (m, 2H), 7.99-7.89 (m, 2H), 7.65 (s, 1H), 7.27 (s, 1H), 7.09 (s, 1H), 5.27-5.17 (m, 1H), 1.40-1.33 (m, 1H).

### Example 34-36.

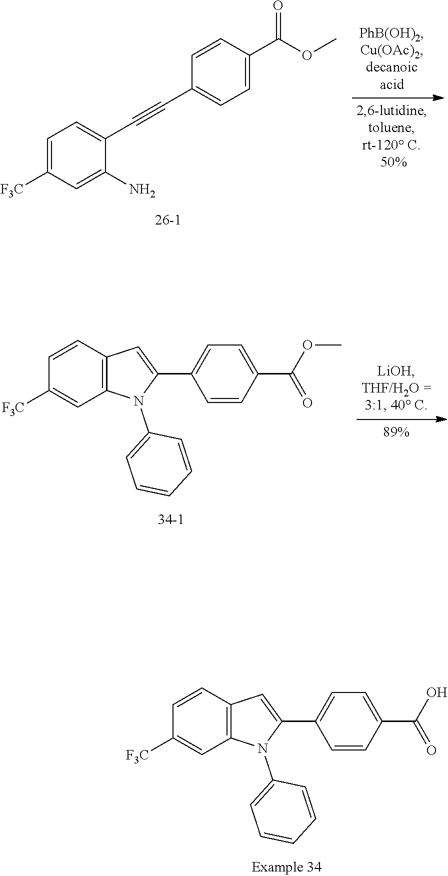 [SMILES: COC(=O)c1ccc(C#Cc2ccc(C(F)(F)F)cc2N)cc1 ; COC(=O)c1ccc(-c2cc3ccc(C(F)(F)F)cc3n2-c2ccccc2)cc1 ; O=C(O)c1ccc(-c2cc3ccc(C(F)(F)F)cc3n2-c2ccccc2)cc1]

Step 1: To a solution of methyl 4-((2-amino-4-(trifluoromethyl)phenyl) ethynyl)benzoate (26-1)(30.0 mg, 0.09 mmol), phenylboronic acid (17.0 mmol, 0.14 mmol), Cu(OAc)₂ (1.6 mg, 0.009 mmol), and decanoic acid (3.0 mg, 0.02 mmol) in toluene (2 mL) was added a solution of 2,6-lutidine (11.0 mg, 0.099 mmol) in toluene (3 mL). The mixture was stirred at room temperature for 8 h, and heated to 120° C. and stirred for 16 h. After being cooled to room temperature, the mixture was diluted with ethyl acetate (10 mL), filtered through a plug of silica gel, and concentrated. The residue was purified by chromatography on silica gel to give 34-1 (17.8 mg. 50% yield) as yellow solid. LC-MS, ES⁻ (m/z): 396.11 (M+1). ¹H NMR (400 MHZ, Chloroform-d) δ 7.92 (d, J=8.7 Hz, 2H), 7.77 (d, J=8.3 Hz, 1H), 7.52 (s, 1H), 7.50-7.38 (m, 4H), 7.32 (d, J=8.7 Hz, 2H), 7.26-7.21 (m, 2H), 6.93 (s, 1H), 3.89 (s, 3H).

Step 2: To a solution of 34-1 (9.0 mg, 0.022 mmol) in THF (0.9 mL) and H₂O(0.3 mL) was added LiOH (3.0 mg. 0.11 mmol), and the mixture was stirred overnight at 40° C. The reaction mixture was acidified with 1M HCl and extracted with EA for three times. The combined organic layers were dried over anhydrous Na₂SO₄, filtered, and concentrated in vacuum to give Example 34 (7.5 mg, 89% yield). LC-MS, ES⁻ (m/z): 380.10 (M−1). ¹H NMR (400 MHZ, Methanol-d₄) δ 7.88 (d, J=8.3 Hz, 2H), 7.81 (d, J=8.3 Hz, 1H), 7.86-7.44 (m, 3H), 7.43-7.35 (m, 4H), 7.28 (d, J=7.3 Hz, 2H), 7.02 (s, 1H).

The following examples were prepared employing the same protocol as described in Example 34.

| Example | Structure | NMR | MS |
|---|---|---|---|
| 35 | 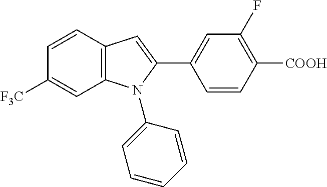 [SMILES: O=C(O)c1ccc(-c2cc3ccc(C(F)(F)F)cc3n2-c2ccccc2)cc1F] | ¹H NMR (400 MHz, Methanol-d₄) δ 7.82 (s, 1H), 7.59 (d, J = 8.0 Hz, 1H), 7.46-7.30 (m, 5H), 7.28 (s, 1H), 7.22- 7.15 (m, 2H), 7.10-7.02 (m, 2H). | ES⁻ (m/z): 397.89 (M − 1) |
| 36 | 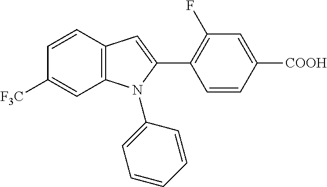 [SMILES: O=C(O)c1ccc(-c2cc3ccc(C(F)(F)F)cc3n2-c2ccccc2)c(F)c1] | ¹H NMR (400 MHz, Methanol-d₄) δ 7.82 (d, J = 8.4 Hz, 1H), 7.76 (d, J = 10.0 Hz, 1H), 7.67-7.58 (m, 2H), 7.41-7.31 (m, 4H), 7.24-7.19 (m, 2H), 7.11-7.04 (m, 2H). | ES⁻ (m/z): 397.89 (M − 1) |

### Example 37

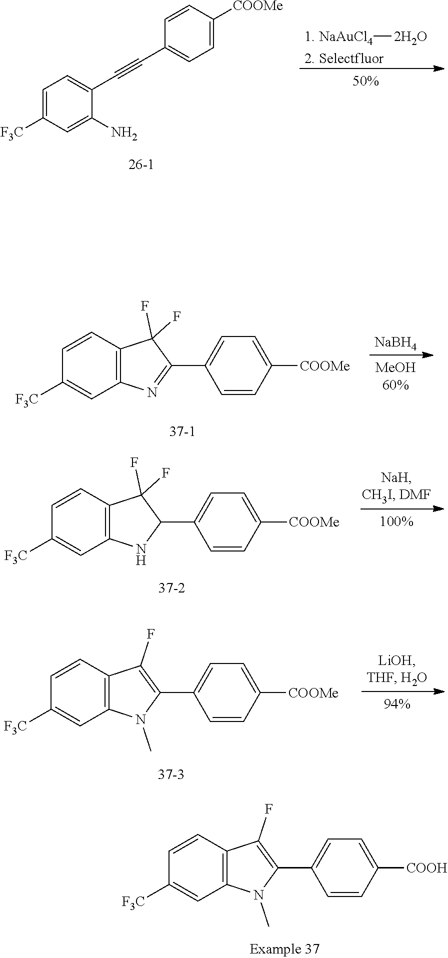

Step 1: To a solution of ethyl 4-((2-amino-4-(trifluoromethyl)phenyl) ethynyl)benzoate (26-1)(20.0 mg, 0.0625 mmol) in EtOH (1.0 mL) and H₂O(0.2 mL) was added NaAuCl₄ (1.1 mg, 0.0031 mmol), and the mixture was refluxed for 1h. Selectfluor (66 mg, 0.188 mmol) was added and the mixture was stirred for 1 h at 80° C. The reaction mixture was concentrated in vacuum, and purified with prep-TLC to give the desired product 37-1 (11.0 mg, 50% yield) as yellow solid. LC-MS, ES⁻ (m/z): 356.06 (M+1). ¹H NMR (400 MHZ, Chloroform-d) δ 8.28-8.15 (m, 4H), 7.81 (s, 1H), 7.71 (d, J=9.4 Hz, 1H), 7.63 (d, J=7.6 Hz, 1H), 3.96 (s, 3H).

Step 2: To a solution of methyl 4-(3,3-difluoro-6-(trifluoromethyl)-3H-indol-2-yl)benzoate (37-1)(5.0 mg, 0.014 mmol) in MeOH (1.0 mL) was added NaBH₄ (2.7 mg, 0.07 mmol), and the mixture was stirred for 1 h at room temperature. The reaction mixture was quenched by NaCl solution and extracted with EtOAc. The organic phase was dried over anhydrous Na₂SO₄, and the filtrate was evaporated in vacuum to provide 37-2 (3.0 mg, 60% yield) as yellow solid. LC-MS, ES⁻ (m/z): 358.08 (M+1). ¹H NMR (400 MHZ, Chloroform-d) δ 8.12-8.03 (m, 2H), 7.54 (d, J=8.3 Hz, 3H), 7.17 (d, J=7.7 Hz, 1H), 7.08 (s, 1H), 5.21-4.99 (m, 1H), 4.57 (s, 1H), 3.92 (s, 3H).

Step 3: To a solution of methyl 4-(3,3-difluoro-6-(trifluoromethyl) indolin-2-yl)benzoate (37-2)(3.0 mg, 0.0084 mmol) in DMF (1.0 mL) at 0° C. was added NaH (1.7 mg, 0.042 mmol), and the mixture was stirred for 30 minutes at 0° C. CH₃I (12.0 mg, 0.084 mmol) was added at 0° C. The mixture was stirred for 1 h at 0° C. The reaction mixture was quenched with NH₄Cl solution and extracted with EtOAc. The organic phase was dried over anhydrous Na₂SO₄, and the filtrate was evaporated in vacuum to afford 37-3 (3.0 mg, 100% yield) as yellow solid. LC-MS, ES⁻ (m/z): 352.09 (M+1).

Step 4: To a solution of methyl 4-(3-fluoro-1-methyl-6-(trifluoromethyl)-1H-indol-2-yl)benzoate (37-3)(3.0 mg, 0.008 mmol) in THF (0.6 mL) and H₂O(0.2 mL) was added LiOH (1.9 mg, 0.08 mmol), and the mixture was stirred for 1 h at room temperature. The reaction mixture was acidified with 1M HCl, and extracted with EtOAc for three times. The combined organic layers were dried over anhydrous Na₂SO₄, filtered, and concentrated in vacuum to give the desired product Example 37 (2.7 mg, 94% yield) as yellow solid. LC-MS, ES⁻ (m/z): 336.07 (M−1). ¹H NMR (400 MHZ, Methanol-d₄) δ 8.19 (d, J=1.9 Hz, 2H), 7.82 (s, 1H), 7.76-7.66 (m, 3H), 7.38 (s, 1H), 3.78 (s, 3H).

### Example 38

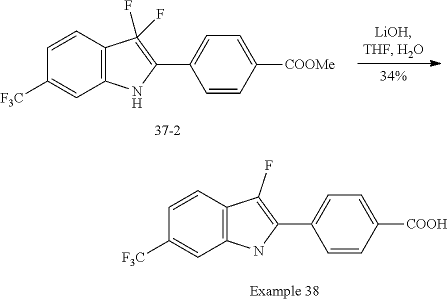

To a solution of methyl 4-(3,3-difluoro-6-(trifluoromethyl) indolin-2-yl)benzoate (37-2)(9.3 mg, 0.026 mmol) in THF (0.9 mL) and H₂O(0.3 mL) was added LiOH (6.3 mg, 0.26 mmol), and the mixture was stirred for 1 h at room temperature. TLC showed complete consumption of the starting material. The reaction mixture was acidified with 1M HCl and extracted with EtOAc for three times. The combined organic layers were dried over anhydrous Na₂SO₄, filtered, and concentrated in vacuum to give Example 38 (3.0 mg, 34% yield) as yellow solid. LC-MS, ES⁻ (m/z): 322.06 (M−1). ¹H NMR (400 MHZ, Methanol-d₄) δ 8.06 (d, J=8.4 Hz, 2H), 7.85 (d, J=8.3 Hz, 2H), 7.72-7.62 (m, 2H), 7.30 (d, J=8.3 Hz, 1H).

### Example 39

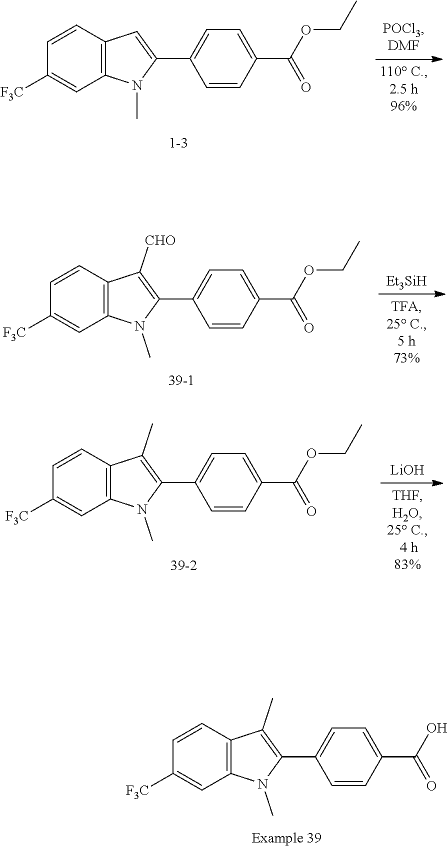 [SMILES: "Et3SiH" ; CCOC(=O)c1ccc(-c2cc3ccc(C(F)(F)F)cc3n2C)cc1 ; CCOC(=O)c1ccc(-c2c(C=O)c3ccc(C(F)(F)F)cc3n2C)cc1 ; CCOC(=O)c1ccc(-c2c(C)c3ccc(C(F)(F)F)cc3n2C)cc1 ; "LiOH" ; Cc1c(-c2ccc(C(=O)O)cc2)n(C)c2cc(C(F)(F)F)ccc12]

Step 1: To a solution of Compound 1-3 (20 mg, 0.058 mmol) in DMF (1 mL) was added POCl₃ (35.6 mg, 0.232 mmol), and the mixture was stirred at 115° C. for 2.5 h. The reaction mixture was added to water dropwise and extracted with EtOAc for three times. The combined organic layers were dried over anhydrous Na₂SO₄, filtered, and concentrated under reduced pressure. The residue was purified by flash column chromatography with EtOAc to give 39-1 (21 mg, 96.3%).

Step 2: To a solution of compound 39-1 (5 mg, 0.013 mmol) in TFA (1 mL) was added Et₃SiH (6.1 mg, 0.053 mmol), and the mixture was stirred at 25° C. for 5 h. Then the reaction mixture was poured onto ice water. The aqueous layer was adjusted to pH=8 with saturated NaHCO₃, and extracted with EtOAc. The combined organic layer was dried over anhydrous Na₂SO₄, filtered, and concentrated under reduced pressure. The residue was purified by column chromatography to give 39-2 (3.5 mg, 72.9% yield) as a yellow solid. ¹H NMR (400 MHz, Chloroform-d) δ 8.23-8.17 (m, 2H), 7.68 (d, J=8.4 Hz, 1H), 7.62 (s, 1H), 7.52-7.47 (m, 2H), 7.40 (dd, J=8.4, 1.6 Hz, 1H), 4.44 (q, J=7.2 Hz, 2H), 3.67 (s, 3H), 2.30 (s, 3H), 1.43 (d, J=4.0 Hz, 3H).

Step 3: To a solution of 39-2 (3.3 mg, 0.009 mmol) in THF (0.6 mL) and H₂O(0.2 mL) was added LiOH (1.1 mg, 0.046 mmol). The mixture was stirred at 25° C. for 4 h. The reaction mixture was acidified with AcOH and extracted with EtOAc for three times. The combined organic layers were dried over anhydrous Na₂SO₄, filtered, and concentrated under reduced pressure. The residue was purified by column chromatography to give Example 39 (2.5 mg, 83.3% yield) as a yellow solid. ES⁻ (m/z): 332.10 (M-1). ¹H NMR (400 MHZ, Chloroform-d) δ 8.29-8.23 (m, 2H), 7.69 (d, J=8.4 Hz, 1H), 7.63 (s, 1H), 7.55-7.51 (m, 2H), 7.40 (dd, J=8.4, 1.6 Hz, 1H), 3.69 (s, 3H), 2.32 (s, 3H).

### Example 40

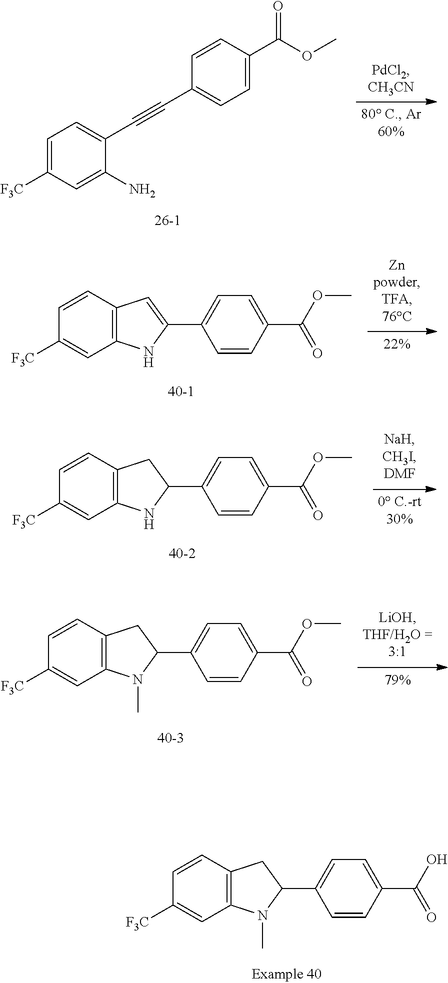 [SMILES: COC(=O)c1ccc(C#Cc2ccc(C(F)(F)F)cc2N)cc1 ; COC(=O)c1ccc(-c2cc3ccc(C(F)(F)F)cc3[nH]2)cc1 ; COC(=O)c1ccc(C2Cc3ccc(C(F)(F)F)cc3N2)cc1 ; COC(=O)c1ccc(C2Cc3ccc(C(F)(F)F)cc3N2C)cc1 ; CN1c2cc(C(F)(F)F)ccc2CC1c1ccc(C(=O)O)cc1]

Step 1: To a solution of methyl 4-((2-amino-4-(trifluoromethyl)phenyl) ethynyl)benzoate (26-1)(150.0 mg, 0.47 mmol) in MeCN(6.0 mL) was added PdCl₂ (8.3 mg, 0.047 mmol), and the mixture was stirred for overnight at 80° C. The reaction mixture was concentrated in vacuum and the residue was purified by prep-TLC to give 40-1 (90.0 mg, 60% yield) as yellow solid. LC-MS, ES⁻ (m/z): 320.08 (M+1).

Step 2: To a solution of methyl 4-(6-(trifluoromethyl)-1H-indol-2-yl)benzoate (40-1)(30.0 mg, 0.09 mmol) in TFA (1.0 mL) was added Zn powder (7.3 mg, 0.11 mmol) and the mixture was stirred overnight at 76° C. The reaction mixture was quenched by NaCl solution and extracted with EtOAc. The organic phase was dried over anhydrous Na₂SO₄, filtered, and concentrated in vacuo. Purification of the residue with prep-TLC provided 40-2 (6.4 mg, 22% yield) as brown oil. LC-MS, ES⁻ (m/z): 322.10 (M+1).

¹H NMR (400 MHZ, Chloroform-d) δ 8.01 (d, J=8.3 Hz, 2H), 7.47 (d, J=8.5 Hz, 2H), 7.18-7.08 (m, 1H), 7.03-6.95 (m, 1H), 6.85 (s, 1H), 5.07 (s, 1H), 3.91 (s, 3H), 3.58-3.44 (m, 1H), 3.03-2.88 (m, 1H).

Step 3: To a solution of methyl 4-(6-(trifluoromethyl) indolin-2-yl)benzoate (40-2)(6.4 mg, 0.02 mmol) in DMF (1.0 mL) was added NaH (4.0 mg, 0.1 mmol), and the mixture was stirred for 30 minutes at 0° C. CH₃I (30 mg, 0.2 mmol) was added at 0° C. The mixture was stirred at 0° C. for 1 h. The reaction mixture was quenched by NH₄Cl solution and extracted with EtOAc. The organic phase was dried over anhydrous Na₂SO₄, filtrated, and concentrated to afford 40-3 (2.0 mg, 30% yield) as colorless oil. LC-MS, ES⁻ (m/z): 336.11 (M+1).

Step 4: To a solution of methyl 4-(1-methyl-6-(trifluoromethyl) indolin-2-yl)benzoate (40-3)(2.0 mg, 0.0059 mmol) in THF (0.3 mL) and H₂O(0.1 mL) was added LiOH (1.4 mg, 0.059 mmol), and the mixture was stirred for overnight at 40° C. The reaction mixture was acidified with 1M HCl and extracted with EtOAc for three times. The combined organic layers were dried over anhydrous Na₂SO₄, filtered, and concentrated in vacuum to give Example 40 (1.5 mg, 79% yield) as colorless oil. LC-MS, ES⁻ (m/z): 322.10 (M+1). ¹H NMR (400 MHZ, Methanol-d₄) δ 8.02 (d, J=8.4 Hz, 2H), 7.53 (d, J=8.2 Hz, 2H), 7.14 (d, J=8.3 Hz, 1H), 6.93 (d, J=7.8 Hz, 1H), 6.70 (s, 1H), 4.63-4.50 (m, 1H), 3.50-3.38 (m, 1H), 2.95-2.78 (m, 1H), 2.63 (s, 3H).

### Example 41.

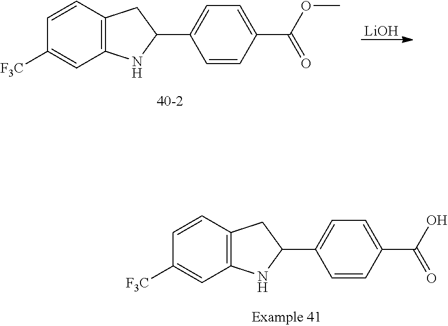

To a solution of methyl 4-(6-(trifluoromethyl) indolin-2-yl)benzoate (40-2)(6.0 mg, 0.018 mmol) in THF (0.6 mL) and H₂O(0.2 mL) was added LiOH (4.3 mg, 0.180 mmol). The mixture was stirred at r.t. overnight. The reaction mixture was acidified with AcOH and extracted with EtOAc for three times. The combined organic layers were dried over anhydrous Na₂SO₄, filtered, and concentrated in vacuum to give Example 41 (3.3 mg, 60.0% yield) as yellow solid. LC-MS, ES⁺ (m/z): 322.10 (M+1). ¹H NMR (400 MHz, Chloroform-d) δ 8.09 (d, J=8.4 Hz, 2H), 7.52 (d, J=8.0 Hz, 2H), 7.15 (d, J=7.6 Hz, 1H), 7.04-6.97 (m, 1H), 6.88 (d, J=1.6 Hz, 1H), 5.11 (t, J=9.2 Hz, 1H), 3.54 (dd, J=16.4, 9.6 Hz, 1H), 2.99 (dd, J=16.4, 8.8 Hz, 1H).

### Examples 42-47

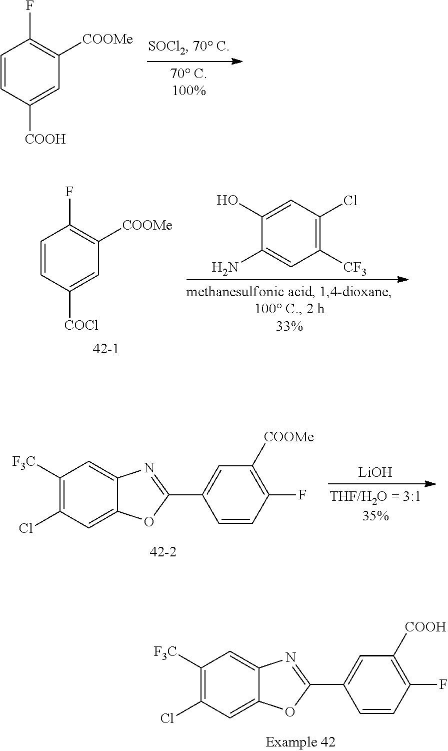 [SMILES: COC(=O)c1cc(C(=O)O)ccc1F ; COC(=O)c1cc(C(=O)Cl)ccc1F ; Nc1cc(C(F)(F)F)c(Cl)cc1O ; COC(=O)c1cc(-c2nc3cc(C(F)(F)F)c(Cl)cc3o2)ccc1F ; "LiOH" ; O=C(O)c1cc(-c2nc3cc(C(F)(F)F)c(Cl)cc3o2)ccc1F]

Step 1: To a 4 mL vials charged with 4-fluoro-3-(methoxycarbonyl)benzoic acid (20 mg, 0.10 mmol) was added SOCl₂ (1.0 mL) and the reaction solution was refluxed for 1 h. The reaction solution was cooled to room temperature and the excess thionyl chloride was removed in vacuo to give 42-1 (21.9 mg, 100% yield) as yellow solid. LC-MS, ES⁻ (m/z): 217.00 (M+1).

Step 2: A mixture of methyl 5-(chlorocarbonyl)-2-fluorobenzoate (42-1)(21.9 mg, 0.10 mmol), 2-amino-5-chloro-4-(trifluoromethyl) phenol (21.2 mg, 0.10 mmol), and methanesulfonic acid (28.8 mg, 0.30 mmol) in dioxane (1 mL) was stirred at 100° C. for 2 h. The solution was evaporated in vacuum, and water (2 mL) was added. The mixture was extracted with EtOAc (3 mL×2), and the EtOAc solution was washed with water (3 mL) and brine (3 mL). The EtOAc solution was dried over anhydrous Na₂SO₄, and the filtrate was evaporated in vacuum. The crude product was purified by silica gel flash chromatography to obtain 42-2 (12.5 mg, 33% yield) as yellow oil. LC-MS, ES⁻ (m/z): 374.01 (M+1).

Step 3: To a solution of 42-2 (12.5 mg, 0.033 mmol) in THF (0.9 mL) and H₂O(0.3 mL) was added LiOH (4.0 mg, 0.16 mmol), and the mixture was stirred for 3 h at room temperature. The reaction mixture was acidified with 1M HCl and extracted with EtOAc for three times. The combined organic layers were dried over anhydrous Na₂SO₄, filtered, and concentrated in vacuum to give Example 42 (4.2 mg, 35% yield) as white solid. LC-MS, ES⁻ (m/z): 358.00 (M−1). ¹H NMR (400 MHZ, DMSO-d₆) δ 8.47 (s, 1H), 8.30 (s, 2H), 8.15 (s, 1H), 7.35 (s, 1H).

The following examples were prepared employing the same protocol as described in Example 42

| Example | Structure | NMR | MS |
|---|---|---|---|
| 43 | 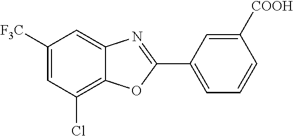 [SMILES: O=C(O)c1cccc(-c2nc3cc(C(F)(F)F)cc(Cl)c3o2)c1] | ¹H NMR (400 MHz, DMSO-d₆) δ 8.69 (s, 1H), 8.42 (d, J = 6.6 Hz, 1H), 8.27- 8.17 (m, 2H), 7.98 (s, 1H), 7.85-7.73 (m, 1H). | ES⁻ (m/z): 339.89 (M − 1) |
| 44 | 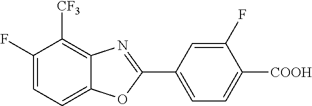 [SMILES: O=C(O)c1ccc(-c2nc3c(C(F)(F)F)c(F)ccc3o2)cc1F] | ¹H NMR (400 MHz, DMSO-d₆) δ 8.18 (dd, J = 9.2, 4.4 Hz, 1 H), 7.99 (d, J = 8.0 Hz, 1 H), 7.92-7.81 (m, 2 H), 7.62-7.50 (m, 1 H). | ES⁻ (m/z): 341.96 (M − 1) |
| 45 | 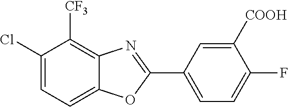 [SMILES: O=C(O)c1cc(-c2nc3c(C(F)(F)F)c(Cl)ccc3o2)ccc1F] | ¹H NMR (400 MHz, DMSO-d₆) δ 8.42 (s, 1H), 8.06 (d, 2H), 7.69 (s, 1H), 7.23 (s, 1H). | ES⁻ (m/z): 357.93 (M − 1) |
| 46 | 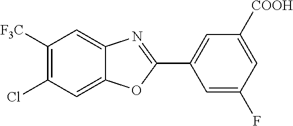 [SMILES: O=C(O)c1cc(F)cc(-c2nc3cc(C(F)(F)F)c(Cl)cc3o2)c1] | ¹H NMR (400 MHz, DMSO-d₆) δ 8.57 (s, 1H), 8.40(d, 2H), 8.05 (s, 1H), 7.85 (s, 1H). | ES⁻ (m/z): 357.94 (M − 1) |
| 47 | 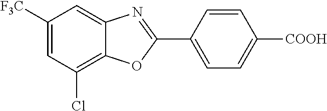 [SMILES: O=C(O)c1ccc(-c2nc3cc(C(F)(F)F)cc(Cl)c3o2)cc1] | ¹H NMR (400 MHz, DMSO-d₆) δ 8.31 (d, J = 8.7 Hz, 2H), 8.26 (s, 1H), 8.15 (d, J = 8.2 Hz, 2H), 8.00 (s, 1H). | ES⁻ (m/z): 339.89 (M − 1) |

### Examples 48-49

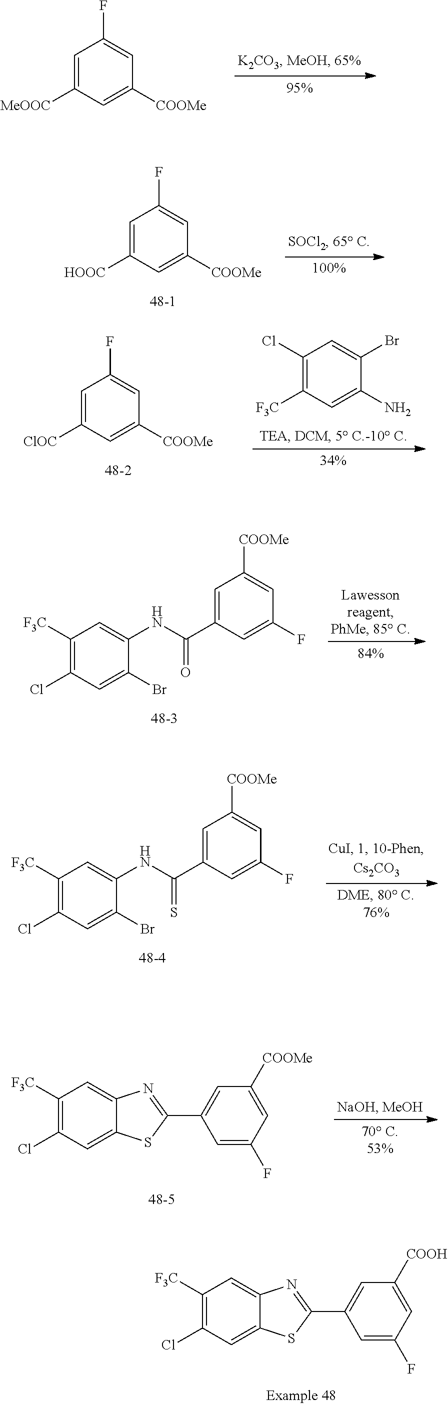

Step 1: Dimethyl 5-fluoroisophthalate (250 mg, 1.18 mmol) and K₂CO₃ (179 mg, 1.30 mmol) were dissolved in CH₃OH (5.0 mL) and the mixture was stirred at 65° C. for 3 h. The solvent was evaporated under vacuum. The crude was acidified with 1 M HCl and extracted with EtOAc for three times. The combined organic layers were dried over anhydrous Na₂SO₄, filtered, and concentrated in vacuum. Purification of the residue by silica column afforded 48-1 (221 mg, 95% yield) as colorless oil. LC-MS, ES⁻(m/z): 199.03 (M+1).

Step 2: To a 20 ml vials charged with 3-fluoro-5-(methoxycarbonyl)benzoic acid (48-1)(200 mg, 1.0 mmol) was added SOCl₂ (4.0 mL) and the reaction solution was refluxed for 1 h. The reaction solution was cooled to room temperature and the excess thionyl chloride was removed in vacuo to give 48-2 (219 mg, 100% yield) as yellow solid. LC-MS, ES⁻ (m/z): 217.00 (M+1).

Step 3:2-bromo-4-chloro-5-(trifluoromethyl) aniline (150 mg, 0.55 mmol) and TEA (166 mg, 1.64 mmol) were dissolved in DCM (3 mL). The reaction mixture was stirred at 0° C. for 10 min. methyl 3-(chlorocarbonyl)-5-fluorobenzoate (48-2)(118 mg, 0.55 mmol) was added at 0° C. and the reaction was stirred at 5° C.-10° C. for 3 h. The solution was evaporated in vacuum, and water (2 mL) was added to the residue. The mixture was extracted with EtOAc (5 mL×2). The combined EtOAc layer was washed with water (5 mL) and brine (5 mL), dried over anhydrous Na₂SO₄, and the filtrate was evaporated in vacuum. The crude product was purified by silica gel flash chromatography to afford 48-3 (84 mg, 34% yield) as yellow oil. LC-MS, ES⁻ (m/z): 453.94 (M+1). ¹H NMR (400 MHZ, Chloroform-d) δ 8.68 (s, 1H), 8.21-8.10 (m, 1H), 7.97-7.86 (m, 1H), 7.69 (d, J=8.7 Hz, 1H), 7.50 (d, J=8.7 Hz, 1H), 3.99 (s, 3H).

Step 4: Methyl 3-((2-bromo-4-chloro-5-(trifluoromethyl)phenyl) carbamoyl)-5-fluorobenzoate (48-3)(40 mg, 0.09 mmol) and Lawesson reagent (43 mg, 0.11 mmol) were dissolved in PhMe (2.0 mL) and the mixture was stirred at 85° C. for 12 h. Solvent was removed under vacuum. The crude was purified by silica column to afford 48-4 (35 mg, 84% yield) as yellow oil. LC-MS, ES⁻(m/z) 469.92 (M+1). ¹H NMR (400 MHZ, Chloroform-d) δ 9.24 (s, 1H), 8.96 (s, 1H), 8.27 (s, 1H), 7.92-7.80 (m, 3H), 3.96 (s, 3H).

Step 5: Methyl 3-((2-bromo-4-chloro-5-(trifluoromethyl)phenyl)carbamothioyl)-5-fluorobenzoate (48-4)(35 mg, 0.074 mmol), CuI (1.3 mg, 0.007 mmol), 1,10-Phen (1.3 mg, 0.007 mmol) and Cs₂CO₃ (49.0 mg, 0.15 mmol) were dissolved in 2 mL of DME. The reaction mixture was heated at 80° C. for 12 h. The reaction mixture was cooled to room temperature, diluted with 5 mL of EtOAc, and washed with H₂O(5 mL) and brine (5 mL). The organic layer was dried with anhydrous MgSO₄, filtered, and concentrated in vacuum. The residue was purified by silica gel chromatography to give 48-5 (22 mg, 76% yield) as yellow solid. LC-MS, ES⁻(m/z) 390.00 (M+1).

Step 6: To a solution of methyl 3-(6-chloro-5-(trifluoromethyl)benzo[d]thiazol-2-yl)-5-fluorobenzoate (48-5)(20 mg, 0.05 mmol) in MeOH (1.0 mL) was added NaOH (4.1 mg, 0.10 mmol), and the mixture was stirred for 2 h at 70° C. The reaction mixture was acidified with 1M HCl and extracted with EtOAc for three times. The combined organic layers were dried over anhydrous Na₂SO₄, filtered, and concentrated in vacuum. The residue was purified by prep-TLC to give Example 48 (10.2 mg, 53% yield) as white solid. LC-MS, ES⁻ (m/z): 373.91 (M−1). ¹H NMR (400 MHZ, DMSO-d₆) δ 8.68 (s, 1H), 8.57 (s, 1H), 8.46 (q, J=1.8 Hz, 1H), 8.18 (dt, J=9.0, 2.1 Hz, 1H), 7.88 (dt, J=8.5, 2.2 Hz, 1H).

The following example was prepared employing the same protocol as described in Example 48.

| Example | Structure | NMR | MS |
|---|---|---|---|
| 49 | 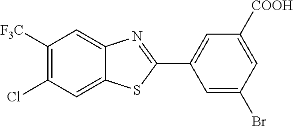 [SMILES: O=C(O)c1cc(Br)cc(-c2nc3cc(C(F)(F)F)c(Cl)cc3s2)c1] | ¹H NMR (400 MHz, DMSO-d₆) δ 8.68 (s, 1H), 8.60-8.53 (m, 2H), 8.47 (q, J = 1.7 Hz, 1H), 8.20 (q, J = 1.7 Hz, 1H). | ES⁻ (m/z): 435.85 (M − 1) |

### Examples 50-51

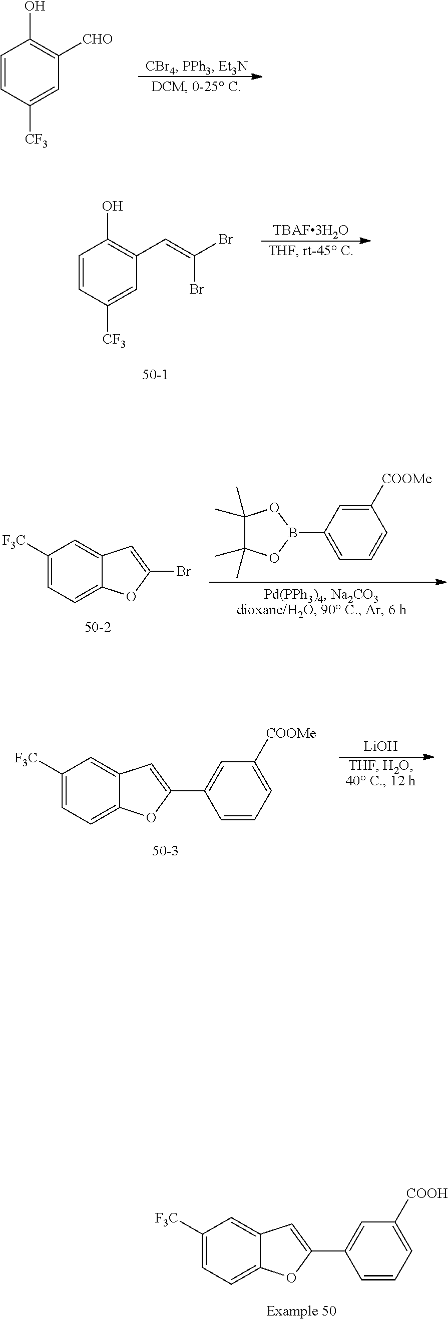

Step 1: To a solution of PPh₃ (2.48 g, 9.468 mmol) in DCM (10 mL) at 0° C. was slowly added a solution of CBr₄ (1.57 g, 4.734 mmol) in DCM (10 mL) at 0° C. over 10 min. Et₃N(958 mg, 9.468 mmol) was slowly added, followed by addition of a solution of 2-hydroxy-5-(trifluoromethyl)benzaldehyde (300 mg, 1.578 mmol) in DCM (10 mL). The mixture was stirred at 25° C. for 3 h. The reaction mixture was concentrated under reduced pressure to give 50-1 (280 mg, 51.3% yield) as yellow oil. ¹H NMR (400 MHz, CDCl₃) δ 7.81 (s, 1H), 7.53 (s, 1H), 7.47 (d, J=8.4 Hz, 1H), 6.90 (d, J=8.4 Hz, 1H).

Step 2: To a solution of 50-1 (200 mg, 0.578 mmol) in THF (5 mL) was added TBAF·3H₂O (182 mg, 0.578 mmol) at room temperature. The mixture was stirred at 45° C. for 12 h. The reaction mixture was diluted with water (5 mL) and extracted with EtOAc for three times. The combined organic layers were dried over anhydrous Na₂SO₄, filtered, and concentrated under reduced pressure. The residue was purified by column chromatography to give 50-2 (15 mg, 9.8% yield) as yellow oil. ¹H NMR (400 MHZ, CDCl₃) δ 7.79 (s, 1H), 7.53-7.49 (m, 2H), 6.80 (s, 1H).

Step 3: A mixture of 50-2 (15 mg, 0.057 mmol), 3-Methoxycarbonylphenylboronic acid pinacol ester (17.8 mg, 0.068 mmol), Na₂CO₃ (18 mg, 0.171 mmol), and Pd(PPh₃)₄ (6.6 mg, 0.006 mmol) in 1,4-dioxane (0.8 mL) and H₂O(0.2 mL) was stirred at 90° C. for 6 h under argon atmosphere. After cooling to room temperature, the reaction mixture was diluted with water (3 mL) and extracted with EtOAc for three times. The combined organic layers were dried over anhydrous Na₂SO₄, filtered, and concentrated under reduced pressure. The residue was purified by column chromatography to give 50-3 (16 mg, 87.9% yield) as yellow oil. ¹H NMR (400 MHZ,) δ 8.52 (s, 1H), 8.04 (d, J=9.2 Hz, 2H), 7.88 (s, 1H), 7.61 (d, J=8.8 Hz, 1H), 7.59-7.50 (m, 2H), 7.15 (s, 1H), 3.97 (s, 3H).

Step 4: To a solution of 50-3 (16 mg, 0.050 mmol) in THF (0.9 mL) and H₂O(0.3 mL) was added LiOH (6.0 mg, 0.25 mmol) and the mixture was stirred at 40° C. for 12 h. The reaction mixture was acidified with 1M HCl and extracted with EtOAc for three times. The combined organic layers were dried over anhydrous Na₂SO₄, filtered, and concentrated under reduced pressure. The residue was purified by column chromatography to give Example 50 (15 mg, 79.8% yield) as white solid. LC-MS (ESI): m/z=304.89 [M−1]⁺. ¹H NMR (400 MHZ, DMSO-d₆) δ 8.44 (t, J=1.6 Hz, 1H), 8.22-8.16 (m, 1H), 8.08 (s, 1 H), 8.00-7.95 (m, 1H), 7.87 (d, J=8.8 Hz, 1H), 7.68-7.61 (m, 3H).

The following examples were prepared employing the same protocol as described in Example 50.

| | | | |
|---|---|---|---|
| Example | Structure | NMR | MS |

| | | | |
|---|---|---|---|
| 51 | 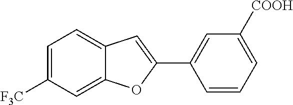 [SMILES: O=C(O)c1cccc(-c2cc3ccc(C(F)(F)F)cc3o2)c1] | ¹H NMR (400 MHz, DMSO-d₆) δ 8.46 (s, 1H), 8.19 (d, J = 9.5 Hz, 1H), 8.10 (s, 1H), 7.98 (d, J = 9.1 Hz, 1H), 7.86 (d, J = 8.2 Hz, 1H), 7.72-7.54 (m, 3H). | ES⁻ (m/z): 304.87 (M − 1) |

### Examples 52-65

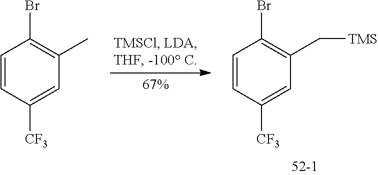 [SMILES: Cc1cc(C(F)(F)F)ccc1Br ; C[Si](C)(C)Cc1cc(C(F)(F)F)ccc1Br]

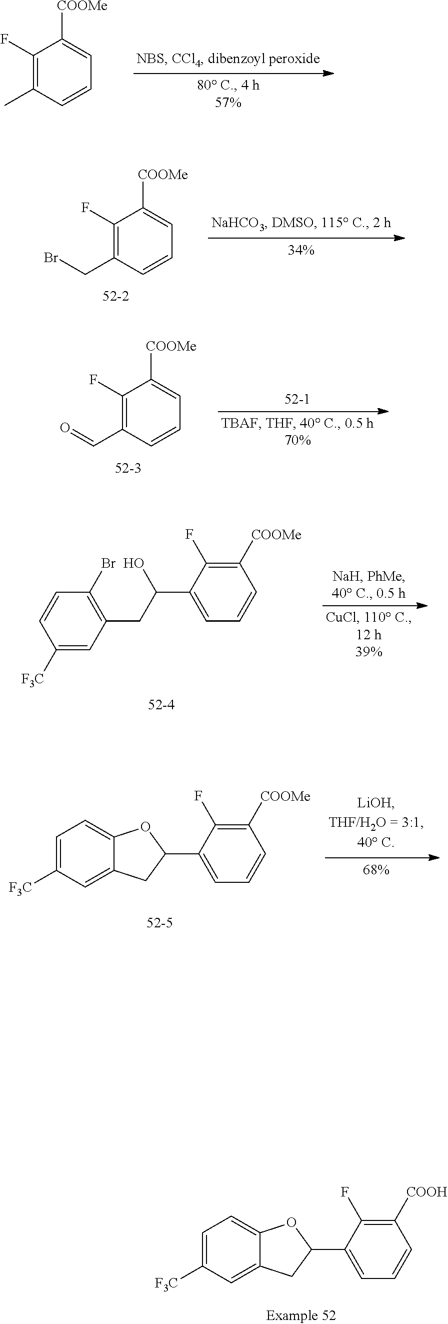 [SMILES: COC(=O)c1cccc(C)c1F ; COC(=O)c1cccc(CBr)c1F ; COC(=O)c1cccc(C=O)c1F ; COC(=O)c1cccc(C(O)Cc2cc(C(F)(F)F)ccc2Br)c1F ; COC(=O)c1cccc(C2Cc3cc(C(F)(F)F)ccc3O2)c1F ; O=C(O)c1cccc(C2Cc3cc(C(F)(F)F)ccc3O2)c1F]

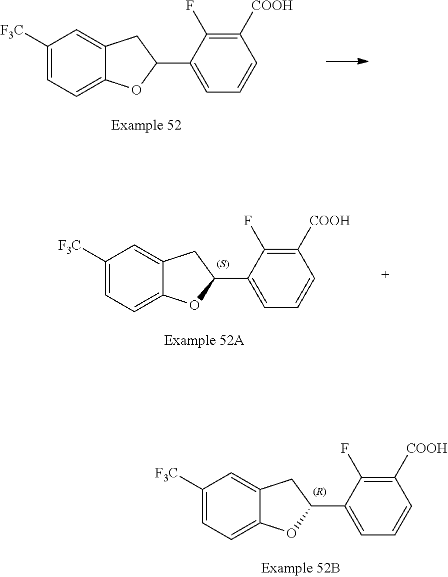

Step 1: To a solution of LDA (2.09 mL, 0.4 mmol) in THF/n-hexane (1:1) at −100° C. was added solution of 1-bromo-2-methyl-4-(trifluoromethyl)benzene (1.0 g, 0.4 mmol) and chlorotrimethylsilane (0.55 g, 0.5 mmol) in THF (10 mL) over 20 min. After the addition was complete, the mixture was stirred for additional 20 min at −100° C., warmed to room temperature over 1 h, and stirred at room temperature for 24 h. The reaction was quenched with 10 mL of water. The organic fraction was separated, and the aqueous fraction was extracted three times with diethyl ether. The combined organic layer was washed with 2 M HCl, 1 M NaHCO₃, and water. The organic fraction was dried with MgSO₄ and concentrated. The residue was subjected to fractional distillation. The product 52-1 (874 mg, 67% yield) was collected at 90° C. at 9 mbar as colorless oil. LC-MS, ES⁻ (m/z): 311.00 (M+1).

Step 2: To a mixture of methyl 2-fluoro-3-methylbenzoate (100 mg, 0.59 mmol) and N-bromosuccinimide (117 mg, 0.65 mmol) in CCl₄ (2 mL) at room temperature was added benzoyl peroxide (2.9 mg, 0.01 mmol) and the mixture was heated at reflux for 2.5 h. After the starting material was consumed, the mixture was concentrated under reduced pressure. The residue was purified by column chromatography to afford 52-2 (84 mg, 57% yield) as yellow oil. LC-MS, ES⁻(m/z) 246.97 (M+1). 1H NMR (400 MHZ, Chloroform-d) δ 7.97-7.82 (m, 1H), 7.63-7.51 (m, 1H), 7.19 (t, J=7.7 Hz, 1H), 4.52 (s, 2H), 3.93 (s, 3H).

Step 3: To a solution of methyl 3-(bromomethyl)-2-fluorobenzoate (52-2)(84 mg, 0.34 mmol) in DMSO(2 mL) was added NaHCO₃ (245 mg, 2.9 mmol). The mixture was heated at 115° C. under argon for 2 h. The reaction mixture was diluted with ethyl acetate and washed with brine. The organic layer was dried with Na₂SO₄, filtered, and concentrated under reduced pressure. The residue was purified by column chromatography to afford 52-3 (21 mg, 34% yield) as yellow oil. LC-MS, ES⁻ (m/z): 183.04 (M+1). ¹H NMR (400 MHZ, Chloroform-d) δ 10.42 (s, 1H), 8.27-8.16 (m, 1H), 8.11-8.00 (m, 1H), 7.34 (t, J=8.0 Hz, 1H), 3.97 (s, 3H).

Step 4: Methyl 2-fluoro-3-formylbenzoate (52-3)(21 mg, 0.12 mmol) and (2-bromo-5-(trifluoromethyl)benzyl)trimethylsilane (52-1)(109 mg, 0.35 mmol) were dissolved in THF (2 mL). TBAF solution (1 M in THF, 0.018 mL) was added in one portion. The mixture was stirred at 35-40° C. for 30 min. After addition of 0.12 mL of TBAF to the system, the solvent was evaporated under vacuum. The crude was acidified with 1M HCl and extracted with EtOAc for three times. The combined organic layers were dried over anhydrous Na₂SO₄, filtered, concentrated in vacuum. The residue was purified by prep-TLC to afford 52-4 (35.7 mg, 70% yield) as yellow solid. LC-MS, ES⁻ (m/z): 421.00 (M+1). ¹H NMR (400 MHz, Chloroform-d) δ 7.91-7.84 (m, 1H), 7.76-7.65 (m, 2H), 7.47 (s, 1H), 7.36 (d, J=8.2 Hz, 1H), 7.22 (d, J=7.7 Hz, 1H), 5.40 (d, J=4.2 Hz, 1H), 3.92 (s, 3H), 3.35-3.26 (m, 1H), 3.19-3.08 (m, 1H).

Step 5: Methyl 3-(2-(2-bromo-5-(trifluoromethyl)phenyl)-1-hydroxyethyl)-2-fluorobenzoate (52-4)(35.7 mg, 0.085 mmol) was dissolved in 2 mL of toluene. Under the nitrogen atmosphere, NaH (6.8 mg, 0.17 mmol) was added to the system with stirring. After stirring at 35-40° C. for 30 min, CuCl (0.4 mg, 0.004 mmol) was added. The system was refluxed for 6-8 h and then cooled to r.t. The reaction mixture was diluted with 5 mL EtOAc and washed with H₂O(2 mL) and brine (5 mL). The organic layer was dried with MgSO₄ and concentrated. The residue was purified by silica gel chromatography to give 52-5 (11.3 mg, 39% yield) as yellow oil. LC-MS, ES⁻ (m/z): 341.07 (M+1). ¹H NMR (400 MHZ, Chloroform-d) δ 7.94-7.85 (m, 1H), 7.68-7.59 (m, 1H), 7.49-7.40 (m, 2H), 7.24-7.18 (m, 1H), 6.95 (d, J=8.3 Hz, 1H), 6.12 (s, 1H), 3.93 (s, 3H), 3.85-3.73 (m, 1H), 3.33-3.04 (m, 1H).

Step 6: To a solution of 52-5 (11.3 mg, 0.033 mmol) in THF (0.9 mL) and H₂O(0.3 mL) was added LiOH (4.0 mg, 0.166 mmol), and the mixture was stirred at 40° C. overnight. The reaction mixture was acidified with 1M HCl and extracted with EtOAc for three times. The combined organic layers were dried over anhydrous Na₂SO₄, filtered, and concentrated in vacuum to give Example 52 (7.4 mg, 68% yield) as red solid. LC-MS, ES⁻ (m/z): 325.06 (M−1). ¹H NMR (400 MHZ, Methanol-d₄) δ 7.88 (s, 1H), 7.65 (s, 1H), 7.55-7.43 (m, 2H), 7.25 (t, J=7.7 Hz, 1H), 6.98 (d, J=8.3 Hz, 1H), 6.22-6.05 (m, 1H), 3.91-3.72 (m, 1H), 3.26-3.12 (m, 1H).

Example 52A and 52B: Compounds Example 52A and 52B were obtained by HPLC chiral separation of Example 52. Chiral Pak IE, n-Hexane/EtOH 0.1% TFA=90/10 (V/V), Rt (52A)=4.0 min; Rt (52B)=3.7 min. The stereochemistry of Example 52A was assigned to be(S) by X-ray crystallography.

The following examples were prepared employing the same protocol as described in Example 52.

| Example | Structure | NMR | MS |
|---|---|---|---|
| 53 | 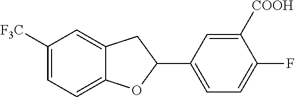 [SMILES: O=C(O)c1cc(C2Cc3cc(C(F)(F)F)ccc3O2)ccc1F] | ¹H NMR (400 MHz, Methanol-d₄) δ 7.95 (d, J = 6.6 Hz, 1H), 7.60 (s, 1H), 7.53-7.41 (m, 2H), 7.29-7.16 (m, 1H), 6.94 (d, J = 8.4 Hz, 1H), 5.90 (t, J = 8.7 Hz, 1H), 3.82-3.67 (m, 1H), 3.25-3.10 (m, 1H). | ES⁻ (m/z): 324.80 (M − 1) |
| 54 | 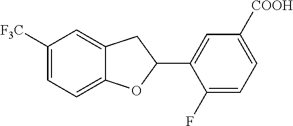 [SMILES: O=C(O)c1ccc(F)c(C2Cc3cc(C(F)(F)F)ccc3O2)c1] | ¹H NMR (400 MHz, Methanol-d₄) δ 8.11 (d, J = 9.1 Hz, 1H), 8.07-7.99 (m, 1H), 7.54-7.43 (m, 2H), 7.31-7.20 (m, 1H), 6.99 (d, J = 8.4 Hz, 1H), 6.15-6.04 (m, 1H), 3.91-3.72 (m, 1H), 3.26-3.16 (m, 1H). | ES⁻ (m/z): 324.80 (M − 1) |
| 55 | 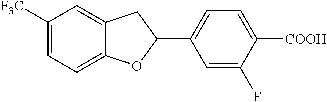 [SMILES: O=C(O)c1ccc(C2Cc3cc(C(F)(F)F)ccc3O2)cc1F] | ¹H NMR (400 MHz, Methanol-d₄) δ 7.93 (t, J = 7.6 Hz, 1 H), 7.52-7.44 (m, 2 H), 7.30-7.20 (m, 2 H), 6.98 (d, J = 8.4 Hz, 1 H), 5.99-5.88 (m, 1 H), 3.85-3.73 (m, 1 H), 3.23-3.13 (m, 1 H). | ES⁻ (m/z): 324.80 (M − 1) |
| 56 | 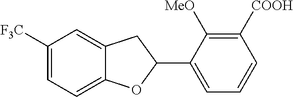 [SMILES: COc1c(C(=O)O)cccc1C1Cc2cc(C(F)(F)F)ccc2O1] | ¹H NMR (400 MHz, Methanol-d₄) δ 7.77 (s, 1H), 7.56 (d, J = 7.7 Hz, 1H), 7.51-7.40 (m, 2H), 7.16 (t, J = 7.7 Hz, 1H), 6.94 (d, J = 8.4 Hz, 1H), 6.19- 6.07 (m, 1H), 3.87-3.71 (m, 4H), 3.21-3.08 (m, 1H). | ES⁻ (m/z): 336.86 (M − 1) |
| 57 | 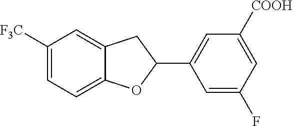 [SMILES: O=C(O)c1cc(F)cc(C2Cc3cc(C(F)(F)F)ccc3O2)c1] | ¹H NMR (400 MHz, Methanol-d₄) δ 7.86 (s, 1H), 7.63 (d, J = 7.6 Hz, 1H), 7.52-7.44 (m, 2H), 7.38 (d, J = 9.2 Hz, 1H), 6.98 (d, J = 8.4 Hz, 1H), 6.00-5.91 (m, 1H), 3.85-3.74 (m, 1H), 3.23-3.13 (m, 1H). | ES⁻ (m/z): 324.86 (M − 1) |
| 58 | 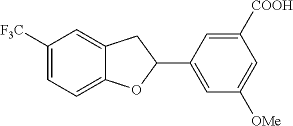 [SMILES: COc1cc(C(=O)O)cc(C2Cc3cc(C(F)(F)F)ccc3O2)c1] | ¹H NMR (400 MHz, Methanol-d₄) δ 7.62 (s, 1H), 7.52-7.43 (m, 3H), 7.15 (s, 1H), 6.96 (d, J = 8.3 Hz, 1H), 5.98-5.83 (m, 1H), 3.82 (s, 3H), 3.80-3.69 (m, 1H), 3.24-3.13 (m, 1H). | ES⁻ (m/z): 336.86 (M − 1) |
| 59 | 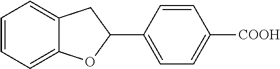 [SMILES: O=C(O)c1ccc(C2Cc3ccccc3O2)cc1] | ¹H NMR (400 MHz, Methanol-d₄) δ 7.96 (s, 2H), 7.42 (s, 2H), 7.17-7.05 (m, 2H), 6.82 (d, J = 6.5 Hz, 2H), 5.77 (d, J = 9.5 Hz, 1H), 3.73-3.49 (m, 1H), 3.23- 3.02 (m, 1H). | ES⁻ (m/z): 239.08 (M − 1) |
| 60 | 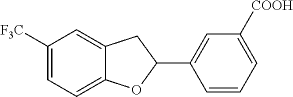 [SMILES: O=C(O)c1cccc(C2Cc3cc(C(F)(F)F)ccc3O2)c1] | ¹H NMR (400 MHz, Methanol-d₄) δ 8.04 (s, 1H), 7.97 (d, J = 9.1 Hz, 1H), 7.61 (d, J = 7.7 Hz, 1H), 7.47 (d, J = 7.6 Hz, 3H), 6.95 (d, J = 8.4 Hz, 1H), 5.94 (t, J = 8.9 Hz, 1H), 3.82-3.71 (m, 1H), 3.24-3.13 (m, 1H). | ES⁻ (m/z): 306.90 (M − 1) |
| 61 | 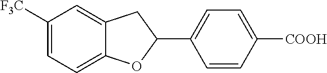 [SMILES: O=C(O)c1ccc(C2Cc3cc(C(F)(F)F)ccc3O2)cc1] | ¹H NMR (400 MHz, Methanol-d₄) δ 8.02 (d, J = 8.4 Hz, 2 H), 7.51-7.44 (m, 4 H), 6.96 (d, J = 8.4 Hz, 1 H), 6.01-5.89 (m, 1 H), 3.85-3.70 (m, 1 H), 3.22-3.14 (m, 1 H). | ES⁻ (m/z): 306.82 (M − 1) |
| 62 | 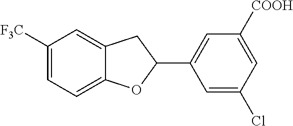 [SMILES: O=C(O)c1cc(Cl)cc(C2Cc3cc(C(F)(F)F)ccc3O2)c1] | ¹H NMR (400 MHz, Methanol-d₄) δ 7.95 (s, 1H), 7.93-7.90 (m, 1H), 7.62 (t, J = 1.6 Hz, 1H), 7.52-7.44 (m, 2H), 6.98 (d, J = 8.4 Hz, 1H), 5.98-5.89 (m, 1H), 3.84-3.73 (m, 1H), 3.23-3.13 (m, 1H). | ES⁻ (m/z): 340.86 (M − 1) |
| 63 | 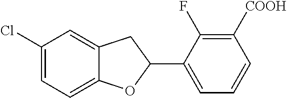 [SMILES: O=C(O)c1cccc(C2Cc3cc(Cl)ccc3O2)c1F] | ¹H NMR (400 MHz, DMSO-d₆) δ 13.33 (s, 1H), 7.85 (td, J = 7.4, 1.9 Hz, 1H), 7.70-7.62 (m, 1H), 7.34-7.29 (m, 2H), 7.20 (dd, J = 8.5, 2.3 Hz, 1H), 6.91 (d, J = 8.5 Hz, 1H), 6.10 (dd, J = 9.8, 7.4 Hz, 1H), 3.75 (dd, J= 16.3, 9.8 | ES⁻ (m/z): 291.28 (M − 1) |
| | | Hz, 1H), 3.19 (dd, J = 16.3, 7.4 Hz, | |
| | | 1H). | |
| 64 | 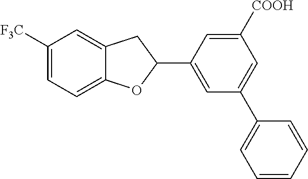 [SMILES: O=C(O)c1cc(-c2ccccc2)cc(C2Cc3cc(C(F)(F)F)ccc3O2)c1] | ¹H NMR (400 MHz, Methanol-d₄) δ 8.20 (s, 1H), 8.03 (s, 1H), 7.86 (s, 1H), 7.65-7.60 (m, 2H), 7.52 (s, 1H), 7.49- 7.43 (m, 3H), 7.39-7.33 (m, 1H), 7.02- 6.97 (m, 1H), 6.06-5.98 (m, 1H), 3.82 (dd, J = 16.0, 9.6 Hz, 1H), 3.27-3.22 (m, 1H) | ES⁻ (m/z): 382.95 (M − 1) |
| 65 | 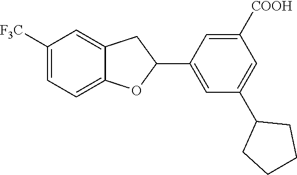 [SMILES: O=C(O)c1cc(C2CCCC2)cc(C2Cc3cc(C(F)(F)F)ccc3O2)c1] | ¹H NMR (400 MHz, Methanol-d₄) δ 7.88-7.82 (m, 2H), 7.52-7.48 (m, 2H), 7.46 (d, J = 8.4 Hz, 1H), 6.95 (d, J = 8.4 Hz, 1H), 5.95-5.87 (m, 1H), 3.81-3.69 (m, 1H), 3.23-3.13 (m, 1H), 3.12-3.00 (m, 1H), 2.13-2.02 (m, 2H), 1.87-1.77 (m, 2H), 1.76-1.67 (m, 2H), 1.62-1.54 (m, 2H). | ES⁻ (m/z): 375.00 (M − 1) |

### Examples 66-69

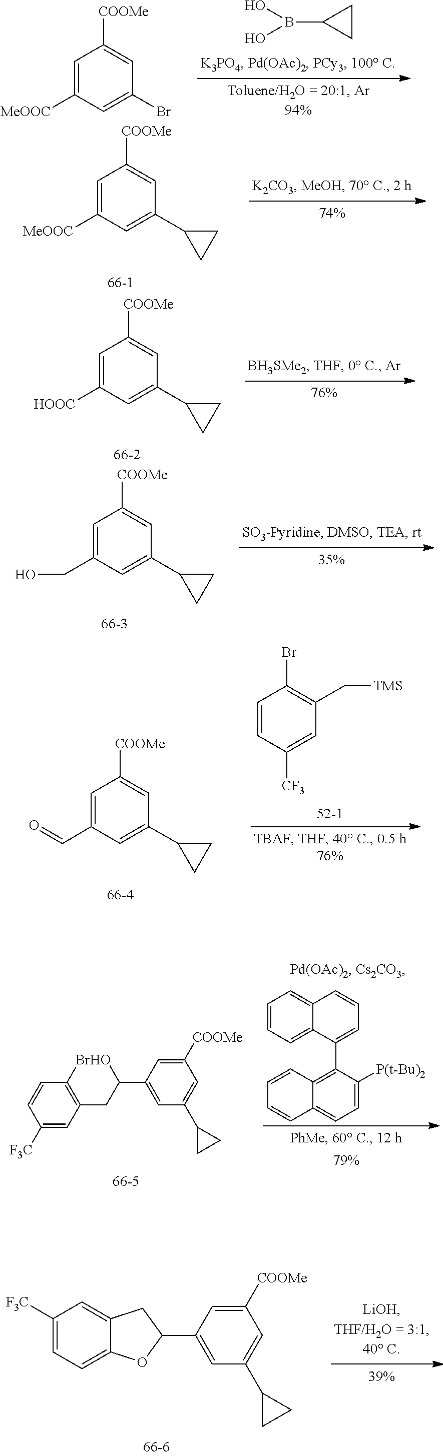

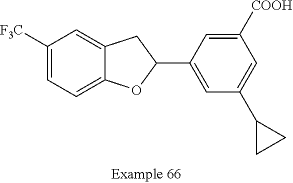 [SMILES: O=C(O)c1cc(C2CC2)cc(C2Cc3cc(C(F)(F)F)ccc3O2)c1]

Step 1: To a solution of dimethyl 5-bromoisophthalate (500 mg, 1.8 mmol), cyclopropylboronic acid (250 mg, 2.9 mmol) and potassium phosphate (1140 mg, 5.4 mmol) in toluene (10 mL) and H₂O(0.5 mL) under argon was added Pd(OAc)₂ (120 mg, 0.54 mmol) and tricyclohexylphosphine (150 mg, 0.54 mmol). The mixture was stirred at 100° C. for 12 h. The reaction mixture was quenched by NaCl solution and extracted with EtOAc. The organic phase was dried over anhydrous Na₂SO₄, and the filtrate was evaporated in vacuum. The residue was purified by silica gel flash chromatography to afford 66-1 (407 mg, 94% yield) as yellow solid. LC-MS, ES⁻(m/z) 235.25 (M+1). ¹H

NMR (400 MHZ, Chloroform-d) δ 8.44 (s, 1H), 7.91 (s, 2H), 3.92 (s, 6H), 2.06-1.90 (m, 1H), 1.08-0.99 (m, 2H), 0.82-0.73 (m, 2H).

Step 2: Dimethyl 5-cyclopropylisophthalate (66-1)(200 mg, 0.86 mmol) and K₂CO₃ (142 mg, 1.02 mmol) were dissolved in CH₃OH (4.0 mL) and the mixture was stirred at 70° C. for 12 h. Solvent was evaporated under high vacuum. The crude was acidified with 1M HCl and extracted with EtOAc for three times. The combined organic layers were dried over anhydrous Na₂SO₄, filtered, and concentrated in vacuum. The residue was purified by silica column to afford 66-2 (140 mg, 74% yield) as colorless oil. LC-MS, ES⁻(m/z) 221.07 (M+1). ¹H NMR (400 MHZ, Methanol-d₄) δ 8.37 (s, 1H), 7.91 (s, 2H), 3.90 (s, 3H), 2.12-1.94 (m, 1H), 1.12-1.01 (m, 2H), 0.80-0.68 (m, 2H).

Step 3: To a solution of 3-cyclopropyl-5-(methoxycarbonyl)benzoic acid (66-2)(70.0 mg, 0.32 mmol) in THF (2 mL) under Ar atmosphere at 0° C. was added BH₃SMe₂ (0.054 mL, 0.54 mmol). The mixture was warmed to room temperature and stirred under Ar for 12 h. The reaction was quenched with MeOH and concentrated under high vacuum. The crude was dissolved in EtOAc and washed with NaHCO₃(aq.) and brine. The organic layer was dried over anhydrous Na₂SO₄, filtered, and concentrated in vacuum. The residue was purified by silica column to afford 66-3 (50.3 mg, 76% yield) as white solid. LC-MS, ES⁻ (m/z): 207.09 (M+1).

Step 4: To a solution of methyl 3-cyclopropyl-5-(hydroxymethyl)benzoate (66-3)(50.3 mg, 0.24 mmol) in DMSO(2 mL) was added SO₃-pyridine (124 mg, 0.78 mmol) and TEA (296 mg, 2.93 mmol). The mixture was stirred at room temperature for 2 h. The reaction was quenched with H₂O and extracted with EtOAc. The combined organic layers were dried over anhydrous Na₂SO₄, filtered, and concentrated in vacuum. The residue was purified by silica column to afford 66-4 (17.8 mg, 35% yield) as colorless oil. LC-MS, ES⁻ (m/z): 205.08 (M+1). ¹H NMR (400 MHZ, Chloroform-d) δ 10.00 (s, 1H), 8.26 (s, 1H), 7.97 (s, 1H), 7.74 (s, 1H), 3.93 (s, 3H), 2.02-1.95 (m, 1H), 1.11-1.03 (m, 2H), 0.83-0.74 (m, 2H).

Step 5: Methyl 3-cyclopropyl-5-formylbenzoate (66-4)(17.8 mg, 0.087 mmol) and (2-bromo-5-(trifluoromethyl)benzyl)trimethylsilane 52-1 (81.4 mg, 0.26 mmol) were dissolved in THF (1.0 mL). TBAF solution (1 M in THF, 0.013 mL) was added in one portion. The mixture was stirred at 40° C. for 1 h. After addition of another 0.087 mL of TBAF to the system, the solvent was evaporated under high vacuum. The crude was acidified with 1M HCl and extracted with EtOAc for three times. The combined organic layers were dried over anhydrous Na₂SO₄, filtered, and concentrated in vacuum. The residue was purified by prep-TLC to afford 66-5 (29.3 mg, 76% yield) as colorless oil. LC-MS, ES⁻ (m/z): 443.04 (M+1). ¹H NMR (400 MHZ, Chloroform-d) δ 7.80 (s, 1H), 7.69 (s, 1H), 7.63 (s, 1H), 7.41 (s, 1H), 7.37-7.32 (m, 1H), 7.24 (s, 1H), 5.11-4.94 (m, 1H), 3.89 (s, 3H), 3.25-3.06 (m, 2H), 1.96-1.87 (m, 1H), 1.02-0.95 (m, 2H), 0.76-0.62 (m, 2H).

Step 6: Pd(OAc)₂ (1.3 mg, 0.006 mmol), rac-2-(Di-t-Butylphosphino)-1,1-Binaphthyl (2.4 mg, 0.006 mmol) and Cs₂CO₃ (32.3 mg, 0.099 mmol) were dissolved in 2 mL of toluene. A solution of methyl 3-(2-(2-bromo-5-(trifluoromethyl)phenyl)-1-hydroxyethyl)-5-cyclopropylbenzoate (66-5)(29.3 mg, 0.066 mmol) in 1 mL toluene was added under nitrogen atmosphere. The reaction was heated at 60° C. for 12 h, and then cooled to room temperature. The reaction mixture was diluted with 5 mL EtOAc, washed with H₂O(5 mL) and brine (5 mL). The organic layer was dried (anhydrous MgSO₄) and concentrated. The residue was purified by chromatography on silica gel to give 66-6 (18.9 mg, 79% yield) as colorless oil. LC-MS, ES⁻ (m/z): 363.11 (M+1). ¹H NMR (400 MHZ, Chloroform-d) δ 7.81 (s, 1H), 7.66 (s, 1H), 7.44 (d, J=9.7 Hz, 2H), 7.32 (s, 1H), 6.92 (d, J=8.2 Hz, 1H), 5.83 (t, J=9.0 Hz, 1H), 3.89 (s, 3H), 3.73-3.61 (m, 1H), 3.29-3.17 (m, 1H), 1.99-1.87 (m, 1H), 1.03-0.98 (m, 2H), 0.77-0.71 (m, 2H).

Step 7: To a solution of methyl 3-cyclopropyl-5-(5-(trifluoromethyl)-2,3-dihydrobenzofuran-2-yl)benzoate (66-6)(18.9 mg, 0.052 mmol) in THF (0.9 mL) and H₂O(0.3 mL) was added LiOH (6.3 mg, 0.26 mmol), and the mixture was stirred overnight at 40° C. The reaction mixture was acidified with 1 M HCl and extracted with EtOAc for three times. The combined organic layers were dried over anhydrous Na₂SO₄, filtered, and concentrated in vacuum. The residue was purified by TLC to give Example 66 (7.0 mg, 39% yield) as white solid. LC-MS, ES⁻ (m/z): 347.10 (M−1). ¹H NMR (400 MHZ, Chloroform-d) δ 7.87 (s, 1H), 7.73 (s, 1H), 7.45 (d. J=9.4 Hz, 2H), 7.37 (s, 1H), 6.99-6.89 (m, 1H), 5.86 (t, J=8.9 Hz, 1H), 3.80-3.61 (m, 1H), 3.31-3.14 (m, 1H), 1.96 (s, 1H), 1.08-0.98 (m, 2H), 0.92-0.82 (m, 2H).

The following examples were prepared employing the same protocol as described in Example 66.

| Example | Structure | NMR | MS |
|---|---|---|---|
| 67 | 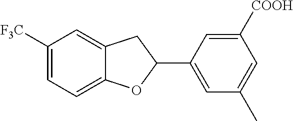 [SMILES: Cc1cc(C(=O)O)cc(C2Cc3cc(C(F)(F)F)ccc3O2)c1] | ¹H NMR (400 MHz, Methanol-d₄) δ 7.85-7.76 (m, 2H), 7.53-7.35 (m, 3H), 6.94 (d, J = 8.4 Hz, 1H), 5.96-5.80 (m, 1H), 3.79-3.68 (m, 1H), 3.22-3.11 (m, 1H), 2.37 (s, 3H). | ES⁻ (m/z): 320.87 (M − 1) |
| 68 | 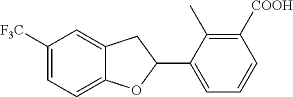 [SMILES: Cc1c(C(=O)O)cccc1C1Cc2cc(C(F)(F)F)ccc2O1] | ¹H NMR (400 MHz, Chloroform-d) δ 7.91 (d, J = 8.0 Hz, 1H), 7.70-7.59 (m, 1H), 7.52-7.39 (m, 2H), 7.29 (d, J = 7.6 Hz, 1H), 6.98 (d, J = 8.2 Hz, 1H), 6.18- 6.07 (m, 1H), 3.84-3.71 (m, 1H), 3.11- 3.01 (m, 1H), 2.60 (s, 3H). | ES⁻ (m/z): 321.08 (M − 1) |
| 69 | 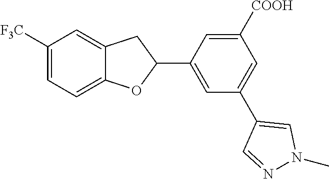 [SMILES: Cn1cc(-c2cc(C(=O)O)cc(C3Cc4cc(C(F)(F)F)ccc4O3)c2)cn1] | ¹H NMR (400 MHz, Methanol-d₄) δ 8.12 (s, 1H), 8.03 (s, 1H), 7.88 (s, 1H), 7.85 (s, 1H), 7.78 (s, 1H), 7.52 (s, 1H), 7.47 (d, J = 8.8 Hz, 1H), 6.98 (d, J = 8.6 Hz, 1H), 6.02-5.86 (m, 1H), 3.91 (s, 3H), 3.83-3.70 (m, 1H), 3.23 (s, 1H). | ES⁻ (m/z): 387.10 (M − 1) |

### Examples 70-71

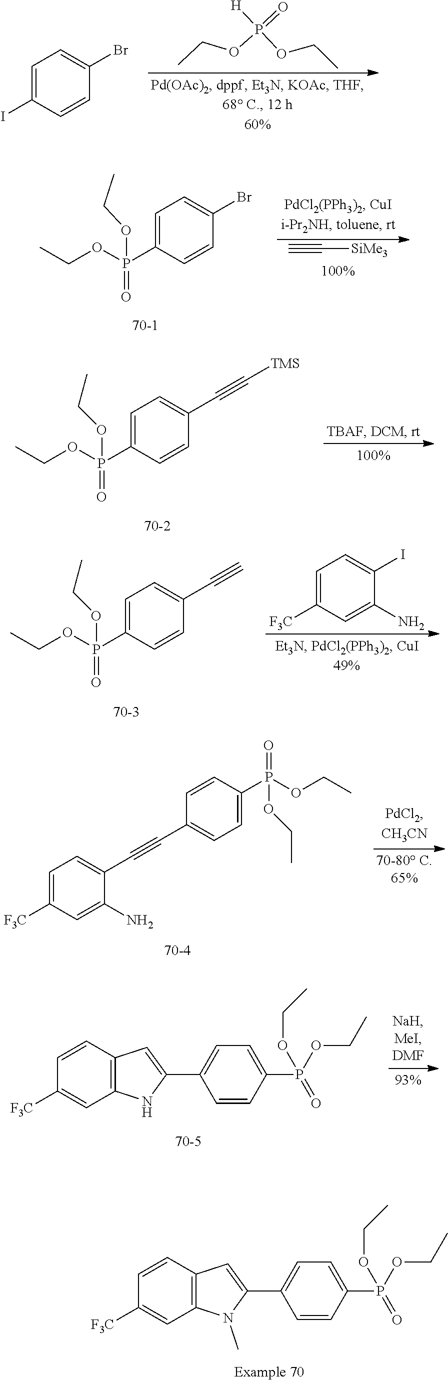

Step 1: Pd(OAc)₂ (8 mg, 0.04 mmol), dppf (39 mg, 0.07 mmol), KOAc (17 mg, 0.17 mmol) and Et₃N(215 mg, 2.1 mmol) was dissolved in THF (10 mL). The solution was heated at 68° C. under Ar atmosphere for 15 minutes and diethyl phosphonate (244 mg, 1.77 mmol) and 1-bromo-4-iodobenzene (500 mg, 1.77 mmol) were added. The mixture was heated at 68° C. for 12 hours. Solvent was removed under reduced pressure. The crude was purified by silica gel chromatography to give 70-1 (312 mg, 60% yield) as white solid. LC-MS, ES⁻ (m/z): 292.99 (M+1). ¹H NMR (400 MHZ, Chloroform-d) δ 7.76-7.53 (m, 4H), 4.21-3.95 (m, 4H), 1.31 (t, J=7.1 Hz, 6H).

Step 2: Diethyl(4-bromophenyl)phosphonate (70-1)(307.0 mg, 1.05 mmol), PdCl₂ (PPh₃)₂ (14.0 mg, 0.02 mmol), CuI (4.0 mg, 0.02 mmol), and i-Pr₂NH (530 mg, 5.25 mmol) were suspended in toluene (6 mL) under Ar atmosphere. Ethynyltrimethylsilane (154 mg, 1.57 mmol) were added and the mixture was stirred overnight under Ar atmosphere at room temperature. Solvent was removed under reduced pressure and H₂O was added. The mixture was extracted with CH₂Cl₂, and the organic layer was dried over Na₂SO₄ and concentrated. The crude product was purified by column chromatography to give 70-2 (325 mg, 100% yield) as brown oil. LC-MS, ES⁻ (m/z): 311.12 (M+1). ¹H NMR (400 MHZ, Chloroform-d) δ 7.76-7.50 (m, 4H), 4.28-3.90 (m, 4H), 1.30 (t, J=7.1 Hz, 6H), 0.17 (s, 9H).

Step 3: To a solution of diethyl(4-((trimethylsilyl) ethynyl)phenyl)phosphonate (70-2)(325 mg, 1.05 mmol) in DCM (10 mL) was added TBAF (663.0 mg, 2.10 mmol). The reaction mixture was stirred at room temperature for 2 h. The reaction was quenched by water and extracted with DCM. The organic phase was dried over anhydrous Na₂SO₄, filtered, and concentrated under reduced pressure. The crude product was purified by silica gel flash chromatography to afford 70-3 (250 mg, 100% yield) as yellow oil. LC-MS, ES⁻(m/z) 239.08 (M+1).

Step 4:2-Iodo-5-(trifluoromethyl) aniline (50.0 mg, 0.17 mmol), PdCl₂ (PPh₃)₂ (2.4 mg, 0.003 mmol), and CuI (1.3 mg, 0.007 mmol) were dissolved in Et₃N(3 mL). The solution was stirred under argon for 30 minutes at room temperature. Diethyl(4-ethynylphenyl)phosphonate (70-3)(108 mg, 0.34 mmol) was added and the mixture was stirred overnight under Ar atmosphere at room temperature. The reaction was quenched with NH₄Cl solution and extracted with EtOAc. The organic phase was dried over anhydrous Na₂SO₄, and the filtrate was evaporated in vacuum. The crude product was purified by silica gel flash chromatography to provide 70-4 (33.4 mg, 49% yield) as yellow solid. LC-MS, ES⁻ (m/z): 398.11 (M+1).

¹H NMR (400 MHZ, Chloroform-d) δ 7.85-7.76 (m, 2H), 7.62-7.57 (m, 2H), 7.45 (t, J=6.7 Hz, 2H), 6.95 (s, 1H), 4.46 (s, 2H), 4.24-4.00 (m, 4H), 1.32 (t, J=7.1 Hz, 6H).

To a Step 5: solution of diethyl(4-((2-amino-4-(trifluoromethyl)phenyl) ethynyl)phenyl)phosphonate (70-4)(31.4 mg, 0.08 mmol) in MeCN(1.5 mL) was added PdCl₂ (1.4 mg, 0.008 mmol), and the mixture was stirred at 80° C. overnight. The reaction mixture was concentrated in vacuum and the residue was purified by TLC to give 70-5 (20.6 mg, 65% yield) as yellow oil. LC-MS, ES⁻ (m/z): 398.11 (M+1). ¹H NMR (400 MHZ, Chloroform-d) δ 10.68 (s, 1H), 7.83-7.73 (m, 4H), 7.69 (d, J=8.3 Hz, 1H), 7.45 (t, J=8.9 Hz, 1H), 7.32 (d, J=9.7 Hz, 1H), 6.91 (s, 1H), 4.31-3.93 (m, 4H), 1.33 (t, J=7.1 Hz, 6H).

Step 6: To a solution of diethyl(4-(6-(trifluoromethyl)-1H-indol-2-yl)phenyl)phosphonate (70-6)(5 mg, 0.013 mmol) in DMF (1.0 mL) at 0° C. was added NaH (2.5 mg, 0.063 mmol). The mixture was stirred at 0° C. for 30 minutes and CH₃I (18.0 mg, 0.13 mmol) was added. The reaction mixture was stirred at 0° C. for 3 h, quenched with NH₄Cl solution, and extracted with EtOAc. The organic phase was dried over anhydrous Na₂SO₄, and the filtrate was evaporated in vacuum to give Example 70 (5.0 mg, 93% yield). LC-MS, ES⁻ (m/z): 412.12 (M+1). ¹H NMR (400 MHZ, Chloroform-d) δ 7.99-7.87 (m, 2H), 7.71 (d, J=8.2 Hz, 1H), 7.66-7.57 (m, 3H), 7.38 (d, J=8.3 Hz, 1H), 6.66 (s, 1H), 4.27-4.07 (m, 4H), 3.81 (s, 3H), 1.36 (t, J=7.6 Hz, 6H).

### Example 71

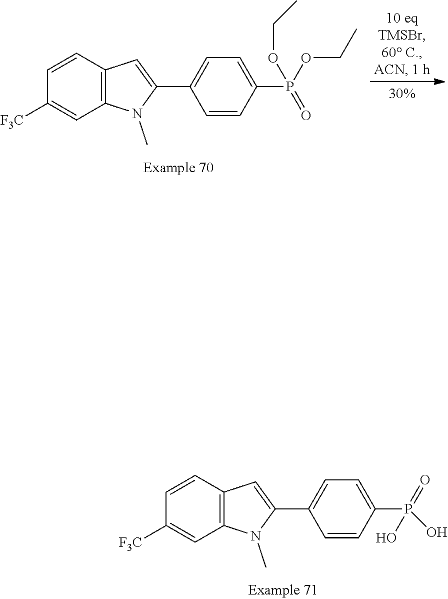 [SMILES: CCOP(=O)(OCC)c1ccc(-c2cc3ccc(C(F)(F)F)cc3n2C)cc1 ; Cn1c(-c2ccc(P(=O)(O)O)cc2)cc2ccc(C(F)(F)F)cc21]

To a solution of diethyl(4-(1-methyl-6-(trifluoromethyl)-1H-indol-2-yl)phenyl)phosphonate (Example 70)(6.1 mg, 0.015 mmol) in MeCN(1.0 mL) was added TMSBr (23.0 mg, 0.15 mmol), and the mixture was stirred at 60° C. for 1 h. The reaction mixture was quenched with MeOH and extracted with EtOAc for three times. The combined organic layers were dried over anhydrous Na₂SO₄, filtered, and concentrated in vacuum to give Example 71 (1.6 mg, 30% yield) as yellow oil. LC-MS, ES⁻ (m/z): 354.06 (M-1). ¹H NMR (400 MHZ, Methanol-d₄) δ 7.98-7.87 (m, 2H), 7.80-7.65 (m, 4H), 7.32 (d, J=8.4 Hz, 1H), 6.70 (s, 1H), 3.82 (s, 3H).

### Example 72

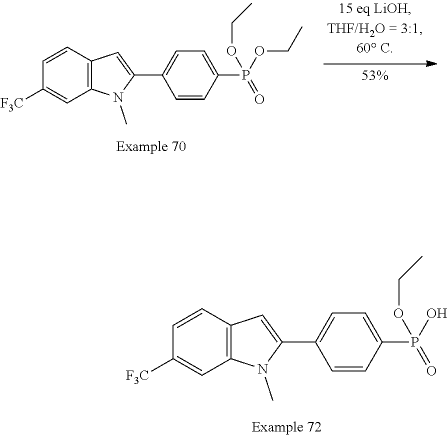 [SMILES: CCOP(=O)(OCC)c1ccc(-c2cc3ccc(C(F)(F)F)cc3n2C)cc1 ; CCOP(=O)(O)c1ccc(-c2cc3ccc(C(F)(F)F)cc3n2C)cc1]

To a solution of diethyl(4-(1-methyl-6-(trifluoromethyl)-1H-indol-2-yl)phenyl)phosphonate (Example 70)(8.7 mg, 0.021 mmol) in THF (1.5 mL) and H₂O(0.5 mL) was added LiOH (7.6 mg, 0.32 mmol). The mixture was stirred at 60° C. for 12 h. The reaction mixture was acidified with 1M HCl and extracted with EtOAc for three times. The combined organic layers were dried over anhydrous Na₂SO₄, filtered, and concentrated in vacuum to give Example 72 (4.3 mg, 53% yield) as white solid. LC-MS, ES⁻ (m/z): 382.09 (M−1). ¹H NMR (400 MHZ, Methanol-d₄) δ 7.95-7.83 (m, 2H), 7.77-7.67 (m, 2H), 7.60 (d, J=8.2 Hz, 2H), 7.30 (d, J=9.7 Hz, 1H), 6.65 (s, 1H), 3.81 (s, 5H), 1.19 (t, J=7.1 Hz, 3H).

### Example 73

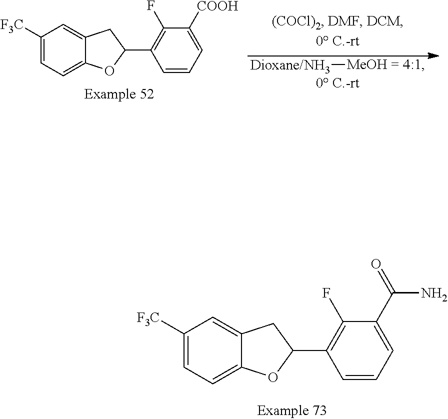

To a solution of Example 52 (12 mg, 0.037 mmol) in DCM (1.0 mL) and DMF (a drop) at 0° C. was added (COCl)₂ (47.0 mg, 0.37 mmol). The reaction was stirred at 0° C. for 15 min, and at room temperature for 2 h. Solvent was removed in vacuum. The residue was dissolved in dioxane (1.0 mL). At 0° C. NH₃-MeOH (0.25 mL) was added to the above solution and the reaction solution was stirred at room temperature for 2 h. Water was added and the mixture was extracted with EtOAc for three times. The combined organic layers were dried over anhydrous Na₂SO₄, filtered, concentrated in vacuum. The residue was purified by prep-TLC to give Example 73 (12.0 mg, 100% yield) as white solid. LC-MS, ES⁻ (m/z): 324.07 (M−1). ¹H NMR (400 MHZ, Chloroform-d) δ 8.18-7.97 (m, 1H), 7.61 (t, J=8.3 Hz, 1H), 7.52-7.39 (m, 2H), 7.28 (d, J=7.7 Hz, 1H), 6.95 (d, J=8.2 Hz, 1H), 6.62 (s, 1H), 6.08 (d, J=8.0 Hz, 2H), 3.87-3.72 (m, 1H), 3.29-3.15 (m, 1H).

The following examples were prepared employing the same protocol as described in Example 73.

| Example | Structure | NMR | MS |
|---|---|---|---|
| 74 | 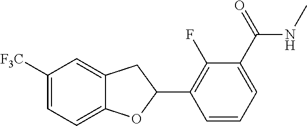 [SMILES: CNC(=O)c1cccc(C2Cc3cc(C(F)(F)F)ccc3O2)c1F] | ¹H NMR (400 MHz, Chloroform-d) δ 8.04 (t, J = 7.7 Hz, 1H), 7.55 (t, J = 8.3 Hz, 1H), 7.49-7.41 (m, 2H), 7.27 (s, 1H), 6.94 (d, J = 8.3 Hz, 1H), 6.67 (s, 1H), 6.17-5.97 (m, 1H), 3.82-3.71 (m, 1H), 3.26-3.16 (m, 1H), 3.05-3.01 (m, 3H). | ES⁻ (m/z): 340.04 (M + 1) |
| 75 | 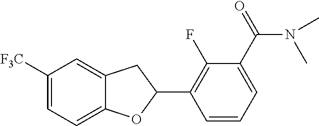 [SMILES: CN(C)C(=O)c1cccc(C2Cc3cc(C(F)(F)F)ccc3O2)c1F] | ¹H NMR (400 MHz, Chloroform-d) δ 7.53-7.40 (m, 3H), 7.37-7.29 (m, 1H), 7.20 (t, J = 7.7 Hz, 1H), 6.94 (d, J = 8.3 Hz, 1H), 6.14-6.02 (m, 1H), 3.80-3.69 (m, 1H), 3.22-3.11 (m, 4H), 2.94 (s, 3H). | ES⁻ (m/z): 354.06 (M + 1) |

### Example 76

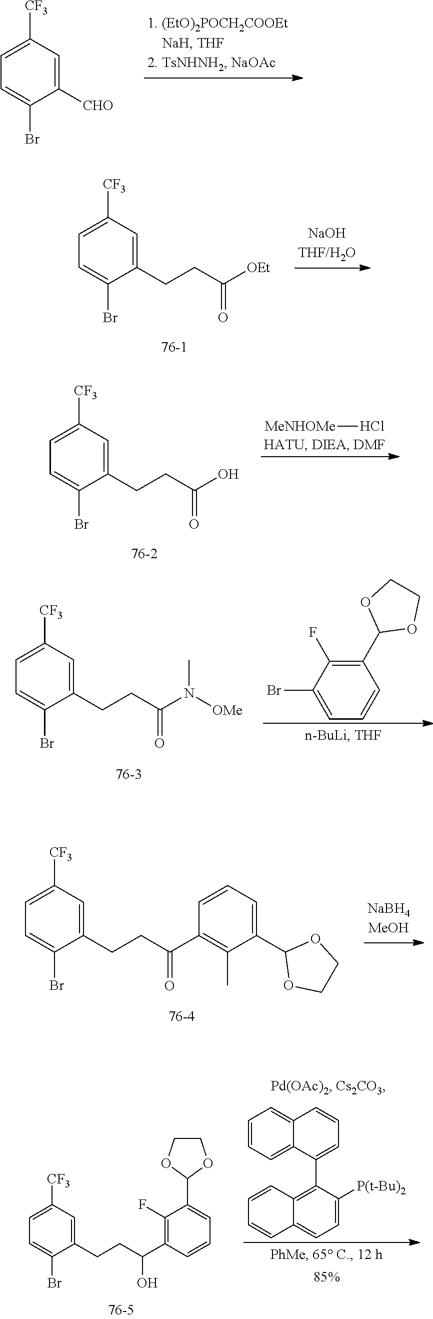

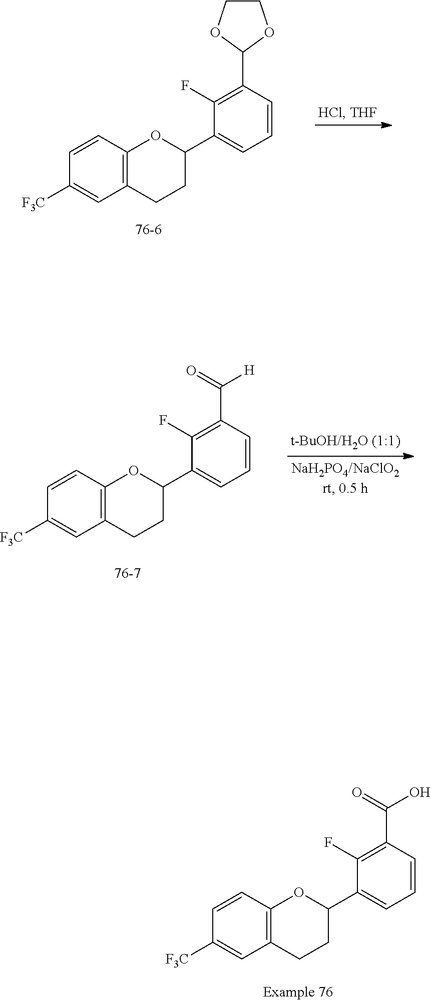 [SMILES: Fc1c(C2CCc3cc(C(F)(F)F)ccc3O2)cccc1C1OCCO1 ; O=Cc1cccc(C2CCc3cc(C(F)(F)F)ccc3O2)c1F ; O=C(O)c1cccc(C2CCc3cc(C(F)(F)F)ccc3O2)c1F]

Step 1: To a solution of NaH (40 mg, 2.18 mmol) in THF (10 ml) at 0° C. was added triethyl phosphonoacetate (531 mg, 2.37 mmol) dropwise at a rate of 0.25 mL/min. After addition was complete, the solution was warmed to room temperature over 25 min. The reaction was cooled back to 0° C. and 2-bromo-5-(trifluoromethyl)-benzaldehyde (500 mg, 1.97 mmol) was added. The reaction was warmed to room temperature and stirred for 30 min. The mixture was quenched with sat. aq NH₄Cl and extracted with Et₂O. The combined organic extracts were washed with brine, dried with MgSO₄, filtered, and concentrated in vacuo. The residue was dissolved in THF/HO(1:1, 10 mL). p-Toluenesulfonyl hydrazide (735 mg, 3.95 mmol) and NaOAc (486 mg, 5.93 mmol) were added. The mixture was heated to reflux for 20 h. The solution was cooled to room temperature, diluted with water, and extracted with EtOAc. The combined organic extracts were washed with brine, dried with MgSO₄, filtered, and concentrated in vacuo. Purification of the residue by silica flash chromatography (9:1 hexanes/EtOAc with 0.5% Et₃N) provided compound 76-1 (400 mg, 62% yield). ¹H NMR (400 MHZ, CDCl₃) δ 7.65-7.67 (dd, 1H), 7.51 (d, 1H), 7.32-7.35 (dd, 1H), 4.13 (q, 2H), 3.12 (t, 2H), 2.66 (t, 2H), 1.24 (t, 3H).

Step 2: To a solution of compound 76-1 (400 mg, 1.23 mmol) in THF/H₂O(1:1, 20 mL) was added NaOH (50 mg, 1.25 mmol). The mixture was stirred at 50° C. for 2 h. The reaction mixture was cooled to room temperature, adjusted to pH 2 with 1 M HCl, and extracted with EtOAc (2 x). The combined organic layer was washed the brine and concentrated in vacuo. The crude product 76-2 (400 mg) was used for next step without purification.

Step 3: To a solution of compound 76-2 (400 mg, 1.35 mmol) in DMF (2 ml) was added MeNHOMe-HCl (160 mg, 1.65 mmol), HATU (1.02 g, 2.68 mmol) and DIEA (1.024 g, 7.94 mmol). The reaction was stirred at room temperature for over 20 h. The mixture was diluted with EtOAc, washed with brine, and concentrated in vacuo. Purification of the residue with prep-TLC (PE: EtOAc=4:1) afforded compound 76-3 (280 mg, 60.7% yield).

Step 4: To a solution of 2-(3-bromo-2-fluorophenyl)-1,3-dioxolane (169 mg, 0.688 mmol) in THF (2 ml) at −78° C. under argon was added n-BuLi (0.7 ml, 1.6M) dropwise. The mixture was stirred at −78° C. for 1 h and compound 76-3 (280 mg,0.83 mmol) was added. The mixture was stirred at −78° C. for 5 min, then warmed to room temperature, and stirred for 3 h. The reaction was quenched with sat. aq. NH₄Cl and extracted with 5 mL of EtOAc. The organic layer was washed with H₂O(5 mL) and brine (5 mL), dried with anhydrous MgSO₄, and concentrated in vacuo. The residue was purified by chromatography on silica gel to give the ketone compound 76-4 (60 mg, 18.9% yield) as colorless oil. ¹H NMR (400 MHZ, CDCl₃) δ 7.88-7.93 (m, 1H), 7.73-7.77 (m, 1H), 7.66-7.68 (d, 1H), 7.56 (d, 1H), 7.25-7.29 (d, 1H), 6.12 (s, 1H), 4.16 (m, 2H), 4.08 (m, 2H), 3.37 (m, 2H), 3.24 (m, 2H).

Step 5: To a solution of compound 76-4 (60 mg, 0.135 mmol) in THF (0.5 ml) at 0° C. was added NaBH₄ (24 mg, 0.63 mmol). The mixture was stirred at room temperature overnight. The reaction mixture was quenched by careful addition of the aqueous MeOH and extracted with 5 mL of EtOAc. The organic layer was washed with H₂O(5 mL) and brine (5 mL), dried with anhydrous MgSO₄, and concentrated in vacuo. The residue was purified by chromatography on silica gel to give the alcohol compound 76-5 (51 mg, 88.5% yield) as colorless oil. ¹H NMR (400 MHZ, CDCl₃) δ 7.67-7.69 (d, 1H), 7.55-7.60 (m, 1H), 7.49-7.52 (m, 2H), 7.33-7.35 (d, 1H), 7.30-7.31 (d, 1H), 7.21-7.25 (t, 1H), 6.12 (s, 1H), 4.19 (m, 2H), 4.10 (m, 2H), 3.04 (m, 1H), 2.92 (m, 1H), 2.12 (m, 2H).

Step 6: Pd(OAc)₂ (15 mg, 0.067 mmol), rac-2-(di-t-butylphosphino)-1,1-binaphthyl (23 mg, 0.068 mmol) and Cs₂CO₃ (110 mg, 0.337 mmol) were dissolved in toluene (1 mL). A solution of compound 76-5 (51 mg, 0.114 mmol) in toluene (1 mL) was added under nitrogen atmosphere. The reaction mixture was heated at 65° C. for 12 h. The mixture was cooled to room temperature, diluted with EtOAc, and washed with H₂O and brine. The organic layer was dried with anhydrous MgSO₄ and concentrated in vacuo. The residue was purified by chromatography on silica gel to give compound 76-6 (36 mg, 85.1% yield). 1H NMR (400 MHZ, CDCl₃) δ 7.53-7.59 (m, 2H), 7.41-7.44 (m, 2H), 7.24-7.27 (t, 1H), 7.01-7.03 (d, 1H), 6.17 (s, 1H), 4.22 (m, 2H), 4.12 (m, 2H), 3.03-3.12 (m, 1H), 2.83-2.90 (m, 1H), 2.32-2.39 (m, 1H), 2.05-2.15 (m, 1H).

Step 7: To a solution of compound 76-6 (36 mg, 0.097 mmol) in THF (0.5 ml) at 0° C. was added conc. HCl (12 M, 1 mL). The mixture was stirred at room temperature for 1 h. The reaction mixture was quenched with water, and extracted with EtOAc. The organic layer was washed with water and brine, dried with anhydrous MgSO₄, and concentrated in vacuo. The residue was purified by chromatography on silica gel to give the compound 76-7 (27 mg, 85.9% yield) as colorless oil.

Step 8: To a solution of compound 76-7 (27 mg, 0.083 mmol) in t-BuOH/H₂O(1:1, 1 mL) was added 2-methyl-2-butene (113 mg, 1.61 mmol), NaH₂PO₄ (52 mg, 0.43 mmol) and NaClO₂ (39 mg, 0.43 mmol). The resulting mixture was stirred at room temperature for 0.5 h. The reaction mixture was diluted with EtOAc and washed with H₂O and brine. The collected organic layer was dried with anhydrous MgSO₄ and concentrated in vacuo. The residue was purified by chromatography on silica gel to give Example 76 (14 mg, 52% yield) as a white solid. LC-MS, ES⁻ (m/z): 339.30 (M−1). ¹H NMR (400 MHZ, DMSO) δ 7.84-7.86 (m, 1H), 7.72-7.75 (m, 1H), 7.71 (s, 1H), 7.49-7.56 (t, 1H), 7.47-7.34 (t, 1H), 7.04-7.06 (d, 1H), 6.17 (s, 1H), 4.22 (m, 2H), 4.12 (m, 2H), 3.03-3.12 (m, 1H), 2.83-2.90 (m, 1H), 2.32-2.39 (m, 1H), 2.05-2.15 (m, 1H).

### Examples 77-85

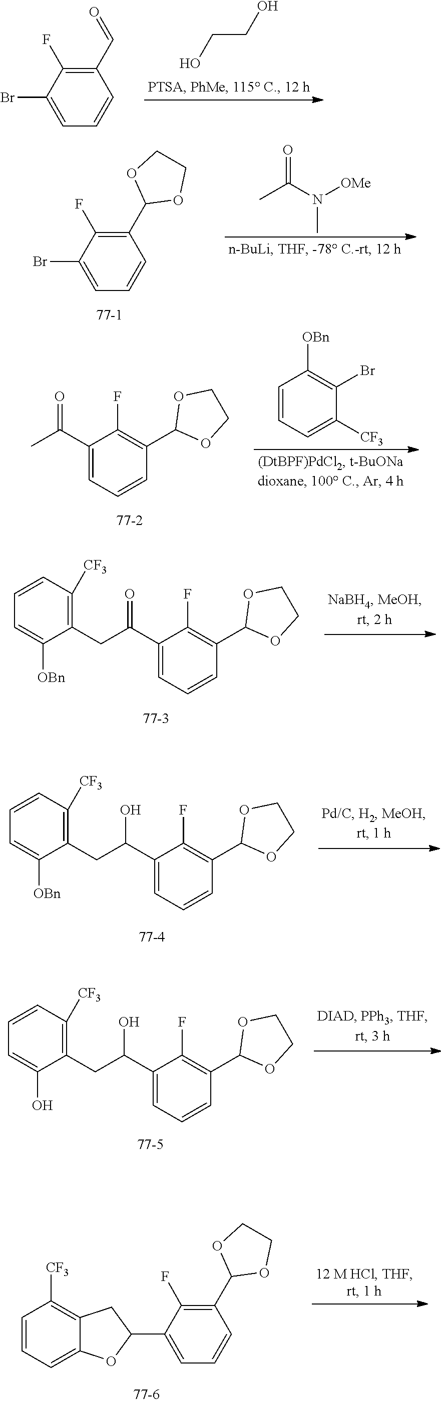

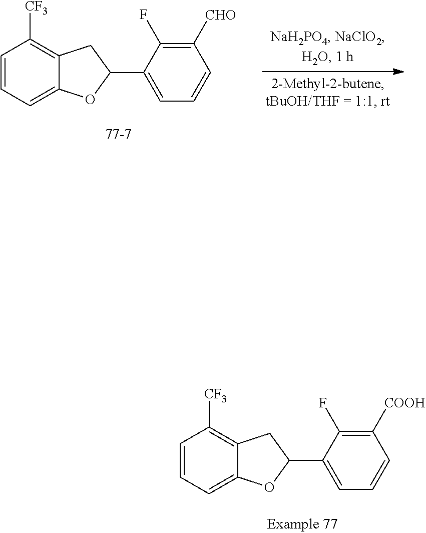 [SMILES: O=Cc1cccc(C2Cc3c(cccc3C(F)(F)F)O2)c1F ; O=C(O)c1cccc(C2Cc3c(cccc3C(F)(F)F)O2)c1F]

Step 1:3-Bromo-2-fluorobenzaldehyde (2 g, 9.85 mmol), PTSA·H₂O(187 mg, 0.985 mmol) and ethylene glycol (2.44 g, 39.4 mmol) were dissolved in toluene (30 mL) and the resulting solution was heated under reflux for 12 h. After complete conversion, the reaction mixture was cooled to room temperature. The reaction was quenched with water and extracted with EtOAc for three times. The combined organic layer was dried over anhydrous Na₂SO₄, filtered, and concentrated in vacuo. Purification of the residue via column chromatography afforded 77-1 (2.216 g, 91% yield) as colorless oil. LC-MS, ES⁻ (m/z): 247.00 (M+1).

Step 2:2 (3-bromo-2-fluorophenyl)-1,3-dioxolane (77-1)(5.0 g, 20.24 mmol) was dissolved in THF (30 mL) and cooled down to −78° C. under Ar atmosphere. A solution of n-BuLi (2.5 M in hexane, 16.2 mL, 40.49 mmol) was added dropwise to the above solution at −78° C. The resulting solution was stirred for 1 h at this temperature. N-methoxy-N-methylacetamide (4.17 g, 40.49 mmol, dissolved in 30 mL THF) was added at −78° C. After stirring at −78° C. for 30 min, the solution was warmed to room temperature and stirred overnight. The reaction was quenched with sat. NH₄Cl (30 mL) and extracted with EtOAc three times. The organic fractions were combined, washed with NaCl, and concentrated in vacuum. The residue was purified by chromatography on silica gel to afford 77-2 (2.15 g, 50% yield) as yellow oil. LC-MS, ES⁻(m/z) 211.07 (M+1). ¹H NMR (400 MHZ, Chloroform-d) δ 7.93-7.83 (m, 1H), 7.77-7.68 (m, 1H), 7.22 (d, J=7.7 Hz, 1H), 6.12 (s, 1H), 4.19-4.12 (m, 2H), 4.10-4.03 (m, 2H), 2.65 (d, J=5.2 Hz, 3H).

Step 3:3-(DtBPF) PdCl₂ (62 mg, 0.095 mmol), t-BuONa (230 mg, 2.38 mmol), 1-(3-(1,3-dioxolan-2-yl)-2-fluorophenyl)ethan-1-one (77-2)(200 mg, 0.95 mmol) and 1-(benzyloxy)-2-bromo-3-(trifluoromethyl)benzene (380 mg, 1.14 mmol) were dissolved in 8 mL of 1,4-dioxane. The mixture was heated at 100° C. for 4 h, and then cooled to room temperature. The mixture was diluted with 10 mL of EtOAc and washed with H₂O(10 mL) and brine (10 mL). The organic layer was dried over anhydrous MgSO₄ and concentrated in vacuo. The residue was purified by chromatography on silica gel to give 77-3 (238 mg, 54% yield) as brown oil. LC-MS, ES⁻(m/z) 461.13 (M+1). ¹H NMR (400 MHZ, Chloroform-d) δ 7.74-7.69 (m, 2H), 7.32 (d, J=10.9 Hz, 2H), 7.27-7.16 (m, 6H), 7.14-7.07 (m, 1H), 6.14 (s, 1H), 5.07 (s, 2H), 4.54 (s, 2H), 4.19-4.13 (m, 2H), 4.09 (d, J=7.1 Hz, 2H).

Step 4: Compound 77-3 (260 mg, 0.57 mmol) was dissolved in CH₃OH (6.0 mL). NaBH₄ (64 mg, 1.70 mmol) was added. The mixture was stirred at room temperature for 2 h, quenched with sat. NaCl (6 mL), and extracted with EtOAc for three times. The combined organic layers were dried over anhydrous Na₂SO₄, filtered, and concentrated in vacuo. The residue was purified by prep-TLC to afford 77-4 (69.3 mg, 26% yield) as yellow oil. LC-MS, ES⁻ (m/z): 463.15 (M+1). ¹H NMR (400 MHZ, Chloroform-d) δ 7.49-7.30 (m, 9H), 7.19-7.05 (m, 2H), 6.06 (s, 1H), 5.28 (d, J=9.9 Hz, 1H), 5.21-5.05 (m, 2H), 4.13 (d, J=2.8 Hz, 2H), 4.03 (s, 2H), 3.43-3.22 (m, 2H).

Step 5: To a solution of compound 77-4 (69.3 mg, 0.15 mmol) in MeOH (5 mL) was added 5% Pd/C (95 mg, 0.045 mmol) and the mixture was stirred at room temperature under H₂ atmosphere for 1 h. TLC showed complete consumption of the starting material. The reaction mixture was filtered and the filtrate was concentrated in vacuum. The residue was purified by prep-TLC to give 77-5 (27.0 mg, 48% yield) as white solid. LC-MS, ES⁻ (m/z): 373.10 (M+1). ¹H NMR (400 MHZ, Chloroform-d) δ 7.60-7.44 (m, 2H), 7.27-7.10 (m, 4H), 6.07 (s, 1H), 5.29 (d, J=9.9 Hz, 1H), 4.17-4.09 (m, 2H), 4.06-3.99 (m, 2H), 3.27-3.07 (m, 2H).

Step 6: To a solution of compound 77-5 (27.0 mg, 0.073 mmol) and PPh₃ (23.0 mg, 0.087 mmol) in THF (2 mL) was added DIAD (18.0 mg, 0.087 mmol) under Ar atmosphere. The mixture was stirred at room temperature for three hours. Solvent was removed in vacuum. The residue was taken up in water (1 mL) and extracted with EtOAc (2 mL×2). The combined organic layer was washed with water (2 mL) and brine (2 mL), dried over anhydrous Na₂SO₄, and concentrated in vacuum. The crude product was purified by silica gel flash chromatography to afford 77-6 (11.6 mg, 45% yield) as yellow oil. LC-MS, ES⁻ (m/z): 355.09 (M+1). ¹H NMR (400 MHZ, Chloroform-d) δ 7.56-7.42 (m, 2H), 7.30-7.23 (m, 1H), 7.21-7.09 (m, 2H), 7.04 (d, J=8.0 Hz, 1H), 6.17-5.97 (m, 2H), 4.16 (t, J=6.9 Hz, 2H), 4.08-4.03 (m, 2H), 3.93-3.80 (m, 1H), 3.36-3.25 (m, 1H).

Step 7: To a solution of compound 77-6 (11.6 mg, 0.033 mmol) in THF (1 mL) was added 12 M HCl (2 drops) and the mixture was stirred at room temperature for 1 h. TLC showed complete consumption of the starting material. The reaction mixture was quenched with sat. NaCl and was extracted with EtOAc for three times. The combined organic layers were dried over anhydrous Na₂SO₄, filtered, and concentrated in vacuum. The residue was purified by prep-TLC to give 77-7 (9.7 mg, 95% yield) as yellow solid. LC-MS, ES⁻ (m/z): 311.06 (M+1). ¹H NMR (400 MHZ, Chloroform-d) δ 10.38 (s, 1H), 7.84 (t, J=8.1 Hz, 1H), 7.73 (t, J=8.2 Hz, 1H), 7.33-7.26 (m, 2H), 7.16 (d, J=7.8 Hz, 1H), 7.07 (d, J=8.1 Hz, 1H), 6.22-5.90 (m, 1H), 3.98-3.88 (m, 1H), 3.38-3.26 (m, 1H).

Step 8: Compound 77-7 (9.7 mg, 0.031 mmol) and 2-Methyl-2-butene (22.0 mg, 0.31 mmol) was dissolved in THF (0.5 mL) and t-BuOH (0.5 mL). A solution of NaH₂PO₄ (19.0 mg, 0.156 mmol) and NaClO₂ (14.0 mg, 0.156 mmol) in 0.25 mL of H₂O was added to the above solution at 0° C. The reaction was stirred at room temperature for 1 h. The reaction mixture was diluted with 2 mL EtOAc, washed with H₂O(2 mL) and brine (2 mL), dried with anhydrous MgSO₄, and concentrated. The residue was purified by prep-TLC to give Example 77 (2.8 mg, 28% yield) as white solid. LC-MS, ES⁻(m/z) 325.00 (M−1). 1H NMR (400 MHZ, Methanol-d₄) δ 7.89 (d, J=7.6 Hz, 1H), 7.64 (t, J=7.3 Hz, 1H), 7.33 (t, J=7.8 Hz, 1H), 7.25 (t, J=7.8 Hz, 1H), 7.15 (d, J=7.7 Hz, 1H), 7.10 (d, J=8.0 Hz, 1H), 6.16-6.06 (m, 1H), 3.96-3.84 (m, 1H), 3.27-3.21 (m, 1H).

The following examples were prepared employing the same protocol as described in Example 77.

| Example | Structure | NMR | MS |
|---|---|---|---|
| 78 | 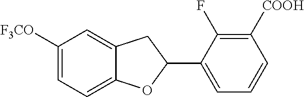 [SMILES: O=C(O)c1cccc(C2Cc3cc(OC(F)(F)F)ccc3O2)c1F] | ¹H NMR (400 MHz, Methanol-d₄) δ 7.87 (t, J = 7.8 Hz, 1H), 7.65 (t, J = 7.7 Hz, 1H), 7.24 (t, J = 7.8 Hz, 1H), 7.13 (s, 1H), 7.05 (d, J = 10.3 Hz, 1H), 6.87 (d, J = 8.7 Hz, 1H), 6.12-6.04 (m, 1H), 3.83-3.71 (m, 1H), 3.21-3.12 (m, 1H). | ES⁻ (m/z): 340.97 (M − 1) |
| 79 | 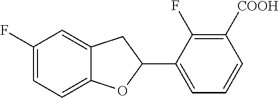 [SMILES: O=C(O)c1cccc(C2Cc3cc(F)ccc3O2)c1F] | ¹H NMR (400 MHz, Methanol-d₄) δ 7.85 (td, J = 7.6, 2.0 Hz, 1H), 7.68-7.61 (m, 1H), 7.23 (t, J = 7.6 Hz, 1H), 6.94 (dd, J = 8.4, 2.8 Hz, 1H), 6.85 (td, J = 8.8, 2.8 Hz, 1H), 6.79 (dd, J = 8.8, 4.2 Hz, 1H), 6.07-5.98 (m, 1H), 3.74 (dd, J = 16.0, 9.6 Hz, 1H), 3.12 (dd, J = 16.0, 7.6 Hz, 1H). | ES⁻ (m/z): 274.93 (M − 1) |
| 80 | 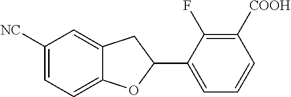 [SMILES: N#Cc1ccc2c(c1)CC(c1cccc(C(=O)O)c1F)O2] | ¹H NMR (400 MHz, Methanol-d₄) δ 7.88 (td, J = 7.6, 2.0 Hz, 1H), 7.65-7.59 (m, 1H), 7.58- 7.52 (m, 2H), 7.25 (t, J = 7.6 Hz, 1H), 6.98 (d, J = 8.4 Hz, 1H), 6.18-6.10 (m, 1H), 3.80 (dd, J = 16.4, 10.0 Hz, 1H), 3.21 (dd, J = 16.4, 8.0 Hz, 1H). | ES⁻ (m/z): 281.93 (M − 1) |
| 81 | 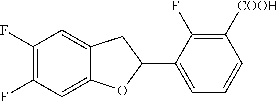 [SMILES: O=C(O)c1cccc(C2Cc3cc(F)c(F)cc3O2)c1F] | ¹H NMR (400 MHz, Methanol-d₄) δ 7.93-7.82 (m, 1H), 7.68-7.60 (m, 1H), 7.24 (t, J = 7.7 Hz, 1H), 7.14-7.06 (m, 1H), 6.82-6.72 (m, 1H), 6.12-6.01 (m, 1H), 3.77-3.67 (m, 1H), 3.16- 3.07 (m, 1H). | ES⁻ (m/z): 292.93 (M − 1) |
| 82 | 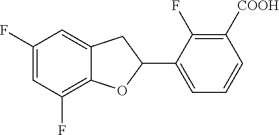 [SMILES: O=C(O)c1cccc(C2Cc3cc(F)cc(F)c3O2)c1F] | ¹H NMR (400 MHz, Methanol-d₄) δ 7.93-7.85 (m, 1H), 7.66 (t, J = 7.2 Hz, 1H), 7.26 (t, J = 7.7 Hz, 1H), 6.82 (t, J = 9.0 Hz, 2H), 6.17-6.08 (m, 1H), 3.84-3.75 (m, 1H), 3.25-3.15 (m, 1H). | ES⁻ (m/z): 292.94 (M − 1) |
| 83 | 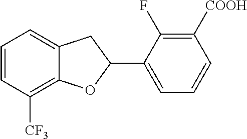 [SMILES: O=C(O)c1cccc(C2Cc3cccc(C(F)(F)F)c3O2)c1F] | ¹H NMR (400 MHz, Methanol-d₄) δ 7.88 (t, J = 8.2 Hz, 1H), 7.61 (t, J = 8.0 Hz, 1H), 7.40 (dd, J = 17.9, 7.7 Hz, 2H), 7.24 (t, J = 7.8 Hz, 1H), 6.99 (t, J = 7.7 Hz, 1H), 6.21-6.09 (m, 1H), 3.91 -3.74 (m, 1H), 3.24-3.11 (m, 1H). | ES⁻ (m/z): 325.00 (M − 1) |
| 84 | 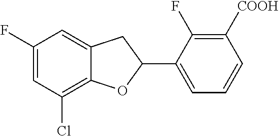 [SMILES: O=C(O)c1cccc(C2Cc3cc(F)cc(Cl)c3O2)c1F] | ¹H NMR (400 MHz, Methanol-d₄) δ 7.93-7.85 (m, 1H), 7.68-7.60 (m, 1H), 7.26 (t, J = 7.7 Hz, 1H), 7.01-6.91 (m, 2H), 6.16-6.09 (m, 1H), 3.89-3.78 (m, 1H), 3.27-3.18 (m, 1H). | ES⁻ (m/z): 308.93 (M − 1) |
| 85 | 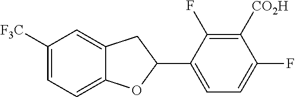 [SMILES: O=C(O)c1c(F)ccc(C2Cc3cc(C(F)(F)F)ccc3O2)c1F] | ¹H NMR (400 MHz, Methanol-d₄) δ 7.95-7.82 (m, 1H), 7.64 (t, J = 7.4 Hz, 1H), 7.53-7.41 (m, 1H), 7.24 (t, J = 7.8 Hz, 1H), 6.97 (d, J = 8.4 Hz, 1H), 6.20-6.06 (m, 1H), 3.87-3.74 (m, 1H), 3.26-3.14 (m, 1H). | ES⁻ (m/z): 343.05 (M − 1) |

### Example 86

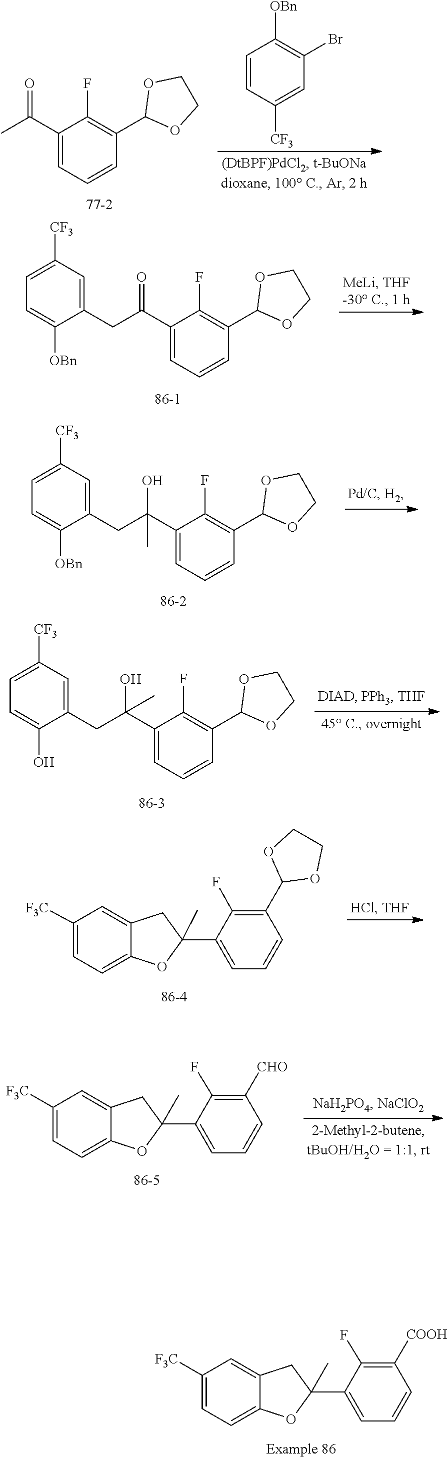

Step 1:2-(DtBPF) PdCl₂ (15.5 mg, 0.024 mmol), t-BuONa (228.3 mg, 2.38 mmol), 1-(3-(1,3-dioxolan-2-yl)-2-fluorophenyl)ethan-1-one (77-2)(239.7 mg, 1.14 mmol) and 1-(benzyloxy)-2-bromo-4-(trifluoromethyl)benzene (314.7 mg, 0.95 mmol) were dissolved in 8 mL of 1,4-dioxane. The mixture was heated at 100° C. for 2 h, and then cooled to room temperature. The mixture was diluted with 10 mL of EtOAc and washed with H₂O(10 mL) and brine (10 mL). The organic layer was dried over anhydrous MgSO₄ and concentrated in vacuo. The residue was purified by chromatography on silica gel to give 86-1 (277.6 mg, 63% yield) as brown oil. LC-MS, ES⁺ (m/z): 461.13 (M+1).

Step 2: Compound 86-1 (42.0 mg, 0.091 mmol) was dissolved in anhydrous THF (8.0 mL). The solution was cooled to −30° C., then 1.6 M MeLi in THF (171 uL, 0.23 mmol) was added dropwise. The mixture was stirred at −30° C. for 1 h, quenched with H₂O(8 mL), and extracted with EtOAc for three times. The combined organic layers were dried over anhydrous Na₂SO₄, filtered, and concentrated in vacuo. The crude product 86-2 was used in next step without further purification. ¹H NMR (400 MHZ, Chloroform-d) δ 7.27-7.23 (m, 9H), 7.11 (m, 2H), 6.89 (m, 2H), 6.79 (m, 2H), 5.94 (s, 1H), 4.97 (s, 2H), 3.98 (m, 2H), 3.90 (m, 2H), 1.46 (s, 3H).

Step 3: To a solution of compound 86-2 (33.6 mg, 0.07 mmol) in MeOH (5 mL) was added 5% Pd/C (10 mg) and the mixture was stirred at room temperature under H₂ atmosphere for 1 h. TLC showed complete consumption of the starting material. The reaction mixture was filtered and the filtrate was concentrated in vacuum. The residue was purified by prep-TLC to give 86-3 (23.2 mg, 83% yield) as yellow oil. LC-MS, ES⁺ (m/z): 459.24 (M-18+1). ¹H NMR (400 MHZ, Chloroform-d) δ 7.47 (m, 2H), 7.32 (dd, J=8, 4 Hz, 2H), 7.19 (d, J=4 Hz, 1H), 7.08 (t, J=8 Hz, 1H), 6.91 (d, J=8 Hz, 2H), 6.12 (s, 1H), 4.12 (m, 2H), 4.06 (m, 2H), 3.30 (m, 1H), 3.17 (m, 1H), 1.67 (s, 3H).

Step 4: To a solution of compound 86-3 (23.2 mg, 0.060 mmol) and PPh₃ (18.9 mg, 0.072 mmol) in THF (3 mL) was added DIAD (14.6 mg, 0.072 mmol) under Ar atmosphere. The mixture was stirred at 45° C. overnight. Solvent was removed in vacuum. The residue was taken up in water (1 mL) and extracted with EtOAc (2 mL×2). The combined organic layer was washed with water (2 mL) and brine (2 mL), dried over anhydrous Na₂SO₄, and concentrated in vacuum. The crude product was purified by s prep-TLC to give 86-4 (14.1 mg, 63% yield) as yellow oil. ¹H NMR (400 MHZ, Chloroform-d) δ 7.59 (m, 2H), 7.48 (m, 2H), 7.38 (m, 3H), 7.15 (t, J=8 Hz, 1H), 6.94 (d, J=8 Hz, 1H), 6.12 (s, 1H), 4.12 (m, 2H), 4.06 (m, 2H), 3.51 (s, 2H), 1.80 (s, 3H).

Step 5: To a solution of compound 86-4 (31.5 mg, 0.085 mmol) in THF (3 mL) was added 12 M HCl (100 uL) and the mixture was stirred at room temperature for 1 h. TLC showed complete consumption of the starting material. The reaction mixture was quenched with sat. NaCl and extracted with EtOAc for three times. The combined organic layers were dried over anhydrous Na₂SO₄, filtered, and concentrated in vacuum. The residue was purified by prep-TLC to give 86-5 (26.6 mg, 95% yield) as yellow solid. ¹H NMR (400 MHZ, Chloroform-d) δ 10.04 (s, 1H), 7.90 (m, 3H), 7.50 (m, 4H), 6.97 (m, 1H), 3,74 (m, 2H), 3.58 (m, 2H), 1.87 (s, 3H).

Step 6: Compound 86-5 (26.6 mg, 0.082 mmol) and 2-Methyl-2-butene (86.0 mg, 1.23 mmol) was dissolved in H₂O(1.5 mL) and t-BuOH (1.5 mL). NaH₂PO₄ (39.4 mg, 0.328 mmol) and NaClO₂ (30.0 mg, 0.328 mmol) was added to the above solution at room temperature. The reaction was stirred at room temperature for 1 h. The reaction mixture was diluted with 2 mL EtOAc, washed with H₂O(2 mL) and brine (2 mL), dried with anhydrous MgSO₄, and concentrated. The residue was purified by prep-TLC to give Example 86 (9.4 mg, 33% yield) as yellow solid. LC-MS, ES⁻ (m/z): 339.03 (M−1). ¹H NMR (400 MHZ, 400 MHZ, Chloroform-d) δ 7.98 (m, 1H), 7.90 (m, 1H), 7.45 (d, J=8 Hz, 1H), 7.41 (s, 1H), 7.25 (m, 1H), 6.97 (d, J=12.0 Hz, 1H), 3.57 (m, 2H), 1.83 (s, 3H).

### Example 87

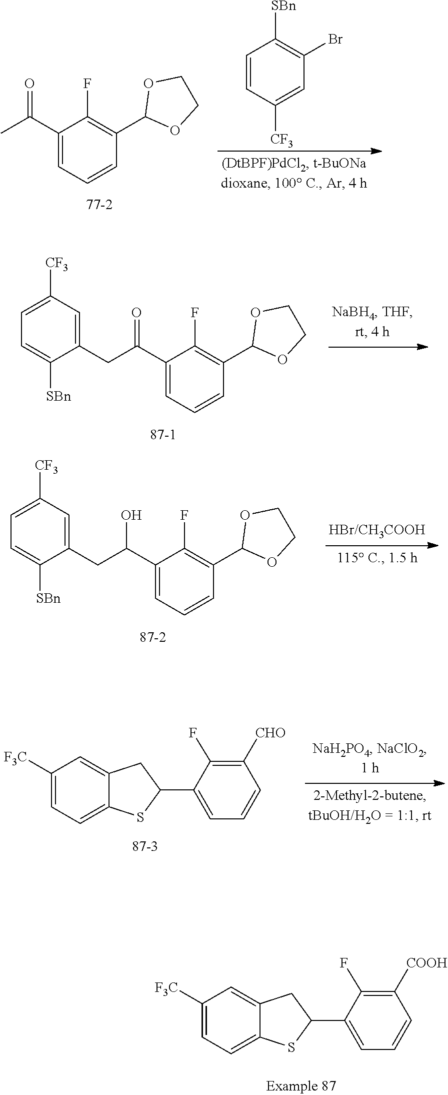

Step 1:3-(DtBPF) PdCl₂ (28 mg, 0.043 mmol), t-BuONa (207 mg, 2.161 mmol), 1-(3-(1,3-dioxolan-2-yl)-2-fluorophenyl)ethan-1-one (77-2)(218 mg, 1.037 mmol) and 1-benzylsulfanyl-2-bromo-4-(trifluoromethyl)benzene (300 mg, 0.864 mmol) were dissolved in 10 mL of 1,4-dioxane. The mixture was heated at 100° C. for 3 h, and then cooled to room temperature. The mixture was quenched with saturated ammonium chloride aqueous solution, then extracted with EtOAc for three times. The combined organic layer was dried over anhydrous Na₂SO₄, filtered and concentrated in vacuo. The residue was purified by chromatography on silica gel to give 87-1 (193 mg, 57% yield.

Step 2: Compound 87-1 (193 mg, 0.405 mmol) was dissolved in THF (10.0 mL). NaBH₄ (61 mg, 1.622 mmol) was added. The mixture was stirred at room temperature for 4h, quenched with H₂O, and extracted with EtOAc for three times. The combined organic layers were dried over anhydrous Na₂SO₄, filtered, and concentrated in vacuo. The residue was purified by chromatography on silica gel to give 87-2 (160 mg, 82% yield) as yellow oil.

Step 3: Hydrogen bromide (33 wt % solution in acetic acid, 5 ml) was added to compound 87-2 (160 mg, 0.335 mmol) under argon. The mixture was heated at 115° C. for 2 h, and cooled to room temperature. The mixture was concentrated in vacuo, then 5 ml of H₂O was added. The resulting mixture was extracted with EtOA for three times. The combined organic layers were dried over anhydrous Na₂SO₄, filtered and concentrated in vacuo. The residue was purified by chromatography on silica gel to give 87-3 (80.8 mg, 74% yield). ¹H NMR (400 MHZ, Chloroform-d) δ 10.38 (s, 1H), 7.80 (m, 1H), 7.73 (m, 1H), 7.44 (m, 2H), 7.32 (m, 1H), 7.22 (m, 1H), 5.41 (q, J-8.0 Hz, 1H), 3.85-3.79 (m, 1H), 3.52-3.45 (m, 1H).

Step 4: Compound 87-3 (80.8 mg, 0.245 mmol) and 2-Methyl-2-butene (22.0 mg, 0.31 mmol) were dissolved in t-BuOH (2.5 mL) and H₂O(2.5 ml). NaH₂PO₄ (117.7 mg, 0.981 mmol) and NaClO₂ (88.7 mg, 0.981 mmol) were added. More 2-Methyl-2-butene (257.7 mg, 3.675 mmol) was added. The reaction was stirred at room temperature for 1.5 h, quenched with water, and extracted with EtOAc for three times. The combined organic layers were dried over anhydrous Na₂SO₄, filtered, and concentrated in vacuo. The residue was purified by chromatography on silica gel to give Example 87 (36 mg, 43% yield) as white solid. LC-MS, ES⁻(m/z) 341.00 (M−1). ¹H NMR (400 MHz, Dimethyl Sulfoxide-D6) δ 13.3 (s, 1H), 7.77 (m, 1H), 7.65 (m, 2H), 7.50 (m, 2H), 7.24 (t, J=8.0 Hz, 1H), 5.45 (q, J=8.0 Hz, 1H), 3.85-3.74 (m, 1H), 3.67-3.60 (m, 1H).

### Examples 88-91

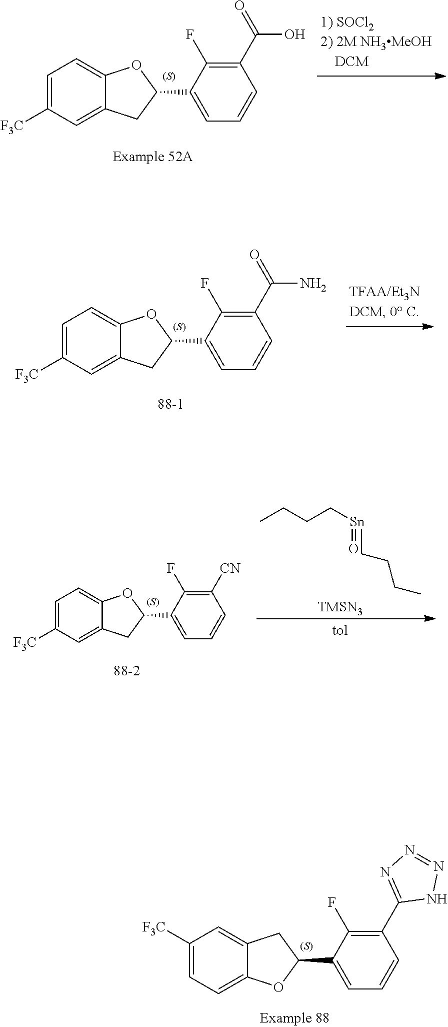

Step 1: Compound Example 52A (500 mg, 1.533 mmol) was dissolved in SOCl₂ (5 mL) and the solution was refluxed at 60° C. overnight. The reaction mixture was concentrated under reduced pressure. The residue of(S)-2-fluoro-3-(5-(trifluoromethyl)-2,3-dihydrobenzofuran-2-yl)benzoyl chloride was dissolved in DCM (5 mL), cooled to 0° C., and a solution of 2M NH₃ in MeOH (1.5 mL, 3.067 mmol) was added. The mixture was stirred at rt for 10 min, quenched with H₂O, and extracted with EtOAc for three times. The combined organic layers were dried over anhydrous Na₂SO₄, filtered, and concentrated under reduced pressure. Compound 88-1 (526 mg, 105% yield) was obtain as colorless oil and used without further purification.

Step 2: To a solution of 88-1 (526 mg, 1.618 mmol) in DCM (5 mL) at 0° C. was added TEA (706 mg, 6.472 mmol) and TFAA (548 mg, 1.942 mmol) dropwise. The mixture was stirred at rt for 2 hours, quenched with H₂O, and extracted with EtOAc for three times. The combined organic layers were dried over anhydrous Na₂SO₄, filtered, and concentrated under reduced pressure. The residue was purified by column chromatography to give the compound 88-2 (440 mg, 88.20% yield) as yellow oil. ¹H NMR (400 MHZ, Chloroform-d) δ 7.77 (dddd, J=7.8, 7.0, 1.8, 0.7 Hz, 1H), 7.65 (ddd, J=7.7, 6.0, 1.7 Hz, 1H), 7.55-7.47 (m, 2H), 7.37-7.28 (m, 1H), 7.01 (d, J=8.3 Hz, 1H), 6.13 (dd, J=9.8, 7.5 Hz, 1H), 3.86 (dd, J=16.0, 9.8 Hz, 1H), 3.27-3.17 (m, 1H).

Step 3: To a solution of 88-2 (440 mg, 1.429 mmol) in toluene (5 mL) was added TMSN₃ (332 mg, 2.858 mmol) and dibutyltin oxide (36 mg, 0.1429 mmol) under N₂. The mixture was stirred at 110° C. for 12 hours. MeOH (5 mL) was added. The mixture was stirred for 30 min, washed with H₂O, and extracted with EtOAc for three times. The combined organic layers were dried over anhydrous Na₂SO₄, filtered, and concentrated under reduced pressure. The residue was purified by column chromatography to give compound Example 88 (200 mg, 40% yield) as a white solid. LC-MS (ESI): m/z=349.27 [M−H]. ¹H NMR (400 MHZ, Methanol-d₄) δ 8.19-8.11 (m, 1H), 7.75 (td, J=7.5, 1.8 Hz, 1H), 7.60-7.42 (m, 3H), 7.04 (d, J=8.4 Hz, 1H), 6.24 (dd, J=9.9, 7.6 Hz, 1H), 3.91 (dd, J=16.2, 9.9 Hz, 1H), 3.38 (dd, J=8.5 Hz, 1H). In some embodiments, compound 88 was further reacted with NaOH in H₂O to provide a sodium salt

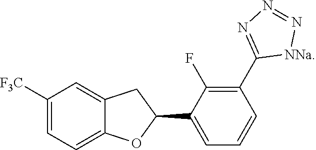

Various salts including pharmaceutically acceptable salts for various compounds herein can be similarly prepared, or using other available suitable technologies in accordance with the present disclosure.

The following examples were synthesized employing similar protocol as described in Example 88:

| Example | Structure | NMR | MS |
|---|---|---|---|
| 89 | 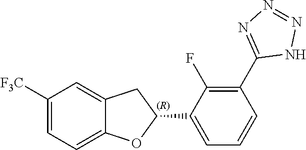 | ¹H NMR (400 MHz, Methanol-d₄) δ 8.19- 8.11 (m, 1H), 7.75 (td, J = 7.5, 1.8 Hz, 1H), 7.60-7.42 (m, 3H), 7.04 (d, J = 8.4 Hz, 1H), 6.24 (dd, J = 9.9, 7.6 Hz, 1H), 3.91 (dd, J = 16.2, 9.9 Hz, 1H), 3.38 (dd, J = 8.5 Hz, 1H). | 349.27 [M − H]⁻ |
| 90 | 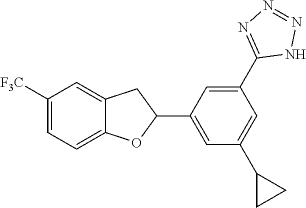 [SMILES: FC(F)(F)c1ccc2c(c1)CC(c1cc(-c3nnn[nH]3)cc(C3CC3)c1)O2] | ¹H NMR (400 MHz, Methanol-d₄) δ 7.84 (t, J = 1.6 Hz, 1H), 7.67 (t, J = 1.6 Hz, 1H), 7.50 (s, 1H), 7.47 (d, J = 8.4 Hz, 1H), 7.35-7.32 (m, 1H), 6.97 (d, J = 8.4 Hz, 1H), 5.97-5.89 (m, 1H), 3.78 (dd, J= 16.0, 9.6 Hz, 1H), 3.23 (dd, J = 16.0, 8.4 Hz, 1H), 2.05-1.98 (m, 1H), 1.07-1.02 (m, 2H), 0.81-0.76 (m, 2H). | 371.10 [M − 1]⁻ |
| 91 | 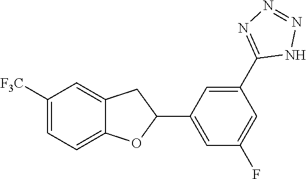 [SMILES: Fc1cc(-c2nnn[nH]2)cc(C2Cc3cc(C(F)(F)F)ccc3O2)c1] | ¹H NMR (400 MHz, Methanol-d₄) δ 7.94 (s, 1H), 7.77-7.70 (m, 1H), 7.54-7.45 (m, 2H), 7.37 (d, J = 9.6 Hz, 1H), 7.01 (d, J = 8.4 Hz, 1H), 6.04-5.95 (m, 1H), 3.83 (dd, J = 16.1, 9.6 Hz, 1H), 3.27-3.21 (m, 1H). | 349.28 [M − 1]⁻. |

### Example 91a: Another Synthesis of 88-2

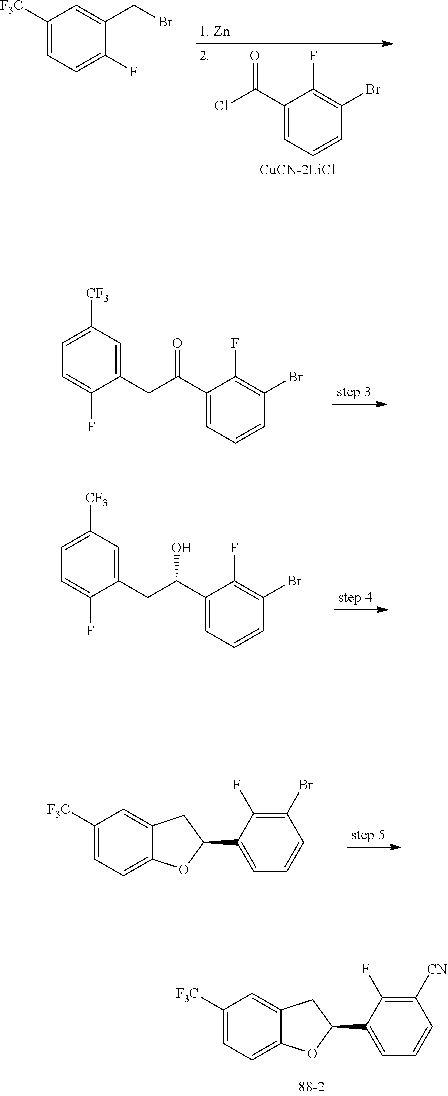

Step 1: A suspension of Zn powder (560 mg, 8.54 mmol) in anhydrous THF (6 mL) was heated to reflux. 1,2-Dibromoethane (72 mg, 0.39 mmol) was added followed by addition of TMSCl (9 mg, 0.082 mmol) to activate Zn. After cooling to 10-20° C., 2-(bromomethyl)-1-fluoro-4-(trifluoromethyl)benzene (2 g, 7.78 mmol) was dropwise added at the same temperature. After completion of the addition, the mixture was rigorously stirred at 10-20° C. for 5 h. The resulting solution was used in the next step.

Step 2: To a 0.6 M solution of CuCN·2LiCl (14.2 mL, 8.54 mmol) precooled to −30° C. was added the above obtained benzylzine bromide solution (7.78 mmol) during which the temperature was maintained between −30 to −20° C. The resulting mixture was stirred at the same temperature for 30 min and then cooled to −40° C., followed by addition of 3-bromo-2-fluorobenzoyl chloride (4.56 mmol) at −40 to −30° C. After addition, the mixture was allowed to warm slowly to room temperature overnight. Then the reaction was quenched with NH₄Cl (0.84 g) in water (2 mL) at 10-20° C. The suspension was stirred for 3 h at rt before filtration through a pad of Celite. The filtrate was concentrated to dryness. The filter cake was washed with DCM. The concentration residue was redissolved in the DCM filtrate, washed with water, dried over Na₂SO₄, filtered and concentrated. The crude product was purified by column chromatography to provide 1-(3-bromo-2-fluorophenyl)-2-(2-fluoro-5-(trifluoromethyl)phenyl)ethan-1-one (1.58 g, 92%) as a white solid. ¹H NMR (400 MHZ, Chloroform-d) δ 7.89 (ddd, J=8.0, 6.4, 1.7 Hz, 1H), 7.84 (ddd, J=8.1, 6.4, 1.8 Hz, 1H), 7.64 (ddd, J=8.0, 4.7, 2.3 Hz, 1H), 7.57 (dd, J=6.7, 2.3 Hz, 1H), 7.30-7.18 (m, 2H), 4.44 (dd, J=3.1, 1.2 Hz, 2H).

Step 3:1-(3-Bromo-2-fluorophenyl)-2-(2-fluoro-5-(trifluoromethyl)phenyl)ethan-1-one (714 mg, 1.88 mmol) and RuCl (p-cymene) [(S,S)-Ts-DPEN](6 mg, 0.0095 mmol) were charged into a 10 mL flask which was then evacuated and backfilled with N₂ for 3 times. THF (3.6 mL) and TEA (1.71 g, 16.9 mmol) were added. The mixture was cooled in an ice-water bath followed by dropwise addition of HCO₂H (780 mg, 17 mmol) at <20° C. Then the mixture was stirred at 40° C. for 15 h. The reaction was quenched with water and extracted with EA. The combined organic phases were dried over Na₂SO₄, filtered and concentrated. The residue was purified by column chromatography to give(S)-1-(3-bromo-2-fluorophenyl)-2-(2-fluoro-5-(trifluoromethyl)phenyl)ethan-1-ol (714 mg, 99%) as a white solid. The enantiomeric ratio was determined to be 92:8 by chiral HPLC. ¹H NMR (400 MHZ, Chloroform-d) δ 7.54-7.40 (m, 5H), 7.06 (td, J=7.9, 1.0 Hz, 1H), 5.38-5.30 (m, 1H), 3.28 (dd, J=13.8, 4.3 Hz, 1H), 3.14 (dd, J=13.8, 8.8 Hz, 1H).

Step 4: A mixture of(S)-1-(3-bromo-2-fluorophenyl)-2-(2-fluoro-5-(trifluoromethyl)phenyl)ethan-1-ol (690 mg, 1.81 mmol), NaOH particle (145 mg, 3.62 mmol) and 15-crown-5 (0.8 g, 3.62 mmol) in toluene (7 mL) was stirred at 60° C. for 1 h. After cooling to room temperature, the mixture was quenched with ice water (2 mL). The aqueous phase was extracted once with toluene. The combined organic layers were washed with half-saturated brine for 3 times and then concentrated to dryness. The residue was purified by column chromatography to afford(S)-2-(3-bromo-2-fluorophenyl)-5-(trifluoromethyl)-2,3-dihydrobenzofuran (0.62 g, 95%) as a colorless oil which solidified upon standing at room temperature. ¹H NMR (400 MHZ, Chloroform-d) δ 7.52 (ddd, J=8.1, 6.6, 1.6 Hz, 1H), 7.49-7.42 (m, 2H), 7.39 (ddd, J=8.1, 6.4, 1.7 Hz, 1H), 7.04 (td, J=7.9, 1.0 Hz, 1H), 6.95 (d, J=8.3 Hz, 1H), 6.07 (dd, J=9.8, 7.6 Hz, 1H), 3.78 (dd, J=16.0, 9.8 Hz, 1H), 3.19 (dd, J=16.0, 7.6 Hz, 1H).

Step 5: A mixture of(S)-2-(3-bromo-2-fluorophenyl)-5-(trifluoromethyl)-2,3-dihydrobenzofuran (0.56 g, 1.55 mmol) and CuCN(210 mg, 2.33 mmol) in NMP(2.5 mL) was heated at 160-165° C. for 6 h. After cooling to room temperature, the mixture was quenched with 20% ammonia (7.5 mL). Then EA (4 mL) was added and the mixture was vigorously stirred for 3 h followed by filtration through Celite and washing with EA. The organic layer of the filtrate was washed with water for three times. Concentration under vacuum gave(S)-2-fluoro-3-(5-(trifluoromethyl)-2,3-dihydrobenzofuran-2-yl)benzonitrile (88-2, 476 mg, 100%) as a brown oil. ¹H NMR (400 MHZ, Chloroform-d) δ 7.80-7.71 (m, 1H), 7.64 (ddd, J=7.7, 5.9, 1.7 Hz, 1H), 7.59-7.35 (m, 2H), 7.35-7.27 (m, 1H), 7.00 (d, J=8.3 Hz, 1H), 6.12 (dd, J=9.8, 7.5 Hz, 1H), 3.85 (dd, J=16.0, 9.9 Hz, 1H), 3.21 (dd, J=16.0, 7.5 Hz, 1H).

### Examples 92-97

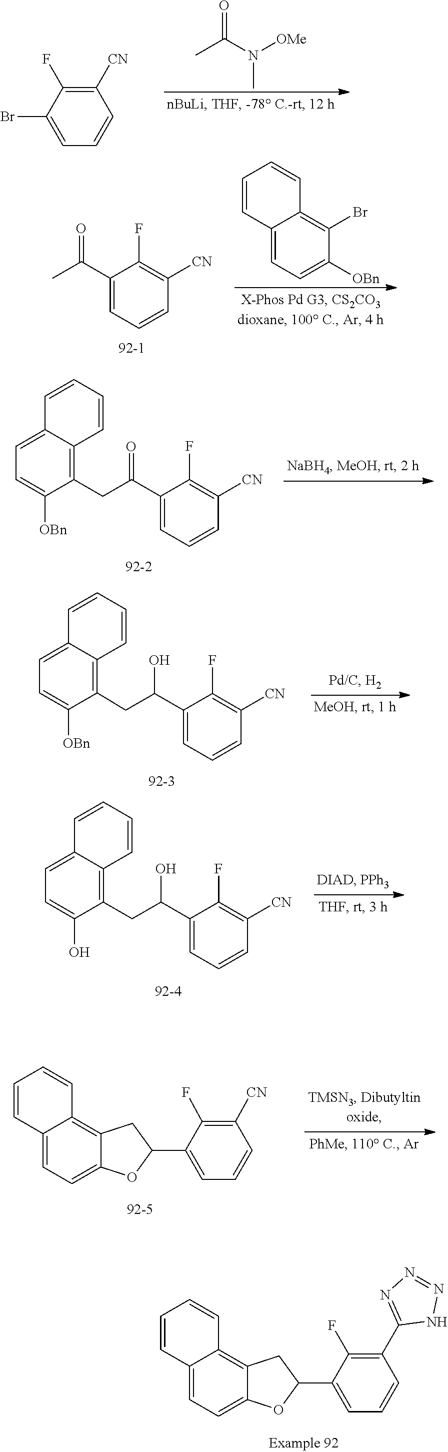

Step 1:3-Bromo-2-fluorobenzonitrile (2 g, 10 mmol) was dissolved in THF (20 mL) and cooled to −78° C. under Ar atmosphere. A solution of n-BuLi (2.5 M in hexane, 4.4 mL, 11 mmol) was added dropwise to the above solution at −78° C. The solution was stirred for 1 h at this temperature. N-methoxy-N-methylacetamide (1.34 g, 13 mmol, dissolved in 2 mL THF) was added at −78° C. After stirring at −78° C. for 30 min, the solution was warmed to room temperature and stirred overnight. The reaction was quenched with sat. NH₄Cl (30 mL), and extracted three times with EA. The organic fractions were combined, washed with NaCl, and concentrated in vacuum. The residue was purified by chromatography on silica gel to afford 92-1 (288 mg, 18% yield) as yellow oil. ES⁻ (m/z): 164.04 (M+1). ¹H NMR (400 MHZ, Chloroform-d) δ 8.13 (ddd, J=7.8, 7.1, 1.6 Hz, 1H), 7.81 (ddd, J=7.7, 5.8, 2.0 Hz, 1H), 7.37 (t, J=7.6 Hz, 1H), 2.68 (d, J=4.8 Hz, 3H).

Step 2: X-Phos Pd G3 (299 mg, 0.353 mmol), Cs₂CO₃ (1.15 g, 3.534 mmol), 3-acetyl-2-fluorobenzonitrile (288 mg, 1.767 mmol) and 2-(benzyloxy)-1-bromonaphthalene (664 mg, 2.120 mmol) were dissolved in 20 mL 1,4-dioxane. The reaction was heated at 100° C. for 4 h, and then cooled to room temperature. The reaction mixture was diluted with 10 mL EA and washed with H₂O(10 mL) and brine (10 mL). The organic layer was dried with anhydrous MgSO₄. After evaporation of the solvent, the residue was purified by chromatography on silica gel to give the 92-2 (71 mg, 10% yield) as yellow oil. LC-MS, ES⁻(m/z) 396.13 (M+1). ¹H NMR (400 MHZ, Chloroform-d) δ 7.97-7.92 (m, 1H), 7.81 (d, J=8.8 Hz, 3H), 7.78-7.72 (m, 2H), 7.44-7.27 (m, 9H), 5.16 (s, 2H), 4.75 (d, J=2.4 Hz, 2H).

Step 3: Compound 92-2 (162 mg, 0.410 mmol) was dissolved in THF (8 mL). NaBH₄ (47 mg, 1.230 mmol) was added. The mixture was stirred at rt for 2 h. The reaction was quenched with brine (6 mL), and extracted with EA for three times. The combined organic layer was dried over anhydrous Na₂SO₄, filtered, and concentrated in vacuum. The residue was purified by prep-TLC to afford 92-3 (89 mg, 55% yield) as yellow oil. ES⁻ (m/z): 398.15 (M+1). ¹H NMR (400 MHZ, Chloroform-d) δ 7.83-7.72 (m, 3H), 7.51-7.34 (m, 10H), 7.18 (t, J=7.6 Hz, 1H), 6.97 (s, 1H), 5.45-5.36 (m, 1H), 5.34-5.21 (m, 2H), 3.62-3.43 (m, 2H).

Step 4: To a solution of 92-3 (89 mg, 0.224 mmol) in MeOH (5 mL), 5% Pd/C (48 mg, 0.022 mmol) was added and the mixture was stirred for under H₂ atmosphere for 1 hour at rt. TLC showed complete consumption of the starting material. The reaction mixture was filtered and concentrated in vacuum. The residue was purified by prep-TLC to give 92-4 (40 mg, 58% yield) as yellow solid. ES⁻ (m/z): 308.10 (M+1). ¹H NMR (400 MHZ, Chloroform-d) δ 7.86-7.75 (m, 3H), 7.71 (d, J=8.8 Hz, 1H), 7.56 (ddd, J=7.8, 6.1, 1.6 Hz, 1H), 7.47 (ddd, J=8.4, 6.8, 1.6 Hz, 1H), 7.37-7.30 (m, 2H), 7.21 (d, J=8.8 Hz, 1H), 5.53 (d, J=10.0 Hz, 1H), 3.56 (d, J=15.6 Hz, 1H), 3.39 (dd, J=15.1, 9.2 Hz, 1H).

Step 5: A solution of 92-4 (58 mg, 0.181 mmol) and PPh₃ (57 mg, 0.217 mmol) in THF (4 mL) was stirred for 10 min at room temperature under Ar atmosphere for 10 min. DIAD (44 mg, 0.217 mmol) was added under Ar atmosphere and the mixture was stirred for three hours, Solvent was evaporated in vacuum and water (1 mL) was added to the residue. The mixture was extracted with EA (2 mL×2). The combined organic layer was washed with water (2 mL) and brine (2 mL), dried over anhydrous Na₂SO₄, filtered, and concentrated in vacuum. The crude product was purified by silica gel flash chromatography to afford 92-5 (21 mg, 40% yield) as white solid. ES⁻ (m/z): 290.09 (M+1). 1H NMR (400 MHZ, Chloroform-d) δ 7.85-7.73 (m, 4H), 7.61-7.53 (m, 2H), 7.51-7.45 (m, 1H), 7.34 (ddd, J=8.1, 6.8, 1.2 Hz, 1H), 7.24-7.21 (m, 1H), 6.21 (dd, J=11.3, 7.2 Hz, 1H), 4.07 (dd, J=15.4, 9.6 Hz, 1H), 3.39 (dd, J=15.9, 7.2 Hz, 1H).

Step 6: Compound 92-5 (21 mg, 0.066 mmol), TMSN₃ (76 mg, 0.658 mmol) and dibutyltinoxide (16.4 mg, 0.066 mmol) were dissolved in PhMe (1.5 ml). The resulting mixture was stirred at 100° C. overnight. The reaction mixture was quenched with brine and extracted with EA for three times. The combined organic layers were dried over anhydrous Na₂SO₄, filtered, and concentrated in vacuum. The residue was purified by TLC to give the desired product Example 92 (7.4 mg, 34% yield) as a white solid. ES⁻ (m/z): 331.11 (M−1). ¹H NMR (400 MHZ, DMSO-d₆) δ 8.05-7.98 (m, 1H), 7.88 (d, J=8.4 Hz, 1H), 7.81 (d, J=8.8 Hz, 1H), 7.72-7.66 (m, 1H), 7.62 (d, J=8.4 Hz, 1H), 7.47 (t, J=7.6 Hz, 1H), 7.41 (t, J=7.6 Hz, 1H), 7.32 (t, J=7.6 Hz, 1H), 7.25 (d, J=8.8 Hz, 1H), 6.29 (dd, J=10.2, 7.6 Hz, 1H), 4.04 (dd, J=15.7, 10.4 Hz, 1H), 3.49 (dd, J=15.8, 7.2 Hz, 1H).

| Example | Structure | NMR | MS |
|---|---|---|---|
| 93 | 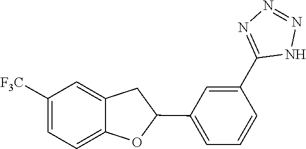 [SMILES: FC(F)(F)c1ccc2c(c1)CC(c1cccc(-c3nnn[nH]3)c1)O2] | ¹H NMR (400 MHz, Methanol-d₄) δ 8.12- 8.07 (m, 1H), 8.00-7.96 (m, 1H), 7.62-7.58 (m, 2H), 7.53-7.44 (m, 2H), 6.98 (d, J = 8.4 Hz, 1H), 6.02-5.95 (m, 1H), 3.81 (dd, J = 16.0, 9.6 Hz, 1H), 3.27-3.20 (m, 1H). | 31.06 [M − 1] |
| 94 | 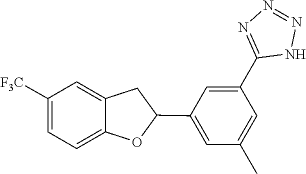 [SMILES: Cc1cc(-c2nnn[nH]2)cc(C2Cc3cc(C(F)(F)F)ccc3O2)c1] | ¹H NMR (400 MHz, Methanol-d₄) δ 7.87 (s, 1H), 7.80 (s, 1H), 7.56-7.34 (m, 3H), 7.02- 6.89 (m, 1H), 6.07-5.82 (m, 1H), 3.86-3.73 (m, 1H), 3.26-3.17 (m, 1H), 2.44 (s, 3H) | 344.99 (M + 1) |
| 95 | 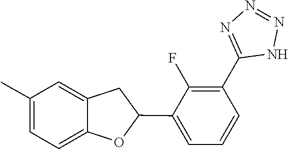 [SMILES: Cc1ccc2c(c1)CC(c1cccc(-c3nnn[nH]3)c1F)O2] | ¹H NMR (400 MHz, Methanol-d₄) δ 8.07- 7.99 (m, 1H), 7.66 (t, J = 7.6 Hz, 1H), 7.37 (t, J = 7.6 Hz, 1H), 7.01 (s, 1H), 6.94 (d, J = 8.0 Hz, 1H), 6.73 (d, J = 8.0 Hz, 1H), 6.07-5.99 (m, 1H), 3.73 (dd, J = 16.0, 9.6 Hz, 1H), 3.14 (dd, J = 16.0, 7.8 Hz, 1H), 2.25 (s, 3H). | 295.05 [M − 1]⁻ |
| 96 | 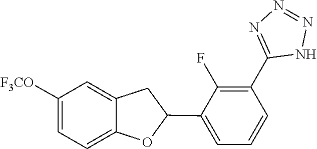 [SMILES: Fc1c(-c2nnn[nH]2)cccc1C1Cc2cc(OC(F)(F)F)ccc2O1] | ¹H NMR (400 MHz, Methanol-d₄) δ 8.03 (t, J = 7.3 Hz, 1H), 7.65 (t, J = 7.3 Hz, 1H), 7.37 (t, J = 7.8 Hz, 1H), 7.15 (d, J = 2.3 Hz, 1H), 7.06 (d, J = 8.8 Hz, 1H), 6.89 (d, J = 8.7 Hz, 1H), 6.23-6.07 (m, 1H), 3.86-3.79 (m, 1H), 3.27-3.21 (m, 1H) | 365.0 [M − H] |
| 97 | 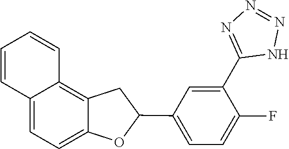 [SMILES: Fc1ccc(C2Cc3c(ccc4ccccc34)O2)cc1-c1nnn[nH]1] | ¹H NMR (400 MHz, Methanol-d₄) δ 8.18 (dd, J = 6.8, 2.4 Hz, 1H), 7.81 (d, J = 8.3 Hz, 1H), 7.74 (d, J = 8.8 Hz, 1H), 7.71-7.65 (m, 1H), 7.61 (d, J = 8.3 Hz, 1H), 7.47-7.41 (m, 1H), 7.36 (dd, J = 10.5, 8.7 Hz, 1H), 7.31-7.25 (m, 1H), 7.17 (d, J = 8.8 Hz, 1H), 6.09-6.01 (m, 1H), 4.07-3.95 (m, 1H), 3.48-3.38 (m, 1H) | 331.1 [M − H] |

### Examples 98-104

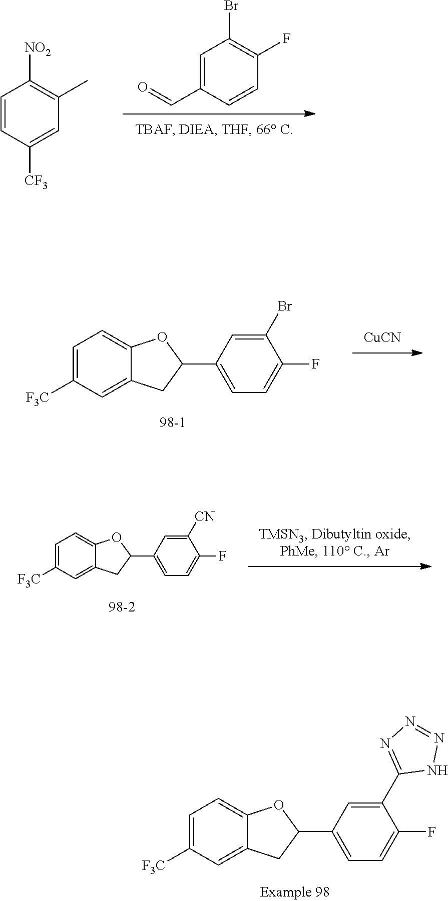

Step 1: A mixture of 2-methyl-1-nitro-4-(trifluoromethyl)benzene (150 mg, 0.732 mmol), 3-bromo-4-fluorobenzaldehyde (178 mg, 0.878 mmol), DIEA (283 mg, 2.196 mmol), and TBAF (1464 uL, 1.464 mmol) in THF (8 mL) was stirred at 66° C. for 12 h. The reaction mixture was cooled to room temperature, washed with H₂O, and extracted with EtOAc for three times. The combined organic layer was dried over anhydrous Na₂SO₄, filtered, and concentrated under reduced pressure. The residue was purified by column chromatography to give 98-1 (120 mg, 45% yield) as yellow solid. LC-MS, ES⁻ (m/z): 362.13 (M+1). ¹H NMR (400 MHZ, Chloroform-d) δ 7.58 (dd, J=6.5, 2.0 Hz, 1H), 7.44 (d, J=10.4 Hz, 2H), 7.29 (ddd, J=9.0, 4.9, 2.4 Hz, 1H), 7.12 (t, J=8.4 Hz, 1H), 6.91 (d, J=8.4 Hz, 1H), 5.87-5.66 (m, 1H), 3.78-3.55 (m, 1H), 3.19 (dd, J=15.9, 8.0 Hz, 1H).

Step 2: A mixture of 98-1 (40 mg, 0.111 mmol) and CuCN(12 mg, 0.133 mmol) in toluene (4 mL) was stirred at 110° C. for 3 h. Solvent was removed under reduced pressure. The residue was taken up with aq. NaCl, and extracted with EA for three times. The combined organic layer was dried over anhydrous Na₂SO₄, filtered, and concentrated in vacuum. The residue was purified by prep-TLC to give 98-2 (12 mg, 35% yield) as yellow solid. LC-MS, ES⁻ (m/z): 308.25 (M+1). ¹H NMR (400 MHZ, Chloroform-d) δ 7.67-7.56 (m, 1H), 7.49-7.42 (m, 2H), 7.23 (d, J=8.4 Hz, 1H), 7.09 (dt, J=24.5, 8.4 Hz, 1H), 6.97-6.88 (m, 1H), 5.88-5.70 (m, 1H), 3.78-3.60 (m, 1H), 3.18 (dt, J=16.1, 8.8 Hz, 1H).

Step 3:98-2 (12 mg, 0.039 mmol), TMSN₃ (45 mg, 0.391 mmol) and dibutyltinoxide (10 mg, 0.039 mmol) were dissolved in toluene (1 ml) and stirred at 100° C. overnight. The reaction mixture was quenched with aq. NaCl and extracted with EA for three times. The combined organic layer was dried over anhydrous Na₂SO₄, filtered, and concentrated in vacuum. The residue was purified by prep-TLC to give compound Example 98 (4.7 mg, 34% yield) as white solid. LC-MS, ES⁻ (m/z): 349.28 (M-1). ¹H NMR (400 MHz, Methanol-d₄) δ 8.17 (dd, J=6.7, 2.4 Hz, 1H), 7.67 (ddd, J=8.6, 5.1, 2.4 Hz, 1H), 7.52-7.44 (m, 2H), 7.40 (dd, J=10.5, 8.8 Hz, 1H), 6.97 (d, J=8.4 Hz, 1H), 6.03-5.92 (m, 1H), 3.80 (dd, J=16.2, 9.6 Hz, 1H), 3.27-3.21 (m, 1H).

| Example | Structure | NMR | MS |
|---|---|---|---|
| 99 | 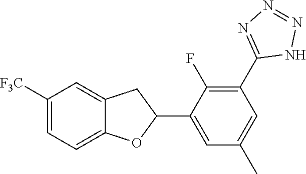 [SMILES: Cc1cc(-c2nnn[nH]2)c(F)c(C2Cc3cc(C(F)(F)F)ccc3O2)c1] | ¹H NMR (400 MHz, Chloroform-d) δ 7.51- 7.40 (m, 2H), 7.31 (d, J = 8.2 Hz, 1H), 7.14 (d, J = 8.3 Hz, 1H), 6.99-6.91 (m, 1H), 6.22-5.84 (m, 1H), 3.85-3.64 (m, 1H), 3.25-3.02 (m, 1H), 2.28 (s, 3H) | 363.0 [M − 1]⁻ |
| 100 | 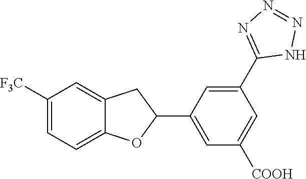 [SMILES: O=C(O)c1cc(-c2nnn[nH]2)cc(C2Cc3cc(C(F)(F)F)ccc3O2)c1] | ¹H NMR (400 MHz, Methanol-d₄) δ 7.92-7.80 (m, 3H), 7.69-7.64 (m, 1H), 7.56-7.53 (m, 2H), 7.51-7.46 (m, 2H), 7.40-7.37 (m, 1H), 7.23 (t, J = 7.7 Hz, 1H), 6.85 (d, J = 8.5 Hz, 1H), 6.05 (dd, J = 9.7, 7.5 Hz, 1H), 3.77 (dd, J = 15.9, 9.6 Hz, 1H), 3.20-3.08 (m, 1H). | 376.1 [M − 1]⁻ |
| 101 | 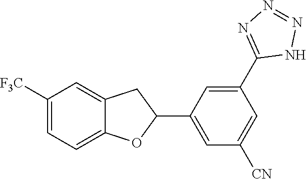 [SMILES: N#Cc1cc(-c2nnn[nH]2)cc(C2Cc3cc(C(F)(F)F)ccc3O2)c1] | ¹H NMR (400 MHz, DMSO-d₆) δ 8.35 (d, J = 13.1 Hz, 2H), 7.90 (s, 1H), 7.63 (s, 1H), 7.56 (d, J = 8.4 Hz, 1H), 7.11 (d, J = 8.4 Hz, 1H), 6.11 (t, J = 8.7 Hz, 1H), 3.84 (dd, J = 16.3, 9.6 Hz, 1H), 3.27 (dd, J = 16.3, 7.8 Hz, 1H). | 356.3 [M − 1] |

The following examples were obtained by chiral resolution of Example 92, 97, and 98.

| Example | Structure | NMR | MS |
|---|---|---|---|
| 102 | 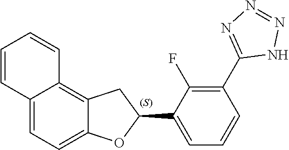 | ¹H NMR (400 MHz, DMSO-d₆) δ 8.05-7.98 (m, 1H), 7.88 (d, J = 8.4 Hz, 1H), 7.81 (d, J = 8.8 Hz, 1H), 7.72-7.66 (m, 1H), 7.62 (d, J = 8.4 Hz, 1H), 7.47 (t, J = 7.6 Hz, 1H), 7.41 (t, J = 7.6 Hz, 1H), 7.32 (t, J = 7.6 Hz, 1H), 7.25 (d, J = 8.8 Hz, 1H), 6.29 (dd, J = 10.2, 7.6 Hz, 1H), 4.04 (dd, J = 15.7, 10.4 Hz, 1H), 3.49 (dd, J = 15.8, 7.2 Hz, 1H). | 331.11 [M − 1]. |
| 103 | 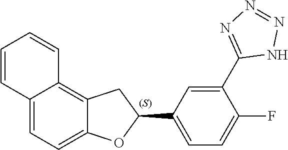 | ¹H NMR (400 MHz, Methanol-d₄) δ 8.18 (dd, J = 6.8, 2.4 Hz, 1H), 7.81 (d, J = 8.3 Hz, 1H), 7.74 (d, J = 8.8 Hz, 1H), 7.71-7.65 (m, 1H), 7.61 (d, J = 8.3 Hz, 1H), 7.47-7.41 (m, 1H), 7.36 (dd, J = 10.5, 8.7 Hz, 1H), 7.31-7.25 (m, 1H), 7.17 (d, J = 8.8 Hz, 1H), 6.09-6.01 (m, 1H), 4.07-3.95 (m, 1H), 3.48-3.38 (m, 1H) | 331.1 [M − H] |
| 104 | 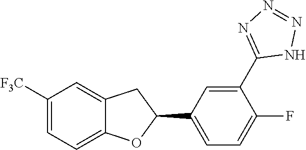 [SMILES: Fc1ccc([C@@H]2Cc3cc(C(F)(F)F)ccc3O2)cc1-c1nnn[nH]1] | ¹H NMR (400 MHz, Methanol-d₄) δ 8.17 (dd, J = 6.7, 2.4 Hz, 1H), 7.67 (ddd, J = 8.6, 5.1, 2.4 Hz, 1H), 7.52-7.44 (m, 2H), 7.40 (dd, J = 10.5, 8.8 Hz, 1H), 6.97 (d, J = 8.4 Hz, 1H), 6.03- 5.92 (m, 1H), 3.80 (dd, J = 16.2, 9.6 Hz, 1H), 3.27-3.21 (m, 1H). | 349.28 [M − 1] |

### Example 105

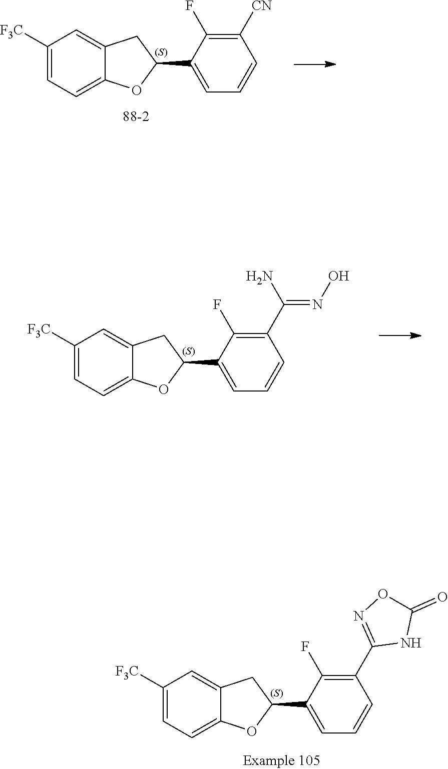

Step 1: To a solution of(S)-2-fluoro-3-(5-(trifluoromethyl)-2,3-dihydrobenzofuran-2-yl)benzonitrile (3.5 g, 11.4 mmol)(88-2) in EtOH (30 mL), was added NH₂OH·HCl (1.18 g, 17 mmol) and Et₃N(1.73 g, 17 mmol). The mixture was stirred at 80° C. for 4 hours. The reaction mixture was cooled to rt, diluted with H₂O, and extracted with EtOAc for three times. The combined organic layer was dried over anhydrous Na₂SO₄, filtered, and concentrated under reduced pressure. The residue was purified by column chromatography to give(S)-2-fluoro-N′-hydroxy-3-(5-(trifluoromethyl)-2,3-dihydrobenzofuran-2-yl)benzimidamide (3.1 g, 80% yield) as colorless oil. LC-MS (ESI): m/z=339 [M-H]

Step 2: To a solution of(S)-2-fluoro-N′-hydroxy-3-(5-(trifluoromethyl)-2,3-dihydrobenzofuran-2-yl)benzimidamide (3.1 g, 9.1 mmol) in dioxane (31 mL), was added CDI (1.77 g, 10.9 mmol) and DBU (1.52 g,10 mmol). The mixture was stirred at 100° C. for 3 hours. The reaction mixture was diluted with water, adjusted to pH=1-2 with HCl, and extracted with EtOAc for three times. The combined organic layer was dried over anhydrous Na₂SO₄, filtered, and concentrated under reduced pressure. The residue was purified via recrystallization in DCM/MTBE to give Example 105 (700 mg, 21% yield) as white solid. LC-MS (ESI): m/z=365 [M-H]. ¹H NMR (400 MHZ, Methanol-d₄) δ 7.82-7.67 (m, 2H), 7.53 (s, 1H), 7.51-7.45 (m, 1H), 7.38 (t, J=7.8 Hz, 1H), 7.00 (d, J=8.4 Hz, 1H), 6.16 (dd, J=9.9, 7.6 Hz, 1H), 3.85 (dd, J=16.1, 9.9 Hz, 1H), 3.29-3.22 (m, 1H).

### Example 106

Provided technologies can reduce MRGPRX4 activation.

Among other things, provided technologies can reduce MRGPRX4 activation. Various technologies can be utilized in accordance with the present disclosure to access activities of provided compounds. An example is described below.

HEK293T cells stably expressing human MRGPRX4 were cultured in DMEM medium supplements with 10% FBS, 100 U/mL penicillin-streptomycin, 20 mM HEPES and 1 μg/mL puromycin at 37° C. with 5% (v/v) CO₂. One day before test, the cells were detached using TrypLE™ Express and counted using cell counter. In a 384-well plate cells were seeded at a density of 20,000 cells/well in 20 uL/well planting medium (DMEM medium supplements with 5% FBS, 100 U/mL penicillin-streptomycin and 20 mM HEPES), and incubated overnight at 37° C. with 5% (v/v) CO₂. On the day of experiments, the dye solution, prepared following the manufacture's instruction of the Screen Quest™ Fluo-8 No Wash Calcium Assay Kit, was added to each well (20 μL/well) of the cell plate. The cell plate was incubated at 25° C. for 1 hour. Test compounds in different doses were transferred to the cell plate by FLIPR and the plate was kept in the dark at 25° C. for 30 minutes. The agonist solution, 0.2 μM of deoxycholic acid-3-phosphoric acid in assay buffer, was added to each well of the plate. The plate was read for 160 sec with 1 sec interval to obtain data of antagonist mode in FLIPR. For various compounds, IC₅₀ values were calculated by fitting % inhibition against log of compound concentrations (top conc. 30 μM, ten 3-fold serial dilutions) with Hill equation using XLfit. MRGPRX4 antagonist activities of certain compounds tested in this assay were listed in Table 1 as examples. Certain compounds were assessed at a single concentration of 2 μM, and if such compounds showed low inhibition (40%), such compounds may be listed in Table 1 with “C” activity. Those skilled in the art appreciate that IC₅₀ of various compounds can be assessed in accordance with the present disclosure. In some embodiments, inhibition of various compounds were assessed at certain concentrations, e.g., about 0.5 μM, about 2 μM, etc; certain data were presented in the Table below as examples.

TABLE 1
Certain compounds and data as examples (A = IC₅₀ < 1 μM; B = 1 μM <= IC₅₀ <= 10
μM; C = IC₅₀ > 10 μM or <40% inhibition at 2 μM).

| Example | Structure | Antagonist activity |
|---|---|---|
| 1 | 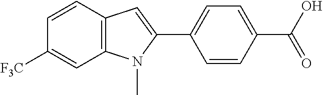 [SMILES: Cn1c(-c2ccc(C(=O)O)cc2)cc2ccc(C(F)(F)F)cc21] | B |
| 2 | 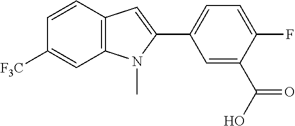 [SMILES: Cn1c(-c2ccc(F)c(C(=O)O)c2)cc2ccc(C(F)(F)F)cc21] | C |
| 3 | 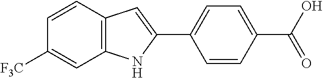 [SMILES: O=C(O)c1ccc(-c2cc3ccc(C(F)(F)F)cc3[nH]2)cc1] | C |
| 4 | 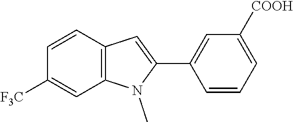 [SMILES: Cn1c(-c2cccc(C(=O)O)c2)cc2ccc(C(F)(F)F)cc21] | C |
| 5 | 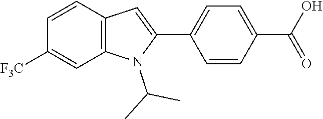 [SMILES: CC(C)n1c(-c2ccc(C(=O)O)cc2)cc2ccc(C(F)(F)F)cc21] | B |
| 6 | 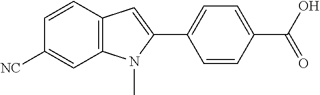 [SMILES: Cn1c(-c2ccc(C(=O)O)cc2)cc2ccc(C#N)cc21] | C |
| 7 | 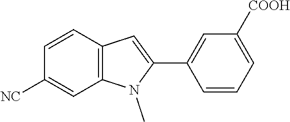 [SMILES: Cn1c(-c2cccc(C(=O)O)c2)cc2ccc(C#N)cc21] | C |
| 8 | 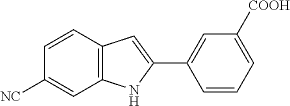 [SMILES: N#Cc1ccc2cc(-c3cccc(C(=O)O)c3)[nH]c2c1] | C |
| 9 | 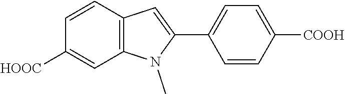 [SMILES: Cn1c(-c2ccc(C(=O)O)cc2)cc2ccc(C(=O)O)cc21] | C |
| 10 | 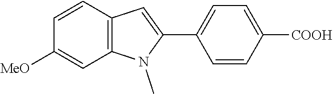 [SMILES: COc1ccc2cc(-c3ccc(C(=O)O)cc3)n(C)c2c1] | C |
| 11 | 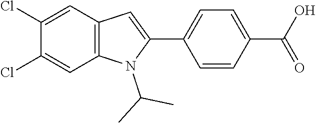 [SMILES: CC(C)n1c(-c2ccc(C(=O)O)cc2)cc2cc(Cl)c(Cl)cc21] | C |
| 12 | 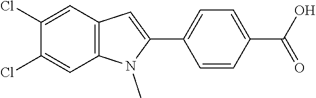 [SMILES: Cn1c(-c2ccc(C(=O)O)cc2)cc2cc(Cl)c(Cl)cc21] | C |
| 13 | 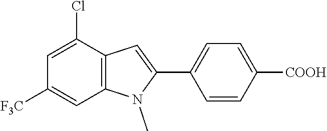 [SMILES: Cn1c(-c2ccc(C(=O)O)cc2)cc2c(Cl)cc(C(F)(F)F)cc21] | B |
| 14 | 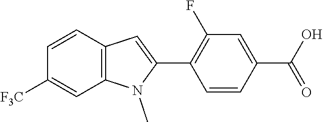 [SMILES: Cn1c(-c2ccc(C(=O)O)cc2F)cc2ccc(C(F)(F)F)cc21] | B |
| 15 | 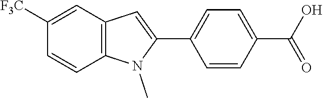 [SMILES: Cn1c(-c2ccc(C(=O)O)cc2)cc2cc(C(F)(F)F)ccc21] | B |
| 16 | 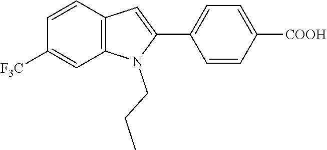 [SMILES: CCCn1c(-c2ccc(C(=O)O)cc2)cc2ccc(C(F)(F)F)cc21] | B |
| 17 | 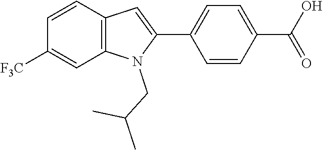 [SMILES: CC(C)Cn1c(-c2ccc(C(=O)O)cc2)cc2ccc(C(F)(F)F)cc21] | B |
| 18 | 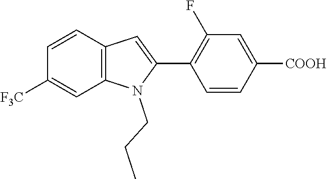 [SMILES: CCCn1c(-c2ccc(C(=O)O)cc2F)cc2ccc(C(F)(F)F)cc21] | A |
| 19 | 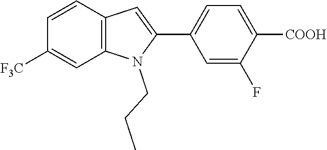 [SMILES: CCCn1c(-c2ccc(C(=O)O)c(F)c2)cc2ccc(C(F)(F)F)cc21] | B |
| 20 | 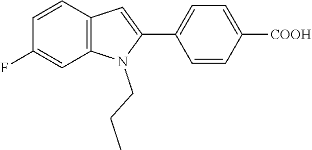 [SMILES: CCCn1c(-c2ccc(C(=O)O)cc2)cc2ccc(F)cc21] | C |
| 21 | 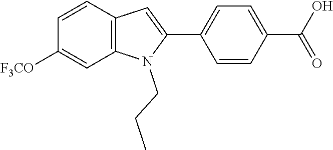 [SMILES: CCCn1c(-c2ccc(C(=O)O)cc2)cc2ccc(OC(F)(F)F)cc21] | C |
| 22 | 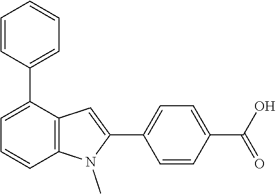 [SMILES: Cn1c(-c2ccc(C(=O)O)cc2)cc2c(-c3ccccc3)cccc21] | C |
| 23 | 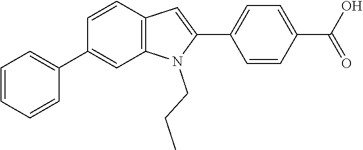 [SMILES: CCCn1c(-c2ccc(C(=O)O)cc2)cc2ccc(-c3ccccc3)cc21] | C |
| 24 | 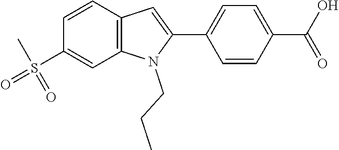 [SMILES: CCCn1c(-c2ccc(C(=O)O)cc2)cc2ccc(S(C)(=O)=O)cc21] | C |
| 25 | 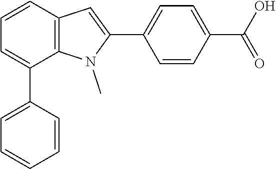 [SMILES: Cn1c(-c2ccc(C(=O)O)cc2)cc2cccc(-c3ccccc3)c21] | C |
| 26 | 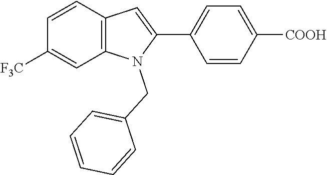 [SMILES: O=C(O)c1ccc(-c2cc3ccc(C(F)(F)F)cc3n2Cc2ccccc2)cc1] | C |
| 27 | 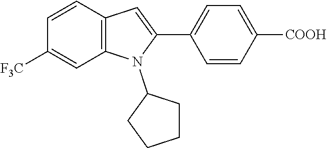 [SMILES: O=C(O)c1ccc(-c2cc3ccc(C(F)(F)F)cc3n2C2CCCC2)cc1] | B |
| 28 | 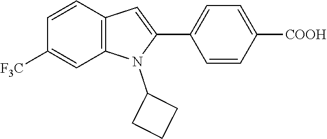 [SMILES: O=C(O)c1ccc(-c2cc3ccc(C(F)(F)F)cc3n2C2CCC2)cc1] | B |
| 29 | 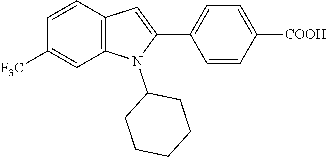 [SMILES: O=C(O)c1ccc(-c2cc3ccc(C(F)(F)F)cc3n2C2CCCCC2)cc1] | B |
| 30 | 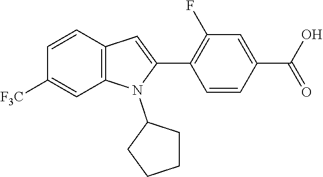 [SMILES: O=C(O)c1ccc(-c2cc3ccc(C(F)(F)F)cc3n2C2CCCC2)c(F)c1] | A |
| 31 | 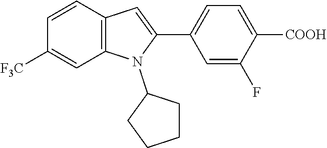 [SMILES: O=C(O)c1ccc(-c2cc3ccc(C(F)(F)F)cc3n2C2CCCC2)cc1F] | B |
| 32 | 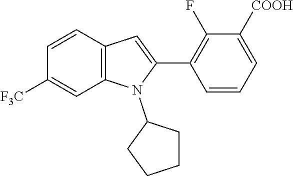 [SMILES: O=C(O)c1cccc(-c2cc3ccc(C(F)(F)F)cc3n2C2CCCC2)c1F] | C |
| 33 | 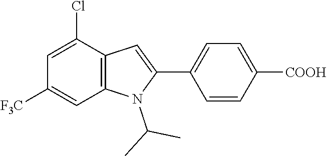 [SMILES: CC(C)n1c(-c2ccc(C(=O)O)cc2)cc2c(Cl)cc(C(F)(F)F)cc21] | C |
| 34 | 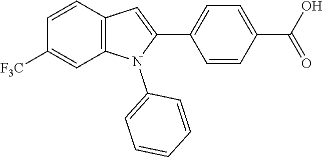 [SMILES: O=C(O)c1ccc(-c2cc3ccc(C(F)(F)F)cc3n2-c2ccccc2)cc1] | B |
| 35 | 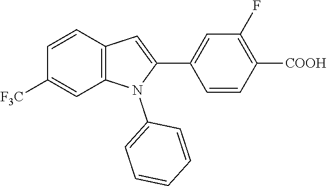 [SMILES: O=C(O)c1ccc(-c2cc3ccc(C(F)(F)F)cc3n2-c2ccccc2)cc1F] | C |
| 36 | 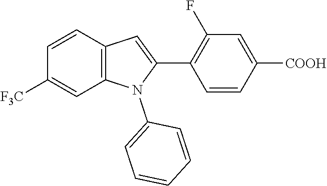 [SMILES: O=C(O)c1ccc(-c2cc3ccc(C(F)(F)F)cc3n2-c2ccccc2)c(F)c1] | C |
| 37 | 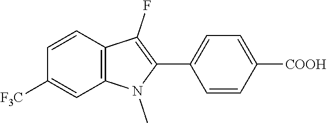 [SMILES: Cn1c(-c2ccc(C(=O)O)cc2)c(F)c2ccc(C(F)(F)F)cc21] | C |
| 38 | 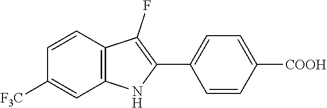 [SMILES: O=C(O)c1ccc(-c2[nH]c3cc(C(F)(F)F)ccc3c2F)cc1] | C |
| 39 | 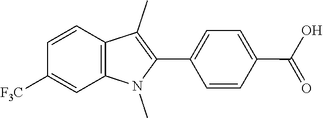 [SMILES: Cc1c(-c2ccc(C(=O)O)cc2)n(C)c2cc(C(F)(F)F)ccc12] | C |
| 40 | 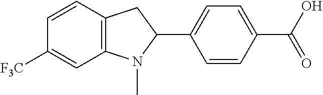 [SMILES: CN1c2cc(C(F)(F)F)ccc2CC1c1ccc(C(=O)O)cc1] | C |
| 41 | 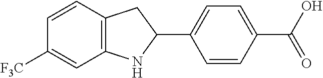 [SMILES: O=C(O)c1ccc(C2Cc3ccc(C(F)(F)F)cc3N2)cc1] | C |
| 42 | 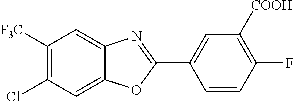 [SMILES: O=C(O)c1cc(-c2nc3cc(C(F)(F)F)c(Cl)cc3o2)ccc1F] | C |
| 43 | 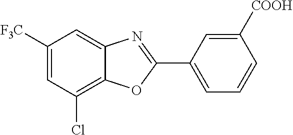 [SMILES: O=C(O)c1cccc(-c2nc3cc(C(F)(F)F)cc(Cl)c3o2)c1] | C |
| 44 | 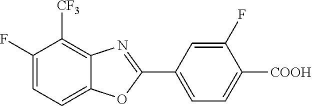 [SMILES: O=C(O)c1ccc(-c2nc3c(C(F)(F)F)c(F)ccc3o2)cc1F] | C |
| 45 | 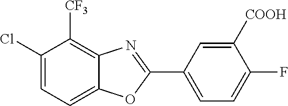 [SMILES: O=C(O)c1cc(-c2nc3c(C(F)(F)F)c(Cl)ccc3o2)ccc1F] | C |
| 46 | 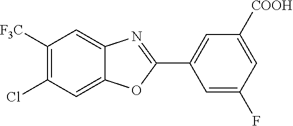 [SMILES: O=C(O)c1cc(F)cc(-c2nc3cc(C(F)(F)F)c(Cl)cc3o2)c1] | C |
| 47 | 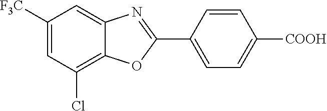 [SMILES: O=C(O)c1ccc(-c2nc3cc(C(F)(F)F)cc(Cl)c3o2)cc1] | C |
| 48 | 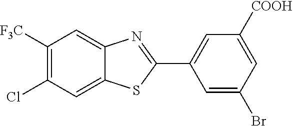 [SMILES: O=C(O)c1cc(Br)cc(-c2nc3cc(C(F)(F)F)c(Cl)cc3s2)c1] | C |
| 49 | 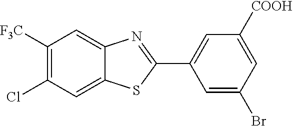 [SMILES: O=C(O)c1cc(Br)cc(-c2nc3cc(C(F)(F)F)c(Cl)cc3s2)c1] | C |
| 50 | 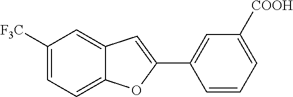 [SMILES: O=C(O)c1cccc(-c2cc3cc(C(F)(F)F)ccc3o2)c1] | C |
| 51 | 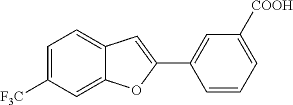 [SMILES: O=C(O)c1cccc(-c2cc3ccc(C(F)(F)F)cc3o2)c1] | C |
| 52 | 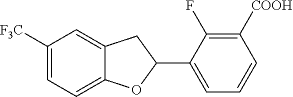 [SMILES: O=C(O)c1cccc(C2Cc3cc(C(F)(F)F)ccc3O2)c1F] | A |
| 52A | 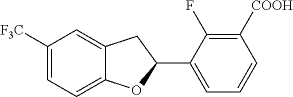 [SMILES: O=C(O)c1cccc([C@@H]2Cc3cc(C(F)(F)F)ccc3O2)c1F] | A |
| 52B | 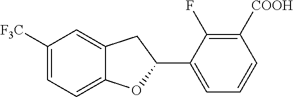 [SMILES: O=C(O)c1cccc([C@H]2Cc3cc(C(F)(F)F)ccc3O2)c1F] | C |
| 53 | 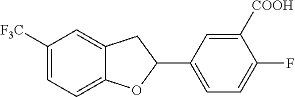 [SMILES: O=C(O)c1cc(C2Cc3cc(C(F)(F)F)ccc3O2)ccc1F] | B |
| 54 | 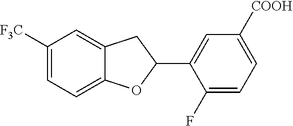 [SMILES: O=C(O)c1ccc(F)c(C2Cc3cc(C(F)(F)F)ccc3O2)c1] | C |
| 55 | 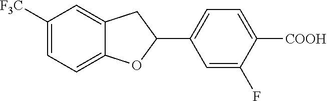 [SMILES: O=C(O)c1ccc(C2Cc3cc(C(F)(F)F)ccc3O2)cc1F] | C |
| 56 | 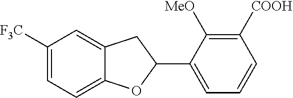 [SMILES: COc1c(C(=O)O)cccc1C1Cc2cc(C(F)(F)F)ccc2O1] | C |
| 57 | 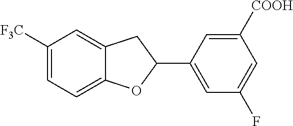 [SMILES: O=C(O)c1cc(F)cc(C2Cc3cc(C(F)(F)F)ccc3O2)c1] | B |
| 58 | 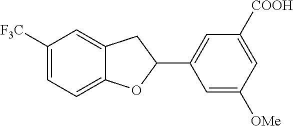 [SMILES: COc1cc(C(=O)O)cc(C2Cc3cc(C(F)(F)F)ccc3O2)c1] | C |
| 59 | 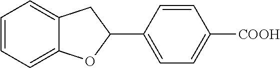 [SMILES: O=C(O)c1ccc(C2Cc3ccccc3O2)cc1] | C |
| 60 | 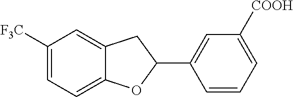 [SMILES: O=C(O)c1cccc(C2Cc3cc(C(F)(F)F)ccc3O2)c1] | B |
| 61 | 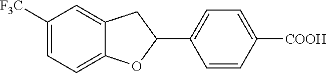 [SMILES: O=C(O)c1ccc(C2Cc3cc(C(F)(F)F)ccc3O2)cc1] | C |
| 62 | 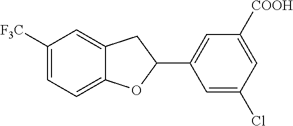 [SMILES: O=C(O)c1cc(Cl)cc(C2Cc3cc(C(F)(F)F)ccc3O2)c1] | B |
| 63 | 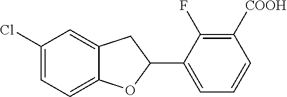 [SMILES: O=C(O)c1cccc(C2Cc3cc(Cl)ccc3O2)c1F] | B |
| 64 | 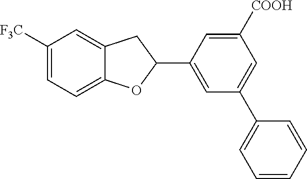 [SMILES: O=C(O)c1cc(-c2ccccc2)cc(C2Cc3cc(C(F)(F)F)ccc3O2)c1] | C |
| 65 | 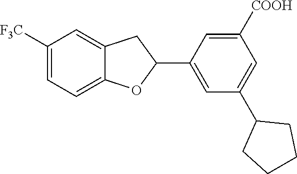 [SMILES: O=C(O)c1cc(C2CCCC2)cc(C2Cc3cc(C(F)(F)F)ccc3O2)c1] | C |
| 66 | 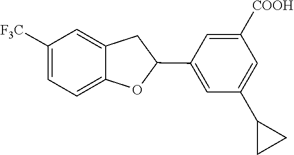 [SMILES: O=C(O)c1cc(C2CC2)cc(C2Cc3cc(C(F)(F)F)ccc3O2)c1] | B |
| 67 | 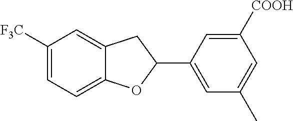 [SMILES: Cc1cc(C(=O)O)cc(C2Cc3cc(C(F)(F)F)ccc3O2)c1] | B |
| 68 | 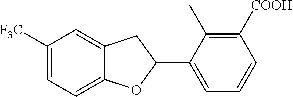 [SMILES: Cc1c(C(=O)O)cccc1C1Cc2cc(C(F)(F)F)ccc2O1] | C |
| 69 | 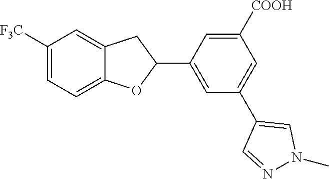 [SMILES: Cn1cc(-c2cc(C(=O)O)cc(C3Cc4cc(C(F)(F)F)ccc4O3)c2)cn1] | C |
| 70 | 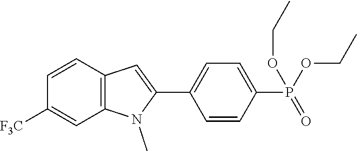 [SMILES: CCOP(=O)(OCC)c1ccc(-c2cc3ccc(C(F)(F)F)cc3n2C)cc1] | C |
| 71 | 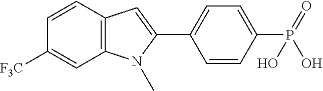 [SMILES: Cn1c(-c2ccc(P(=O)(O)O)cc2)cc2ccc(C(F)(F)F)cc21] | C |
| 72 | 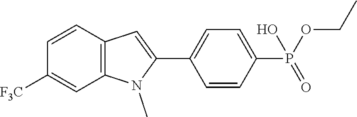 [SMILES: CCOP(=O)(O)c1ccc(-c2cc3ccc(C(F)(F)F)cc3n2C)cc1] | C |
| 73 | 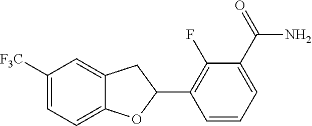 [SMILES: NC(=O)c1cccc(C2Cc3cc(C(F)(F)F)ccc3O2)c1F] | C |
| 74 | 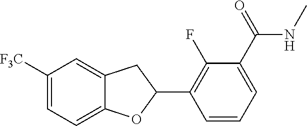 [SMILES: CNC(=O)c1cccc(C2Cc3cc(C(F)(F)F)ccc3O2)c1F] | C |
| 75 | 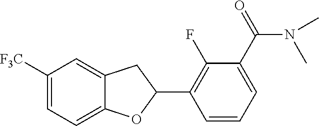 [SMILES: CN(C)C(=O)c1cccc(C2Cc3cc(C(F)(F)F)ccc3O2)c1F] | C |
| 76 | 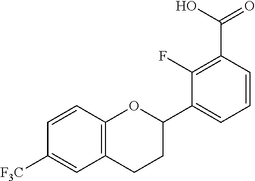 [SMILES: O=C(O)c1cccc(C2CCc3cc(C(F)(F)F)ccc3O2)c1F] | C |
| 77 | 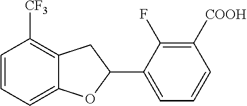 [SMILES: O=C(O)c1cccc(C2Cc3c(cccc3C(F)(F)F)O2)c1F] | C |
| 78 | 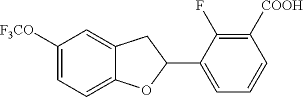 [SMILES: O=C(O)c1cccc(C2Cc3cc(OC(F)(F)F)ccc3O2)c1F] | B |
| 79 | 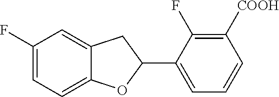 [SMILES: O=C(O)c1cccc(C2Cc3cc(F)ccc3O2)c1F] | C |
| 80 | 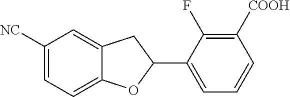 [SMILES: N#Cc1ccc2c(c1)CC(c1cccc(C(=O)O)c1F)O2] | C |
| 81 | 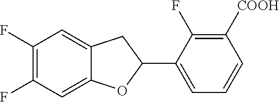 [SMILES: O=C(O)c1cccc(C2Cc3cc(F)c(F)cc3O2)c1F] | C |
| 82 | 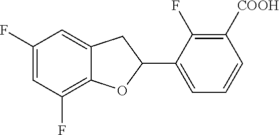 [SMILES: O=C(O)c1cccc(C2Cc3cc(F)cc(F)c3O2)c1F] | C |
| 83 | 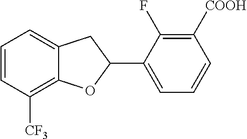 [SMILES: O=C(O)c1cccc(C2Cc3cccc(C(F)(F)F)c3O2)c1F] | C |
| 84 | 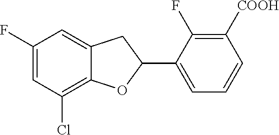 [SMILES: O=C(O)c1cccc(C2Cc3cc(F)cc(Cl)c3O2)c1F] | C |
| 85 | 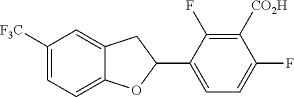 [SMILES: O=C(O)c1c(F)ccc(C2Cc3cc(C(F)(F)F)ccc3O2)c1F] | C |
| 86 | 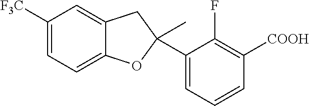 [SMILES: CC1(c2cccc(C(=O)O)c2F)Cc2cc(C(F)(F)F)ccc2O1] | B |
| 87 | 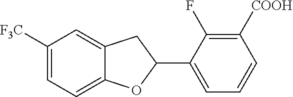 [SMILES: O=C(O)c1cccc(C2Cc3cc(C(F)(F)F)ccc3O2)c1F] | C |
| 88 | 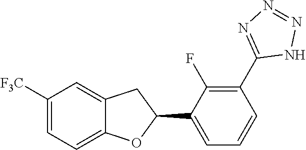 [SMILES: Fc1c(-c2nnn[nH]2)cccc1[C@@H]1Cc2cc(C(F)(F)F)ccc2O1] | A |
| 89 | 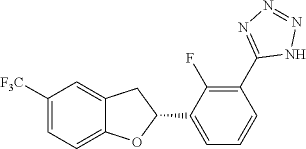 [SMILES: Fc1c(-c2nnn[nH]2)cccc1[C@H]1Cc2cc(C(F)(F)F)ccc2O1] | 61% inhibition at 2 μM |
| 90 | 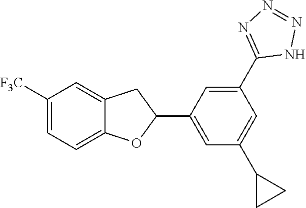 [SMILES: FC(F)(F)c1ccc2c(c1)CC(c1cc(-c3nnn[nH]3)cc(C3CC3)c1)O2] | 27% inhibition at 0.5 μM |
| 91 | 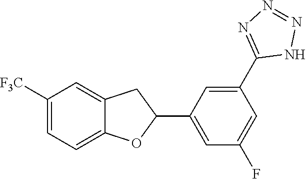 [SMILES: Fc1cc(-c2nnn[nH]2)cc(C2Cc3cc(C(F)(F)F)ccc3O2)c1] | A |
| 92 | 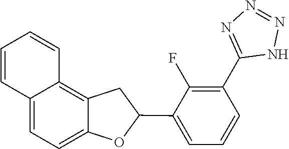 [SMILES: Fc1c(-c2nnn[nH]2)cccc1C1Cc2c(ccc3ccccc23)O1] | A |
| 93 | 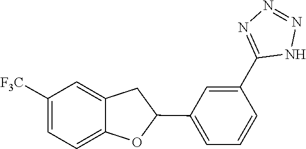 [SMILES: FC(F)(F)c1ccc2c(c1)CC(c1cccc(-c3nnn[nH]3)c1)O2] | 24% inhibition at 0.5 μM |
| 94 | 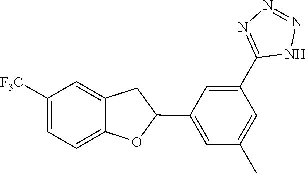 [SMILES: Cc1cc(-c2nnn[nH]2)cc(C2Cc3cc(C(F)(F)F)ccc3O2)c1] | A |
| 95 | 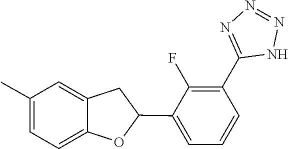 [SMILES: Cc1ccc2c(c1)CC(c1cccc(-c3nnn[nH]3)c1F)O2] | 8.6% inhibition at 0.5 μM |
| 96 | 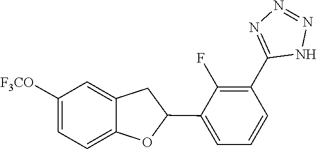 [SMILES: Fc1c(-c2nnn[nH]2)cccc1C1Cc2cc(OC(F)(F)F)ccc2O1] | 3.7% inhibition at 0.5 μM |
| 97 | 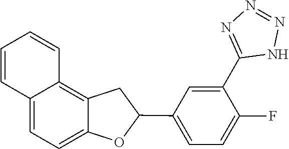 [SMILES: Fc1ccc(C2Cc3c(ccc4ccccc34)O2)cc1-c1nnn[nH]1] | A |
| 98 | 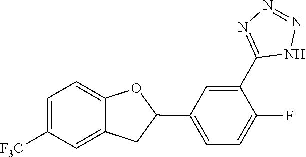 [SMILES: Fc1ccc(C2Cc3cc(C(F)(F)F)ccc3O2)cc1-c1nnn[nH]1] | A |
| 99 | 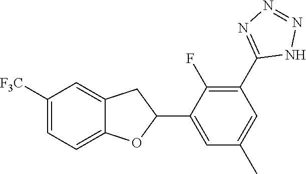 [SMILES: Cc1cc(-c2nnn[nH]2)c(F)c(C2Cc3cc(C(F)(F)F)ccc3O2)c1] | A |
| 100 | 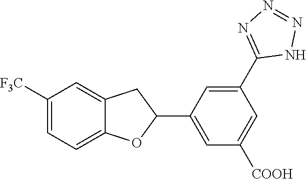 [SMILES: O=C(O)c1cc(-c2nnn[nH]2)cc(C2Cc3cc(C(F)(F)F)ccc3O2)c1] | 7.5% inhibition at 0.5 μM |
| 101 | 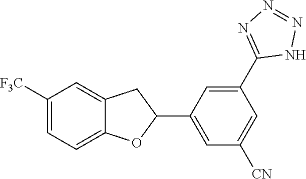 [SMILES: N#Cc1cc(-c2nnn[nH]2)cc(C2Cc3cc(C(F)(F)F)ccc3O2)c1] | A |
| 102 | 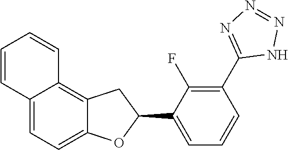 [SMILES: Fc1c(-c2nnn[nH]2)cccc1[C@@H]1Cc2c(ccc3ccccc23)O1] | A |
| 103 | 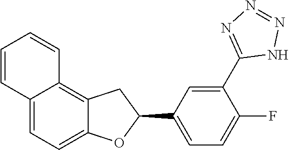 [SMILES: Fc1ccc([C@@H]2Cc3c(ccc4ccccc34)O2)cc1-c1nnn[nH]1] | A |
| 104 | 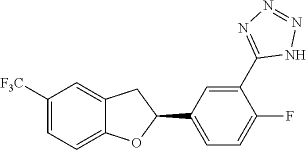 [SMILES: Fc1ccc([C@@H]2Cc3cc(C(F)(F)F)ccc3O2)cc1-c1nnn[nH]1] | A |
| 105 | 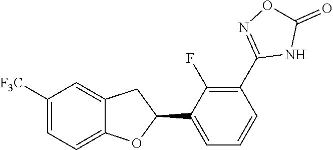 [SMILES: O=c1[nH]c(-c2cccc([C@@H]3Cc4cc(C(F)(F)F)ccc4O3)c2F)no1] | A |

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. A compound having the structure of Formula I: 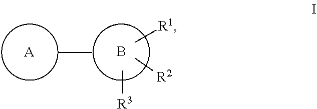
or a pharmaceutically acceptable salt thereof, wherein:
R¹ is 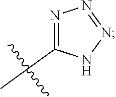 [SMILES: *c1nnn[nH]1]
each of R² and R³ is independently R′, —OR′, halogen, —CN, —NO₂, or —N(R′)₂;
Ring A is 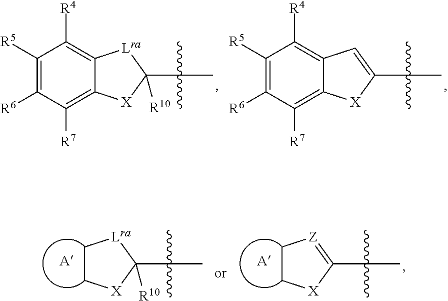
wherein Ring A′ is an optionally substituted 5-10 membered aromatic ring having 0-4 heteroatoms;
L^{ra} is optionally substituted —(CH₂)ₙ—;
n is 1, 2 or 3;
X is —O—, —S—, —N(R⁸)- or optionally substituted —CH₂—;
Z is —N═ or —C(R⁹)═;
each of R⁴, R⁵, R⁶, R⁷ and R⁹ is independently R′, —OR′, halogen, —CN, —NO₂, or —N(R′)₂;
Ring B is an optionally substituted ring selected from a 6-10 membered aryl ring and a 5-10 membered heteroaryl ring having 1-6 heteroatoms;
each of R⁸, R¹⁰, R¹, R¹², R¹³, R¹⁴, R¹⁵, R¹⁶, and R¹⁷ is independently R′;
each R¹ is independently R, OR, C(O)R, C(O)OR, or —S(O)₂R;
each R is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

2. A compound having the structure of Formula I: 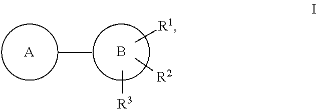
or a pharmaceutically acceptable salt thereof, wherein:
R¹ is —C(O)OH or an isostere thereof, optionally protected-CHO or R^{d6}; or R¹ is —C(O)OR¹¹, —P(O)(OR¹²)(OR¹³), —C(O)N(R¹⁴)SO₂R¹⁵, —C(O)NR¹⁶R¹⁷, —CN, 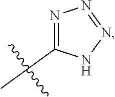 [SMILES: *c1nnn[nH]1]
halogen, or 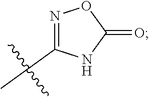 [SMILES: *c1noc(=O)[nH]1]
R^{d6} is —CH(OR)₂;
each of R² and R³ is independently R′, —OR′, halogen, —CN, —NO₂, or —N(R′)₂;
Ring A is 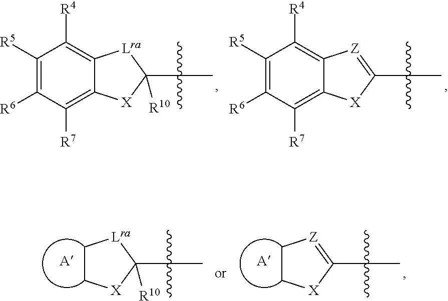
wherein Ring A′ is an optionally substituted 5-10 membered aromatic ring having 0-4 heteroatoms;
L^{ra} is optionally substituted —(CH₂)ₙ—;
n is 1, 2 or 3;
X is —O—, —S—, —N(R⁸)- or optionally substituted —CH₂—;
Z is —N═ or —C(R⁹)═;
each of R⁴, R⁵, R⁶, R⁷ and R° is independently R′, —OR′, halogen, —CN, —NO₂, or —N(R′)₂;
Ring B is an optionally substituted ring selected from a 6-10 membered aryl ring and a 5-10 membered heteroaryl ring having 1-6 heteroatoms;
each of R⁸, R¹⁰, R¹, R¹², R¹³, R¹⁴, R¹⁵, R¹⁶, and R¹⁷ is independently R′;
each R¹ is independently R, —OR, —C(O)R, —C(O) OR, or —S(O)₂R;
each R is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

3. The compound of claim 1, wherein Ring A is

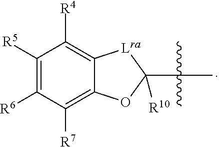

4. The compound of claim 1, wherein Ring A is

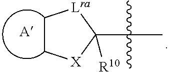

5. The compound of any one of claims 1-4, wherein L^{ra} is —CH₂—.

6. The compound of claim 5, wherein R¹⁰ is H.

7. The compound of claim 1, wherein Ring A is.

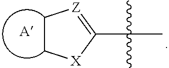

8. The compound of any one of claims 4-7, wherein Ring A′ is an optionally substituted 5-6 membered aromatic ring having 1, 2, 3 or 4 heteroatoms.

9. The compound of any one of claims 4-7, wherein Ring A′ is an optionally substituted phenyl ring.

10. The compound of claim 1, wherein Ring A is

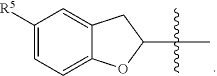

11. The compound claim 10, wherein R⁵ is halogen or optionally substituted C₁-C₆ alkyl.

12. The compound claim 11, wherein R⁵ is —CF₃.

13. The compound of any one claims 10-12, wherein Ring B is

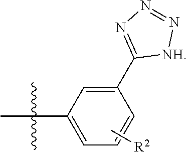

14. The compound of any one claims 10-12, wherein Ring B is

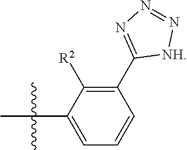

15. The compound of claim 14, wherein R² is halogen.

16. The compound of claim 14, wherein R² is —F.

17. The compound of any one of claims 1-14, wherein R² is R.

18. The compound of any one of claims 1-14, wherein R² is H.

19. The compound of any one of claims 1-14, wherein R² is —C(O)OR wherein R is H or optionally substituted C₁₋₆ aliphatic.

20. The compound of any one of claims 1-14, wherein R² is —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms.

21. The compound of any one of claims 1-12, wherein R³ is halogen.

22. The compound of any one of claims 1-12, wherein R³ is R.

23. The compound of any one of claims 1-12, wherein R³ is H.

24. The compound of any one of claims 1-12, wherein R³ is —C(O)OR wherein R is H or optionally substituted C₁₋₆ aliphatic.

25. The compound of any one of claims 1-12, wherein R³ is —OR wherein R is optionally substituted C₁-C₆ alkyl, optionally substituted 6-10 membered aryl, optionally substituted C₃-C₈ cycloalkyl, or optionally substituted 5-10 membered heteroaryl having 1-6 heteroatoms.

26. A compound, wherein the compound is a compound selected from Table 1 or a salt thereof.

27. A compound, wherein the compound is 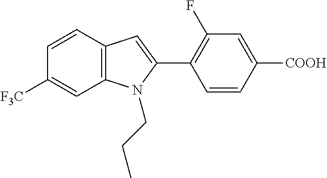 [SMILES: CCCn1c(-c2ccc(C(=O)O)cc2F)cc2ccc(C(F)(F)F)cc21]
or a pharmaceutically acceptable salt thereof.

28. A compound, wherein the compound is 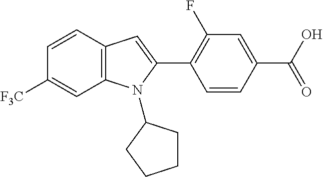 [SMILES: O=C(O)c1ccc(-c2cc3ccc(C(F)(F)F)cc3n2C2CCCC2)c(F)c1]
or a pharmaceutically acceptable salt thereof.

29. A compound, wherein the compound is 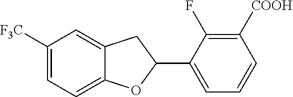 [SMILES: O=C(O)c1cccc(C2Cc3cc(C(F)(F)F)ccc3O2)c1F]
or a pharmaceutically acceptable salt thereof.

30. A compound, wherein the compound is 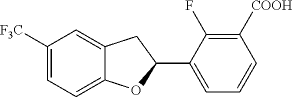 [SMILES: O=C(O)c1cccc([C@@H]2Cc3cc(C(F)(F)F)ccc3O2)c1F]
or a pharmaceutically acceptable salt thereof.

31. A compound, wherein the compound is 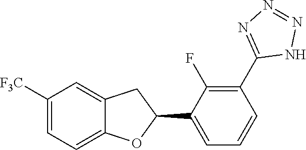 [SMILES: Fc1c(-c2nnn[nH]2)cccc1[C@@H]1Cc2cc(C(F)(F)F)ccc2O1]
or a pharmaceutically acceptable salt thereof.

32. A compound, wherein the compound is 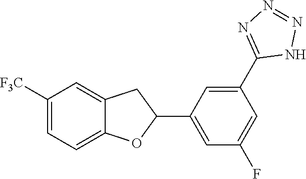 [SMILES: Fc1cc(-c2nnn[nH]2)cc(C2Cc3cc(C(F)(F)F)ccc3O2)c1]
or a pharmaceutically acceptable salt thereof.

33. A compound, wherein the compound is 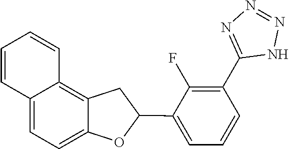 [SMILES: Fc1c(-c2nnn[nH]2)cccc1C1Cc2c(ccc3ccccc23)O1]
or a pharmaceutically acceptable salt thereof.

34. A compound, wherein the compound is 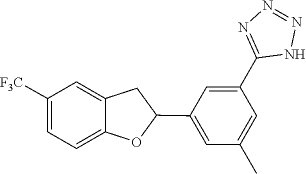 [SMILES: Cc1cc(-c2nnn[nH]2)cc(C2Cc3cc(C(F)(F)F)ccc3O2)c1]
or a pharmaceutically acceptable salt thereof.

35. A compound, wherein the compound is 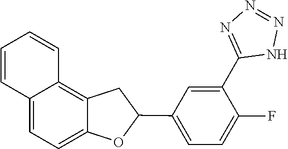 [SMILES: Fc1ccc(C2Cc3c(ccc4ccccc34)O2)cc1-c1nnn[nH]1]
or a pharmaceutically acceptable salt thereof.

36. A compound, wherein the compound is 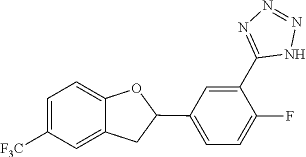 [SMILES: Fc1ccc(C2Cc3cc(C(F)(F)F)ccc3O2)cc1-c1nnn[nH]1]
or a pharmaceutically acceptable salt thereof.

37. A compound, wherein the compound is 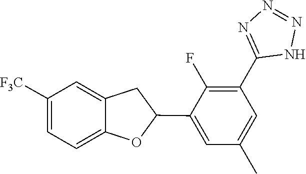 [SMILES: Cc1cc(-c2nnn[nH]2)c(F)c(C2Cc3cc(C(F)(F)F)ccc3O2)c1]
or a pharmaceutically acceptable salt thereof.

38. A compound, wherein the compound is 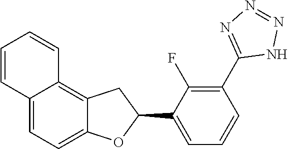 [SMILES: Fc1c(-c2nnn[nH]2)cccc1[C@@H]1Cc2c(ccc3ccccc23)O1]
or a pharmaceutically acceptable salt thereof.

39. A compound, wherein the compound is 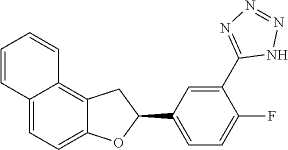 [SMILES: Fc1ccc([C@@H]2Cc3c(ccc4ccccc34)O2)cc1-c1nnn[nH]1]
or a pharmaceutically acceptable salt thereof.

40. A compound, wherein the compound is 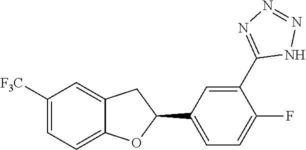 [SMILES: Fc1ccc([C@@H]2Cc3cc(C(F)(F)F)ccc3O2)cc1-c1nnn[nH]1]
or a pharmaceutically acceptable salt thereof.

41. A compound, wherein the compound is 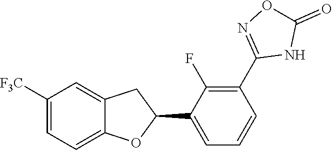 [SMILES: O=c1[nH]c(-c2cccc([C@@H]3Cc4cc(C(F)(F)F)ccc4O3)c2F)no1]
or a pharmaceutically acceptable salt thereof.

42. The compound of any one of the preceding claims, wherein the compound is a pharmaceutically acceptable salt.

43. The compound of any one of the preceding claims, wherein the compound is sodium salt.

44. A pharmaceutical composition comprising a compound of any one of the preceding claims and a pharmaceutically acceptable carrier.

45. The composition of claim 44, wherein the composition is a topical composition.

46. A method for modulating a Mas-related G-protein coupled receptor X4 (MRGPRX4) activity, comprising contacting MRGPRX4 with an effective amount of the compound or pharmaceutical composition of any one of the preceding claims, or
a method for modulating MRGPRX4 activity in a system, comprising MRGPRX4, comprising administering or delivering to the system an effective amount of a compound or a pharmaceutical composition of any one of the preceding claims.

47. A method for treating a condition, disorder or disease, comprising administering to a subject suffering therefrom an effective amount of the compound or pharmaceutical composition of any one of claims 1-45.

48. A method for treating a condition, disorder or disease, comprising delivering to a subject suffering therefrom an effective amount of the compound or pharmaceutical composition of any one of claims 1-45.

49. The method of any one of claims 47-48, wherein the condition, disorder or disease is associated with MRGPRX4.

50. The method of any one of claims 47-48, wherein the condition, disorder or disease is or comprises itch.

51. The method of any one of claims 47-48, wherein the condition, disorder or disease is or comprises pruritus.

52. The method of any one of claims 47-48, wherein the condition, disorder or disease is or comprises chronic itch, cholestatic pruritus, contact dermatitis, allergic blepharitis, anemia, atopic dermatitis, bullous pemphigoid, candidiasis, chicken pox, cholestasis, end-stage renal failure, hemodialysis, contact dermatitis, dermatitis herpetiformis, diabetes, drug allergy, dry skin, dyshidrotic dermatitis, ectopic eczema, eczema, erythrasma, folliculitis, fungal skin infection, hemorrhoids, herpes, HIV infection, Hodgkin's disease, hyperthyroidism, iron deficiency anemia, kidney disease, leukemia, liver disease, lymphoma, malignancy, multiple myeloma, neurodermatitis, onchocerciasis, Paget's disease, pediculosis, polycythemia rubra vera, pruritus ani, pseudorabies, psoriasis, rectal prolapse, scabies, schistosomiasis, scleroderma, severe stress, stasis dermatitis, swimmer's itch, thyroid disease, tinea cruris, uremic pruritus, or urticaria.

53. The method of claim 51, wherein pruritus is an acute or chronic pruritus associated a liver condition, disorder or disease.

54. The method of any one of claims 47-48, wherein the condition, disorder or disease is a liver condition, disorder or disease.

55. The method of claim 53 or 54, wherein the liver condition, disorder or disease is intrahepatic cholestasis of pregnancy (ICP), estrogen-, progesterone- or testosterone-induced cholestasis, toxin- or other drug induced hepatocellular cholestasis, benign recurrent intrahepatic cholestasis (BRIC), progressive familial intrahepatic cholestasis (PFIC), chronic viral hepatitis C, chronic hepatitis B, alcoholic or nonalcoholic fatty liver disease (NAFLD), nonalcoholic steatohepatitis (NASH), primary biliary cholangitis (PBC), primary sclerosing cholangitis (PSC), secondary sclerosing cholangitis (SSC), sarcoidosis, ABCB4 deficiency, alagille syndrome, drug-induce small duct cholangiopathies, gallstone disease, IgG4-associated cholangitis, biliary atresia, cholangiocellular carcinoma, benign bile duct adenoma, or other obstructive cholestasis.

56. The method of any one of claims 47-48, wherein the condition, disorder or disease is or comprises primary biliary cholangitis (PBC).

57. The method of any one of claims 47-56, wherein the compound or composition is utilized with another therapeutic agent.

58. The method of claim 57, wherein the another therapeutic agent is or delivers ursodeoxycholic acid or a pharmaceutically acceptable salt thereof.

59. The method of any one of claims 57-58, wherein the compound or composition is administered concurrently with, prior to or subsequent to the another therapeutic agent.

60. A compound, composition, or method described in the specification, or of any one of Embodiments 1-1178.
